SEQUENCE LISTING (743 sequences)

SEQ ID NO: 1 - protein, 412 aa, unidentified
  REGION 1..412: note = Description of Unknown: Adenosine A2A sequence
MPIMGSSVYITVELAIAVLAILGNVLVCWAVWLNSNLQNVTNYFVVSLAAADIAVGVLAIPFAITISTGFCAACHGCLFIACFVLVLTQSSIFSLLAIAIDRYIAIRIPLRYNGLVTGTRAKGIIAICWVLSFAIGLTPMLGWNNCGQPKEGKNHSQGCGEGQVACLFEDVVPMNYMVYFNFFACVLVPLLLMLGVYLRIFLAARRQLKQMESQPLPGERARSTLQKEVHAAKSLAIIVGLFALCWLPLHIINCFTFFCPDCSHAPLWLMYLAIVLSHTNSVVNPFIYAYRIREFRQTFRKIIRSHVLRQQEPFKAAGTSARVLAAHGSDGEQVSLRLNGHPPGVWANGSAPHPERRPNGYALGLVSGGSAQESQGNTGLPDVELLSHELKGVCPEPPGLDDPLAQDGAGVS

SEQ ID NO: 2 - DNA, 62 bp, synthetic construct
  misc_feature 1..62: note = Description of Artificial Sequence: Synthetic oligonucleotide
  modified_base 51..52: mod_base = OTHER; note = Thymidine-succinyl hexamide CED phosphoramidite
  source 1..62: mol_type = other DNA
agacaatcaaccatttggggtggacagccttgacctctagacttcggcattttttttttttt

SEQ ID NO: 3 - DNA, 112 bp, synthetic construct
  misc_feature 1..112: note = Description of Artificial Sequence: Synthetic polynucleotide
  modified_base 101..102: mod_base = OTHER; note = Thymidine-succinyl hexamide CED phosphoramidite
  source 1..112: mol_type = other DNA
cgggatccttatcgtcatcgtcgtacagatcccgacccatttgctgtccaccagtcatgctagccataccatgatgatgatgatgatgagaaccccgcattttttttttttt

SEQ ID NO: 4 - DNA, 19 bp, synthetic construct
  misc_feature 1..19: note = Description of Artificial Sequence: Synthetic primer
  source 1..19: mol_type = other DNA
atgcggggttctcatcatc

SEQ ID NO: 5 - DNA, 20 bp, synthetic construct
  misc_feature 1..20: note = Description of Artificial Sequence: Synthetic primer
  source 1..20: mol_type = other DNA
cgggatccttatcgtcatcg

SEQ ID NO: 6 - protein, 8 aa, synthetic construct
  REGION 1..8: note = Description of Artificial Sequence: Synthetic peptide
GGSISSSN

SEQ ID NO: 7 - protein, 7 aa, synthetic construct
  REGION 1..7: note = Description of Artificial Sequence: Synthetic peptide
GYTFTGY

SEQ ID NO: 8 - protein, 7 aa, synthetic construct
  REGION 1..7: note = Description of Artificial Sequence: Synthetic peptide
GYTFTGY

SEQ ID NO: 9 - protein, 7 aa, synthetic construct
  REGION 1..7: note = Description of Artificial Sequence: Synthetic peptide
GFTFSDY

SEQ ID NO: 10 - protein, 7 aa, synthetic construct
  REGION 1..7: note = Description of Artificial Sequence: Synthetic peptide
GFTFSSY

SEQ ID NO: 11 - protein, 7 aa, synthetic construct
  REGION 1..7: note = Description of Artificial Sequence: Synthetic peptide
GFTFSNY

SEQ ID NO: 12 - protein, 7 aa, synthetic construct
  REGION 1..7: note = Description of Artificial Sequence: Synthetic peptide
GFTFSSY

SEQ ID NO: 13 - protein, 8 aa, synthetic construct
  REGION 1..8: note = Description of Artificial Sequence: Synthetic peptide
GYSITSGY

SEQ ID NO: 14 - protein, 8 aa, synthetic construct
  REGION 1..8: note = Description of Artificial Sequence: Synthetic peptide
GYSITSGY

SEQ ID NO: 15 - protein, 8 aa, synthetic construct
  REGION 1..8: note = Description of Artificial Sequence: Synthetic peptide
GYSITSGY

SEQ ID NO: 16 - protein, 7 aa, synthetic construct
  REGION 1..7: note = Description of Artificial Sequence: Synthetic peptide
GFTFSDY

SEQ ID NO: 17 - protein, 7 aa, synthetic construct
  REGION 1..7: note = Description of Artificial Sequence: Synthetic peptide
GFTFNDY

SEQ ID NO: 18 - protein, 7 aa, synthetic construct
  REGION 1..7: note = Description of Artificial Sequence: Synthetic peptide
GFTFSDF

SEQ ID NO: 19 - protein, 7 aa, synthetic construct
  REGION 1..7: note = Description of Artificial Sequence: Synthetic peptide
GFTFSDY

SEQ ID NO: 20 - protein, 7 aa, synthetic construct
  REGION 1..7: note = Description of Artificial Sequence: Synthetic peptide
GFTFSTY

SEQ ID NO: 21 - protein, 7 aa, synthetic construct
  REGION 1..7: note = Description of Artificial Sequence: Synthetic peptide
GFTFNNY

SEQ ID NO: 22 - protein, 7 aa, synthetic construct
  REGION 1..7: note = Description of Artificial Sequence: Synthetic peptide
GFNIGNT

SEQ ID NO: 23 - protein, 7 aa, synthetic construct
  REGION 1..7: note = Description of Artificial Sequence: Synthetic peptide
GFSFSGY

SEQ ID NO: 24 - protein, 7 aa, synthetic construct
  REGION 1..7: note = Description of Artificial Sequence: Synthetic peptide
GFTFSGY

SEQ ID NO: 25 - protein, 7 aa, synthetic construct
  REGION 1..7: note = Description of Artificial Sequence: Synthetic peptide
GFAFSNY

SEQ ID NO: 26 - protein, 9 aa, synthetic construct
  REGION 1..9: note = Description of Artificial Sequence: Synthetic peptide
GGSISSGGY

SEQ ID NO: 27 - protein, 7 aa, synthetic construct
  REGION 1..7: note = Description of Artificial Sequence: Synthetic peptide
GYTFTEY

SEQ ID NO: 28 - protein, 7 aa, synthetic construct
  REGION 1..7: note = Description of Artificial Sequence: Synthetic peptide
GFTFSTY

SEQ ID NO: 29 - protein, 7 aa, synthetic construct
  REGION 1..7: note = Description of Artificial Sequence: Synthetic peptide
GFNIGNT

SEQ ID NO: 30 - protein, 7 aa, synthetic construct
  REGION 1..7: note = Description of Artificial Sequence: Synthetic peptide
GFTFGNY

SEQ ID NO: 31 - protein, 7 aa, synthetic construct
  REGION 1..7: note = Description of Artificial Sequence: Synthetic peptide
GFTFSTY

SEQ ID NO: 32 - protein, 7 aa, synthetic construct
  REGION 1..7: note = Description of Artificial Sequence: Synthetic peptide
GFTFSSY

SEQ ID NO: 33 - protein, 7 aa, synthetic construct
  REGION 1..7: note = Description of Artificial Sequence: Synthetic peptide
GFTFSSQ

SEQ ID NO: 34 - protein, 7 aa, synthetic construct
  REGION 1..7: note = Description of Artificial Sequence: Synthetic peptide
GYSFTGY

SEQ ID NO: 35 - protein, 7 aa, synthetic construct
  REGION 1..7: note = Description of Artificial Sequence: Synthetic peptide
GYRLTGY

SEQ ID NO: 36 - protein, 7 aa, synthetic construct
  REGION 1..7: note = Description of Artificial Sequence: Synthetic peptide
GYLFTDY

SEQ ID NO: 37 - protein, 7 aa, synthetic construct
  REGION 1..7: note = Description of Artificial Sequence: Synthetic peptide
GFTFIDY

SEQ ID NO: 38 - protein, 7 aa, synthetic construct
  REGION 1..7: note = Description of Artificial Sequence: Synthetic peptide
GFPFSSY

SEQ ID NO: 39 - protein, 7 aa, synthetic construct
  REGION 1..7: note = Description of Artificial Sequence: Synthetic peptide
GFNFNTY

SEQ ID NO: 40 - protein, 7 aa, synthetic construct
  REGION 1..7: note = Description of Artificial Sequence: Synthetic peptide
GFTFSTY

SEQ ID NO: 41 - protein, 7 aa, synthetic construct
  REGION 1..7: note = Description of Artificial Sequence: Synthetic peptide
GFNIGNT

SEQ ID NO: 42 - protein, 7 aa, synthetic construct
  REGION 1..7: note = Description of Artificial Sequence: Synthetic peptide
GYTFTSY

SEQ ID NO: 43 - protein, 7 aa, synthetic construct
  REGION 1..7: note = Description of Artificial Sequence: Synthetic peptide
GFSLTSY

SEQ ID NO: 44 - protein, 7 aa, synthetic construct
  REGION 1..7: note = Description of Artificial Sequence: Synthetic peptide
GFTFSNY

SEQ ID NO: 45 - protein, 7 aa, synthetic construct
  REGION 1..7: note = Description of Artificial Sequence: Synthetic peptide
GFNFRSY

SEQ ID NO: 46 - protein, 7 aa, synthetic construct
  REGION 1..7: note = Description of Artificial Sequence: Synthetic peptide
GFSLSIY

SEQ ID NO: 47 - protein, 7 aa, synthetic construct
  REGION 1..7: note = Description of Artificial Sequence: Synthetic peptide
GYTFTSY

SEQ ID NO: 48 - protein, 7 aa, synthetic construct
  REGION 1..7: note = Description of Artificial Sequence: Synthetic peptide
GFTFSSY

SEQ ID NO: 49 - protein, 7 aa, synthetic construct
  REGION 1..7: note = Description of Artificial Sequence: Synthetic peptide
GFTFSAY

SEQ ID NO: 50 - protein, 7 aa, synthetic construct
  REGION 1..7: note = Description of Artificial Sequence: Synthetic peptide
GYSFTGY

SEQ ID NO: 51 - protein, 8 aa, synthetic construct
  REGION 1..8: note = Description of Artificial Sequence: Synthetic peptide
GGYISSSN

SEQ ID NO: 52 - protein, 6 aa, synthetic construct
  REGION 1..6: note = Description of Artificial Sequence: Synthetic peptide
GTFTDY

SEQ ID NO: 53 - protein, 7 aa, synthetic construct
  REGION 1..7: note = Description of Artificial Sequence: Synthetic peptide
GYTFTSS

SEQ ID NO: 54 - protein, 7 aa, synthetic construct
  REGION 1..7: note = Description of Artificial Sequence: Synthetic peptide
GYTFNDD

SEQ ID NO: 55 - protein, 7 aa, synthetic construct
  REGION 1..7: note = Description of Artificial Sequence: Synthetic peptide
GYSFTGY

SEQ ID NO: 56 - protein, 7 aa, synthetic construct
  REGION 1..7: note = Description of Artificial Sequence: Synthetic peptide
GYTFNDY

SEQ ID NO: 57 - protein, 7 aa, synthetic construct
  REGION 1..7: note = Description of Artificial Sequence: Synthetic peptide
GYTFNDY

SEQ ID NO: 58 - protein, 7 aa, synthetic construct
  REGION 1..7: note = Description of Artificial Sequence: Synthetic peptide
GFNIIDD

SEQ ID NO: 59 - protein, 7 aa, synthetic construct
  REGION 1..7: note = Description of Artificial Sequence: Synthetic peptide
GYTFTDY

SEQ ID NO: 60 - protein, 7 aa, synthetic construct
  REGION 1..7: note = Description of Artificial Sequence: Synthetic peptide
GYEFSSS

SEQ ID NO: 61 - protein, 7 aa, synthetic construct
  REGION 1..7: note = Description of Artificial Sequence: Synthetic peptide
GFTFSSY

SEQ ID NO: 62 - protein, 7 aa, synthetic construct
  REGION 1..7: note = Description of Artificial Sequence: Synthetic peptide
GFIFSDF

SEQ ID NO: 63 - protein, 7 aa, synthetic construct
  REGION 1..7: note = Description of Artificial Sequence: Synthetic peptide
GFNIKDY

SEQ ID NO: 64 - protein, 7 aa, synthetic construct
  REGION 1..7: note = Description of Artificial Sequence: Synthetic peptide
GYTFTDY

SEQ ID NO: 65 - protein, 7 aa, synthetic construct
  REGION 1..7: note = Description of Artificial Sequence: Synthetic peptide
GYTFTEY

SEQ ID NO: 66 - protein, 7 aa, synthetic construct
  REGION 1..7: note = Description of Artificial Sequence: Synthetic peptide
GFTFSTY

SEQ ID NO: 67 - protein, 7 aa, synthetic construct
  REGION 1..7: note = Description of Artificial Sequence: Synthetic peptide
GYTFTNY

SEQ ID NO: 68 - protein, 7 aa, synthetic construct
  REGION 1..7: note = Description of Artificial Sequence: Synthetic peptide
GFTFSDH

SEQ ID NO: 69 - protein, 7 aa, synthetic construct
  REGION 1..7: note = Description of Artificial Sequence: Synthetic peptide
GFTFDDY

SEQ ID NO: 70 - protein, 7 aa, synthetic construct
  REGION 1..7: note = Description of Artificial Sequence: Synthetic peptide
GSAFSAS

SEQ ID NO: 71 - protein, 7 aa, synthetic construct
  REGION 1..7: note = Description of Artificial Sequence: Synthetic peptide
GFTFSSY

SEQ ID NO: 72 - protein, 7 aa, synthetic construct
  REGION 1..7: note = Description of Artificial Sequence: Synthetic peptide
GHTITSY

SEQ ID NO: 73 - protein, 7 aa, synthetic construct
  REGION 1..7: note = Description of Artificial Sequence: Synthetic peptide
GYTFSGY

SEQ ID NO: 74 - protein, 8 aa, synthetic construct
  REGION 1..8: note = Description of Artificial Sequence: Synthetic peptide
GGYISSSN

SEQ ID NO: 75 - protein, 7 aa, synthetic construct
  REGION 1..7: note = Description of Artificial Sequence: Synthetic peptide
GSNIKDY

SEQ ID NO: 76 - protein, 8 aa, synthetic construct
  REGION 1..8: note = Description of Artificial Sequence: Synthetic peptide
GGSISSSN

SEQ ID NO: 77 - protein, 7 aa, synthetic construct
  REGION 1..7: note = Description of Artificial Sequence: Synthetic peptide
GFTISTY

SEQ ID NO: 78 - protein, 7 aa, synthetic construct
  REGION 1..7: note = Description of Artificial Sequence: Synthetic peptide
GFTVSTY

SEQ ID NO: 79 - protein, 7 aa, synthetic construct
  REGION 1..7: note = Description of Artificial Sequence: Synthetic peptide
GFTFTTY

SEQ ID NO: 80 - protein, 8 aa, synthetic construct
  REGION 1..8: note = Description of Artificial Sequence: Synthetic peptide
GGSISSSN

SEQ ID NO: 81 - protein, 7 aa, synthetic construct
  REGION 1..7: note = Description of Artificial Sequence: Synthetic peptide
GFTFSSY

SEQ ID NO: 82 - protein, 8 aa, synthetic construct
  REGION 1..8: note = Description of Artificial Sequence: Synthetic peptide
GGSISSSN

SEQ ID NO: 83 - protein, 7 aa, synthetic construct
  REGION 1..7: note = Description of Artificial Sequence: Synthetic peptide
GFTFSAY

SEQ ID NO: 84 - protein, 7 aa, synthetic construct
  REGION 1..7: note = Description of Artificial Sequence: Synthetic peptide
GFTFDDY

SEQ ID NO: 85 - protein, 7 aa, synthetic construct
  REGION 1..7: note = Description of Artificial Sequence: Synthetic peptide
GFTISTY

SEQ ID NO: 86 - protein, 8 aa, synthetic construct
  REGION 1..8: note = Description of Artificial Sequence: Synthetic peptide
GGSISSSN

SEQ ID NO: 87 - protein, 8 aa, synthetic construct
  REGION 1..8: note = Description of Artificial Sequence: Synthetic peptide
GYSITSGY

SEQ ID NO: 88 - protein, 7 aa, synthetic construct
  REGION 1..7: note = Description of Artificial Sequence: Synthetic peptide
GYAFSSY

SEQ ID NO: 89 - protein, 7 aa, synthetic construct
  REGION 1..7: note = Description of Artificial Sequence: Synthetic peptide
GYTFTEY

SEQ ID NO: 90 - protein, 7 aa, synthetic construct
  REGION 1..7: note = Description of Artificial Sequence: Synthetic peptide
GFSLTAY

SEQ ID NO: 91 - protein, 7 aa, synthetic construct
  REGION 1..7: note = Description of Artificial Sequence: Synthetic peptide
GYSITSD

SEQ ID NO: 92 - protein, 7 aa, synthetic construct
  REGION 1..7: note = Description of Artificial Sequence: Synthetic peptide
GFNIKNT

SEQ ID NO: 93 - protein, 7 aa, synthetic construct
  REGION 1..7: note = Description of Artificial Sequence: Synthetic peptide
GFTFRTY

SEQ ID NO: 94 - protein, 7 aa, synthetic construct
  REGION 1..7: note = Description of Artificial Sequence: Synthetic peptide
GFTFNNY

SEQ ID NO: 95 - protein, 5 aa, synthetic construct
  REGION 1..5: note = Description of Artificial Sequence: Synthetic peptide
YPSGN

SEQ ID NO: 96 - protein, 6 aa, synthetic construct
  REGION 1..6: note = Description of Artificial Sequence: Synthetic peptide
NPNSGG

SEQ ID NO: 97 - protein, 6 aa, synthetic construct
  REGION 1..6: note = Description of Artificial Sequence: Synthetic peptide
NPNSGG

SEQ ID NO: 98 - protein, 6 aa, synthetic construct
  REGION 1..6: note = Description of Artificial Sequence: Synthetic peptide
SSSGST

SEQ ID NO: 99 - protein, 6 aa, synthetic construct
  REGION 1..6: note = Description of Artificial Sequence: Synthetic peptide
SSSSSY

SEQ ID NO: 100 - protein, 6 aa, synthetic construct
  REGION 1..6: note = Description of Artificial Sequence: Synthetic peptide
SSSSSY

SEQ ID NO: 101 - protein, 6 aa, synthetic construct
  REGION 1..6: note = Description of Artificial Sequence: Synthetic peptide
SGSGGS

SEQ ID NO: 102 - protein, 5 aa, synthetic construct
  REGION 1..5: note = Description of Artificial Sequence: Synthetic peptide
SYDGS

SEQ ID NO: 103 - protein, 5 aa, synthetic construct
  REGION 1..5: note = Description of Artificial Sequence: Synthetic peptide
RYDGS

SEQ ID NO: 104 - protein, 5 aa, synthetic construct
  REGION 1..5: note = Description of Artificial Sequence: Synthetic peptide
SYDGS

SEQ ID NO: 105 - protein, 6 aa, synthetic construct
  REGION 1..6: note = Description of Artificial Sequence: Synthetic peptide
NYDGSS

SEQ ID NO: 106 - protein, 6 aa, synthetic construct
  REGION 1..6: note = Description of Artificial Sequence: Synthetic peptide
NYDGSS

SEQ ID NO: 107 - protein, 6 aa, synthetic construct
  REGION 1..6: note = Description of Artificial Sequence: Synthetic peptide
SSGSST

SEQ ID NO: 108 - protein, 6 aa, synthetic construct
  REGION 1..6: note = Description of Artificial Sequence: Synthetic peptide
SSGSGT

SEQ ID NO: 109 - protein, 6 aa, synthetic construct
  REGION 1..6: note = Description of Artificial Sequence: Synthetic peptide
SGSGGS

SEQ ID NO: 110 - protein, 6 aa, synthetic construct
  REGION 1..6: note = Description of Artificial Sequence: Synthetic peptide
SGSGGD

SEQ ID NO: 111 - protein, 6 aa, synthetic construct
  REGION 1..6: note = Description of Artificial Sequence: Synthetic peptide
NPNYGT

SEQ ID NO: 112 - protein, 6 aa, synthetic construct
  REGION 1..6: note = Description of Artificial Sequence: Synthetic peptide
SGSGGS

SEQ ID NO: 113 - protein, 6 aa, synthetic construct
  REGION 1..6: note = Description of Artificial Sequence: Synthetic peptide
SGSGAS

SEQ ID NO: 114 - protein, 6 aa, synthetic construct
  REGION 1..6: note = Description of Artificial Sequence: Synthetic peptide
YPKSGS

SEQ ID NO: 115 - protein, 6 aa, synthetic construct
  REGION 1..6: note = Description of Artificial Sequence: Synthetic peptide
NPNSGN

SEQ ID NO: 116 - protein, 6 aa, synthetic construct
  REGION 1..6: note = Description of Artificial Sequence: Synthetic peptide
HPSSGS

SEQ ID NO: 117 - protein, 6 aa, synthetic construct
  REGION 1..6: note = Description of Artificial Sequence: Synthetic peptide
SGSGGS

SEQ ID NO: 118 - protein, 6 aa, synthetic construct
  REGION 1..6: note = Description of Artificial Sequence: Synthetic peptide
NPNYGT

SEQ ID NO: 119 - protein, 6 aa, synthetic construct
  REGION 1..6: note = Description of Artificial Sequence: Synthetic peptide
DPANGD

SEQ ID NO: 120 - protein, 6 aa, synthetic construct
  REGION 1..6: note = Description of Artificial Sequence: Synthetic peptide
SGSAGS

SEQ ID NO: 121 - protein, 6 aa, synthetic construct
  REGION 1..6: note = Description of Artificial Sequence: Synthetic peptide
SGSGGS

SEQ ID NO: 122 - protein, 6 aa, synthetic construct
  REGION 1..6: note = Description of Artificial Sequence: Synthetic peptide
SGSGVS

SEQ ID NO: 123 - protein, 6 aa, synthetic construct
  REGION 1..6: note = Description of Artificial Sequence: Synthetic peptide
YPGSGN

SEQ ID NO: 124 - protein, 6 aa, synthetic construct
  REGION 1..6: note = Description of Artificial Sequence: Synthetic peptide
DPASGD

SEQ ID NO: 125 - protein, 5 aa, synthetic construct
  REGION 1..5: note = Description of Artificial Sequence: Synthetic peptide
YPGTG

SEQ ID NO: 126 - protein, 6 aa, synthetic construct
  REGION 1..6: note = Description of Artificial Sequence: Synthetic peptide
NPNYGT

SEQ ID NO: 127 - protein, 6 aa, synthetic construct
  REGION 1..6: note = Description of Artificial Sequence: Synthetic peptide
SGSGGR

SEQ ID NO: 128 - protein, 6 aa, synthetic construct
  REGION 1..6: note = Description of Artificial Sequence: Synthetic peptide
YPGNSD

SEQ ID NO: 129 - protein, 6 aa, synthetic construct
  REGION 1..6: note = Description of Artificial Sequence: Synthetic peptide
SGSGGS

SEQ ID NO: 130 - protein, 6 aa, synthetic construct
  REGION 1..6: note = Description of Artificial Sequence: Synthetic peptide
NPNYGT

SEQ ID NO: 131 - protein, 6 aa, synthetic construct
  REGION 1..6: note = Description of Artificial Sequence: Synthetic peptide
NHDGSN

SEQ ID NO: 132 - protein, 6 aa, synthetic construct
  REGION 1..6: note = Description of Artificial Sequence: Synthetic peptide
DPETDD

SEQ ID NO: 133 - protein, 6 aa, synthetic construct
  REGION 1..6: note = Description of Artificial Sequence: Synthetic peptide
NPNNGG

SEQ ID NO: 134 - protein, 6 aa, synthetic construct
  REGION 1..6: note = Description of Artificial Sequence: Synthetic peptide
SGGGGS

SEQ ID NO: 135 - protein, 6 aa, synthetic construct
  REGION 1..6: note = Description of Artificial Sequence: Synthetic peptide
SPGSGS

SEQ ID NO: 136 - protein, 6 aa, synthetic construct
  REGION 1..6: note = Description of Artificial Sequence: Synthetic peptide
SSGGDS

SEQ ID NO: 137 - protein, 6 aa, synthetic construct
  REGION 1..6: note = Description of Artificial Sequence: Synthetic peptide
SDGGSY

SEQ ID NO: 138 - protein, 5 aa, synthetic construct
  REGION 1..5: note = Description of Artificial Sequence: Synthetic peptide
GTAGD

SEQ ID NO: 139 - protein, 6 aa, synthetic construct
  REGION 1..6: note = Description of Artificial Sequence: Synthetic peptide
LPGSGG

SEQ ID NO: 140 - protein, 6 aa, synthetic construct
  REGION 1..6: note = Description of Artificial Sequence: Synthetic peptide
EQDGSE

SEQ ID NO: 141 - protein, 6 aa, synthetic construct
  REGION 1..6: note = Description of Artificial Sequence: Synthetic peptide
LPGSGG

SEQ ID NO: 142 - protein, 6 aa, synthetic construct
  REGION 1..6: note = Description of Artificial Sequence: Synthetic peptide
YPRDGS

SEQ ID NO: 143 - protein, 6 aa, synthetic construct
  REGION 1..6: note = Description of Artificial Sequence: Synthetic peptide
NPNNGA

SEQ ID NO: 144 - protein, 6 aa, synthetic construct
  REGION 1..6: note = Description of Artificial Sequence: Synthetic peptide
YPKDGS

SEQ ID NO: 145 - protein, 6 aa, synthetic construct
  REGION 1..6: note = Description of Artificial Sequence: Synthetic peptide
NPNNGA

SEQ ID NO: 146 - protein, 6 aa, synthetic construct
  REGION 1..6: note = Description of Artificial Sequence: Synthetic peptide
NPNNGG

SEQ ID NO: 147 - protein, 6 aa, synthetic construct
  REGION 1..6: note = Description of Artificial Sequence: Synthetic peptide
TDTGEP

SEQ ID NO: 148 - protein, 6 aa, synthetic construct
  REGION 1..6: note = Description of Artificial Sequence: Synthetic peptide
DPANGD

SEQ ID NO: 149 - protein, 6 aa, synthetic construct
  REGION 1..6: note = Description of Artificial Sequence: Synthetic peptide
YPGTGN

SEQ ID NO: 150 - protein, 6 aa, synthetic construct
  REGION 1..6: note = Description of Artificial Sequence: Synthetic peptide
DPGTGG

SEQ ID NO: 151 - protein, 5 aa, synthetic construct
  REGION 1..5: note = Description of Artificial Sequence: Synthetic peptide
DPEDG

SEQ ID NO: 152 - protein, 6 aa, synthetic construct
  REGION 1..6: note = Description of Artificial Sequence: Synthetic peptide
NPNNGG

SEQ ID NO: 153 - protein, 6 aa, synthetic construct
  REGION 1..6: note = Description of Artificial Sequence: Synthetic peptide
NINNGG

SEQ ID NO: 154 - protein, 6 aa, synthetic construct
  REGION 1..6: note = Description of Artificial Sequence: Synthetic peptide
NFDGSS

SEQ ID NO: 155 - protein, 6 aa, synthetic construct
  REGION 1..6: note = Description of Artificial Sequence: Synthetic peptide
YPGDTD

SEQ ID NO: 156 - protein, 6 aa, synthetic construct
  REGION 1..6: note = Description of Artificial Sequence: Synthetic peptide
NPNNGG

SEQ ID NO: 157 - protein, 6 aa, synthetic construct
  REGION 1..6: note = Description of Artificial Sequence: Synthetic peptide
NPNSGI

SEQ ID NO: 158 - protein, 6 aa, synthetic construct
  REGION 1..6: note = Description of Artificial Sequence: Synthetic peptide
NTNTGN

SEQ ID NO: 159 - protein, 6 aa, synthetic construct
  REGION 1..6: note = Description of Artificial Sequence: Synthetic peptide
DPDNGD

SEQ ID NO: 160 - protein, 6 aa, synthetic construct
  REGION 1..6: note = Description of Artificial Sequence: Synthetic peptide
YPKDGS

SEQ ID NO: 161 - protein, 6 aa, synthetic construct
  REGION 1..6: note = Description of Artificial Sequence: Synthetic peptide
LPGSGT

SEQ ID NO: 162 - protein, 6 aa, synthetic construct
  REGION 1..6: note = Description of Artificial Sequence: Synthetic peptide
DPSDSF

SEQ ID NO: 163 - protein, 8 aa, synthetic construct
  REGION 1..8: note = Description of Artificial Sequence: Synthetic peptide
KTKTDGGT

SEQ ID NO: 164 - protein, 5 aa, synthetic construct
  REGION 1..5: note = Description of Artificial Sequence: Synthetic peptide
SDGGS

SEQ ID NO: 165 - protein, 5 aa, synthetic construct
  REGION 1..5: note = Description of Artificial Sequence: Synthetic peptide
YHSGS

SEQ ID NO: 166 - protein, 5 aa, synthetic construct
  REGION 1..5: note = Description of Artificial Sequence: Synthetic peptide
GTAGD

SEQ ID NO: 167 - protein, 5 aa, synthetic construct
  REGION 1..5: note = Description of Artificial Sequence: Synthetic peptide
GTAGD

SEQ ID NO: 168 - protein, 5 aa, synthetic construct
  REGION 1..5: note = Description of Artificial Sequence: Synthetic peptide
GTAGD

SEQ ID NO: 169 - protein, 5 aa, synthetic construct
  REGION 1..5: note = Description of Artificial Sequence: Synthetic peptide
YHSGN

SEQ ID NO: 170 - protein, 5 aa, synthetic construct
  REGION 1..5: note = Description of Artificial Sequence: Synthetic peptide
GTAGD

SEQ ID NO: 171 - protein, 5 aa, synthetic construct
  REGION 1..5: note = Description of Artificial Sequence: Synthetic peptide
YHSGN

SEQ ID NO: 172 - protein, 5 aa, synthetic construct
  REGION 1..5: note = Description of Artificial Sequence: Synthetic peptide
GTAGD

SEQ ID NO: 173 - protein, 6 aa, synthetic construct
  REGION 1..6: note = Description of Artificial Sequence: Synthetic peptide
TWNGDR

SEQ ID NO: 174 - protein, 5 aa, synthetic construct
  REGION 1..5: note = Description of Artificial Sequence: Synthetic peptide
GTAGD

SEQ ID NO: 175 - protein, 5 aa, synthetic construct
  REGION 1..5: note = Description of Artificial Sequence: Synthetic peptide
YHSGS

SEQ ID NO: 176 - protein, 5 aa, synthetic construct
  REGION 1..5: note = Description of Artificial Sequence: Synthetic peptide
SYGGS

SEQ ID NO: 177 - protein, 6 aa, synthetic construct
  REGION 1..6: note = Description of Artificial Sequence: Synthetic peptide
YPGDGD

SEQ ID NO: 178 - protein, 6 aa, synthetic construct
  REGION 1..6: note = Description of Artificial Sequence: Synthetic peptide
SGGGSY

SEQ ID NO: 179 - protein, 5 aa, synthetic construct
  REGION 1..5: note = Description of Artificial Sequence: Synthetic peptide
WTGGG

SEQ ID NO: 180 - protein, 5 aa, synthetic construct
  REGION 1..5: note = Description of Artificial Sequence: Synthetic peptide
NYSGS

SEQ ID NO: 181 - protein, 6 aa, synthetic construct
  REGION 1..6: note = Description of Artificial Sequence: Synthetic peptide
DPANGN

SEQ ID NO: 182 - protein, 6 aa, synthetic construct
  REGION 1..6: note = Description of Artificial Sequence: Synthetic peptide
SAEGSN

SEQ ID NO: 183 - protein, 6 aa, synthetic construct
  REGION 1..6: note = Description of Artificial Sequence: Synthetic peptide
SYGGSD

SEQ ID NO: 184 - protein, 4 aa, synthetic construct
  REGION 1..4: note = Description of Artificial Sequence: Synthetic peptide
DEGY

SEQ ID NO: 185 - protein, 6 aa, synthetic construct
  REGION 1..6: note = Description of Artificial Sequence: Synthetic peptide
GGPFDY

SEQ ID NO: 186 - protein, 8 aa, synthetic construct
  REGION 1..8: note = Description of Artificial Sequence: Synthetic peptide
VYSYGFDY

SEQ ID NO: 187 - protein, 7 aa, synthetic construct
  REGION 1..7: note = Description of Artificial Sequence: Synthetic peptide
DNWAFDL

SEQ ID NO: 188 - protein, 10 aa, synthetic construct
  REGION 1..10: note = Description of Artificial Sequence: Synthetic peptide
TWYSSSPFDY

SEQ ID NO: 189 - protein, 11 aa, synthetic construct
  REGION 1..11: note = Description of Artificial Sequence: Synthetic peptide
DSGSYYDWFDP

SEQ ID NO: 190 - protein, 7 aa, synthetic construct
  REGION 1..7: note = Description of Artificial Sequence: Synthetic peptide
YSNYFDY

SEQ ID NO: 191 - protein, 12 aa, synthetic construct
  REGION 1..12: note = Description of Artificial Sequence: Synthetic peptide
VHHYYGSSYFDY

SEQ ID NO: 192 - protein, 12 aa, synthetic construct
  REGION 1..12: note = Description of Artificial Sequence: Synthetic peptide
VHHYYGSSYFDY

SEQ ID NO: 193 - protein, 12 aa, synthetic construct
  REGION 1..12: note = Description of Artificial Sequence: Synthetic peptide
DPYYYGSSYFDY

SEQ ID NO: 194 - protein, 11 aa, synthetic construct
  REGION 1..11: note = Description of Artificial Sequence: Synthetic peptide
EYYYGSSSFAY

SEQ ID NO: 195 - protein, 11 aa, synthetic construct
  REGION 1..11: note = Description of Artificial Sequence: Synthetic peptide
EYYYGSSSFAY

SEQ ID NO: 196 - protein, 5 aa, synthetic construct
  REGION 1..5: note = Description of Artificial Sequence: Synthetic peptide
REFAY

SEQ ID NO: 197 - protein, 11 aa, synthetic construct
  REGION 1..11: note = Description of Artificial Sequence: Synthetic peptide
PNYHGSSPFAY

SEQ ID NO: 198 - protein, 15 aa, synthetic construct
  REGION 1..15: note = Description of Artificial Sequence: Synthetic peptide
ARGKWRWRLGRRYDY

SEQ ID NO: 199 - protein, 15 aa, synthetic construct
  REGION 1..15: note = Description of Artificial Sequence: Synthetic peptide
ARGYWRWRLLRRYDY

SEQ ID NO: 200 - protein, 9 aa, synthetic construct
  REGION 1..9: note = Description of Artificial Sequence: Synthetic peptide
DYGSSSFDY

SEQ ID NO: 201 - protein, 15 aa, synthetic construct
  REGION 1..15: note = Description of Artificial Sequence: Synthetic peptide
ARGYPRWRLGRRYDY

SEQ ID NO: 202 - protein, 15 aa, synthetic construct
  REGION 1..15: note = Description of Artificial Sequence: Synthetic peptide
ARGYKRWRLGRRYDY

SEQ ID NO: 203 - protein, 12 aa, synthetic construct
  REGION 1..12: note = Description of Artificial Sequence: Synthetic peptide
LYGYDLHWYFDV

SEQ ID NO: 204 - protein, 11 aa, synthetic construct
  REGION 1..11: note = Description of Artificial Sequence: Synthetic peptide
DEVAAAGLFDY

SEQ ID NO: 205 - protein, 15 aa, synthetic construct
  REGION 1..15: note = Description of Artificial Sequence: Synthetic peptide
HEVEYYGPSSSWFAY

SEQ ID NO: 206 - protein, 15 aa, synthetic construct
  REGION 1..15: note = Description of Artificial Sequence: Synthetic peptide
ARGKWRWRLGRRYDY

SEQ ID NO: 207 - protein, 9 aa, synthetic construct
  REGION 1..9: note = Description of Artificial Sequence: Synthetic peptide
DYGSSSFDY

SEQ ID NO: 208 - protein, 12 aa, synthetic construct
  REGION 1..12: note = Description of Artificial Sequence: Synthetic peptide
EGDNSNYYAMDY

SEQ ID NO: 209 - protein, 15 aa, synthetic construct
  REGION 1..15: note = Description of Artificial Sequence: Synthetic peptide
ARGHWRWRLGRRYDY

SEQ ID NO: 210 - protein, 15 aa, synthetic construct
  REGION 1..15: note = Description of Artificial Sequence: Synthetic peptide
ARGYWRWRLWRRYDY

SEQ ID NO: 211 - protein, 15 aa, synthetic construct
  REGION 1..15: note = Description of Artificial Sequence: Synthetic peptide
ARGRWRWRLGRRYDY

SEQ ID NO: 212 - protein, 10 aa, synthetic construct
  REGION 1..10: note = Description of Artificial Sequence: Synthetic peptide
EDDYGWYFGV

SEQ ID NO: 213 - protein, 14 aa, synthetic construct
  REGION 1..14: note = Description of Artificial Sequence: Synthetic peptide
HEDPIYYGNYVFAY

SEQ ID NO: 214 - protein, 13 aa, synthetic construct
  REGION 1..13: note = Description of Artificial Sequence: Synthetic peptide
LYYGSSWERYFDV

SEQ ID NO: 215 - protein, 11 aa, synthetic construct
  REGION 1..11: note = Description of Artificial Sequence: Synthetic peptide
QGSNYGGYFDV

SEQ ID NO: 216 - protein, 15 aa, synthetic construct
  REGION 1..15: note = Description of Artificial Sequence: Synthetic peptide
ARGYWRWRLGRRADY

SEQ ID NO: 217 - protein, 14 aa, synthetic construct
  REGION 1..14: note = Description of Artificial Sequence: Synthetic peptide
VIYYYGSSDYTLDY

SEQ ID NO: 218 - protein, 15 aa, synthetic construct
  REGION 1..15: note = Description of Artificial Sequence: Synthetic peptide
ARGKWRWRLGRRYDY

SEQ ID NO: 219 - protein, 9 aa, synthetic construct
  REGION 1..9: note = Description of Artificial Sequence: Synthetic peptide
DYGSSSFDY

SEQ ID NO: 220 - protein, 8 aa, synthetic construct
  REGION 1..8: note = Description of Artificial Sequence: Synthetic peptide
SMITRFAY

SEQ ID NO: 221 - protein, 10 aa, synthetic construct
  REGION 1..10: note = Description of Artificial Sequence: Synthetic peptide
YYYGSSAFAY

SEQ ID NO: 222 - protein, 11 aa, synthetic construct
  REGION 1..11: note = Description of Artificial Sequence: Synthetic peptide
AYYSNYGVMYF

SEQ ID NO: 223 - protein, 15 aa, synthetic construct
  REGION 1..15: note = Description of Artificial Sequence: Synthetic peptide
ARGGWRWRLGRRYDY

SEQ ID NO: 224 - protein, 14 aa, synthetic construct
  REGION 1..14: note = Description of Artificial Sequence: Synthetic peptide
PYYYGSSRYYAMDY

SEQ ID NO: 225 - protein, 11 aa, synthetic construct
  REGION 1..11: note = Description of Artificial Sequence: Synthetic peptide
DYYGSSWHFDV

SEQ ID NO: 226 - protein, 15 aa, synthetic construct
  REGION 1..15: note = Description of Artificial Sequence: Synthetic peptide
YIWYYGSSWSWYFDA

SEQ ID NO: 227 - protein, 8 aa, synthetic construct
  REGION 1..8: note = Description of Artificial Sequence: Synthetic peptide
GYNWIFDL

SEQ ID NO: 228 - protein, 8 aa, synthetic construct
  REGION 1..8: note = Description of Artificial Sequence: Synthetic peptide
GNYDAMDY

SEQ ID NO: 229 - protein, 11 aa, synthetic construct
  REGION 1..11: note = Description of Artificial Sequence: Synthetic peptide
GEYSRLWYFDL

SEQ ID NO: 230 - protein, 8 aa, synthetic construct
  REGION 1..8: note = Description of Artificial Sequence: Synthetic peptide
PYDYDFDY

SEQ ID NO: 231 - protein, 10 aa, synthetic construct
  REGION 1..10: note = Description of Artificial Sequence: Synthetic peptide
TVVADWYFDV

SEQ ID NO: 232 - protein, 12 aa, synthetic construct
  REGION 1..12: note = Description of Artificial Sequence: Synthetic peptide
KGDGGSYAAMDY

SEQ ID NO: 233 - protein, 10 aa, synthetic construct
  REGION 1..10: note = Description of Artificial Sequence: Synthetic peptide
TVVADWYFDV

SEQ ID NO: 234 - protein, 11 aa, synthetic construct
  REGION 1..11: note = Description of Artificial Sequence: Synthetic peptide
NYGSSYYALDY

SEQ ID NO: 235 - protein, 11 aa, synthetic construct
  REGION 1..11: note = Description of Artificial Sequence: Synthetic peptide
QGSNYGGYFDV

SEQ ID NO: 236 - protein, 8 aa, synthetic construct
  REGION 1..8: note = Description of Artificial Sequence: Synthetic peptide
DYIYAMDY

SEQ ID NO: 237 - protein, 11 aa, synthetic construct
  REGION 1..11: note = Description of Artificial Sequence: Synthetic peptide
GDYGSSYAMDY

SEQ ID NO: 238 - protein, 10 aa, synthetic construct
  REGION 1..10: note = Description of Artificial Sequence: Synthetic peptide
YYYGSSAFAY

SEQ ID NO: 239 - protein, 10 aa, synthetic construct
  REGION 1..10: note = Description of Artificial Sequence: Synthetic peptide
IYYDYSAMDY

SEQ ID NO: 240 - protein, 10 aa, synthetic construct
  REGION 1..10: note = Description of Artificial Sequence: Synthetic peptide
DYYGSSYLDY

SEQ ID NO: 241 - protein, 14 aa, synthetic construct
  REGION 1..14: note = Description of Artificial Sequence: Synthetic peptide
DYYGSFHRRWYFDV

SEQ ID NO: 242 - protein, 13 aa, synthetic construct
  REGION 1..13: note = Description of Artificial Sequence: Synthetic peptide
DYHGSSFYWYFDV

SEQ ID NO: 243 - protein, 11 aa, synthetic construct
  REGION 1..11: note = Description of Artificial Sequence: Synthetic peptide
YYDSSYYAMDY

SEQ ID NO: 244 - protein, 10 aa, synthetic construct
  REGION 1..10: note = Description of Artificial Sequence: Synthetic peptide
GIAVAGTFDY

SEQ ID NO: 245 - protein, 12 aa, synthetic construct
  REGION 1..12: note = Description of Artificial Sequence: Synthetic peptide
HALLWYYYAMDY

SEQ ID NO: 246 - protein, 9 aa, synthetic construct
  REGION 1..9: note = Description of Artificial Sequence: Synthetic peptide
VSYSGSLHY

SEQ ID NO: 247 - protein, 8 aa, synthetic construct
  REGION 1..8: note = Description of Artificial Sequence: Synthetic peptide
SNWNYFDY

SEQ ID NO: 248 - protein, 10 aa, synthetic construct
  REGION 1..10: note = Description of Artificial Sequence: Synthetic peptide
PRDSGPSFAS

SEQ ID NO: 249 - protein, 15 aa, synthetic construct
  REGION 1..15: note = Description of Artificial Sequence: Synthetic peptide
SRGYYYGSSYGYYDV

SEQ ID NO: 250 - protein, 6 aa, synthetic construct
  REGION 1..6: note = Description of Artificial Sequence: Synthetic peptide
NWGFAY

SEQ ID NO: 251 - protein, 10 aa, synthetic construct
  REGION 1..10: note = Description of Artificial Sequence: Synthetic peptide
DYGSSYEFTY

SEQ ID NO: 252 - protein, 8 aa, synthetic construct
  REGION 1..8: note = Description of Artificial Sequence: Synthetic peptide
GYSGSVDY

SEQ ID NO: 253 - protein, 8 aa, synthetic construct
  REGION 1..8: note = Description of Artificial Sequence: Synthetic peptide
DATGTFAY

SEQ ID NO: 254 - protein, 13 aa, synthetic construct
  REGION 1..13: note = Description of Artificial Sequence: Synthetic peptide
EVVSGMIGTVFDY

SEQ ID NO: 255 - protein, 8 aa, synthetic construct
  REGION 1..8: note = Description of Artificial Sequence: Synthetic peptide
GYNWIFDY

SEQ ID NO: 256 - protein, 8 aa, synthetic construct
  REGION 1..8: note = Description of Artificial Sequence: Synthetic peptide
GYNWIFDF

SEQ ID NO: 257 - protein, 8 aa, synthetic construct
  REGION 1..8: note = Description of Artificial Sequence: Synthetic peptide
GYNWIFDF

SEQ ID NO: 258 - protein, 13 aa, synthetic construct
  REGION 1..13: note = Description of Artificial Sequence: Synthetic peptide
EVVSGMIGTIFDY

SEQ ID NO: 259 - protein, 8 aa, synthetic construct
  REGION 1..8: note = Description of Artificial Sequence: Synthetic peptide
GYNWIFDF

SEQ ID NO: 260 - protein, 13 aa, synthetic construct
  REGION 1..13: note = Description of Artificial Sequence: Synthetic peptide
EVVSGMIGTIFDY

SEQ ID NO: 261 - protein, 8 aa, synthetic construct
  REGION 1..8: note = Description of Artificial Sequence: Synthetic peptide
GYNWVFDL

SEQ ID NO: 262 - protein, 9 aa, synthetic construct
  REGION 1..9: note = Description of Artificial Sequence: Synthetic peptide
DGLTGIFDY

SEQ ID NO: 263 - protein, 8 aa, synthetic construct
  REGION 1..8: note = Description of Artificial Sequence: Synthetic peptide
GYNWIFDY

SEQ ID NO: 264 - protein, 13 aa, synthetic construct
  REGION 1..13: note = Description of Artificial Sequence: Synthetic peptide
EVVSGLYGTIFDY

SEQ ID NO: 265 - protein, 7 aa, synthetic construct
  REGION 1..7: note = Description of Artificial Sequence: Synthetic peptide
DYDYFDY

SEQ ID NO: 266 - skipped

SEQ ID NO: 267 - protein, 11 aa, synthetic construct
  REGION 1..11: note = Description of Artificial Sequence: Synthetic peptide
PNYSGSSPFAY

SEQ ID NO: 268 - protein, 15 aa, synthetic construct
  REGION 1..15: note = Description of Artificial Sequence: Synthetic peptide
SRGYYYGSSYGYFDV

SEQ ID NO: 269 - protein, 10 aa, synthetic construct
  REGION 1..10: note = Description of Artificial Sequence: Synthetic peptide
KLDWDGYFDV

SEQ ID NO: 270 - protein, 14 aa, synthetic construct
  REGION 1..14: note = Description of Artificial Sequence: Synthetic peptide
GSPYGYDGHYVMDY

SEQ ID NO: 271 - protein, 46 aa, synthetic construct
  REGION 1..46: note = Description of Artificial Sequence: Synthetic polypeptide
DGRGSLPRPKGGFIGALSFHWPFGRWLGGSYGTYDSSEDSGGAFDI

SEQ ID NO: 272 - protein, 46 aa, synthetic construct
  REGION 1..46: note = Description of Artificial Sequence: Synthetic polypeptide
DGRGSLPRPKGGFIGDLSFHWPFGRWLGKSYGTYDSSEDSGGAFDI

SEQ ID NO: 273 - protein, 16 aa, synthetic construct
  REGION 1..16: note = Description of Artificial Sequence: Synthetic peptide
RSSQSLVYSDGNTYLN

SEQ ID NO: 274 - protein, 11 aa, synthetic construct
  REGION 1..11: note = Description of Artificial Sequence: Synthetic peptide
KASQDIDDDMN

SEQ ID NO: 275 - protein, 17 aa, synthetic construct
  REGION 1..17: note = Description of Artificial Sequence: Synthetic peptide
KSSQSVLYSSNNKNYLA

SEQ ID NO: 276 - protein, 11 aa, synthetic construct
  REGION 1..11: note = Description of Artificial Sequence: Synthetic peptide
RASQSVSSNLA

SEQ ID NO: 277 - protein, 11 aa, synthetic construct
  REGION 1..11: note = Description of Artificial Sequence: Synthetic peptide
KASQDIDDDMN

SEQ ID NO: 278 - protein, 11 aa, synthetic construct
  REGION 1..11: note = Description of Artificial Sequence: Synthetic peptide
RASQGISSWLA

SEQ ID NO: 279 - protein, 11 aa, synthetic construct
  REGION 1..11: note = Description of Artificial Sequence: Synthetic peptide
KASQDVDDDMN

SEQ ID NO: 280 - protein, 11 aa, synthetic construct
  REGION 1..11: note = Description of Artificial Sequence: Synthetic peptide
KASQNVGTNVA

SEQ ID NO: 281 - protein, 11 aa, synthetic construct
  REGION 1..11: note = Description of Artificial Sequence: Synthetic peptide
KASQNVGSSVA

SEQ ID NO: 282 - protein, 11 aa, synthetic construct
  REGION 1..11: note = Description of Artificial Sequence: Synthetic peptide
RASQSISDYLH

SEQ ID NO: 283 - protein, 11 aa, synthetic construct
  REGION 1..11: note = Description of Artificial Sequence: Synthetic peptide
KASRNVGTNVA

SEQ ID NO: 284 - protein, 11 aa, synthetic construct
  REGION 1..11: note = Description of Artificial Sequence: Synthetic peptide
RASQSISDYLH

SEQ ID NO: 285 - protein, 11 aa, synthetic construct
  REGION 1..11: note = Description of Artificial Sequence: Synthetic peptide
KASQNVGTNVA

SEQ ID NO: 286 - protein, 11 aa, synthetic construct
  REGION 1..11: note = Description of Artificial Sequence: Synthetic peptide
RASQSISNYLH

SEQ ID NO: 287 - protein, 11 aa, synthetic construct
  REGION 1..11: note = Description of Artificial Sequence: Synthetic peptide
RASQSIGRYLN

SEQ ID NO: 288 - protein, 11 aa, synthetic construct
  REGION 1..11: note = Description of Artificial Sequence: Synthetic peptide
RASQSIGTYLN

SEQ ID NO: 289 - protein, 11 aa, synthetic construct
  REGION 1..11: note = Description of Artificial Sequence: Synthetic peptide
KASQSVRNDVV

SEQ ID NO: 290 - protein, 11 aa, synthetic construct
  REGION 1..11: note = Description of Artificial Sequence: Synthetic peptide
RASQSVTTYLN

SEQ ID NO: 291 - protein, 11 aa, synthetic construct
  REGION 1..11: note = Description of Artificial Sequence: Synthetic peptide
RASQSISDYLN

SEQ ID NO: 292 - protein, 15 aa, synthetic construct
  REGION 1..15: note = Description of Artificial Sequence: Synthetic peptide
KASHSVDYDGDNYMN

SEQ ID NO: 293 - protein, 17 aa, synthetic construct
  REGION 1..17: note = Description of Artificial Sequence: Synthetic peptide
KSSQSVLYSSNNKNYFA

SEQ ID NO: 294 - protein, 11 aa, synthetic construct
  REGION 1..11: note = Description of Artificial Sequence: Synthetic peptide
RASQDIGRSLS

SEQ ID NO: 295 - protein, 11 aa, synthetic construct
  REGION 1..11: note = Description of Artificial Sequence: Synthetic peptide
RASQSIGRYLN

SEQ ID NO: 296 - protein, 11 aa, synthetic construct
  REGION 1..11: note = Description of Artificial Sequence: Synthetic peptide
KASQSVRNDVV

SEQ ID NO: 297 - protein, 15 aa, synthetic construct
  REGION 1..15: note = Description of Artificial Sequence: Synthetic peptide
KASQSVDYDGDSYMN

SEQ ID NO: 298 - protein, 11 aa, synthetic construct
  REGION 1..11: note = Description of Artificial Sequence: Synthetic peptide
RASQTISRYLN

SEQ ID NO: 299 - protein, 11 aa, synthetic construct
  REGION 1..11: note = Description of Artificial Sequence: Synthetic peptide
RASQSIGSYLN

SEQ ID NO: 300 - protein, 11 aa, synthetic construct
  REGION 1..11: note = Description of Artificial Sequence: Synthetic peptide
RASRSISSYLN

SEQ ID NO: 301 - protein, 11 aa, synthetic construct
  REGION 1..11: note = Description of Artificial Sequence: Synthetic peptide
KVSQDVRTAVA

SEQ ID NO: 302 - protein, 11 aa, synthetic construct
  REGION 1..11: note = Description of Artificial Sequence: Synthetic peptide
GGGNDIGSSMY

SEQ ID NO: 303 - protein, 11 aa, synthetic construct
  REGION 1..11: note = Description of Artificial Sequence: Synthetic peptide
RASQSISDYLN

SEQ ID NO: 304 - protein, 11 aa, synthetic construct
  REGION 1..11: note = Description of Artificial Sequence: Synthetic peptide
GGGNDIGSSMY

SEQ ID NO: 305 - protein, 15 aa, synthetic construct
  REGION 1..15: note = Description of Artificial Sequence: Synthetic peptide
RASESVDSFGNNFMN

SEQ ID NO: 306 - protein, 10 aa, synthetic construct
  REGION 1..10: note = Description of Artificial Sequence: Synthetic peptide
RASSSVTYIH

SEQ ID NO: 307 - protein, 11 aa, synthetic construct
  REGION 1..11: note = Description of Artificial Sequence: Synthetic peptide
RASQSIGRYLN

SEQ ID NO: 308 - protein, 11 aa, synthetic construct
  REGION 1..11: note = Description of Artificial Sequence: Synthetic peptide
KASQSVRNDVV

SEQ ID NO: 309 - protein, 15 aa, synthetic construct
  REGION 1..15: note = Description of Artificial Sequence: Synthetic peptide
KASHSVDYDGDNYMN

SEQ ID NO: 310 - protein, 15 aa, synthetic construct
  REGION 1..15: note = Description of Artificial Sequence: Synthetic peptide
RASESVNSYGNSFMH

SEQ ID NO: 311 - protein, 16 aa, synthetic construct
  REGION 1..16: note = Description of Artificial Sequence: Synthetic peptide
RSSKSLLHSSGNAYVY

SEQ ID NO: 312 - protein, 11 aa, synthetic construct
  REGION 1..11: note = Description of Artificial Sequence: Synthetic peptide
RASQSIGTYLN

SEQ ID NO: 313 - protein, 12 aa, synthetic construct
  REGION 1..12: note = Description of Artificial Sequence: Synthetic peptide
RASSRVSSSYLY

SEQ ID NO: 314 - protein, 11 aa, synthetic construct
  REGION 1..11: note = Description of Artificial Sequence: Synthetic peptide
GASQSIGTIIH

SEQ ID NO: 315 - protein, 11 aa, synthetic construct
  REGION 1..11: note = Description of Artificial Sequence: Synthetic peptide
RASENIYVPLN

SEQ ID NO: 316 - protein, 12 aa, synthetic construct
  REGION 1..12: note = Description of Artificial Sequence: Synthetic peptide
RASQSVSSSYLA

SEQ ID NO: 317 - protein, 16 aa, synthetic construct
  REGION 1..16: note = Description of Artificial Sequence: Synthetic peptide
KSSQSLLYSGEKTYPY

SEQ ID NO: 318 - protein, 11 aa, synthetic construct
  REGION 1..11: note = Description of Artificial Sequence: Synthetic peptide
QSSQDIFNYLE

SEQ ID NO: 319 - protein, 11 aa, synthetic construct
  REGION 1..11: note = Description of Artificial Sequence: Synthetic peptide
RSTRNILSNMP

SEQ ID NO: 320 - protein, 11 aa, synthetic construct
  REGION 1..11: note = Description of Artificial Sequence: Synthetic peptide
RASQDISNNLH

SEQ ID NO: 321 - protein, 11 aa, synthetic construct
  REGION 1..11: note = Description of Artificial Sequence: Synthetic peptide
SASQSMSNNLH

SEQ ID NO: 322 - protein, 11 aa, synthetic construct
  REGION 1..11: note = Description of Artificial Sequence: Synthetic peptide
RASQDISNNLH

SEQ ID NO: 323 - protein, 10 aa, synthetic construct
  REGION 1..10: note = Description of Artificial Sequence: Synthetic peptide
SASSSLSYMH

SEQ ID NO: 324 - protein, 10 aa, synthetic construct
  REGION 1..10: note = Description of Artificial Sequence: Synthetic peptide
SASSSVSYMN

SEQ ID NO: 325 - protein, 11 aa, synthetic construct
  REGION 1..11: note = Description of Artificial Sequence: Synthetic peptide
KASQNMGSNVA

SEQ ID NO: 326 - protein, 11 aa, synthetic construct
  REGION 1..11: note = Description of Artificial Sequence: Synthetic peptide
KASQNGGTNVD

SEQ ID NO: 327 - protein, 11 aa, synthetic construct
  REGION 1..11: note = Description of Artificial Sequence: Synthetic peptide
RASENIYVPLN

SEQ ID NO: 328 - protein, 11 aa, synthetic construct
  REGION 1..11: note = Description of Artificial Sequence: Synthetic peptide
KASQNMGSNVA

SEQ ID NO: 329 - protein, 11 aa, synthetic construct
  REGION 1..11: note = Description of Artificial Sequence: Synthetic peptide
KASQNGGTNVD

SEQ ID NO: 330 - protein, 11 aa, synthetic construct
  REGION 1..11: note = Description of Artificial Sequence: Synthetic peptide
KASQGVDTNVA

SEQ ID NO: 331 - protein, 11 aa, synthetic construct
  REGION 1..11: note = Description of Artificial Sequence: Synthetic peptide
KASQDVGTAIT

SEQ ID NO: 332 - protein, 11 aa, synthetic construct
  REGION 1..11: note = Description of Artificial Sequence: Synthetic peptide
KASQDVGTSVA

SEQ ID NO: 333 - protein, 11 aa, synthetic construct
  REGION 1..11: note = Description of Artificial Sequence: Synthetic peptide
RASQVIDDDIN

SEQ ID NO: 334 - protein, 11 aa, synthetic construct
  REGION 1..11: note = Description of Artificial Sequence: Synthetic peptide
RASQEISGYLT

SEQ ID NO: 335 - protein, 11 aa, synthetic construct
  REGION 1..11: note = Description of Artificial Sequence: Synthetic peptide
RASQSISRYLN

SEQ ID NO: 336 - protein, 11 aa, synthetic construct
  REGION 1..11: note = Description of Artificial Sequence: Synthetic peptide
RASQGISSWLD

SEQ ID NO: 337 - protein, 11 aa, synthetic construct
  REGION 1..11: note = Description of Artificial Sequence: Synthetic peptide
KASQNMGSNVA

SEQ ID NO: 338 - protein, 11 aa, synthetic construct
  REGION 1..11: note = Description of Artificial Sequence: Synthetic peptide
RASQGILGYLN

SEQ ID NO: 339 - protein, 15 aa, synthetic construct
  REGION 1..15: note = Description of Artificial Sequence: Synthetic peptide
RASESVDNYGISFMS

SEQ ID NO: 340 - protein, 16 aa, synthetic construct
  REGION 1..16: note = Description of Artificial Sequence: Synthetic peptide
KSSQSLLYSGEKTYPY

SEQ ID NO: 341 - protein, 11 aa, synthetic construct
  REGION 1..11: note = Description of Artificial Sequence: Synthetic peptide
RASQGLRHDLG

SEQ ID NO: 342 - protein, 15 aa, synthetic construct
  REGION 1..15: note = Description of Artificial Sequence: Synthetic peptide
HASESVSVAGTSLLH

SEQ ID NO: 343 - protein, 11 aa, synthetic construct
  REGION 1..11: note = Description of Artificial Sequence: Synthetic peptide
RVSQGISNYLN

SEQ ID NO: 344 - protein, 11 aa, synthetic construct
  REGION 1..11: note = Description of Artificial Sequence: Synthetic peptide
RASQSVSSNLA

SEQ ID NO: 345 - protein, 11 aa, synthetic construct
  REGION 1..11: note = Description of Artificial Sequence: Synthetic peptide
RASQSVSSNLA

SEQ ID NO: 346 - protein, 12 aa, synthetic construct
  REGION 1..12: note = Description of Artificial Sequence: Synthetic peptide
RASQSVSSSYLA

SEQ ID NO: 347 - protein, 11 aa, synthetic construct
  REGION 1..11: note = Description of Artificial Sequence: Synthetic peptide
RASQSISSYLN

SEQ ID NO: 348 - protein, 11 aa, synthetic construct
  REGION 1..11: note = Description of Artificial Sequence: Synthetic peptide
RASQSVSSNLA

SEQ ID NO: 349 - protein, 11 aa, synthetic construct
  REGION 1..11: note = Description of Artificial Sequence: Synthetic peptide
RASQRISSYLN

SEQ ID NO: 350 - protein, 12 aa, synthetic construct
  REGION 1..12: note = Description of Artificial Sequence: Synthetic peptide
RAIQSVSSSYLA

SEQ ID NO: 351 - protein, 11 aa, synthetic construct
  REGION 1..11: note = Description of Artificial Sequence: Synthetic peptide
RASQSVSSYLA

SEQ ID NO: 352 - protein, 11 aa, synthetic construct
  REGION 1..11: note = Description of Artificial Sequence: Synthetic peptide
RASQSVSSNLA

SEQ ID NO: 353 - protein, 11 aa, synthetic construct
  REGION 1..11: note = Description of Artificial Sequence: Synthetic peptide
RASQSISSYLN

SEQ ID NO: 354 - protein, 11 aa, synthetic construct
  REGION 1..11: note = Description of Artificial Sequence: Synthetic peptide
KASQSVSNDVA

SEQ ID NO: 355 - protein, 11 aa, synthetic construct
  REGION 1..11: note = Description of Artificial Sequence: Synthetic peptide
KASQNVGTNVA

SEQ ID NO: 356 - protein, 10 aa, synthetic construct
  REGION 1..10: note = Description of Artificial Sequence: Synthetic peptide
SASSSVSYMY

SEQ ID NO: 357 - protein, 11 aa, synthetic construct
  REGION 1..11: note = Description of Artificial Sequence: Synthetic peptide
RASQSISDYLH

SEQ ID NO: 358 - protein, 11 aa, synthetic construct
  REGION 1..11: note = Description of Artificial Sequence: Synthetic peptide
HASQNINVWLN

SEQ ID NO: 359 - protein, 11 aa, synthetic construct
  REGION 1..11: note = Description of Artificial Sequence: Synthetic peptide
KASQNVGSNVA

SEQ ID NO: 360 - protein, 13 aa, synthetic construct
  REGION 1..13: note = Description of Artificial Sequence: Synthetic peptide
SGISSNIGNNYVS

SEQ ID NO: 361 - protein, 13 aa, synthetic construct
  REGION 1..13: note = Description of Artificial Sequence: Synthetic peptide
SGSSSNIGNHYVS

SEQ ID NO: 362 - protein, 7 aa, synthetic construct
  REGION 1..7: note = Description of Artificial Sequence: Synthetic peptide
KVSNRDS

SEQ ID NO: 363 - protein, 7 aa, synthetic construct
  REGION 1..7: note = Description of Artificial Sequence: Synthetic peptide
EATTLVP

SEQ ID NO: 364 - protein, 7 aa, synthetic construct
  REGION 1..7: note = Description of Artificial Sequence: Synthetic peptide
WASTRES

SEQ ID NO: 365 - protein, 7 aa, synthetic construct
  REGION 1..7: note = Description of Artificial Sequence: Synthetic peptide
GASTRAT

SEQ ID NO: 366 - protein, 7 aa, synthetic construct
  REGION 1..7: note = Description of Artificial Sequence: Synthetic peptide
EATTLVP

SEQ ID NO: 367 - protein, 7 aa, synthetic construct
  REGION 1..7: note = Description of Artificial Sequence: Synthetic peptide
AASSLQS

SEQ ID NO: 368 - protein, 7 aa, synthetic construct
  REGION 1..7: note = Description of Artificial Sequence: Synthetic peptide
EATTLVP

SEQ ID NO: 369 - protein, 7 aa, synthetic construct
  REGION 1..7: note = Description of Artificial Sequence: Synthetic peptide
SASYRYS

SEQ ID NO: 370 - protein, 7 aa, synthetic construct
  REGION 1..7: note = Description of Artificial Sequence: Synthetic peptide
STSYRYS

SEQ ID NO: 371 - protein, 7 aa, synthetic construct
  REGION 1..7: note = Description of Artificial Sequence: Synthetic peptide
YASQSIS

SEQ ID NO: 372 - protein, 7 aa, synthetic construct
  REGION 1..7: note = Description of Artificial Sequence: Synthetic peptide
SASYRYS

SEQ ID NO: 373 - protein, 7 aa, synthetic construct
  REGION 1..7: note = Description of Artificial Sequence: Synthetic peptide
YASQSIS

SEQ ID NO: 374 - protein, 7 aa, synthetic construct
  REGION 1..7: note = Description of Artificial Sequence: Synthetic peptide
SASYRYS

SEQ ID NO: 375 - protein, 7 aa, synthetic construct
  REGION 1..7: note = Description of Artificial Sequence: Synthetic peptide
YASQSIS

SEQ ID NO: 376 - protein, 7 aa, synthetic construct
  REGION 1..7: note = Description of Artificial Sequence: Synthetic peptide
AASSLHS

SEQ ID NO: 377 - protein, 7 aa, synthetic construct
  REGION 1..7: note = Description of Artificial Sequence: Synthetic peptide
GASTLHS

SEQ ID NO: 378 - protein, 7 aa, synthetic construct
  REGION 1..7: note = Description of Artificial Sequence: Synthetic peptide
RGNTLRP

SEQ ID NO: 379 - protein, 7 aa, synthetic construct
  REGION 1..7: note = Description of Artificial Sequence: Synthetic peptide
SASSLQS

SEQ ID NO: 380 - protein, 7 aa, synthetic construct
  REGION 1..7: note = Description of Artificial Sequence: Synthetic peptide
TASTLQS

SEQ ID NO: 381 - protein, 7 aa, synthetic construct
  REGION 1..7: note = Description of Artificial Sequence: Synthetic peptide
WASTRLT

SEQ ID NO: 382 - protein, 7 aa, synthetic construct
  REGION 1..7: note = Description of Artificial Sequence: Synthetic peptide
DAPNRAT

SEQ ID NO: 383 - protein, 7 aa, synthetic construct
  REGION 1..7: note = Description of Artificial Sequence: Synthetic peptide
DASRFIS

SEQ ID NO: 384 - protein, 7 aa, synthetic construct
  REGION 1..7: note = Description of Artificial Sequence: Synthetic peptide
AASSLHS

SEQ ID NO: 385 - protein, 7 aa, synthetic construct
  REGION 1..7: note = Description of Artificial Sequence: Synthetic peptide
RGNTLRP

SEQ ID NO: 386 - protein, 7 aa, synthetic construct
  REGION 1..7: note = Description of Artificial Sequence: Synthetic peptide
RANRLVD

SEQ ID NO: 387 - protein, 7 aa, synthetic construct
  REGION 1..7: note = Description of Artificial Sequence: Synthetic peptide
SASTLQS

SEQ ID NO: 388 - protein, 7 aa, synthetic construct
  REGION 1..7: note = Description of Artificial Sequence: Synthetic peptide
GASNLQS

SEQ ID NO: 389 - protein, 7 aa, synthetic construct
  REGION 1..7: note = Description of Artificial Sequence: Synthetic peptide
AASSLPS

SEQ ID NO: 390 - protein, 7 aa, synthetic construct
  REGION 1..7: note = Description of Artificial Sequence: Synthetic peptide
DTSYLAS

SEQ ID NO: 391 - protein, 7 aa, synthetic construct
  REGION 1..7: note = Description of Artificial Sequence: Synthetic peptide
WMSNLAS

SEQ ID NO: 392 - protein, 7 aa, synthetic construct
  REGION 1..7: note = Description of Artificial Sequence: Synthetic peptide
GASPRES

SEQ ID NO: 393 - protein, 7 aa, synthetic construct
  REGION 1..7: note = Description of Artificial Sequence: Synthetic peptide
DASRFIS

SEQ ID NO: 394 - protein, 7 aa, synthetic construct
  REGION 1..7: note = Description of Artificial Sequence: Synthetic peptide
HTSRLNS

SEQ ID NO: 395 - protein, 7 aa, synthetic construct
  REGION 1..7: note = Description of Artificial Sequence: Synthetic peptide
AVSRLDS

SEQ ID NO: 396 - protein, 7 aa, synthetic construct
  REGION 1..7: note = Description of Artificial Sequence: Synthetic peptide
AASSLHS

SEQ ID NO: 397 - protein, 7 aa, synthetic construct
  REGION 1..7: note = Description of Artificial Sequence: Synthetic peptide
RGNTLRP

SEQ ID NO: 398 - protein, 7 aa, synthetic construct
  REGION 1..7: note = Description of Artificial Sequence: Synthetic peptide
DASRFIS

SEQ ID NO: 399 - protein, 7 aa, synthetic construct
  REGION 1..7: note = Description of Artificial Sequence: Synthetic peptide
DASRFIS

SEQ ID NO: 400 - protein, 7 aa, synthetic construct
  REGION 1..7: note = Description of Artificial Sequence: Synthetic peptide
YTSKPNS

SEQ ID NO: 401 - protein, 7 aa, synthetic construct
  REGION 1..7: note = Description of Artificial Sequence: Synthetic peptide
AASSLES

SEQ ID NO: 402 - protein, 7 aa, synthetic construct
  REGION 1..7: note = Description of Artificial Sequence: Synthetic peptide
ATYSLDY

SEQ ID NO: 403 - protein, 7 aa, synthetic construct
  REGION 1..7: note = Description of Artificial Sequence: Synthetic peptide
DTSYLAS

SEQ ID NO: 404 - protein, 7 aa, synthetic construct
  REGION 1..7: note = Description of Artificial Sequence: Synthetic peptide
DASRFIS

SEQ ID NO: 405 - protein, 7 aa, synthetic construct
  REGION 1..7: note = Description of Artificial Sequence: Synthetic peptide
GASSRAT

SEQ ID NO: 406 - protein, 7 aa, synthetic construct
  REGION 1..7: note = Description of Artificial Sequence: Synthetic peptide
WASTRLT

SEQ ID NO: 407 - protein, 7 aa, synthetic construct
  REGION 1..7: note = Description of Artificial Sequence: Synthetic peptide
TASNLDT

SEQ ID NO: 408 - protein, 7 aa, synthetic construct
  REGION 1..7: note = Description of Artificial Sequence: Synthetic peptide
NANTLAE

SEQ ID NO: 409 - protein, 7 aa, synthetic construct
  REGION 1..7: note = Description of Artificial Sequence: Synthetic peptide
EISGWLS

SEQ ID NO: 410 - protein, 7 aa, synthetic construct
  REGION 1..7: note = Description of Artificial Sequence: Synthetic peptide
LASNLGY

SEQ ID NO: 411 - protein, 7 aa, synthetic construct
  REGION 1..7: note = Description of Artificial Sequence: Synthetic peptide
WASTRLT

SEQ ID NO: 412 - protein, 7 aa, synthetic construct
  REGION 1..7: note = Description of Artificial Sequence: Synthetic peptide
GASPRES

SEQ ID NO: 413 - protein, 7 aa, synthetic construct
  REGION 1..7: note = Description of Artificial Sequence: Synthetic peptide
EISGWLS

SEQ ID NO: 414 - protein, 7 aa, synthetic construct
  REGION 1..7: note = Description of Artificial Sequence: Synthetic peptide
SASHRSS

SEQ ID NO: 415 - protein, 7 aa, synthetic construct
  REGION 1..7: note = Description of Artificial Sequence: Synthetic peptide
EISGWLS

SEQ ID NO: 416 - protein, 7 aa, synthetic construct
  REGION 1..7: note = Description of Artificial Sequence: Synthetic peptide
LASYRFT

SEQ ID NO: 417 - protein, 7 aa, synthetic construct
  REGION 1..7: note = Description of Artificial Sequence: Synthetic peptide
AATRLAD

SEQ ID NO: 418 - protein, 7 aa, synthetic construct
  REGION 1..7: note = Description of Artificial Sequence: Synthetic peptide
VASNQGT

SEQ ID NO: 419 - protein, 5 aa, synthetic construct
  REGION 1..5: note = Description of Artificial Sequence: Synthetic peptide
SSSIS

SEQ ID NO: 420 - protein, 7 aa, synthetic construct
  REGION 1..7: note = Description of Artificial Sequence: Synthetic peptide
SASKRNT

SEQ ID NO: 421 - protein, 7 aa, synthetic construct
  REGION 1..7: note = Description of Artificial Sequence: Synthetic peptide
PASYRSS

SEQ ID NO: 422 - protein, 7 aa, synthetic construct
  REGION 1..7: note = Description of Artificial Sequence: Synthetic peptide
LGSNRAP

SEQ ID NO: 423 - protein, 7 aa, synthetic construct
  REGION 1..7: note = Description of Artificial Sequence: Synthetic peptide
SASHRSS

SEQ ID NO: 424 - protein, 7 aa, synthetic construct
  REGION 1..7: note = Description of Artificial Sequence: Synthetic peptide
KASSLER

SEQ ID NO: 425 - protein, 7 aa, synthetic construct
  REGION 1..7: note = Description of Artificial Sequence: Synthetic peptide
TPFSLQS

SEQ ID NO: 426 - protein, 7 aa, synthetic construct
  REGION 1..7: note = Description of Artificial Sequence: Synthetic peptide
EASTRFS

SEQ ID NO: 427 - protein, 7 aa, synthetic construct
  REGION 1..7: note = Description of Artificial Sequence: Synthetic peptide
STSNLLL

SEQ ID NO: 428 - protein, 7 aa, synthetic construct
  REGION 1..7: note = Description of Artificial Sequence: Synthetic peptide
DASRFIS

SEQ ID NO: 429 - protein, 7 aa, synthetic construct
  REGION 1..7: note = Description of Artificial Sequence: Synthetic peptide
EASNRYT

SEQ ID NO: 430 - protein, 7 aa, synthetic construct
  REGION 1..7: note = Description of Artificial Sequence: Synthetic peptide
WASNRES

SEQ ID NO: 431 - protein, 7 aa, synthetic construct
  REGION 1..7: note = Description of Artificial Sequence: Synthetic peptide
AASNRES

SEQ ID NO: 432 - protein, 7 aa, synthetic construct
  REGION 1..7: note = Description of Artificial Sequence: Synthetic peptide
AASSLQS

SEQ ID NO: 433 - protein, 7 aa, synthetic construct
  REGION 1..7: note = Description of Artificial Sequence: Synthetic peptide
GASSRAT

SEQ ID NO: 434 - protein, 7 aa, synthetic construct
  REGION 1..7: note = Description of Artificial Sequence: Synthetic peptide
GASSRAT

SEQ ID NO: 435 - protein, 7 aa, synthetic construct
  REGION 1..7: note = Description of Artificial Sequence: Synthetic peptide
GASSRAT

SEQ ID NO: 436 - protein, 7 aa, synthetic construct
  REGION 1..7: note = Description of Artificial Sequence: Synthetic peptide
AASSLQS

SEQ ID NO: 437 - protein, 7 aa, synthetic construct
  REGION 1..7: note = Description of Artificial Sequence: Synthetic peptide
DASNRAT

SEQ ID NO: 438 - protein, 7 aa, synthetic construct
  REGION 1..7: note = Description of Artificial Sequence: Synthetic peptide
AASSLQS

SEQ ID NO: 439 - protein, 7 aa, synthetic construct
  REGION 1..7: note = Description of Artificial Sequence: Synthetic peptide
GASSRAT

SEQ ID NO: 440 - protein, 7 aa, synthetic construct
  REGION 1..7: note = Description of Artificial Sequence: Synthetic peptide
GASSRAT

SEQ ID NO: 441 - protein, 7 aa, synthetic construct
  REGION 1..7: note = Description of Artificial Sequence: Synthetic peptide
GASTRAT

SEQ ID NO: 442 - protein, 7 aa, synthetic construct
  REGION 1..7: note = Description of Artificial Sequence: Synthetic peptide
AASSLQS

SEQ ID NO: 443 - protein, 7 aa, synthetic construct
  REGION 1..7: note = Description of Artificial Sequence: Synthetic peptide
YASNRYT

SEQ ID NO: 444 - protein, 7 aa, synthetic construct
  REGION 1..7: note = Description of Artificial Sequence: Synthetic peptide
SASYRYS

SEQ ID NO: 445 - protein, 7 aa, synthetic construct
  REGION 1..7: note = Description of Artificial Sequence: Synthetic peptide
DTSNLAS

SEQ ID NO: 446 - protein, 7 aa, synthetic construct
  REGION 1..7: note = Description of Artificial Sequence: Synthetic peptide
YASQSIS

SEQ ID NO: 447 - protein, 7 aa, synthetic construct
  REGION 1..7: note = Description of Artificial Sequence: Synthetic peptide
KASNLHT

SEQ ID NO: 448 - protein, 7 aa, synthetic construct
  REGION 1..7: note = Description of Artificial Sequence: Synthetic peptide
SASYRYS

SEQ ID NO: 449 - protein, 8 aa, synthetic construct
  REGION 1..8: note = Description of Artificial Sequence: Synthetic peptide
DNNKRASG

SEQ ID NO: 450 - protein, 8 aa, synthetic construct
  REGION 1..8: note = Description of Artificial Sequence: Synthetic peptide
DNTKRPSG

SEQ ID NO: 451 - protein, 9 aa, synthetic construct
  REGION 1..9: note = Description of Artificial Sequence: Synthetic peptide
MQGTHWPRT

SEQ ID NO: 452 - protein, 10 aa, synthetic construct
  REGION 1..10: note = Description of Artificial Sequence: Synthetic peptide
LQHDNFPMYT

SEQ ID NO: 453 - protein, 9 aa, synthetic construct
  REGION 1..9: note = Description of Artificial Sequence: Synthetic peptide
QQYYSTPYT

SEQ ID NO: 454 - protein, 9 aa, synthetic construct
  REGION 1..9: note = Description of Artificial Sequence: Synthetic peptide
QQYYSTPLT

SEQ ID NO: 455 - protein, 9 aa, synthetic construct
  REGION 1..9: note = Description of Artificial Sequence: Synthetic peptide
LQHDNFPWT

SEQ ID NO: 456 - protein, 9 aa, synthetic construct
  REGION 1..9: note = Description of Artificial Sequence: Synthetic peptide
QQTNSFPRT

SEQ ID NO: 457 - protein, 9 aa, synthetic construct
  REGION 1..9: note = Description of Artificial Sequence: Synthetic peptide
LQHDNFPWT

SEQ ID NO: 458 - protein, 9 aa, synthetic construct
  REGION 1..9: note = Description of Artificial Sequence: Synthetic peptide
QRFNNYPLT

SEQ ID NO: 459 - protein, 9 aa, synthetic construct
  REGION 1..9: note = Description of Artificial Sequence: Synthetic peptide
QQYNSYPLT

SEQ ID NO: 460 - protein, 9 aa, synthetic construct
  REGION 1..9: note = Description of Artificial Sequence: Synthetic peptide
QNGHSFPLT

SEQ ID NO: 461 - protein, 9 aa, synthetic construct
  REGION 1..9: note = Description of Artificial Sequence: Synthetic peptide
QQYNSYPLT

SEQ ID NO: 462 - protein, 9 aa, synthetic construct
  REGION 1..9: note = Description of Artificial Sequence: Synthetic peptide
QNGHSFPHT

SEQ ID NO: 463 - protein, 9 aa, synthetic construct
  REGION 1..9: note = Description of Artificial Sequence: Synthetic peptide
QQYNIYPLT

SEQ ID NO: 464 - protein, 9 aa, synthetic construct
  REGION 1..9: note = Description of Artificial Sequence: Synthetic peptide
QNGHSFPLT

SEQ ID NO: 465 - protein, 9 aa, synthetic construct
  REGION 1..9: note = Description of Artificial Sequence: Synthetic peptide
QQSYVTPWT

SEQ ID NO: 466 - protein, 9 aa, synthetic construct
  REGION 1..9: note = Description of Artificial Sequence: Synthetic peptide
QQSYSAPWT

SEQ ID NO: 467 - protein, 9 aa, synthetic construct
  REGION 1..9: note = Description of Artificial Sequence: Synthetic peptide
QQYYGIPLT

SEQ ID NO: 468 - protein, 9 aa, synthetic construct
  REGION 1..9: note = Description of Artificial Sequence: Synthetic peptide
QQTYATPWT

SEQ ID NO: 469 - protein, 9 aa, synthetic construct
  REGION 1..9: note = Description of Artificial Sequence: Synthetic peptide
EQSYSTPWT

SEQ ID NO: 470 - protein, 9 aa, synthetic construct
  REGION 1..9: note = Description of Artificial Sequence: Synthetic peptide
LQHIEYPFT

SEQ ID NO: 471 - protein, 9 aa, synthetic construct
  REGION 1..9: note = Description of Artificial Sequence: Synthetic peptide
QQGYTTPYT

SEQ ID NO: 472 - protein, 9 aa, synthetic construct
  REGION 1..9: note = Description of Artificial Sequence: Synthetic peptide
QWSNSWPYT

SEQ ID NO: 473 - protein, 9 aa, synthetic construct
  REGION 1..9: note = Description of Artificial Sequence: Synthetic peptide
QQSYVTPWT

SEQ ID NO: 474 - protein, 9 aa, synthetic construct
  REGION 1..9: note = Description of Artificial Sequence: Synthetic peptide
QQYYGIPLT

SEQ ID NO: 475 - protein, 9 aa, synthetic construct
  REGION 1..9: note = Description of Artificial Sequence: Synthetic peptide
QNGHSFPLT

SEQ ID NO: 476 - protein, 9 aa, synthetic construct
  REGION 1..9: note = Description of Artificial Sequence: Synthetic peptide
QQSYSTPHT

SEQ ID NO: 477 - protein, 9 aa, synthetic construct
  REGION 1..9: note = Description of Artificial Sequence: Synthetic peptide
QQGYSAPRT

SEQ ID NO: 478 - protein, 9 aa, synthetic construct
  REGION 1..9: note = Description of Artificial Sequence: Synthetic peptide
QQSYSTPRT

SEQ ID NO: 479 - protein, 9 aa, synthetic construct
  REGION 1..9: note = Description of Artificial Sequence: Synthetic peptide
QQSYSWSLT

SEQ ID NO: 480 - protein, 9 aa, synthetic construct
  REGION 1..9: note = Description of Artificial Sequence: Synthetic peptide
QQYSTYPFA

SEQ ID NO: 481 - protein, 9 aa, synthetic construct
  REGION 1..9: note = Description of Artificial Sequence: Synthetic peptide
QQDNIWPYT

SEQ ID NO: 482 - protein, 10 aa, synthetic construct
  REGION 1..10: note = Description of Artificial Sequence: Synthetic peptide
QQSNEDPPFT

SEQ ID NO: 483 - protein, 9 aa, synthetic construct
  REGION 1..9: note = Description of Artificial Sequence: Synthetic peptide
QQNNEVPRT

SEQ ID NO: 484 - protein, 9 aa, synthetic construct
  REGION 1..9: note = Description of Artificial Sequence: Synthetic peptide
HQSNEDPYT

SEQ ID NO: 485 - protein, 9 aa, synthetic construct
  REGION 1..9: note = Description of Artificial Sequence: Synthetic peptide
QQSYVTPWT

SEQ ID NO: 486 - protein, 9 aa, synthetic construct
  REGION 1..9: note = Description of Artificial Sequence: Synthetic peptide
QQYYGIPLT

SEQ ID NO: 487 - protein, 8 aa, synthetic construct
  REGION 1..8: note = Description of Artificial Sequence: Synthetic peptide
LRYASYRT

SEQ ID NO: 488 - protein, 9 aa, synthetic construct
  REGION 1..9: note = Description of Artificial Sequence: Synthetic peptide
LQYGESPLT

SEQ ID NO: 489 - protein, 9 aa, synthetic construct
  REGION 1..9: note = Description of Artificial Sequence: Synthetic peptide
QHHYGIPLT

SEQ ID NO: 490 - protein, 9 aa, synthetic construct
  REGION 1..9: note = Description of Artificial Sequence: Synthetic peptide
QQTYNTPWT

SEQ ID NO: 491 - protein, 9 aa, synthetic construct
  REGION 1..9: note = Description of Artificial Sequence: Synthetic peptide
LQHGERPLT

SEQ ID NO: 492 - protein, 9 aa, synthetic construct
  REGION 1..9: note = Description of Artificial Sequence: Synthetic peptide
QQGNTRPWT

SEQ ID NO: 493 - protein, 10 aa, synthetic construct
  REGION 1..10: note = Description of Artificial Sequence: Synthetic peptide
QQYNSFPLYT

SEQ ID NO: 494 - protein, 9 aa, synthetic construct
  REGION 1..9: note = Description of Artificial Sequence: Synthetic peptide
QQYGSSPIT

SEQ ID NO: 495 - protein, 9 aa, synthetic construct
  REGION 1..9: note = Description of Artificial Sequence: Synthetic peptide
QQSNEDSWT

SEQ ID NO: 496 - protein, 11 aa, synthetic construct
  REGION 1..11: note = Description of Artificial Sequence: Synthetic peptide
QQGYSTPPEIT

SEQ ID NO: 497 - protein, 9 aa, synthetic construct
  REGION 1..9: note = Description of Artificial Sequence: Synthetic peptide
LQHWNYPYM

SEQ ID NO: 498 - protein, 10 aa, synthetic construct
  REGION 1..10: note = Description of Artificial Sequence: Synthetic peptide
QQSNSWSLLT

SEQ ID NO: 499 - protein, 10 aa, synthetic construct
  REGION 1..10: note = Description of Artificial Sequence: Synthetic peptide
RQNGHSFPLT

SEQ ID NO: 500 - protein, 9 aa, synthetic construct
  REGION 1..9: note = Description of Artificial Sequence: Synthetic peptide
QQWSDYPFT

SEQ ID NO: 501 - protein, 9 aa, synthetic construct
  REGION 1..9: note = Description of Artificial Sequence: Synthetic peptide
RQMSSYPPT

SEQ ID NO: 502 - protein, 8 aa, synthetic construct
  REGION 1..8: note = Description of Artificial Sequence: Synthetic peptide
LRYASYRT

SEQ ID NO: 503 - protein, 9 aa, synthetic construct
  REGION 1..9: note = Description of Artificial Sequence: Synthetic peptide
QQWNYPRIT

SEQ ID NO: 504 - protein, 9 aa, synthetic construct
  REGION 1..9: note = Description of Artificial Sequence: Synthetic peptide
QHYYSWPPT

SEQ ID NO: 505 - protein, 9 aa, synthetic construct
  REGION 1..9: note = Description of Artificial Sequence: Synthetic peptide
QQINGWPYT

SEQ ID NO: 506 - protein, 9 aa, synthetic construct
  REGION 1..9: note = Description of Artificial Sequence: Synthetic peptide
RQHYSSPPT

SEQ ID NO: 507 - protein, 9 aa, synthetic construct
  REGION 1..9: note = Description of Artificial Sequence: Synthetic peptide
QQYYTYPLT

SEQ ID NO: 508 - protein, 9 aa, synthetic construct
  REGION 1..9: note = Description of Artificial Sequence: Synthetic peptide
AQNRELPFT

SEQ ID NO: 509 - protein, 9 aa, synthetic construct
  REGION 1..9: note = Description of Artificial Sequence: Synthetic peptide
LHHYGTPYT

SEQ ID NO: 510 - protein, 9 aa, synthetic construct
  REGION 1..9: note = Description of Artificial Sequence: Synthetic peptide
QQGSSNPLT

SEQ ID NO: 511 - protein, 9 aa, synthetic construct
  REGION 1..9: note = Description of Artificial Sequence: Synthetic peptide
HQSYTTPHT

SEQ ID NO: 512 - protein, 9 aa, synthetic construct
  REGION 1..9: note = Description of Artificial Sequence: Synthetic peptide
QQWDNNPYT

SEQ ID NO: 513 - protein, 9 aa, synthetic construct
  REGION 1..9: note = Description of Artificial Sequence: Synthetic peptide
LQPNSYPWT

SEQ ID NO: 514 - protein, 9 aa, synthetic construct
  REGION 1..9: note = Description of Artificial Sequence: Synthetic peptide
QHYDDLPLT

SEQ ID NO: 515 - protein, 9 aa, synthetic construct
  REGION 1..9: note = Description of Artificial Sequence: Synthetic peptide
QQYSSYPLR

SEQ ID NO: 516 - protein, 9 aa, synthetic construct
  REGION 1..9: note = Description of Artificial Sequence: Synthetic peptide
RQLSSNPLT

SEQ ID NO: 517 - protein, 9 aa, synthetic construct
  REGION 1..9: note = Description of Artificial Sequence: Synthetic peptide
QQINSWPLT

SEQ ID NO: 518 - protein, 10 aa, synthetic construct
  REGION 1..10: note = Description of Artificial Sequence: Synthetic peptide
QQWSSYPPIA

SEQ ID NO: 519 - protein, 9 aa, synthetic construct
  REGION 1..9: note = Description of Artificial Sequence: Synthetic peptide
QKYSSTPYT

SEQ ID NO: 520 - protein, 9 aa, synthetic construct
  REGION 1..9: note = Description of Artificial Sequence: Synthetic peptide
QHWSSFPLT

SEQ ID NO: 521 - protein, 9 aa, synthetic construct
  REGION 1..9: note = Description of Artificial Sequence: Synthetic peptide
QQSYSTPLT

SEQ ID NO: 522 - protein, 9 aa, synthetic construct
  REGION 1..9: note = Description of Artificial Sequence: Synthetic peptide
QQYGSSPPT

SEQ ID NO: 523 - protein, 9 aa, synthetic construct
  REGION 1..9: note = Description of Artificial Sequence: Synthetic peptide
QQYGSSPLT

SEQ ID NO: 524 - protein, 9 aa, synthetic construct
  REGION 1..9: note = Description of Artificial Sequence: Synthetic peptide
QQYYSTPLT

SEQ ID NO: 525 - protein, 9 aa, synthetic construct
  REGION 1..9: note = Description of Artificial Sequence: Synthetic peptide
QQANSFPIT

SEQ ID NO: 526 - protein, 9 aa, synthetic construct
  REGION 1..9: note = Description of Artificial Sequence: Synthetic peptide
QQYGSSPLT

SEQ ID NO: 527 - protein, 9 aa, synthetic construct
  REGION 1..9: note = Description of Artificial Sequence: Synthetic peptide
QQSYSTPLT

SEQ ID NO: 528 - protein, 9 aa, synthetic construct
  REGION 1..9: note = Description of Artificial Sequence: Synthetic peptide
QQYGSSPLT

SEQ ID NO: 529 - protein, 8 aa, synthetic construct
  REGION 1..8: note = Description of Artificial Sequence: Synthetic peptide
QQYGNSYT

SEQ ID NO: 530 - protein, 9 aa, synthetic construct
  REGION 1..9: note = Description of Artificial Sequence: Synthetic peptide
QQYGSSPPT

SEQ ID NO: 531 - protein, 9 aa, synthetic construct
  REGION 1..9: note = Description of Artificial Sequence: Synthetic peptide
QQSYSTPIT

SEQ ID NO: 532 - protein, 9 aa, synthetic construct
  REGION 1..9: note = Description of Artificial Sequence: Synthetic peptide
QQDYRSPLT

SEQ ID NO: 533 - protein, 9 aa, synthetic construct
  REGION 1..9: note = Description of Artificial Sequence: Synthetic peptide
QQYNSYPLT

SEQ ID NO: 534 - protein, 9 aa, synthetic construct
  REGION 1..9: note = Description of Artificial Sequence: Synthetic peptide
QQWNSNPLT

SEQ ID NO: 535 - protein, 9 aa, synthetic construct
  REGION 1..9: note = Description of Artificial Sequence: Synthetic peptide
QNGHSFPLT

SEQ ID NO: 536 - protein, 9 aa, synthetic construct
  REGION 1..9: note = Description of Artificial Sequence: Synthetic peptide
QQGQSYPLT

SEQ ID NO: 537 - protein, 9 aa, synthetic construct
  REGION 1..9: note = Description of Artificial Sequence: Synthetic peptide
QQYNSYPLT

SEQ ID NO: 538 - protein, 11 aa, synthetic construct
  REGION 1..11: note = Description of Artificial Sequence: Synthetic peptide
GTWDTSLSAGV

SEQ ID NO: 539 - protein, 11 aa, synthetic construct
  REGION 1..11: note = Description of Artificial Sequence: Synthetic peptide
GTWDASLSTWV

SEQ ID NO: 540 - protein, 113 aa, synthetic construct
  REGION 1..113: note = Description of Artificial Sequence: Synthetic polypeptide
QVQLQESGPGLVKPSGTLSLTCAVSGGSISSSNWWSWVRQPPGKGLEWIGEIYPSGNTYYNPSLKSRVTISVDKSKNQFSLKLNSVTAADTAVYYCARDEGYWGQGTLVTVSS

SEQ ID NO: 541 - protein, 115 aa, synthetic construct
  REGION 1..115: note = Description of Artificial Sequence: Synthetic polypeptide
EVQLLESGAEVKKPGASVKVSCKASGYTFTGYYMHWVRQAPGQGLEWMGWINPNSGGTNYAQKFQGRVTMTRDTSISTAYMELSSLRSEDTAVYYCARGGPFDYWGQGTMVTVSS

SEQ ID NO: 542 - protein, 117 aa, synthetic construct
  REGION 1..117: note = Description of Artificial Sequence: Synthetic polypeptide
EVQLLESGAEVKKPGASVKASCKASGYTFTGYYMHWVRQAPGQGLEWMGWINPNSGGTNYAQKFQGRVTMTRDTSISTAYMELSRLRSDDTAVYYCARVYSYGFDYWGQGTLVTVSS

SEQ ID NO: 543 - protein, 116 aa, synthetic construct
  REGION 1..116: note = Description of Artificial Sequence: Synthetic polypeptide
AGQLQESGGGLVKPGGSLRPSCAASGFTFSDYYMSWIRQAPGKGLEWVSYISSSGSTIYYADSVKGRFTISRDNAKNSLYLQMNSLRAEDTAVYYCARDNWAFDLWGQGTLVTVSS

SEQ ID NO: 544 - protein, 119 aa, synthetic construct
  REGION 1..119: note = Description of Artificial Sequence: Synthetic polypeptide
GGALVQSGGGLVQPGGSLRLSCAASGFTFSSYSMNWVRQATGKGLEWVSSISSSSSYIYYADSVKGRFTISRDNAKNSLHLQMNSLRDEDTAVYYCARTWYSSSPFDYWGQGTLVTVSS

SEQ ID NO: 545 - protein, 120 aa, synthetic construct
  REGION 1..120: note = Description of Artificial Sequence: Synthetic polypeptide
EVQLLESGGGLVKPGGSPRLSCAASGFTFSNYSMNWVRQAPGKGLEWVSSISSSSSYIYYADSVNGRFTISRDYAKNSLYLQMNSLRAEDTAVCYCARDSGSYYDWFDPWGQGTLVTVSS

SEQ ID NO: 546 - protein, 116 aa, synthetic construct
  REGION 1..116: note = Description of Artificial Sequence: Synthetic polypeptide
QVQLVQSGGGLVQPGGSLRLSCAASGFTFSSYAMSWVRRAPGKGLEWVSAISGSGGSTYYADSVKGRFTISRDNSKNTLYLQMNSLRAEDTAVYYCAKYSNYFDYWGQGTLVTVSS

SEQ ID NO: 547 - protein, 121 aa, synthetic construct
  REGION 1..121: note = Description of Artificial Sequence: Synthetic polypeptide
EVQLQQPGPGLVKPSLSLSHTCSVTGYSITSGYYWNWIRQFPGKKLEWMGYISYDGSNNYNPSLKNRTSITRDTSKNQFFLKLSSVTTDDTATYYCARVHHYYGSSYFDYWGQGTTLTVSS

SEQ ID NO: 548 - protein, 121 aa, synthetic construct
  REGION 1..121: note = Description of Artificial Sequence: Synthetic polypeptide
EVQLQQSGPGLVKPSQSLSLTCSVTGYSITSGYYWNWIRQFPGKKLEWMGYIRYDGSNNYNPSLKNRISITRDTSKNQFFLKLNSVTTDDTATYYCARVHHYYGSSYFDYWGQGTTLTVSS

SEQ ID NO: 549 - protein, 121 aa, synthetic construct
  REGION 1..121: note = Description of Artificial Sequence: Synthetic polypeptide
EVQLQQSGPGLVKPSQSLSLTCSVTGYSITSGYYWNWIRQFPGNKLEWMGYISYDGSNNYNPSLKNRISITRDTSKNQFFLKLNSVTTEDTATYYCARDPYYYGSSYFDYWGQGTTLTVSS

SEQ ID NO: 550 - protein, 120 aa, synthetic construct
  REGION 1..120: note = Description of Artificial Sequence: Synthetic polypeptide
EVKLVESEGGLVQPGSSMKLSCTASGFTFSDYYMAWVRQVPEKGLEWVANINYDGSSTYYLDSLKSRFTISRDNAKNILYLQMSSLKSEDIATYYCAREYYYGSSSFAYWGQGTTLTVSS

SEQ ID NO: 551 - protein, 120 aa, synthetic construct
  REGION 1..120: note = Description of Artificial Sequence: Synthetic polypeptide
EVNPVESEGGLVQPGSSMKLSCTASGFTFNDYYMAWVRQVPEKGLEWVANINYDGSSTYYLDSLKSRFIISRDNAKNILYLQMSSLKSEDTATYYCAREYYYGSSSFAYWGQGTLVTVSA

SEQ ID NO: 552 - protein, 114 aa, synthetic construct
  REGION 1..114: note = Description of Artificial Sequence: Synthetic polypeptide
GGEVVESGGGLVKPGGSLKLSCAASGFTFSDFGMHWVRQAPEKGLEWVAYISSGSSTIYYADTVKGRFTISRDNAKNTLFLQMTSLRSEDTAMYYCARREFAYWGQGTLVTVSA

SEQ ID NO: 553 - protein, 120 aa, synthetic construct
  REGION 1..120: note = Description of Artificial Sequence: Synthetic polypeptide
EVKLEESGGGLVKPGGSLKLSCAVSGFTFSDYGMHWVRQAPEKGLEWVAYISSGSGTIYYEDTVKGRFTISRDNAKNTLFLQMTSLRSEGTAIYYCARPNYHGSSPFAYWGQGTLVTVSA

SEQ ID NO: 554 - protein, 124 aa, synthetic construct
  REGION 1..124: note = Description of Artificial Sequence: Synthetic polypeptide
EVQLVESGGGLVKPGGSLRLSCAASGFTFSTYGMSWVRQAPGKGLEWVSGISGSGGSTNYADSVKGRFTISRDNSKNTLYLQMNSLRAEDTAVYYCAAARGKWRWRLGRRYDYWGQGTLVTVSS

SEQ ID NO: 555 - protein, 124 aa, synthetic construct
  REGION 1..124: note = Description of Artificial Sequence: Synthetic polypeptide
EVQLVESGGGLVKPGGSLRLSCAASGFTFNNYAMNWVRQAPGKGLEWVSSISGSGGDTYYADSVKGRFTISRDNSKNTLYLQMNSLRAEDTAVYYCAAARGYWRWRLLRRYDYWGQGTLVTVSS

SEQ ID NO: 556 - protein, 118 aa, synthetic construct
  REGION 1..118: note = Description of Artificial Sequence: Synthetic polypeptide
EVQLVESGGGLVKPGGSLRLSCAASGFNIGNTYMHWFRQAPGKGLEWVGVINPNYGTTRYNDSVKGRFTISRDNSKNTLYLQMNSLRAEDTAVYYCARDYGSSSFDYWGQGTLVTVSS

SEQ ID NO: 557 - protein, 124 aa, synthetic construct
  REGION 1..124: note = Description of Artificial Sequence: Synthetic polypeptide
EVQLVESGGGLVKPGGSLRLSCAASGFSFSGYAMSWVRQAPGKGLEWVSVISGSGGSTNYADSVKGRFTISRDNSKNTLYLQMNSLRAEDTAVYYCAAARGYPRWRLGRRYDYWGQGTLVTVSS

SEQ ID NO: 558 - protein, 124 aa, synthetic construct
  REGION 1..124: note = Description of Artificial Sequence: Synthetic polypeptide
EVQLVESGGGLVKPGGSLRLSCAASGFTFSGYAMNWVRQAPGKGLEWVSTISGSGASTYYADSVKGRFTISRDNSKNTLYLQMNSLRAEDTAVYYCAAARGYKRWRLGRRYDYWGQGTLVTVSS

SEQ ID NO: 559 - protein, 121 aa, synthetic construct
  REGION 1..121: note = Description of Artificial Sequence: Synthetic polypeptide
EVQLVESGGGLVKPGGSLRLSCAASGFAFSNYWMNWVRQAPGKGLEWVGWFYPKSGSIKYNDSVKGRFTISRDNSKNTLYLQMNSLRAEDTAVYYCTGLYGYDLHWYFDVWGQGTLVTVSS

SEQ ID NO: 560 - protein, 122 aa, synthetic construct
  REGION 1..122: note = Description of Artificial Sequence: Synthetic polypeptide
QVQLVQSGAEVKKPGASVKVSCKASGGSISSGGYYWNWVRQATGQGLEWMGWMNPNSGNRGSAQKFQGRVTMTRDTSTSTVYMELSSLRSEDTAVYYCARDEVAAAGLFDYWGQGTLVTVSS

SEQ ID NO: 561 - protein, 124 aa, synthetic construct
  REGION 1..124: note = Description of Artificial Sequence: Synthetic polypeptide
EVQLVESGGGLVKPGGSLRLSCAASGYTFTEYITHWVRQAPGKGLEWVGMIHPSSGSISYNDSVKGRFTISRDNSKNTLYLQMNSLRAEDTAVYYCARHEVEYYGPSSSWFAYWGQGTLVTVSS

SEQ ID NO: 562 - protein, 124 aa, synthetic construct; identical to SEQ ID NO: 554
  REGION 1..124: note = Description of Artificial Sequence: Synthetic polypeptide

SEQ ID NO: 563 - protein, 118 aa, synthetic construct; identical to SEQ ID NO: 556
  REGION 1..118: note = Description of Artificial Sequence: Synthetic polypeptide

SEQ ID NO: 564 - protein, 121 aa, synthetic construct
  REGION 1..121: note = Description of Artificial Sequence: Synthetic polypeptide
EVQLVESGGGLVKPGGSLRLSCAASGFTFGNYWMNWVRQAPGKGLEWVGRIDPANGDTKYADSVKGRFTISRDNSKNTLYLQMNSLRAEDTAVYYCAREGDNSNYYAMDYWGQGTLVTVSS

SEQ ID NO: 565 - protein, 124 aa, synthetic construct
  REGION 1..124: note = Description of Artificial Sequence: Synthetic polypeptide
EVQLLESGGGLVQPGGSLRLSCAASGFTFSTYAMGWVRQAPGKGLEWVSGISGSAGSTYYADSVKGRFTISRDNSKNTLYLQMNSLRAEDTAVYYCAAARGHWRWRLGRRYDYWGQGTLVTVSS

SEQ ID NO: 566 - protein, 124 aa, synthetic construct
  REGION 1..124: note = Description of Artificial Sequence: Synthetic polypeptide
EVQLVESGGGLVKPGGSLRLSCAASGFTFSSYAMAWVRQAPGKGLEWVSAISGSGGSTYFADSVKGRFTISRDNSKNTLYLQMNSLRAEDTAVYYCAAARGYWRWRLWRRYDYWGQGTLVTVSS

SEQ ID NO: 567 - protein, 124 aa, synthetic construct
  REGION 1..124: note = Description of Artificial Sequence: Synthetic polypeptide
EVQLVESGGGLVKPGGSLRLSCAASGFTFSSQAMSWVRQAPGKGLEWVSSISGSGVSTYYADSVKGRFTISRDNSKNTLYLQMNSLRAEDTAVYYCAAARGRWRWRLGRRYDYWGQGTLVTVSS

SEQ ID NO: 568 - protein, 119 aa, synthetic construct
  REGION 1..119: note = Description of Artificial Sequence: Synthetic polypeptide
EVQLVESGGGLVKPGGSLRLSCAASGYSFTGYDISWVRQAPGKGLEWVARIYPGSGNTYYDDSVKGRFTISRDNSKNTLYLQMNSLRAEDTAVYYCAREDDYGWYFGVWGQGTLVTVSS

SEQ ID NO: 569 - protein, 123 aa, synthetic construct
  REGION 1..123: note = Description of Artificial Sequence: Synthetic polypeptide
EVQLVESGGGLVKPGGSLRLSCAASGYRLTGYWIEWVRQAPGKGLEWVGRIDPASGDTTYADSVKGRFTISRDNSKNTLYLQMNSLRAEDTAVYYCARHEDPIYYGNYVFAYWGQGTLVTVSS

SEQ ID NO: 570 - protein, 121 aa, synthetic construct
  REGION 1..121: note = Description of Artificial Sequence: Synthetic polypeptide
EVQLVESGGGLVKPGGSLRLSCAASGYLFTDYNMNWVRQAPGKGLEWVGWIYPGTGNTYNDSVKGRFTISRDNSKNTLYLQMNSLRAEDTAVYYCTALYYGSSWERYFDVWGQGTLVTVSS

SEQ ID NO: 571 - protein, 120 aa, synthetic construct
  REGION 1..120: note = Description of Artificial Sequence: Synthetic polypeptide
EVQLVESGGGLVKPGGSLRLSCAASGFTFIDYGMHWVRQAPGKGLEWVGVINPNYGTTRYNDSVKGRFTISRDNSKNTLYLQMNSLRAEDTAVYYCARQGSNYGGYFDVWGQGTLVTVSS

SEQ ID NO: 572 - protein, 124 aa, synthetic construct
  REGION 1..124: note = Description of Artificial Sequence: Synthetic polypeptide
EVQLVESGGGLVKPGGSLRLSCAASGFPFSSYAMTWVRQAPGKGLEWVSGISGSGGRTYYADSVKGRFTISRDNSKNTLYLQMNSLRAEDTAVYYCAAARGYWRWRLGRRADYWGQGTLVTVSS

SEQ ID NO: 573 - protein, 123 aa, synthetic construct
  REGION 1..123: note = Description of Artificial Sequence: Synthetic polypeptide
EVQLVESGGGLVKPGGSLRLSCAASGFNFNTYAMNWVRQAPGKGLEWVGVIYPGNSDTTYNDSVKGRFTISRDNSKNTLYLQMNSLRAEDTAVYYCTGVIYYYGSSDYTLDYWGQGTLVTVSS

SEQ ID NO: 574 - protein, 124 aa, synthetic construct; identical to SEQ ID NO: 554
  REGION 1..124: note = Description of Artificial Sequence: Synthetic polypeptide

SEQ ID NO: 575 - protein, 118 aa, synthetic construct; identical to SEQ ID NO: 556
  REGION 1..118: note = Description of Artificial Sequence: Synthetic polypeptide

SEQ ID NO: 576 - protein, 117 aa, synthetic construct
  REGION 1..117: note = Description of Artificial Sequence: Synthetic polypeptide
EVQLVESGGGLVKPGGSLRLSCAASGYTFTSYWVHWVRQAPGKGLEWVANINHDGSNTYYLDSVKGRFTISRDNSKNTLYLQMNSLRAEDTAVYYCASSMITRFAYWGQGTLVTVSS

SEQ ID NO: 577 - protein, 119 aa, synthetic construct
  REGION 1..119: note = Description of Artificial Sequence: Synthetic polypeptide
EVQLVESGGGLVKPGGSLRLSCAASGFSLTSYNIDWVRQAPGKGLEWVGGVDPETDDTAYNDSVKGRFTISRDNSKNTLYLQMNSLRAEDTAVYYCANYYYGSSAFAYWGQGTLVTVSS

SEQ ID NO: 578 - protein, 120 aa, synthetic construct
  REGION 1..120: note = Description of Artificial Sequence: Synthetic polypeptide
EVQLVESGGGLVKPGGSLRLSCAASGFTFSNYYMSWVRQAPGKGLEWVGDINPNNGGTTYNDSVKGRFTISRDNSKNTLYLQMNSLRAEDTAVYYCTKAYYSNYGVMYFWGQGTLVTVSS

SEQ ID NO: 579 - protein, 124 aa, synthetic construct
  REGION 1..124: note = Description of Artificial Sequence: Synthetic polypeptide
EVQLVESGGGLVKPGGSLRLSCAASGFNFRSYAMSWVRQAPGKGLEWVSVISGGGGSTYYADSVKGRFTISRDNSKNTLYLQMNSLRAEDTAVYYCAAARGGWRWRLGRRYDYWGQGTLVTVSS

SEQ ID NO: 580 - protein, 123 aa, synthetic construct
  REGION 1..123: note = Description of Artificial Sequence: Synthetic polypeptide
EVQLVESGGGLVKPGGSLRLSCAASGFSLSIYGISWVRQAPGKGLEWVGDISPGSGSTNYNDSVKGRFTISRDNSKNTLYLQMNSLRAEDTAVYYCAGPYYYGSSRYYAMDYWGQGTLVTVSS

SEQ ID NO: 581 - protein, 120 aa, synthetic construct
  REGION 1..120: note = Description of Artificial Sequence: Synthetic polypeptide
EVQLVESGGGLVKPGGSLRLSCAASGYTFTSYNINWVRQAPGKGLEWVATISSGGDSIYYADSVKGRFTISRDNSKNTLYLQMNSLRAEDTAVYYCERDYYGSSWHFDVWGQGTLVTVSS

SEQ ID NO: 582 - protein, 124 aa, synthetic construct
  REGION 1..124: note = Description of Artificial Sequence: Synthetic polypeptide
EVQLVESGGGLVKPGGSLRLSCAASGFTFSSYDMYWVRQAPGKGLEWVASISDGGSYTYYVDSVKGRFTISRDNSKNTLYLQMNSLRAEDTAVYYCARYIWYYGSSWSWYFDAWGQGTLVTVSS

SEQ ID NO: 583 - protein, 116 aa, synthetic construct
  REGION 1..116: note = Description of Artificial Sequence: Synthetic polypeptide
EVQLVESGGGVVQPGRSLRLSCAASGFTFSAYDIYWVRQPTGKGLEWVSAIGTAGDTYYPGSVKGRFIISRESAKNSVYLQMNSLRAGDTAVYYCAVGYNWIFDLWGQGTLVTVSS

SEQ ID NO: 584 - protein, 117 aa, synthetic construct
  REGION 1..117: note = Description of Artificial Sequence: Synthetic polypeptide
EVQLVESGGGLVKPGGSLRLSCAASGYSFTGYDISWFRQAPGKGLEWVGEILPGSGGTNYNDSVKGRFTISRDNSKNTLYLQMNSLRAEDTAVYYCTSGNYDAMDYWGQGTLVTVSS

SEQ ID NO: 585 - protein, 121 aa, synthetic construct
  REGION 1..121: note = Description of Artificial Sequence: Synthetic polypeptide
QVQLVQSGAEVKKPGASVKVSCKASGGYISSSNWWSWVRQATGQGLEWMANIEQDGSEKNYVQKFQGRVTMTRDTSTSTVYMELSSLRSEDTAVYYCARGEYSRLWYFDLWGQGTLVTVSS

SEQ ID NO: 586 - protein, 116 aa, synthetic construct
  REGION 1..116: note = Description of Artificial Sequence: Synthetic polypeptide
EVQLVESGGGLVKPGGSLRLSCAASGTFTDYYMKWVRQAPGKGLEWVGEILPGSGGTNYNDSVKGRFTISRDNSKNTLYLQMNSLRAEDTAVYYCARPYDYDFDYWGQGTLVTVSS

SEQ ID NO: 587 - protein, 119 aa, synthetic construct
  REGION 1..119: note = Description of Artificial Sequence: Synthetic polypeptide
EVQLVESGGGLVKPGGSLRLSCAASGYTFTSSWMHWARQAPGKGLEWVGWLYPRDGSTEYNDSVKGRFTISRDNSKNTLYLQMNSLRAEDTAVYYCITTVVADWYFDVWGQGTLVTVSS

SEQ ID NO: 588 - protein, 121 aa, synthetic construct
  REGION 1..121: note = Description of Artificial Sequence: Synthetic polypeptide
EVQLVESGGGLVKPGGSLRLSCAASGYTFNDDYTNWVRQAPGKGLEWVGNINPNNGAMIYNDSVKGRFTISRDNSKNTLYLQMNSLRAEDTAVYYCARKGDGGSYAAMDYWGQGTLVTVSS

SEQ ID NO: 589 - protein, 119 aa, synthetic construct
  REGION 1..119: note = Description of Artificial Sequence: Synthetic polypeptide
EVQLVESGGGLVKPGGSLRLSCAASGYSFTGYDISWVRQAPGKGLEWVGWIYPKDGSTKYNDSVKGRFTISRDNSKNTLYLQMNSLRAEDTAVYYCITTVVADWYFDVWGQGTLVTVSS

SEQ ID NO: 590 - protein, 120 aa, synthetic construct
  REGION 1..120: note = Description of Artificial Sequence: Synthetic polypeptide
EVQLVESGGGLVKPGGSLRLSCAASGYTFNDYYINWVRQAPGKGLEWVGDINPNNGANIYNDSVKGRFTISRDNSKNTLYLQMNSLRAEDTAVYYCARNYGSSYYALDYWGQGTLVTVSS

SEQ ID NO: 591 - protein, 120 aa, synthetic construct
  REGION 1..120: note = Description of Artificial Sequence: Synthetic polypeptide
EVQLVESGGGLVKPGGSLRLSCAASGYTFNDYYINWVRQAPGKGLEWVGDINPNNGGTTYNDSVKGRFTISRDNSKNTLYLQMNSLRAEDTAVYYCARQGSNYGGYFDVWGQGTLVTVSS

SEQ ID NO: 592 - protein, 116 aa, synthetic construct
  REGION 1..116: note = Description of Artificial Sequence: Synthetic polypeptide
EVQLVESGGGLVKPGGSLRLSCAASGFNIIDDYMHWVRQAPGKGLEWVGMITDTGEPTDADSVKGRFTISRDNSKNTLYLQMNSLRAEDTAVYYCVYDYIYAMDYWGQGTLVTVSS

SEQ ID NO: 593 - protein, 120 aa, synthetic construct
  REGION 1..120: note = Description of Artificial Sequence: Synthetic polypeptide
EVQLVESGGGLVKPGGSLRLSCAASGYTFTDYDMYWVRQAPGKGLEWVGRIDPANGDTKYADSVKGRFTISRDNSKNTLYLQMNSLRAEDTAVYYCARGDYGSSYAMDYWGQGTLVTVSS

SEQ ID NO: 594 - protein, 119 aa, synthetic construct
  REGION 1..119: note = Description of Artificial Sequence: Synthetic polypeptide
EVQLVESGGGLVKPGGSLRLSCAASGYEFSSSWMNWVRQAPGKGLEWVGWIYPGTGNTNYNDSVKGRFTISRDNSKNTLYLQMNSLRAEDTAVYYCANYYYGSSAFAYWGQGTLVTVSS

SEQ ID NO: 595 - protein, 119 aa, synthetic construct
  REGION 1..119: note = Description of Artificial Sequence: Synthetic polypeptide
EVQLVESGGGLVKPGGSLRLSCAASGFTFSSYGLPWVRQAPGKGLEWVGAIDPGTGGTASNDSVKGRFTISRDNSKNTLYLQMNSLRAEDTAVYYCARIYYDYSAMDYWGQGTLVTVSS

SEQ ID NO: 596 - protein, 118 aa, synthetic construct
  REGION 1..118: note = Description of Artificial Sequence: Synthetic polypeptide
EVQLVESGGGLVKPGGSLRLSCAASGFIFSDFYMAWVRQAPGKGLEWVGRIDPEDGDEHADSVKGRFTISRDNSKNTLYLQMNSLRAEDTAVYYCARDYYGSSYLDYWGQGTLVTVSS

SEQ ID NO: 597 - protein, 123 aa, synthetic construct
  REGION 1..123: note = Description of Artificial Sequence: Synthetic polypeptide
EVQLVESGGGLVKPGGSLRLSCAASGFNIKDYYMHWVRQAPGKGLEWVGDINPNNGGTTYNDSVKGRFTISRDNSKNTLYLQMNSLRAEDTAVYYCARDYYGSFHRRWYFDVWGQGTLVTVSS

SEQ ID NO: 598 - protein, 122 aa, synthetic construct
  REGION 1..122: note = Description of Artificial Sequence: Synthetic polypeptide
EVQLVESGGGLVKPGGSLRLSCAASGYTFTDYNIDWVRQAPGKGLEWVGDININNGGTTYNDSVKGRFTISRDNSKNTLYLQMNSLRAEDTAVYYCARDYHGSSFYWYFDVWGQGTLVTVSS

SEQ ID NO: 599 - protein, 120 aa, synthetic construct
  REGION 1..120: note = Description of Artificial Sequence: Synthetic polypeptide
EVQLVESGGGLVKPGGSLRLSCAASGYTFTEYITHWVRQAPGKGLEWVANINFDGSSTYYLDSVKGRFTISRDNSKNTLYLQMNSLRAEDTAVYYCARYYDSSYYAMDYWGQGTLVTVSS

SEQ ID NO: 600 - protein, 119 aa, synthetic construct
  REGION 1..119: note = Description of Artificial Sequence: Synthetic polypeptide
QVQLVQSGAEVKKPGASVKVSCKASGFTFSTYIMSWVRQATGQGLEWMGIIYPGDTDTRYSQKFQGRVTMTRDTSTSTVYMELSSLRSEDTAVYYCARGIAVAGTFDYWGQGTLVTVSS

SEQ ID NO: 601 - protein, 121 aa, synthetic construct
  REGION 1..121: note = Description of Artificial Sequence: Synthetic polypeptide
EVQLVESGGGLVKPGGSLRLSCAASGYTFTNYLIEWVRQAPGKGLEWVGDINPNNGGTYYNDSVKGRFTISRDNSKNTLYLQMNSLRAEDTAVYYCVRHALLWYYYAMDYWGQGTLVTVSS

SEQ ID NO: 602 - protein, 118 aa, synthetic construct
  REGION 1..118: note = Description of Artificial Sequence: Synthetic polypeptide
QVQLVQSGAEVKKPGASVKVSCKASGFTFSDHYMTWVRQATGQGLEWMGWMNPNSGITGYAQKFQGRVTMTRDTSTSTVYMELSSLRSEDTAVYYCARVSYSGSLHYWGQGTLVTVSS

SEQ ID NO: 603 - protein, 117 aa, synthetic construct
  REGION 1..117: note = Description of Artificial Sequence: Synthetic polypeptide
QVQLVQSGAEVKKPGASVKVSCKASGFTFDDYAMHWVRQATGQGLEWMGVINTNTGNPTYAQKFQGRVTMTRDTSTSTVYMELSSLRSEDTAVYYCARSNWNYFDYWGQGTLVTVSS

SEQ ID NO: 604 - protein, 119 aa, synthetic construct
  REGION 1..119: note = Description of Artificial Sequence: Synthetic polypeptide
EVQLVESGGGLVKPGGSLRLSCAASGSAFSASWMNLVRQAPGKGLEWVGWVDPDNGDTEYADSVKGRFTISRDNSKNTLYLQMNSLRAEDTAVYYCSKPRDSGPSFASWGQGTLVTVSS

SEQ ID NO: 605 - protein, 124 aa, synthetic construct
  REGION 1..124: note = Description of Artificial Sequence: Synthetic polypeptide
EVQLVESGGGLVKPGGSLRLSCAASGFTFSSYDMYWVRQAPGKGLEWVGWIYPKDGSTKYNDSVKGRFTISRDNSKNTLYLQMNSLRAEDTAVYYCARSRGYYYGSSYGYYDVWGQGTLVTVSS

SEQ ID NO: 606 - protein, 115 aa, synthetic construct
  REGION 1..115: note = Description of Artificial Sequence: Synthetic polypeptide
EVQLVESGGGLVKPGGSLRLSCAASGHTITSYGINWVRQAPGKGLEWVGEILPGSGTSDYNDSVKGRFTISRDNSKNTLYLQMNSLRAEDTAVYYCATNWGFAYWGQGTLVTVSS

SEQ ID NO: 607 - protein, 119 aa, synthetic construct
  REGION 1..119: note = Description of Artificial Sequence: Synthetic polypeptide
EVQLVESGGGLVKPGGSLRLSCAASGYTFSGYTMHWVRQAPGKGLEWVGEIDPSDSFANYNDSVKGRFTISRDNSKNTLYLQMNSLRAEDTAVYYCARDYGSSYEFTYWGQGTLVTVSS

SEQ ID NO: 608 - protein, 120 aa, synthetic construct
  REGION 1..120: note = Description of Artificial Sequence: Synthetic polypeptide
QVQLVQSGAEVKKPGASVKVSCKASGGYISSSNWWSWVRQATGQGLEWMGRIKTKTDGGTIDYAQKFQGRVTMTRDTSTSTVYMELSSLRSEDTAVYYCAKGYSGSVDYWGQGTLVTVSS

SEQ ID NO: 609 - protein, 116 aa, synthetic construct
  REGION 1..116: note = Description of Artificial Sequence: Synthetic polypeptide
EVQLVESGGGLVKPGGSLRLSCAASGSNIKDYYIHWVRQAPGKGLEWVATISDGGSYIFDDSVKGRFTISRDNSKNTLYLQMNSLRAEDTAVYYCARDATGTFAYWGQGTLVTVSS

SEQ ID NO: 610 - protein, 122 aa, synthetic construct
  REGION 1..122: note = Description of Artificial Sequence: Synthetic polypeptide
QVQLQESGPGLVKPSGTLSLTCAVSGGSISSSNWWSWVRQPPGRGLEWIGEIYHSGSTNYNPSLKSRVTISVDKPKNQFSLKLSSVTAADTAVYYCAREVVSGMIGTVFDYWGQGTLVTVSS

SEQ ID NO: 611 - protein, 116 aa, synthetic construct
  REGION 1..116: note = Description of Artificial Sequence: Synthetic polypeptide
QVQLVQSGGGLVQPGGSLRLSCAVSGFTISTYDIYWVRQATGKGLEWVSAIGTAGDTYYPDSVRGRFTISREDARNSLYLQMNSLRTGDTAVYYCATGYNWIFDYWGQGTLVTVSS

SEQ ID NO: 612 - protein, 116 aa, synthetic construct
  REGION 1..116: note = Description of Artificial Sequence: Synthetic polypeptide
QVQLVQSGGGLVQPGGSLRLSCAASGFTVSTYDIYWVRQTTGKGLELVSAIGTAGDTYYPDSVKGRFTISRENARNSLYLQMNSLRAGDTAVYYCAVGYNWIFDFWGQGTLVTVSS

SEQ ID NO: 613 - protein, 116 aa, synthetic construct
  REGION 1..116: note = Description of Artificial Sequence: Synthetic polypeptide
EVQLVESGGGLVQPGGSLRLSCAASGFTFTTYDMYWVRQTTGKGLEWVSAIGTAGDTYYPDSVKGRFTISRESAKNSLYLQMNSLRAGDTAVYYCTVGYNWIFDFWGHGTLVTVSS

SEQ ID NO: 614 - protein, 122 aa, synthetic construct
  REGION 1..122: note = Description of Artificial Sequence: Synthetic polypeptide
QVQLQESGPGLVKPSGTLSLTCAVSGGSISSSNWWSWVRQPPGKGLEWIGEIYHSGNTNYNPSLKSRVTMSVDKSKNQFSLNLHSVTAADTAVYYCAREVVSGMIGTIFDYWGQGTLVTVSS

SEQ ID NO: 615 - protein, 116 aa, synthetic construct
  REGION 1..116: note = Description of Artificial Sequence: Synthetic polypeptide
EVQLVESGGGLVQPGGSLRLSCAASGFTFSSYDMYWVRQPTGKGLEWVSAIGTAGDTYYSGSVKGRFTISRESAKNSLYLQMNSLRAGDTAVYYCAVGYNWIFDFWGQGTLVTVSS

SEQ ID NO: 616 - protein, 122 aa, synthetic construct; identical to SEQ ID NO: 614
  REGION 1..122: note = Description of Artificial Sequence: Synthetic polypeptide

SEQ ID NO: 617 - protein, 116 aa, synthetic construct
  REGION 1..116: note = Description of Artificial Sequence: Synthetic polypeptide
EVQLLESGGGLVQPGGSLRLSCAASGFTFSAYDIYWVRQPTGKGLEWVSAIGTAGDTYYPGSVKGRFIISRESAKNSVYLQMNSLRAGDTAVYYCAVGYNWVFDLWGQGTLVTVSS

SEQ ID NO: 618 - protein, 118 aa, synthetic construct
  REGION 1..118: note = Description of Artificial Sequence: Synthetic polypeptide
QVQLQESGGGVVRPGGSLRLSCAASGFTFDDYGMSWVRQVPGKGLEWVSGITWNGDRSGYADSVKGRFTISRDNAKNSLYLQMNSLRAEDTALYYCVRDGLTGIFDYWGQGTLVTVSS

SEQ ID NO: 619 - protein, 116 aa, synthetic construct
  REGION 1..116: note = Description of Artificial Sequence: Synthetic polypeptide
QVQLVQSGGGLVKPGGSLRLSCAASGFTISTYDIYWVRQATGKGLEWVSAIGTAGDTYYPGSVKGRFTISRENAKNSLYLQMNSLRAGDTAVYYCASGYNWIFDYWGQGTLVTVSS

SEQ ID NO: 620 - protein, 122 aa, synthetic construct
  REGION 1..122: note = Description of Artificial Sequence: Synthetic polypeptide
QVQLQESGPGLVKPSGTLSLTCAVSGGSISSSNWWSWVRQPPGKGLEWIGEIYHSGSTNYNPSLKSRVTISVDKSKNQFSLKLGSVTAADTAVYYCAREVVSGLYGTIFDYWGQGTLVTVSS

SEQ ID NO: 621 - protein, 116 aa, synthetic construct
  REGION 1..116: note = Description of Artificial Sequence: Synthetic polypeptide
EVQLQQSGPGLVKPSQSLSLTCSVTGYSITSGYYWNWIRQFPGNKLEWMGYISYGGSNDYNPSLKNRISITRDSSKNQFFLKLNSVTTEDTATYYCARDYDYFDYWGQGTTLTVSS

SEQ ID NO: 622 - protein, 112 aa, synthetic construct
  REGION 1..112: note = Description of Artificial Sequence: Synthetic polypeptide
EVQRVQSGAELVKPGASVKISCKASGYAFSSYWMNWVKQRPGKGLEWIGQIYPGDGDTNYNGKFEGKATLTADKSSSTAYMQLTSLTSDDSAVYYCARGAYWGQGTTLTVSS

SEQ ID NO: 623 - protein, 120 aa, synthetic construct
  REGION 1..120: note = Description of Artificial Sequence: Synthetic polypeptide
EVQLVESGGGLVKPGGSLRLSCAASGYTFTEYITHWVRQAPGKGLEWVATISGGGSYTNFPDSVKGRFTISRDNAKNTLFLQMTSLRSEDTAMYYCARPNYSGSSPFAYWGQGTLVTVSA

SEQ ID NO: 624 - protein, 123 aa, synthetic construct
  REGION 1..123: note = Description of Artificial Sequence: Synthetic polypeptide
EVQLQQSGPGLVAPSQSLSITCTVSGFSLTAYAISWVRQPPGKGLEWLGVIWTGGGTNYNSALKSRLSISKDNSKSQVFLKMNSLQTDDTARYYCARSRGYYYGSSYGYFDVWGTGTTVTVSS

SEQ ID NO: 625 - protein, 118 aa, synthetic construct
  REGION 1..118: note = Description of Artificial Sequence: Synthetic polypeptide
EVQLQESGPGLAKPSQTLPLTCSVIGYSITSDYWNWIRKFPGNKLEYMGYINYSGSTYYNPSLKSRISITRDTSKNQYYLQLNSVTTEDTATYYCTRKLDWDGYFDVWGTGTTVTVSS

SEQ ID NO: 626 - protein, 123 aa, synthetic construct
  REGION 1..123: note = Description of Artificial Sequence: Synthetic polypeptide
EVQLQQSEAELVRPGAPVKLSCTASGFNIKNTYMHWVKQRPEQGLEWIGRIDPANGNTKYAPKFQGKATITADTSSNTAYLQLSSLASEDSAVYFCARGSPYGYDGHYVMDYWGQGTSVTVSS

SEQ ID NO: 627 - protein, 155 aa, synthetic construct
  REGION 1..155: note = Description of Artificial Sequence: Synthetic polypeptide
EVQLVESGGGLVKPGGSLRLSCAASGFTFRTYGMHWVRQAPGKGLEWVAVISAEGSNKYYADSVKGRFTISRDNSKNTLYLQMNSLRAEDTAVYYCARDGRGSLPRPKGGFIGALSFHWPFGRWLGGSYGTYDSSEDSGGAFDIWGQGTLVTVSS

SEQ ID NO: 628 - protein, 155 aa, synthetic construct
  REGION 1..155: note = Description of Artificial Sequence: Synthetic polypeptide
QVQLVESGGGVVQPGRSLRLSCAASGFTFNNYGMHWVRQAPGKGLEWVAVISYGGSDKYYADSVKGRFTISRDNSKNTLYLQMNSLRAEDTAVYYCARDGRGSLPRPKGGFIGDLSFHWPFGRWLGKSYGTYDSSEDSGGAFDIWGQGTLVTVSS

SEQ ID NO: 629 - protein, 112 aa, synthetic construct
  REGION 1..112: note = Description of Artificial Sequence: Synthetic polypeptide
ELVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPRTFGQGTKVDIK

SEQ ID NO: 630 - protein, 108 aa, synthetic construct
  REGION 1..108: note = Description of Artificial Sequence: Synthetic polypeptide
ELTLTQSPAFMSATPGDKVNISCKASQDIDDDMNWYQQKPGEAAIFIIQEATTLVPGIPPRFSGSGYGTDFTLTINNIESEDAAYYFCLQHDNFPMYTFGQGTKLEIK

SEQ ID NO: 631 - protein, 113 aa, synthetic construct
  REGION 1..113: note = Description of Artificial Sequence: Synthetic polypeptide
ELVLTQSPDSLAVSLGERATFNCKSSQSVLYSSNNKNYLAWYQQKPGQPPKLLIYWASTRESGVPDRFSGSGSGTDFTLTISSLQAEDVAVYYCQQYYSTPYTFGQGTKVDIK

SEQ ID NO: 632 - protein, 107 aa, synthetic construct
  REGION 1..107: note = Description of Artificial Sequence: Synthetic polypeptide
ELTLTQSPATLSVSPGERATLSCRASQSVSSNLAWYQQKPGQAPRLLIYGASTRATGIPARFSGSGSGTEFTLTISSLQAEDVAVYYCQQYYSTPLTFGGGTKVEIK

SEQ ID NO: 633 - protein, 107 aa, synthetic construct
  REGION 1..107: note = Description of Artificial Sequence: Synthetic polypeptide
ELTLTQSPAFMSATPGDKVNISCKASQDIDDDMNWYQQKPGEAAIFIIQEATTLVPGIPPRFSGSGYGTDFTLTINNIESEDAAYYFCLQHDNFPWTFGQGTKVDTK

SEQ ID NO: 634 - protein, 107 aa, synthetic construct
  REGION 1..107: note = Description of Artificial Sequence: Synthetic polypeptide
ELQMTQSPSSVSASVGDKVTITCRASQGISSWLAWYQQKPGKGPKLLIYAASSLQSGVPSRFSGSGSGTDFTPTISSLQPEDFATYYCQQTNSFPRTLGQGTKLEIK

SEQ ID NO: 635 - protein, 107 aa, synthetic construct
  REGION 1..107: note = Description of Artificial Sequence: Synthetic polypeptide
ELTLTQSPAFMSATPGDKVNISCKASQDVDDDMNWYQQKPGEAAIFIIQEATTLVPGIPPRFSGSGYGTDFTLTINNIESEDAAYYFCLQHDNFPWTFGQGTRLEIK

SEQ ID NO: 636 - protein, 107 aa, synthetic construct
  REGION 1..107: note = Description of Artificial Sequence: Synthetic polypeptide
DIVMTQAQKFMSTSVGDRVSVTCKASQNVGTNVAWYQQKPGQSPKALIYSASYRYSGVPDRFTGSGSGTDFTLTVTNVQSEDLAEYFCQRFNNYPLTFGAGTKLEIK

SEQ ID NO: 637 - protein, 107 aa, synthetic construct
  REGION 1..107: note = Description of Artificial Sequence: Synthetic polypeptide
DIVMTQSQKFMSTSVGDRVSATCKASQNVGSSVAWFQQKPGQSPKALIYSTSYRYSGVPDRFTGSGSGTDFTLTISNVQSEDLAEYFCQQYNSYPLTFGAGTKLEIK

SEQ ID NO: 638 - protein, 107 aa, synthetic construct
  REGION 1..107: note = Description of Artificial Sequence: Synthetic polypeptide
DIVMTQAPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLEIK

SEQ ID NO: 639 - protein, 107 aa, synthetic construct
  REGION 1..107: note = Description of Artificial Sequence: Synthetic polypeptide
DIVMTQSQKFMSTSVGDRVSVTCKASRNVGTNVAWYQQKLGQSPKTLIYSASYRYSGVPDRFTGSGSGTDFTLTISNVQSEDLAEYFCQQYNSYPLTFGAGTKLEIK

SEQ ID NO: 640 - protein, 107 aa, synthetic construct
  REGION 1..107: note = Description of Artificial Sequence: Synthetic polypeptide
DIQMTQTPATLSVTPGDRVSLSCRASQSISDYLHWYQQKSHESPRLLIKYASQSISGIPSRFSGSGSGSDFTLSINSVEPEDVGVYYCQNGHSFPHTLGSGTKLEIK

SEQ ID NO: 641 - protein, 107 aa, synthetic construct
  REGION 1..107: note = Description of Artificial Sequence: Synthetic polypeptide
DIQMIQSQKFMSTSVGDRVSVTCKASQNVGTNVAWYQQKPGQSPKALIYSASYRYSGVPDRFTGSGSGTDFTLTIGNVQSEDLVEYFCQQYNIYPLTFGAGTKLELK

SEQ ID NO: 642 - protein, 107 aa, synthetic construct
  REGION 1..107: note = Description of Artificial Sequence: Synthetic polypeptide
DIVMTQSPATLSVTPGDSVSLSCRASQSISNYLHWYQQKSHESPRLLIKYASQSISGIPSRFSGSGSGSDFTLSINSVEPEDVGVYYCQNGHSFPLTFGGGTKLELK

SEQ ID NO: 643 - protein, 107 aa, synthetic construct
  REGION 1..107: note = Description of Artificial Sequence: Synthetic polypeptide
DIQMTQSPSSLSASVGDRVTITCRASQSIGRYLNWYQQKPGKAPKLLIYAASSLHSGVPSRFSGSGSGTDFTLTISSLQPEDFATYYCQQSYVTPWTFGGGTKLEIK

SEQ ID NO: 644 - protein, 107 aa, synthetic construct
  REGION 1..107: note = Description of Artificial Sequence: Synthetic polypeptide
DIQMTQSPSSLSASVGDRVTITCRASQSIGTYLNWYQQKPGKAPKLLIYGASTLHSGVPSRFSGSGSGTDFTLTISSLQPEDFATYYCQQSYSAPWTFGGGTKVEIK

SEQ ID NO: 645 - protein, 107 aa, synthetic construct
  REGION 1..107: note = Description of Artificial Sequence: Synthetic polypeptide
DIQMTQSPSSLSASVGDRVTITCKASQSVRNDVVWYQQKPGKAPKLLIYRGNTLRPGVPSRFSGSGSGTDFTLTISSLQPEDFATYYCQQYYGIPLTFGQGTKLEIK

SEQ ID NO: 646 - protein, 107 aa, synthetic construct
  REGION 1..107: note = Description of Artificial Sequence: Synthetic polypeptide
DIQMTQSPSSLSASVGDRVTITCRASQSVTTYLNWYQQKPGKAPKLLIYSASSLQSGVPSRFSGSGSGTDFTLTISSLQPEDFATYYCQQTYATPWTFGGGTKVEIK

SEQ ID NO: 647 - protein, 107 aa, synthetic construct
  REGION 1..107: note = Description of Artificial Sequence: Synthetic polypeptide
DIQMTQSPSSLSASVGDRVTITCRASQSISDYLNWYQQKPGKAPKLLIYTASTLQSGVPSRFSGSGSGTDFTLTISSLQPEDFATYYCEQSYSTPWTFGGGTKLEIK

SEQ ID NO: 648 - protein, 111 aa, synthetic construct
  REGION 1..111: note = Description of Artificial Sequence: Synthetic polypeptide
DIQMTQSPSSLSASVGDRVTITCKASHSVDYDGDNYMNWYQQKPGKAPKLLIYWASTRLTGVPSRFSGSGSGTDFTLTISSLQPEDFATYYCLQHIEYPFTFGQGTKLEIK

SEQ ID NO: 649 - protein, 113 aa, synthetic construct
  REGION 1..113: note = Description of Artificial Sequence: Synthetic polypeptide
DIQMTQSPSSLSASVGDRVTITCKSSQSVLYSSNNKNYFAWYQQKPGKAPKLLIYDAPNRATGVPSRFSGSGSGTDFTLTISSLQPEDFATYYCQQGYTTPYTFGGGTKVEIK

SEQ ID NO: 650 - protein, 107 aa, synthetic construct
  REGION 1..107: note = Description of Artificial Sequence: Synthetic polypeptide
DIQMTQSPSSLSASVGDRVTITCRASQDIGRSLSWYQQKPGKAPKLLIYDASRFISGVPSRFSGSGSGTDFTLTISSLQPEDFATYYCQWSNSWPYTFGQGTKLEIK

SEQ ID NO: 651 - protein, 107 aa, synthetic construct; identical to SEQ ID NO: 643
  REGION 1..107: note = Description of Artificial Sequence: Synthetic polypeptide

SEQ ID NO: 652 - protein, 107 aa, synthetic construct; identical to SEQ ID NO: 645
  REGION 1..107: note = Description of Artificial Sequence: Synthetic polypeptide

SEQ ID NO: 653 - protein, 111 aa, synthetic construct
  REGION 1..111: note = Description of Artificial Sequence: Synthetic polypeptide
DIQMTQSPSSLSASVGDRVTITCKASQSVDYDGDSYMNWYQQKPGKAPKLLIYRANRLVDGVPSRFSGSGSGTDFTLTISSLQPEDFATYYCQNGHSFPLTFGQGTKLEIK

SEQ ID NO: 654 - protein, 107 aa, synthetic construct
  REGION 1..107: note = Description of Artificial Sequence: Synthetic polypeptide
DIQMTQSPSSLSASVGDRVTITCRASQTISRYLNWYQQKPGKAPKLLIYSASTLQSGVPSRFSGSGSGTDFTLTISSLQPEDFATYYCQQSYSTPHTFGGGTKVEIK

SEQ ID NO: 655 - protein, 107 aa, synthetic construct
  REGION 1..107: note = Description of Artificial Sequence: Synthetic polypeptide
DIQMTQSPSSLSASVGDRVTITCRASQSIGSYLNWYQQKPGKAPKLLIYGASNLQSGVPSRFSGSGSGTDFTLTISSLQPEDFATYYCQQGYSAPRTFGGGTKLEIK

SEQ ID NO: 656 - protein, 107 aa, synthetic construct
  REGION 1..107: note = Description of Artificial Sequence: Synthetic polypeptide
DIQMTQSPSSLSASVGDRVTITCRASRSISSYLNWYQQKPGKAPKLLIYAASSLPSGVPSRFSGSGSGTDFTLTISSLQPEDFATYYCQQSYSTPRTFGGGTKLEIK

SEQ ID NO: 657 - protein, 107 aa, synthetic construct
  REGION 1..107: note = Description of Artificial Sequence: Synthetic polypeptide
DIQMTQSPSSLSASVGDRVTITCKVSQDVRTAVAWYQQKPGKAPKLLIYDTSYLASGVPSRFSGSGSGTDFTLTISSLQPEDFATYYCQQSYSWSLTFGQGTKLEIK

SEQ ID NO: 658 - protein, 107 aa, synthetic construct
  REGION 1..107: note = Description of Artificial Sequence: Synthetic polypeptide
DIQMTQSPSSLSASVGDRVTITCGGGNDIGSSMYWYQQKPGKAPKLLIYWMSNLASGVPSRFSGSGSGTDFTLTISSLQPEDFATYYCQQYSTYPFALGQGTKLEIK

SEQ ID NO: 659 - protein, 107 aa, synthetic construct
  REGION 1..107: note = Description of Artificial Sequence: Synthetic polypeptide
DIQMTQSPSSLSASVGDRVTITCRASQSISDYLNWYQQKPGKAPKLLIYGASPRESGVPSRFSGSGSGTDFTLTISSLQPEDFATYYCQQDNIWPYTFGQGTKLEIK

SEQ ID NO: 660 - protein, 108 aa, synthetic construct
  REGION 1..108: note = Description of Artificial Sequence: Synthetic polypeptide
DIQMTQSPSSLSASVGDRVTITCGGGNDIGSSMYWYQQKPGKAPKLLIYDASRFISGVPSRFSGSGSGTDFTLTISSLQPEDFATYYCQQSNEDPPFTFGQGTKLEIK

SEQ ID NO: 661 - protein, 111 aa, synthetic construct
  REGION 1..111: note = Description of Artificial Sequence: Synthetic polypeptide
DIQMTQSPSSLSASVGDRVTITCRASESVDSFGNNFMNWYQQKPGKAPKLLIYHTSRLNSGVPSRFSGSGSGTDFTLTISSLQPEDFATYYCQQNNEVPRTFGQGTKLEIK

SEQ ID NO: 662 - protein, 106 aa, synthetic construct
  REGION 1..106: note = Description of Artificial Sequence: Synthetic polypeptide
DIQMTQSPSSLSASVGDRVTITCRASSSVTYIHWYQQKPGKAPKLLIYAVSRLDSGVPSRFSGSGSGTDFTLTISSLQPEDFATYYCHQSNEDPYTFGQGTKLEIK

SEQ ID NO: 663 - protein, 107 aa, synthetic construct
  REGION 1..107: note = Description of Artificial Sequence: Synthetic polypeptide
DIQMTQSPSSLSASVGDRVTITCRASQSIGRYLNWYQQKPGKAPKLLIYAASSLHSGVPSRFSGSGSGTDFTLTISSLQPEDFATYYCQQSYVTPWTFGGGTKVEIK

SEQ ID NO: 664 - protein, 107 aa, synthetic construct; identical to SEQ ID NO: 645
  REGION 1..107: note = Description of Artificial Sequence: Synthetic polypeptide

SEQ ID NO: 665 - protein, 110 aa, synthetic construct
  REGION 1..110: note = Description of Artificial Sequence: Synthetic polypeptide
DIQMTQSPSSLSASVGDRVTITCKASHSVDYDGDNYMNWYQQKPGKAPKLLIYDASRFISGVPSRFSGSGSGTDFTLTISSLQPEDFATYYCLRYASYRTFGQGTKLEIK

SEQ ID NO: 666 - protein, 111 aa, synthetic construct
  REGION 1..111: note = Description of Artificial Sequence: Synthetic polypeptide
DIQMTQSPSSLSASVGDRVTITCRASESVNSYGNSFMHWYQQKPGKAPKLLIYDASRFISGVPSRFSGSGSGTDFTLTISSLQPEDFATYYCLQYGESPLTFGQGTKLEIK

SEQ ID NO: 667 - protein, 112 aa, synthetic construct
  REGION 1..112: note = Description of Artificial Sequence: Synthetic polypeptide
DIQMTQSPSSLSASVGDRVTITCRSSKSLLHSSGNAYVYWYQQKPGKAPKLLIYYTSKPNSGVPSRFSGSGSGTDFTLTISSLQPEDFATYYCQHHYGIPLTFGQGTKLEIK

SEQ ID NO: 668 - protein, 107 aa, synthetic construct
  REGION 1..107: note = Description of Artificial Sequence: Synthetic polypeptide
DIQMTQSPSSLSASVGDRVTITCRASQSIGTYLNWYQQKPGKAPKLLIYAASSLESGVPSRFSGSGSGTDFTLTISSLQPEDFATYYCQQTYNTPWTFGGGTKVEIK

SEQ ID NO: 669 - protein, 108 aa, synthetic construct
  REGION 1..108: note = Description of Artificial Sequence: Synthetic polypeptide
DIQMTQSPSSLSASVGDRVTITCRASSRVSSSYLYWYQQKPGKAPKLLIYATYSLDYGVPSRFSGSGSGTDFTLTISSLQPEDFATYYCLQHGERPLTFGQGTKLEIK

SEQ ID NO: 670 - protein, 107 aa, synthetic construct
  REGION 1..107: note = Description of Artificial Sequence: Synthetic polypeptide
DIQMTQSPSSLSASVGDRVTITCGASQSIGTIIHWYQQKPGKAPKLLIYDTSYLASGVPSRFSGSGSGTDFTLTISSLQPEDFATYYCQQGNTRPWTFGQGTKLEIK

SEQ ID NO: 671 - protein, 108 aa, synthetic construct
  REGION 1..108: note = Description of Artificial Sequence: Synthetic polypeptide
DIQMTQSPSSLSASVGDRVTITCRASENIYVPLNWYQQKPGKAPKLLIYDASRFISGVPSRFSGSGSGTDFTLTISSLQPEDFATYYCQQYNSFPLYTFGQGTKLEIK

SEQ ID NO: 672 - protein, 108 aa, synthetic construct
  REGION 1..108: note = Description of Artificial Sequence: Synthetic polypeptide
ELVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGQGTKVDIK

SEQ ID NO: 673 - protein, 112 aa, synthetic construct
  REGION 1..112: note = Description of Artificial Sequence: Synthetic polypeptide
DIQMTQSPSSLSASVGDRVTITCKSSQSLLYSGEKTYPYWYQQKPGKAPKLLIYWASTRLTGVPSRFSGSGSGTDFTLTISSLQPEDFATYYCQQSNEDSWTFGQGTKLEIK

SEQ ID NO: 674 - protein, 109 aa, synthetic construct
  REGION 1..109: note = Description of Artificial Sequence: Synthetic polypeptide
DIQMTQSPSSLSASVGDRVTITCQSSQDIFNYLEWYQQKPGKAPKLLIYTASNLDTGVPSRFSGSGSGTDFTLTISSLQPEDFATYYCQQGYSTPPEITFGGGTKVEIK

SEQ ID NO: 675 - protein, 107 aa, synthetic construct
  REGION 1..107: note = Description of Artificial Sequence: Synthetic polypeptide
DIQMTQSPSSLSASVGDRVTITCRSTRNILSNMPWYQQKPGKAPKLLIYNANTLAEGVPSRFSGSGSGTDFTLTISSLQPEDFATYYCLQHWNYPYMFGQGTKLEIK

SEQ ID NO: 676 - protein, 108 aa, synthetic construct
  REGION 1..108: note = Description of Artificial Sequence: Synthetic polypeptide
DIQMTQSPSSLSASVGDRVTITCRASQDISNNLHWYQQKPGKAPKLLIYEISGWLSGVPSRFSGSGSGTDFTLTISSLQPEDFATYYCQQSNSWSLLTFGQGTKLEIK

SEQ ID NO: 677 - protein, 108 aa, synthetic construct
  REGION 1..108: note = Description of Artificial Sequence: Synthetic polypeptide
DIQMTQSPSSLSASVGDRVTITCSASQSMSNNLHWYQQKPGKAPKLLIYLASNLGYGVPSRFSGSGSGTDFTLTISSLQPEDFATYYCRQNGHSFPLTFGQGTKLEIK

SEQ ID NO: 678 - protein, 107 aa, synthetic construct
  REGION 1..107: note = Description of Artificial Sequence: Synthetic polypeptide
DIQMTQSPSSLSASVGDRVTITCRASQDISNNLHWYQQKPGKAPKLLIYWASTRLTGVPSRFSGSGSGTDFTLTISSLQPEDFATYYCQQWSDYPFTFGQGTKLEIK

SEQ ID NO: 679 - protein, 106 aa, synthetic construct
  REGION 1..106: note = Description of Artificial Sequence: Synthetic polypeptide
DIQMTQSPSSLSASVGDRVTITCSASSSLSYMHWYQQKPGKAPKLLIYGASPRESGVPSRFSGSGSGTDFTLTISSLQPEDFATYYCRQMSSYPPTFGQGTKLEIK

SEQ ID NO: 680 - protein, 105 aa, synthetic construct
  REGION 1..105: note = Description of Artificial Sequence: Synthetic polypeptide
DIQMTQSPSSLSASVGDRVTITCSASSSVSYMNWYQQKPGKAPKLLIYEISGWLSGVPSRFSGSGSGTDFTLTISSLQPEDFATYYCLRYASYRTFGQGTKLEIK

SEQ ID NO: 681 - protein, 107 aa, synthetic construct
  REGION 1..107: note = Description of Artificial Sequence: Synthetic polypeptide
DIQMTQSPSSLSASVGDRVTITCKASQNMGSNVAWYQQKPGKAPKLLIYSASHRSSGVPSRFSGSGSGTDFTLTISSLQPEDFATYYCQQWNYPRITFGQGTKLEIK

SEQ ID NO: 682 - protein, 107 aa, synthetic construct
  REGION 1..107: note = Description of Artificial Sequence: Synthetic polypeptide
DIQMTQSPSSLSASVGDRVTITCKASQNGGTNVDWYQQKPGKAPKLLIYEISGWLSGVPSRFSGSGSGTDFTLTISSLQPEDFATYYCQHYYSWPPTFGQGTKLEIK

SEQ ID NO: 683 - protein, 107 aa, synthetic construct
  REGION 1..107: note = Description of Artificial Sequence: Synthetic polypeptide
DIQMTQSPSSLSASVGDRVTITCRASENIYVPLNWYQQKPGKAPKLLIYLASYRFTGVPSRFSGSGSGTDFTLTISSLQPEDFATYYCQQINGWPYTFGQGTKLEIK

SEQ ID NO: 684 - protein, 107 aa, synthetic construct
  REGION 1..107: note = Description of Artificial Sequence: Synthetic polypeptide
DIQMTQSPSSLSASVGDRVTITCKASQNMGSNVAWYQQKPGKAPKLLIYAATRLADGVPSRFSGSGSGTDFTLTISSLQPEDFATYYCRQHYSSPPTFGQGTKLEIK

SEQ ID NO: 685 - protein, 107 aa, synthetic construct
  REGION 1..107: note = Description of Artificial Sequence: Synthetic polypeptide
DIQMTQSPSSLSASVGDRVTITCKASQNGGTNVDWYQQKPGKAPKLLIYVASNQGTGVPSRFSGSGSGTDFTLTISSLQPEDFATYYCQQYYTYPLTFGQGTKLEIK

SEQ ID NO: 686 - protein, 105 aa, synthetic construct
  REGION 1..105: note = Description of Artificial Sequence: Synthetic polypeptide
DIQMTQSPSSLSASVGDRVTITCKASQGVDTNVAWYQQKPGKAPKLLIYSSSISGVPSRFSGSGSGTDFTLTISSLQPEDFATYYCAQNRELPFTFGQGTKLEIK

SEQ ID NO: 687 - protein, 107 aa, synthetic construct
  REGION 1..107: note = Description of Artificial Sequence: Synthetic polypeptide
DIQMTQSPSSLSASVGDRVTITCKASQDVGTAITWYQQKPGKAPKLLIYSASKRNTGVPSRFSGSGSGTDFTLTISSLQPEDFATYYCLHHYGTPYTFGQGTKLEIK

SEQ ID NO: 688 - protein, 107 aa, synthetic construct
  REGION 1..107: note = Description of Artificial Sequence: Synthetic polypeptide
DIQMTQSPSSLSTSVGDRVTITCKASQDVGTSVAWYQQKPGKAPKLLIYPASYRSSGVPSRFSGSGSGTDFTLTISSLQPEDFATYYCQQGSSNPLTFGQGTKLEIK

SEQ ID NO: 689 - protein, 107 aa, synthetic construct
  REGION 1..107: note = Description of Artificial Sequence: Synthetic polypeptide
DIQMTQSPSSLSASVGDRVTITCRASQVIDDDINWYQQKPGKAPKLLIYLGSNRAPGVPSRFSGSGSGTDFTLTISSLQPEDFATYYCHQSYTTPHTFGGGTKVEIK

SEQ ID NO: 690 - protein, 107 aa, synthetic construct
  REGION 1..107: note = Description of Artificial Sequence: Synthetic polypeptide
DIQMTQSPSSLSASVGDRVTITCRASQEISGYLTWYQQKPGKAPKLLIYSASHRSSGVPSRFSGSGSGTDFTLTISSLQPEDFATYYCQQWDNNPYTFGQGTKLEIK

SEQ ID NO: 691 - protein, 107 aa, synthetic construct
  REGION 1..107: note = Description of Artificial Sequence: Synthetic polypeptide
DIQMTQSPSSLSASVGDRVTITCRASQSISRYLNWYQQKPGKAPKLLIYKASSLERGVPSRFSGSGSGTDFTLTISSLQPEDFATYYCLQPNSYPWTFGGGTKVEIK

SEQ ID NO: 692 - protein, 107 aa, synthetic construct
  REGION 1..107: note = Description of Artificial Sequence: Synthetic polypeptide
DIQMTQSPSSLSASVGDRVTITCRASQGISSWLDWYQQKPGKAPKLLIYTPFSLQSGVPSRFSGSGSGTDFTLTISSLQPEDFATYYCQHYDDLPLTFGGGTKVEIK

SEQ ID NO: 693 - protein, 107 aa, synthetic construct
  REGION 1..107: note = Description of Artificial Sequence: Synthetic polypeptide
DIQMTQSPSSLSASVGDRVTITCKASQNMGSNVAWYQQKPGKAPKLLIYEASTRFSGVPSRFSGSGSGTDFTLTISSLQPEDFATYYCQQYSSYPLRFGQGTKLEIK

SEQ ID NO: 694 - protein, 107 aa, synthetic construct
  REGION 1..107: note = Description of Artificial Sequence: Synthetic polypeptide
DIQMTQSPSSLSASVGDRVTITCRASQGILGYLNWYQQKPGKAPKLLIYSTSNLLLGVPSRFSGSGSGTDFTLTISSLQPEDFATYYCRQLSSNPLTFGQGTKLEIK

SEQ ID NO: 695 - protein, 111 aa, synthetic construct
  REGION 1..111: note = Description of Artificial Sequence: Synthetic polypeptide
DIQMTQSPSSLSASVGDRVTITCRASESVDNYGISFMSWYQQKPGKAPKLLIYDASRFISGVPSRFSGSGSGTDFTLTISSLQPEDFATYYCQQINSWPLTFGQGTKLEIK

SEQ ID NO: 696 - protein, 113 aa, synthetic construct
  REGION 1..113: note = Description of Artificial Sequence: Synthetic polypeptide
DIQMTQSPSSLSASVGDRVTITCKSSQSLLYSGEKTYPYWYQQKPGKAPKLLIYEASNRYTGVPSRFSGSGSGTDFTLTISSLQPEDFATYYCQQWSSYPPIAFGQGTKLEIK

SEQ ID NO: 697 - protein, 107 aa, synthetic construct
  REGION 1..107: note = Description of Artificial Sequence: Synthetic polypeptide
DIQMTQSPSSLSASVGDRVTITCRASQGLRHDLGWYQQKPGKAPKLLIYWASNRESGVPSRFSGSGSGTDFTLTISSLQPEDFATYYCQKYSSTPYTFGGGTKVEIK

SEQ ID NO: 698 - protein, 111 aa, synthetic construct
  REGION 1..111: note = Description of Artificial Sequence: Synthetic polypeptide
DIQMTQSPSSLSASVGDRVTITCHASESVSVAGTSLLHWYQQKPGKAPKLLIYAASNRESGVPSRFSGSGSGTDFTLTISSLQPEDFATYYCQHWSSFPLTFGQGTKLEIK

SEQ ID NO: 699 - protein, 107 aa, synthetic construct
  REGION 1..107: note = Description of Artificial Sequence: Synthetic polypeptide
ELQMTQSPSSLSASVGDRVTITCRVSQGISNYLNWYQQKPGKAPKLLIYAASSLQSGVPSRFSGSGSGTDFTLTISSLQPEDFATYYCQQSYSTPLTFGGGTKVEIK

SEQ ID NO: 700 - protein, 107 aa, synthetic construct
  REGION 1..107: note = Description of Artificial Sequence: Synthetic polypeptide
ELTLTQSPATLSVSPGERATLSCRASQSVSSNLAWYQQKPGQAPRLLIYGASSRATGIPDRFSGSGSGTDFTLTISRLEPEDFAVYYCQQYGSSPPTFGQGTRLEIK

SEQ ID NO: 701 - protein, 107 aa, synthetic construct
  REGION 1..107: note = Description of Artificial Sequence: Synthetic polypeptide
ELTLTQSPATLSVSPGERATLSCRASQSVSSNLAWYQQKPGQAPRLLIYGASSRATGIPDRFSGSGSGTDFTLTISRLEPEDFAVYYCQQYGSSPLTFGPGTKVDIK

SEQ ID NO: 702 - protein, 108 aa, synthetic construct
  REGION 1..108: note = Description of Artificial Sequence: Synthetic polypeptide
ELVMTQSPGTLSLSPGERATLSCRASQSVSSSYLAWYQQKPGQAPRLLIYGASSRATGIPDRFSGSGSGTDFTLTISSLQAEDVAVYYCQQYYSTPLTFGGGTKVEIK

SEQ ID NO: 703 - protein, 107 aa, synthetic construct
  REGION 1..107: note = Description of Artificial Sequence: Synthetic polypeptide
ELVMTQFPSSLSASVGDRVTITCRASQSISSYLNWYQQKPGKAPKLLIYAASSLQSGVPSRFSGSGSGTDFTLTISSLQPEDFATYYCQQANSFPITFGQGTRLEIK

SEQ ID NO: 704 - protein, 107 aa, synthetic construct
  REGION 1..107: note = Description of Artificial Sequence: Synthetic polypeptide
ELVMTQSPATLSVSLGERATLSCRASQSVSSNLAWYQQKPGQAPRLLIYDASNRATGIPARFSGSGSGTDFTLTISRLGPEDFAVYYCQQYGSSPLTFGGGTKVEIK

SEQ ID NO: 705 - protein, 107 aa, synthetic construct
  REGION 1..107: note = Description of Artificial Sequence: Synthetic polypeptide
ELVMTQSPSSLSASVGDRVTITCRASQRISSYLNWYQQKPGKAPKLLIYAASSLQSRVPSRFSGSGSGTDFTLTISSLQPEDFATYYCQQSYSTPLTFGGGTKLETK

SEQ ID NO: 706 - protein, 108 aa, synthetic construct
  REGION 1..108: note = Description of Artificial Sequence: Synthetic polypeptide
ELTLTQSPATLSLSPGERATLSCRAIQSVSSSYLAWYQQKPGQAPRLLIYGASSRATGIPDRFSGSGSGTDFTLTISRLEPEDFAVYYCQQYGSSPLTFGGGTRLEIK

SEQ ID NO: 707 - protein, 106 aa, synthetic construct
  REGION 1..106: note = Description of Artificial Sequence: Synthetic polypeptide
ELTLTQSPATLSVSPGERATLSCRASQSVSSYLAWYQQKPGQAPRLLIYGASSRATGIPDRFSGSGSGTDFTLTISRLEPEDFAVYYCQQYGNSYTFGQGTKVDIK

SEQ ID NO: 708 - protein, 107 aa, synthetic construct
  REGION 1..107: note = Description of Artificial Sequence: Synthetic polypeptide
ELTLTQSPGTLSLSPGERATLSCRASQSVSSNLAWYQQKPGQAPRLLIYGASTRATGTPARFSGSGSGTEFTLTISSLQSEDFAVYYCQQYGSSPPTFGQGTRLEIK

SEQ ID NO: 709 - protein, 107 aa, synthetic construct
  REGION 1..107: note = Description of Artificial Sequence: Synthetic polypeptide
ELVMTQSPSSLSASVGDRVTITCRASQSISSYLNWYQQKPGKAPKLLIYAASSLQSGVPSRFSGSGSGTDFTLTISSLQPEDFATYYCQQSYSTPITFGGGTKLEIK

SEQ ID NO: 710 - protein, 107 aa, synthetic construct
  REGION 1..107: note = Description of Artificial Sequence: Synthetic polypeptide
DIVITQAPKFLLVSAGDRVTITCKASQSVSNDVAWYQQKPGQSPKLLIYYASNRYTGVPDRFSGSGYGTDFTFTISTVQAEDLAVYFCQQDYRSPLTFGAGTKLELK

SEQ ID NO: 711 - protein, 107 aa, synthetic construct
  REGION 1..107: note = Description of Artificial Sequence: Synthetic polypeptide
DIQMKQSQKFMSTSVGDRVSVTCKASQNVGTNVAWYQQKPGQSPKALIYSASYRYSGVPDRFTGSGSGTDFTLTISNVQSGDLAEYFCQQYNSYPLTFGAGTKLEIK

SEQ ID NO: 712 - protein, 106 aa, synthetic construct
  REGION 1..106: note = Description of Artificial Sequence: Synthetic polypeptide
DIVMTQAPAIMSASPGEKVTMTCSASSSVSYMYWYQQKPGSSPRFLIYDTSNLASGVPVRFSGSGSGTSYSLTISSMEAEDAATYYCQQWNSNPLTFSAGTKLEIK

SEQ ID NO: 713 - protein, 107 aa, synthetic construct
  REGION 1..107: note = Description of Artificial Sequence: Synthetic polypeptide
DIVMTQSPATLSVTPGDRVSLSCRASQSISDYLHWYQQKSHESPRLLIKYASQSISGIPSRFSGSGSGTDFTLSINSVEPEDVGVYYCQNGHSFPLTFGAGTKLELK

SEQ ID NO: 714 - protein, 107 aa, synthetic construct
  REGION 1..107: note = Description of Artificial Sequence: Synthetic polypeptide
DIKITQSPSSLSASLGDTITITCHASQNINVWLNRYQQKPGNIPKLLIYKASNLHTGVPSRFSGSGSGTGFTLTISSLQPEDIATYYCQQGQSYPLTFGAGTKLEIK

SEQ ID NO: 715 - protein, 107 aa, synthetic construct
  REGION 1..107: note = Description of Artificial Sequence: Synthetic polypeptide
DIQMNQSQKFMSTSVGDRVSVTCKASQNVGSNVAWYQQKPGQSPKALIYSASYRYSGVPDRFTGSGSGTDFTPTISNVQSEDLAEYFCQQYNSYPLTFGAGTKLELK

SEQ ID NO: 716 - protein, 110 aa, synthetic construct
  REGION 1..110: note = Description of Artificial Sequence: Synthetic polypeptide
QSVLTQPPSVSAAPGQKVTISCSGISSNIGNNYVSWYQQLPGTAPKLLIYDNNKRASGIPDRFSGSKSGTSATLGITGLQTGDEADYYCGTWDTSLSAGVFGGGTKLTVL

SEQ ID NO: 717 - protein, 110 aa, synthetic construct
  REGION 1..110: note = Description of Artificial Sequence: Synthetic polypeptide
QSVLTQPPSVSAAPGQKVTISCSGSSSNIGNHYVSWYQQLPGTAPKLLIYDNTKRPSGIPDRFSGSKSGTSATLGITGLQTGDEADYYCGTWDASLSTWVFGGGTKLTVL

SEQ ID NO: 718 - protein, 15 aa, synthetic construct
  REGION 1..15: note = Description of Artificial Sequence: Synthetic peptide
GGGGSGGGGSGGGGS

SEQ ID NO: 719 - DNA, 30 bp, synthetic construct
  misc_feature 1..30: note = Description of Artificial Sequence: Synthetic primer
  source 1..30: mol_type = other DNA
acagaattcattaaagaggagaaattaacc

SEQ ID NO: 720 - DNA, 20 bp, synthetic construct
  misc_feature 1..20: note = Description of Artificial Sequence: Synthetic primer
  source 1..20: mol_type = other DNA
tgaaccgcctccaccgctag

SEQ ID NO: 721 - protein, 37 aa, synthetic construct
  REGION 1..37: note = Description of Artificial Sequence: Synthetic polypeptide
  VARIANT 15..19: note = Any amino acid
CARDLRELECEEWTXXXXXSRGPCVDPRGVAGSFDVW

SEQ ID NO: 722 - protein, 26 aa, synthetic construct
  REGION 1..26: note = Description of Artificial Sequence: Synthetic peptide
  VARIANT 10..14: note = Any amino acid
CARDMYYDFXXXXXEVVPADDAFDIW

SEQ ID NO: 723 - protein, 35 aa, synthetic construct
  REGION 1..35: note = Description of Artificial Sequence: Synthetic polypeptide
  VARIANT 17..21: note = Any amino acid
CARDGRGSLPRPKGGPXXXXXYDSSEDSGGAFDIW

SEQ ID NO: 724 - protein, 23 aa, synthetic construct
  REGION 1..23: note = Description of Artificial Sequence: Synthetic peptide
  VARIANT 9..13: note = Any amino acid
CARANQHFXXXXXGYHYYGMDVW

SEQ ID NO: 725 - protein, 25 aa, synthetic construct
  REGION 1..25: note = Description of Artificial Sequence: Synthetic peptide
  VARIANT 9..13: note = Any amino acid
CAKHMSMQXXXXXRADLVGDAFDVW

SEQ ID NO: 726 - protein, 11 aa, synthetic construct
  REGION 1..11: note = Description of Artificial Sequence: Synthetic peptide
  VARIANT 6: note = S, D or N
  VARIANT 8: note = Y, A, G or W
  VARIANT 10: note = S, H or G
GFTFSXYXMXW

SEQ ID NO: 727 - protein, 16 aa, synthetic construct
  REGION 1..16: note = Description of Artificial Sequence: Synthetic peptide
  VARIANT 3: note = S or A
  VARIANT 4: note = Y or V
  VARIANT 7: note = Y or P
  VARIANT 8: note = N, S or D
  VARIANT 10: note = G or S
  VARIANT 11: note = I, N or E
  VARIANT 12: note = T or K
  VARIANT 15: note = P or A
WVXXISXXGXXXYYXD

SEQ ID NO: 728 - protein, 25 aa, synthetic construct
  REGION 1..25: note = Description of Artificial Sequence: Synthetic peptide
  VARIANT 4: note = G or D
  VARIANT 5: note = R or E
  VARIANT 6: note = G, L or D
  VARIANT 7: note = Y, N or H
  VARIANT 8: note = Y or R
  VARIANT 9: note = Y or G
  VARIANT 10: note = S, D or I
  VARIANT 11: note = G, S, R or F
  VARIANT 12: note = S, W, Y, T or L
  VARIANT 13: note = R, G, T or Y
  VARIANT 14: note = Y, H, Q or F
  VARIANT 15: note = R, Y, D or N
  VARIANT 16: note = G, H, Y or F
  VARIANT 17: note = Y, G or H
  VARIANT 18: note = K, Y or F
  VARIANT 19: note = Y, G or H
  VARIANT 20: note = R, Y, G or F
  VARIANT 21: note = G or P
CARXXXXXXXXXXXXXXXXXXMDVW

SEQ ID NO: 729 - protein, 23 aa, synthetic construct
  REGION 1..23: note = Description of Artificial Sequence: Synthetic peptide
  VARIANT 5: note = P, K, I or R
  VARIANT 6: note = V, S, D or R
  VARIANT 7: note = V, H, Y or G
  VARIANT 8: note = L, Y, R or W
  VARIANT 9: note = V, D, Y or F
  VARIANT 10: note = G, I, S or D
  VARIANT 11: note = V, L, R or F
  VARIANT 12: note = W, T, Y or S
  VARIANT 13: note = T, G or R
  VARIANT 14: note = Q, H, Y or N
  VARIANT 15: note = R, D, N or Y
  VARIANT 16: note = Y, G or H
  VARIANT 17: note = Y or A
  VARIANT 18: note = Y or R
  VARIANT 19: note = G or T
  VARIANT 20: note = M, L or F
  VARIANT 22: note = V or Y
CAREXXXXXXXXXXXXXXXXDXW

SEQ ID NO: 730 - protein, 19 aa, synthetic construct
  REGION 1..19: note = Description of Artificial Sequence: Synthetic peptide
  VARIANT 4: note = G or D
  VARIANT 5: note = P, K, D, T or R
  VARIANT 6: note = D, S, Q or R
  VARIANT 7: note = G, R, D or S
  VARIANT 8: note = Y, R, W or N
  VARIANT 9: note = T, Y, G or L
  VARIANT 10: note = S, G, D, I or P
  VARIANT 11: note = F, Y, R, W or L
  VARIANT 12: note = Y, L, H, T or W
  VARIANT 13: note = G, R or T
  VARIANT 14: note = Y, H, F or Q
  VARIANT 15: note = P, Y, N or G
CARXXXXXXXXXXXXFDYW

SEQ ID NO: 731 - protein, 14 aa, synthetic construct
  REGION 1..14: note = Description of Artificial Sequence: Synthetic peptide
  VARIANT 4: note = G, D, H or I
  VARIANT 5: note = R, L or D
  VARIANT 6: note = R, D, T or H
  VARIANT 7: note = G, D, Y or N
  VARIANT 8: note = Y, W, G or N
  VARIANT 9: note = F, Y or D
  VARIANT 10: note = G, P or A
CARXXXXXXXFDYW

SEQ ID NO: 732 - protein, 11 aa, synthetic construct
  REGION 1..11: note = Description of Artificial Sequence: Synthetic peptide
  VARIANT 6: note = S, D or N
  VARIANT 8: note = A, Y or W
  VARIANT 10: note = S, H or N
GGTFSXYXMXW

SEQ ID NO: 733 - protein, 16 aa, synthetic construct
  REGION 1..16: note = Description of Artificial Sequence: Synthetic peptide
  VARIANT 4: note = V, R or T
  VARIANT 6: note = N or R
  VARIANT 8: note = G, Y or N
  VARIANT 9: note = N or D
  VARIANT 11: note = E or Y
  VARIANT 13: note = K, Y or H
  VARIANT 15: note = A or N
  VARIANT 16: note = D or Q
WIGXIXPXXGXTXYXX

SEQ ID NO: 734 - protein, 22 aa, synthetic construct
  REGION 1..22: note = Description of Artificial Sequence: Synthetic peptide
  VARIANT 4: note = P, G or H
  VARIANT 5: note = S, F or G
  VARIANT 6: note = E, R or A
  VARIANT 7: note = R, Y or P
  VARIANT 8: note = S, Y or M
  VARIANT 9: note = H, D, S or Y
  VARIANT 10: note = Y, G, T or V
  VARIANT 11: note = Y, S or E
  VARIANT 12: note = A, Y or I
  VARIANT 13: note = T, F or Y
  VARIANT 14: note = S or Y
  VARIANT 15: note = A, F or H
  VARIANT 16: note = P, Y or G
  VARIANT 17: note = Y, G or F
  VARIANT 18: note = Q, A or Y
  VARIANT 19: note = F or M
  VARIANT 20: note = D or A
CARXXXXXXXXXXXXXXXXXYW

SEQ ID NO: 735 - protein, 18 aa, synthetic construct
  REGION 1..18: note = Description of Artificial Sequence: Synthetic peptide
  VARIANT 4: note = D, S, L or H
  VARIANT 5: note = S, F, E, P or T
  VARIANT 6: note = S, G, F or I
  VARIANT 7: note = D, S, G, Y or R
  VARIANT 8: note = Y, N, S or F
  VARIANT 9: note = Y, D, G or T
  VARIANT 10: note = G, V, N, D or Y
  VARIANT 11: note = Y, F, S or G
  VARIANT 12: note = A, Y, N, Q or S
  VARIANT 13: note = G, Y, T or F
  VARIANT 14: note = A, W, G or Y
  VARIANT 16: note = A or D
CARXXXXXXXXXXXFXYW

SEQ ID NO: 736 - protein, 17 aa, synthetic construct
  REGION 1..17: note = Description of Artificial Sequence: Synthetic peptide
  VARIANT 4: note = S, H, D or L
  VARIANT 5: note = Y, L, G or E
  VARIANT 6: note = Y, G, S or L
  VARIANT 7: note = D, S, K, P or W
  VARIANT 8: note = S, Y, N or H
  VARIANT 9: note = F, D, Y, T or N
  VARIANT 10: note = Y, V, G, N or D
  VARIANT 11: note = Y, T, F, L or G
  VARIANT 12: note = Y, A, N, S or H
  VARIANT 13: note = A or Y
CARXXXXXXXXXXMDYW

SEQ ID NO: 737 - protein, 14 aa, synthetic construct
  REGION 1..14: note = Description of Artificial Sequence: Synthetic peptide
  VARIANT 4: note = R, G, D or F
  VARIANT 5: note = G, D, R or Y
  VARIANT 6: note = G, Y, S, H or N
  VARIANT 7: note = G, Y, S, L or W
  VARIANT 8: note = R, G, F, A or Y
  VARIANT 9: note = R, A, F, Y or D
  VARIANT 10: note = Y, S, W, K or A
  VARIANT 11: note = F or L
  VARIANT 12: note = D, A or V
CARXXXXXXXXXYW

SEQ ID NO: 738 - protein, 18 aa, synthetic construct
  REGION 1..18: note = Description of Artificial Sequence: Synthetic peptide
  VARIANT 9: note = H or N
  VARIANT 11: note = N or D
  VARIANT 13: note = N or K
  VARIANT 17: note = A, H or E
CRSSQSLVXSXGXTYLXW

SEQ ID NO: 739 - protein, 9 aa, synthetic construct
  REGION 1..9: note = Description of Artificial Sequence: Synthetic peptide
  VARIANT 3: note = A or V
  VARIANT 7: note = A or F
YKXSNRXSG

SEQ ID NO: 740 - protein, 11 aa, synthetic construct
  REGION 1..11: note = Description of Artificial Sequence: Synthetic peptide
  VARIANT 2: note = Q or F
  VARIANT 4: note = G or S
  VARIANT 5: note = S or Y
  VARIANT 6: note = H, S or E
  VARIANT 7: note = Y, V or T
  VARIANT 9: note = L or F
CXQXXXXPXTF

SEQ ID NO: 741 - protein, 15 aa, synthetic construct
  REGION 1..15: note = Description of Artificial Sequence: Synthetic peptide
  VARIANT 11: note = N or H
CSGSSSNIGSXYVSW

SEQ ID NO: 742 - protein, 9 aa, synthetic construct
  REGION 1..9: note = Description of Artificial Sequence: Synthetic peptide
YRNNQRPSG

SEQ ID NO: 743 - protein, 13 aa, synthetic construct
  REGION 1..13: note = Description of Artificial Sequence: Synthetic peptide
  VARIANT 2: note = A or G
  VARIANT 3: note = T, S or V
  VARIANT 4: note = W, T or R
  VARIANT 6: note = S or G
  VARIANT 7: note = T, R, S or Q
  VARIANT 8: note = S, L or W
  VARIANT 9: note = R or S
  VARIANT 10: note = S, A or Q
CXXXDXXXXXVVF

(19) United States
(12) Patent Application Publication
(10) Pub. No.: US 2025/0145724 A1
(43) Pub. Date: May 8, 2025

(54) Methods and Compositions Relating to Adenosine Receptors

(71) Applicant: Twist Bioscience Corporation, South San Francisco, CA (US)

(72) Inventors: Aaron Sato, Burlingame, CA (US); Qiang Liu, Palo Alto, CA (US); Fumiko Axelrod, Palo Alto, CA (US); Linya Wang, Milpitas, CA (US)

(73) Assignee: Twist Bioscience Corporation, South San Fransico, CA (US)

(21) Appl. No.: 18/975,629

(22) Filed: Dec. 10, 2024

Related U.S. Application Data
(62) Division of application No. 17/580,547, filed on Jan. 20, 2022, now Pat. No. 12,202,905.
(60) Provisional application No. 63/244,976, filed on Sep. 16, 2021.
(60) Provisional application No. 63/209,892, filed on Jun. 11, 2021.
(60) Provisional application No. 63/140,201, filed on Jan. 21, 2021.

(51) Int. Cl. C07K 16/28

(52) U.S. Cl. CPC C07K 16/2869; C07K 16/286; C07K 2317/21; C07K 2317/24; C07K 2317/569; C07K 2317/622; C07K 2317/76; C07K 2317/92; C07K 2318/10

(57) ABSTRACT

Provided herein are methods and compositions relating to adenosine A2A receptor libraries having nucleic acids encoding for a scaffold comprising an adenosine A2A binding domain. adenosine A2A receptor libraries described herein encode for immunoglobulins such as antibodies.

### CROSS-REFERENCE

This application is a divisional of U.S. patent application Ser. No. 17/580,547, filed Jan. 20, 2022, which claims the benefit of U.S. Patent Application No. 63/140,201, filed Jan. 21, 2021, U.S. Patent Application No. 63/209,892, filed Jun. 11, 2021, and U.S. Patent Application No. 63/244,976, filed Sep. 16, 2021, the contents of each of which is entirely incorporated by reference herein.

### REFERENCE TO ELECTRONIC SEQUENCE LISTING

The application contains a Sequence Listing which has been submitted electronically in .XML format and is hereby incorporated by reference in its entirety. Said .XML copy, created on Dec. 9, 2024, is named “01356-0011-01US.xml” and is 1,027,150 bytes in size. The sequence listing contained in this .XML file is part of the specification and is hereby incorporated by reference herein in its entirety.

### BACKGROUND

G protein-coupled receptors (GPCRs) such as adenosine receptors are implicated in a wide variety of diseases. Raising antibodies to GPCRs has been difficult due to problems in obtaining suitable antigen because GPCRs are often expressed at low levels in cells and are very unstable when purified. Thus, there is a need for improved agents for therapeutic intervention which target adenosine receptors.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF SUMMARY

Provided herein are compositions and methods for activating T cells.

Provided herein are methods for activating T cells, comprising administering an antibody or antibody fragment comprising a sequence at least about 90% identical to a sequence set forth in SEQ ID NOs: 6-717. Further provided herein are methods for activating T cells, wherein the antibody or antibody fragment comprises an amino acid sequence at least about 95% identical to that set forth in any one of SEQ ID NOs: 35-44. Further provided herein are methods for activating T cells, wherein the antibody or antibody fragment comprises an amino acid sequence as set forth in any one of SEQ ID NOs: 35-44. Further provided herein are methods for activating T cells, wherein the antibody is a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarity determining region (CDR), a diabody, a fragment comprised of only a single monomeric variable domain, disulfide-linked Fvs (sdFv), an intrabody, an anti-idiotypic (anti-Id) antibody, or ab antigen-binding fragments thereof. Further provided herein are methods for activating T cells, wherein the antibody or antibody fragment binds to adenosine 2A receptor with a K_{D} of less than about 75 nM. Further provided herein are methods for activating T cells, wherein the antibody or antibody fragment binds to adenosine 2A receptor with a K_{D} of less than about 50 nM. Further provided herein are methods for activating T cells, wherein the antibody or antibody fragment binds to adenosine 2A receptor with a K_{D} of less than about 25 nM. Further provided herein are methods for activating T cells, wherein the antibody or antibody fragment binds to adenosine 2A receptor with a K_{D} of less than about 10 nM. Further provided herein are methods for activating T cells, wherein the antibody or antibody fragment comprises an IC₅₀ of less than about 20 nM in a T cell activation assay. Further provided herein are methods for activating T cells, wherein the antibody or antibody fragment comprises an IC₅₀ of less than about 10 nM in a T cell activation assay. Further provided herein are methods for activating T cells, wherein the antibody or antibody fragment comprises an IC₅₀ of less than about 7.5 nM in a T cell activation assay. Further provided herein are methods for activating T cells, wherein the antibody or antibody fragment comprises an IC₅₀ of less than about 5 nM in a T cell activation assay.

Provided herein are antibodies or antibody fragments comprising a sequence at least about 90% identical to a sequence set forth in SEQ ID NOs: 6-717. Further provided herein are antibodies or antibody fragments wherein the antibody or antibody fragment comprises an amino acid sequence at least about 95% identical to that set forth in any one of SEQ ID NOs: 35-44. Further provided herein are antibodies or antibody fragments wherein the antibody or antibody fragment comprises an amino acid sequence as set forth in any one of SEQ ID NOs: 35-44. Further provided herein are antibodies or antibody fragments wherein the antibody is a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarity determining region (CDR), a diabody, a fragment comprised of only a single monomeric variable domain, disulfide-linked Fvs (sdFv), an intrabody, an anti-idiotypic (anti-Id) antibody, or ab antigen-binding fragments thereof. Further provided herein are antibodies or antibody fragments wherein the antibody or antibody fragment binds to adenosine 2A receptor with a KD of less than about 75 nM. Further provided herein are antibodies or antibody fragments wherein the antibody or antibody fragment binds to adenosine 2A receptor with a KD of less than about 50 nM. Further provided herein are antibodies or antibody fragments wherein the antibody or antibody fragment binds to adenosine 2A receptor with a KD of less than about 25 nM. Further provided herein are antibodies or antibody fragments wherein the antibody or antibody fragment binds to adenosine 2A receptor with a KD of less than about 10 nM. Further provided herein are antibodies or antibody fragments wherein the antibody or antibody fragment comprises an IC50 of less than about 20 nM in a T cell activation assay. Further provided herein are antibodies or antibody fragments wherein the antibody or antibody fragment comprises an IC50 of less than about 10 nM in a T cell activation assay. Further provided herein are antibodies or antibody fragments wherein the antibody or antibody fragment comprises an IC50 of less than about 7.5 nM in a T cell activation assay. Further provided herein are antibodies or antibody fragments wherein the antibody or antibody fragment comprises an IC50 of less than about 5 nM in a T cell activation assay.

### BRIEF DESCRIPTION OF THE DRAWINGS

FIG. 1A depicts a first schematic of an immunoglobulin scaffold.

FIG. 1B depicts a second schematic of an immunoglobulin scaffold.

FIG. 2 depicts a schematic of a motif for placement in a scaffold.

FIG. 3 presents a diagram of steps demonstrating an exemplary process workflow for gene synthesis as disclosed herein.

FIG. 4 illustrates an example of a computer system.

FIG. 5 is a block diagram illustrating an architecture of a computer system.

FIG. 6 is a diagram demonstrating a network configured to incorporate a plurality of computer systems, a plurality of cell phones and personal data assistants, and Network Attached Storage (NAS).

FIG. 7 is a block diagram of a multiprocessor computer system using a shared virtual address memory space.

FIG. 8A depicts a schematic of an immunoglobulin scaffold comprising a VH domain attached to a VL domain using a linker.

FIG. 8B depicts a schematic of a full-domain architecture of an immunoglobulin scaffold comprising a VH domain attached to a VL domain using a linker, a leader sequence, and pIII sequence.

FIG. 8C depicts a schematic of four framework elements (FW1, FW2, FW3, FW4) and the variable 3 CDR (L1, L2, L3) elements for a VL or VH domain.

FIG. 9A depicts a structure of Glucagon-like peptide 1 (GLP-1, boxed) in complex with GLP-1 receptor (GLP-1R), PDB entry 5VAI.

FIG. 9B depicts a crystal structure of CXCR4 chemokine receptor in complex with a cyclic peptide antagonist CVX15 (boxed), PDB entry 3OR0.

FIG. 9C depicts a crystal structure of human smoothened with the transmembrane domain and extracellular domain (ECD) (boxed), PDB entry 5L7D. The ECD contacts the TMD through extracellular loop 3 (ECL3).

FIG. 9D depicts a structure of GLP-1R in complex with a Fab (boxed), PDB entry 6LN2.

FIG. 9E depicts a crystal structure of CXCR4 in complex with a viral chemokine antagonist Viral macrophage inflammatory protein 2 (vMIP-II, boxed), PDB entry 4RWS.

FIG. 10 depicts a schema of the GPCR focused library design. Two germline heavy chain VH1-69 and VH3-30; 4 germline light chain IGKV1-39 and IGKV3-15, and IGLV1-51 and IGLV2-14.

FIG. 11 depicts a graph of HCDR3 length distribution in the GPCR-focused library compared to the HCDR3 length distribution in B-cell populations from three healthy adult donors. In total, 2,444,718 unique VH sequences from the GPCR library and 2,481,511 unique VH sequences from human B-cell repertoire were analyzed to generate the length distribution plot. The y-axis is labeled frequency from 0.000 to 0.1400 at 0.0200 unit intervals; the x-axis is length and labeled 0 to 57 at 3 amino acid intervals.

FIG. 12 depicts the clone, ELISA value, Library, ProA value, and K_{D} value for VHH-Fc.

FIG. 13 depicts a schema of design of phage-displayed hyperimmune libraries generated herein.

FIGS. 14A-14B depict graphs of a dose curve (FIG. 14A) and FACS analysis (FIG. 14B) of A2AR-90-007.

FIG. 15A depicts a schema of heavy chain IGHV3-23 design. FIG. 15A discloses SEQ ID NOs: 726-731, respectively, in order of appearance.

FIG. 15B depicts a schema of heavy chain IGHV1-69 design. FIG. 15B discloses SEQ ID NOs: 732-737, respectively, in order of appearance.

FIG. 15C depicts a schema of light chains IGKV 2-28 and IGLV 1-51 design. FIG. 15C discloses SEQ ID NOs: 738-743, respectively, in order of appearance.

FIG. 15D depicts a schema of the theoretical diversity and final diversity of a GPCR library.

FIGS. 16A-16O depict flow cytometry data using variant A2A receptor immunoglobulins A2A90 (FIG. 16A), A2A91 (FIG. 16B), A2A92 (FIG. 16C), A2A93 (FIG. 16D), A2A94 (FIG. 16E), A2A1 (FIG. 16F), A2A95 (FIG. 16G), A2A2 (FIG. 16H), A2A3 (FIG. 16I), A2A4 (FIG. 16J), A2A5 (FIG. 16K), A2A6 (FIG. 16L), A2A96 (FIG. 16M), A2A7 (FIG. 16N), and control (FIG. 16O).

FIGS. 17A-17H depict graphs of binding curves using variant A2A receptor immunoglobulins A2A-94 (FIG. 17A), A2A1 (FIG. 17B), A2A3 (FIG. 17C), A2A4 (FIG. 17D) A2A5 (FIG. 17E), A2A6 (FIG. 17F), A2A7 (FIG. 17G), and control (FIG. 17H). Binding curves are plotted with IgG concentration vs. MFI (mean fluorescence intensity).

FIGS. 18A-18O depict graphs of binding curves using variants from a mouse immune library: A2A97 (FIG. 18A), A2A98 (FIG. 18B), A2A99 (FIG. 18C), A2A100 (FIG. 18D), A2A101 (FIG. 18E), A2A102 (FIG. 18F), A2A103 (FIG. 18G), A2A104 (FIG. 18H), A2A9 (FIG. 18I), A2A10 (FIG. 18J), A2A11 (FIG. 18K), A2A12 (FIG. 18L), A2A13 (FIG. 18M), A2A14 (FIG. 18N), and using a control (FIG. 18O).

FIGS. 19A-19G depict graph of cell binding with adenosine A2aR monoclonal (MAB9497) and selected variants: A2A-9 (FIG. 19A), A2A10 (FIG. 19B), A2A11 (FIG. 19C), A2A12 (FIG. 19D) A2A13 (FIG. 19E), A2A15 (FIG. 19F), and control (FIG. 19G). Binding curves are plotted with IgG concentration vs. MFI (mean fluorescence intensity).

FIGS. 20A-20G depict graphs of cell binding in a titration assay from 100 nM. Graphs depict cell binding of a synthetic library to A2a protein (FIG. 20A), synthetic library to A2a protein+ZM241385 (FIG. 20B), humanized synthetic library to A2a protein (FIG. 20C), humanized synthetic library to A2a protein+ZM241385 (FIG. 20D), immune library to A2a protein (FIG. 20E), immune library to A2a protein+ZM241385 (FIG. 20F), and mouse immune library to A2a protein (FIG. 20G).

FIG. 21 depicts data from an agonist dose-response assay measured using a cAMP assay.

FIG. 22 depicts data from an antagonist dose-response assay measured using a cAMP assay.

FIG. 23 depicts results from a cAMP antagonist titration assay.

FIG. 24 depicts data from variant A2A-1 and A2A-9 from a CAMP assay.

FIG. 25 depicts data for variant A2A9 using a CAMP assay.

FIG. 26 depicts data for variant A2A9 using a cAMP antagonist titration assay.

FIG. 27A depicts data for variant A2A receptor immunoglobulins in an antagonistic CAMP assay. FIG. 27B depicts data for additional variant A2A receptor immunoglobulins in an antagonistic cAMP assay. FIG. 27C depicts data for additional variant A2A receptor immunoglobulins in an antagonistic cAMP assay.

FIG. 28A depicts data for variant A2A receptor immunoglobulins in an allosteric cAMP assay. FIG. 28B depicts data for additional variant A2A receptor immunoglobulins in an allosteric CAMP assay. FIG. 28C depicts data for additional variant A2A receptor immunoglobulins in an allosteric cAMP assay.

FIG. 29A depicts data for variant A2A receptor immunoglobulins in an antagonistic CAMP assay. FIG. 29B depicts data for additional variant A2A receptor immunoglobulins in an antagonistic cAMP assay. FIG. 29C depicts data for additional variant A2A receptor immunoglobulins in an antagonistic cAMP assay.

FIG. 30A depicts data for variant A2A receptor immunoglobulins in an antagonistic CAMP assay. FIG. 30B depicts data for additional variant A2A receptor immunoglobulins in an antagonistic cAMP assay. FIG. 30C depicts data for additional variant A2A receptor immunoglobulins in an antagonistic cAMP assay.

FIGS. 31A-31C depict affinity data (FIG. 31A), additional affinity data (FIG. 31B), and specificity data (FIG. 31C) for variant A2A-77.

FIG. 31D depicts A2A-77 binding to cynomolgus PBMCs.

FIG. 32A depicts T cell activation for variants A2A-81, A2A-51, A2A-53, A2A-77, A2A-31, A2A-24, A2A-78, A2A-74, A2A-75, A2A-52, and A2A-36. FIG. 32B depicts T cell activation for variants A2A-81, A2A-51, A2A-53, A2A-77, A2A-31, and A2A-78.

FIG. 32C depicts T cell activation data for variant A2A-77.

FIGS. 32D-32H depict T cell activation data for variants A2A-81, A2A-51, A2A-77, and A2A-28.

FIG. 33A depicts the result of a cell binding assay for variants A2A-77 and A2A-81.

FIG. 33B depicts the result of an A2A antagonistic CAMP assay for variants A2A-77 and A2A-81.

FIG. 33C depicts specificity data for variants A2A-77 and A2A-81, as well as a control A2a.

FIG. 33D depicts T cell activation data for variants A2A-77 and A2A-81.

FIGS. 34A-34B depicts the mean tumor volume over time (FIGS. 34A and 34C) and the relative tumor volume over time (FIGS. 34B and 34D) of mice treated with variants A2A-77 and A2A-81.

FIG. 34E depicts the experimental schema for combination treatments.

FIGS. 34F-34K depict data from the colon cancer model.

FIGS. 35A-35M depict the proportion amount of cells detected in mice in each of the four treatment groups. FIG. 35A depicts the number of TIL CD45+ cells as a percent of all live cells detected. FIGS. 35B-35G depict the number of total T-cells (FIG. 35B), CD4+ cells (FIG. 35C), CD8+ cells (FIG. 35D), regulatory T-cells (Tregs, FIG. 35E), M1 tumor associated macrophages (TAM, FIG. 35F) and M2 TAM (FIG. 35G). FIG. 35H depicts the number of TIL CD45+ cells as a percent of all live cells detected. FIGS. 351-35J depict the number of total T-cells (FIG. 35I), CD4+ cells (FIG. 35J), CD8+ cells (FIG. 35K), regulatory T-cells (Tregs, FIG. 35L), and M1 tumor associated macrophages (TAM, FIG. 35M).

FIGS. 36A-36C depict a cell profile of lysed whole blood in interim versus terminal sample. The percent of CD45+ cells is depicted as a percent of live cells (FIG. 36A). The amount of CD3+ (FIG. 36B) and CD3− (FIG. 36C) cells is depicted as a percent of CD45+ cells.

FIGS. 37A-37G depict the proportion amount of cells detected in mice in each of the four treatment groups in interim lysed whole blood samples. FIG. 37A depicts the number of TIL CD45+ cells as a percent of all live cells detected. FIGS. 37B-37G depict the number of total T-cells (FIG. 37B), CD4+ cells (FIG. 37C), CD8+ cells (FIG. 37D), regulatory T-cells (Tregs, FIG. 37E), M1 tumor associated macrophages (TAM, FIG. 37F) and M2 TAM (FIG. 37G).

FIGS. 38A-38G depict the proportion amount of cells detected in mice in each of the four treatment groups in interim lysed whole blood samples. FIG. 38A depicts the number of TIL CD45+ cells as a percent of all live cells detected. FIGS. 38B-38G depict the number of total T-cells (FIG. 38B), CD4+ cells (FIG. 38C), CD8+ cells (FIG. 38D), regulatory T-cells (Tregs, FIG. 38E), M1 tumor associated macrophages (TAM, FIG. 38F) and M2 TAM (FIG. 38G).

FIG. 39 depicts cytokine level in peripheral blood after T cell activation.

FIGS. 40A-40G depict levels of interferon gamma (FIG. 40A), interleukin 2 (FIG. 40B), interleukin 4 (FIG. 40C), interleukin 6 (FIG. 40D), interleukin 8 (FIG. 40E), interleukin 10 (FIG. 40F), and TNF alpha (FIG. 40G) detected in terminal blood samples.

FIGS. 41A-41C depict a cell profile of lysed whole blood in interim versus terminal sample. The percent of CD45+ cells is depicted as a percent of live cells (FIG. 41A). The amount of CD3+ (FIG. 41B) and CD3− (FIG. 41C) cells is depicted as a percent of CD45+ cells.

FIGS. 42A-42G depict the proportion amount of cells detected in mice in each of the four treatment groups in interim lysed whole blood samples. FIG. 42A depicts the number of TIL CD45+ cells as a percent of all live cells detected. FIGS. 42B-42G depict the number of total T-cells (FIG. 42B), CD4+ cells (FIG. 42C), CD8+ cells (FIG. 42D), regulatory T-cells (Tregs, FIG. 42E), M1 tumor associated macrophages (TAM, FIG. 42F) and M2 TAM (FIG. 42G).

FIGS. 43A-43G depict the proportion amount of cells detected in mice in each of the four treatment groups in terminal lysed whole blood samples. FIG. 43A depicts the number of TIL CD45+ cells as a percent of all live cells detected. FIGS. 43B-43G depict the number of total T-cells (FIG. 43B), CD4+ cells (FIG. 43C), CD8+ cells (FIG. 43D), regulatory T-cells (Tregs, FIG. 43E), M1 tumor associated macrophages (TAM, FIG. 43F) and M2 TAM (FIG. 43G).

FIGS. 44A-44G depict levels of interferon gamma (FIG. 44A), interleukin 2 (FIG. 44B), interleukin 4 (FIG. 44C), interleukin 6 (FIG. 44D), interleukin 8 (FIG. 44E), interleukin 10 (FIG. 44F), and TNF alpha (FIG. 44G) detected in terminal blood samples.

FIG. 45 depicts hA2b cross binder activity in HEK293T cells.

FIG. 46 depicts the functional cAMP assay used to test the activity of the A2b antibodies.

FIGS. 47A-47D depict the results of the A2b functional cAMP assays.

FIGS. 48A-48E demonstrate primary T cell activation assays (cytokine release) in response to reformatted antibodies (IgG1 or IgG4).

FIGS. 49A-49L depict the proportion amount of cells detected in mice in each of the treatment groups in interim lysed whole blood samples. FIG. 49A depicts the number of LWB CD45+ cells as a percent of all live cells detected. FIGS. 49B-49L depict the number of total CD3+ cells (FIG. 49B), CD8+ cells (FIG. 49C), CD4+ cells (FIG. 49D), CD3− non-T-cells (FIG. 49E), Tregs cells (FIG. 49F), proliferative T-cells (FIG. 49G), proliferative Tregs cells (FIG. 49H), CD11b+ cells (FIG. 49I), CD11c+ cells (FIG. 49J), M1 macrophages (FIG. 49K), and M2 macrophages (FIG. 49L).

FIGS. 50A-50L depict the proportion amount of cells detected in mice in each of the treatment groups in terminal lysed whole blood samples. FIG. 50A depicts the number of LWB CD45+ cells as a percent of all live cells detected. FIGS. 50B-50L depict the number of total CD3+ cells (FIG. 50B), CD4+ cells (FIG. 50C), CD8+ cells (FIG. 50D), CD3− non-T-cells (FIG. 50E), Tregs cells (FIG. 50F), proliferative T-cells (FIG. 50G), proliferative Tregs cells (FIG. 50H), CD11b+ cells (FIG. 50I), CD11c+ cells (FIG. 50J), M1 macrophages (FIG. 50K), and M2 macrophages (FIG. 50L).

FIGS. 51A-51L depict the proportion amount of cells detected in mice in each of the treatment groups in terminal lysed whole blood samples. FIG. 51A depicts the number of TIL CD45+ cells as a percent of all live cells detected. FIGS. 51B-51L depict the number of total CD3+ cells (FIG. 51B), CD4+ cells (FIG. 51C), CD8+ cells (FIG. 51D), CD3− non-T-cells (FIG. 51E), Tregs cells (FIG. 51F), proliferative T-cells (FIG. 51G), proliferative Tregs cells (FIG. 51H), CD11b+ cells (FIG. 51I), CD11c+ cells (FIG. 51J), M1 macrophages (FIG. 51K), and M2 macrophages (FIG. 51L).

FIG. 51M depicts the ratio of TIL M1/M2 macrophages in terminal lysed whole blood samples in mice.

FIGS. 52A-52E depict the proportion amount of LWB CD3+ cells detected in mice in each of the treatment groups in terminal lysed whole blood samples. FIG. 52A depicts the number of LWB CD3+ cells as a percent of all CD45+ cells detected. FIGS. 52B-52E depict the number of total CD3+TNFa+ cells (FIG. 52B), CD3+IFNg+ cells (FIG. 52C), CD3+IL6+ cells (FIG. 52D), and CD3+IL8+ cells (FIG. 52E) as a percent of all CD3+ cells detected.

FIGS. 53A-53E depict the proportion amount of LWB CD4+ cells detected in mice in each of the treatment groups in terminal lysed whole blood samples. FIG. 53A depicts the number of LWB CD4+ cells as a percent of all CD3+ cells detected. FIGS. 53B-53E depict the number of total CD4+TNFa+ cells (FIG. 53B), CD4+IFNg+ cells (FIG. 53C), CD4+IL6+ cells (FIG. 53D), and CD4+IL8+ cells (FIG. 53E) as a percent of all CD4+ cells detected.

FIGS. 54A-54E depict the proportion amount of LWB CD8+ cells detected in mice in each of the treatment groups in terminal lysed whole blood samples. FIG. 54A depicts the number of LWB CD8+ cells as a percent of all CD3+ cells detected. FIGS. 54B-54E depict the number of total CD8+TNFa+ cells (FIG. 54B), CD8+IFNg+ cells (FIG. 54C), CD8+IL6+ cells (FIG. 54D), and CD8+IL8+ cells (FIG. 54E) as a percent of all CD8+ cells detected.

FIGS. 55A-55D depict the amount of MFI CD3+ cells detected in mice in each of the treatment groups in terminal lysed whole blood samples. FIGS. 55A-55D depict the number of total CD3+TNFa+ cells (FIG. 55A), CD3+IFNg+ cells (FIG. 55B), CD3+IL6+ cells (FIG. 55C), and CD3+IL8+ cells (FIG. 55D) as difference of all MFI cells detected.

FIGS. 56A-56D depict the amount of MFI CD4+ cells detected in mice in each of the treatment groups in terminal lysed whole blood samples. FIGS. 56A-56D depict the number of total CD4+TNFa+ cells (FIG. 56A), CD4+IFNg+ cells (FIG. 56B), CD4+IL6+ cells (FIG. 56C), and CD4+IL8+ cells (FIG. 56D) as difference of all MFI cells detected.

FIGS. 57A-57D depict the amount of MFI CD8+ cells detected in mice in each of the treatment groups in terminal lysed whole blood samples. FIGS. 57A-57D depict the number of total CD8+TNFa+ cells (FIG. 57A), CD8+IFNg+ cells (FIG. 57B), CD8+IL6+ cells (FIG. 57C), and CD8+IL8+ cells (FIG. 57D) as difference of all MFI cells detected.

FIGS. 58A-58B depict the proportion amount of LWB CD8+ cells detected in mice in each of the treatment groups in interim lysed whole blood samples. FIGS. 58A-58B depict the number of CD8+ cells (FIG. 58A) and Tregs cells (FIG. 58B) as a proportion of CD45+ cells.

FIG. 58C depicts the ratio of LWB CD8+/Treg cells in interim lysed whole blood samples in mice.

FIGS. 59A-59B depict the proportion amount of LWB CD8+ cells detected in mice in each of the treatment groups in terminal lysed whole blood samples. FIGS. 59A-59B depict the number of CD8+ cells (FIG. 59A) and Tregs cells (FIG. 59B) as a proportion of CD45+ cells.

FIG. 59C depicts the ratio of LWB CD8+/Treg cells in terminal lysed whole blood samples in mice.

FIGS. 60A-60B depict the proportion amount of TIL CD8+ cells detected in mice in each of the treatment groups in terminal lysed whole blood samples. FIGS. 60A-60B depict the number of CD8+ cells (FIG. 60A) and Tregs cells (FIG. 60B) as a proportion of CD45+ cells.

FIG. 60C depicts the ratio of TIL CD8+/Treg cells in terminal lysed whole blood samples in mice.

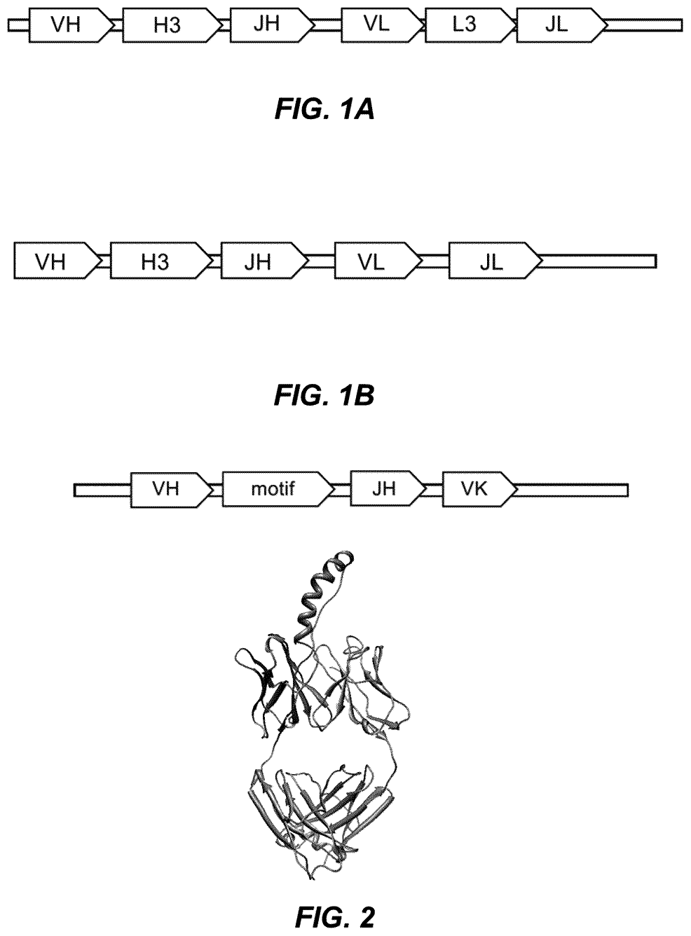

### DETAILED DESCRIPTION

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

Unless specifically stated, as used herein, the term “nucleic acid” encompasses double- or triple-stranded nucleic acids, as well as single-stranded molecules. In double- or triple-stranded nucleic acids, the nucleic acid strands need not be coextensive (i.e., a double-stranded nucleic acid need not be double-stranded along the entire length of both strands). Nucleic acid sequences, when provided, are listed in 5′ to 3′ direction, unless stated otherwise. Methods described herein provide for the generation of isolated nucleic acids. Methods described herein additionally provide for the generation of isolated and purified nucleic acids. A “nucleic acid” as referred to herein can comprise at least 5, 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, 2000, or more bases in length. Moreover, provided herein are methods for the synthesis of any number of polypeptide-segments encoding nucleotide sequences, including sequences encoding non-ribosomal peptides (NRPs), sequences encoding non-ribosomal peptide-synthetase (NRPS) modules and synthetic variants, polypeptide segments of other modular proteins, such as antibodies, polypeptide segments from other protein families, including non-coding DNA or RNA, such as regulatory sequences e.g. promoters, transcription factors, enhancers, siRNA, shRNA, RNAi, miRNA, small nucleolar RNA derived from microRNA, or any functional or structural DNA or RNA unit of interest. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, complementary DNA (cDNA), which is a DNA representation of mRNA, usually obtained by reverse transcription of messenger RNA (mRNA) or by amplification; DNA molecules produced synthetically or by amplification, genomic DNA, recombinant polynucleotides, branched polynucleotides, plasmids, vectors, isolated DNA of any sequence, isolated RNA of any sequence, nucleic acid probes, and primers. cDNA encoding for a gene or gene fragment referred herein may comprise at least one region encoding for exon sequences without an intervening intron sequence in the genomic equivalent sequence.

### Adenosine A2A and A2B Receptor Libraries

Provided herein are methods and compositions relating to G protein-coupled receptor (GPCR) binding libraries for adenosine A2A receptor (ADORA2) comprising nucleic acids encoding for a scaffold comprising an adenosine A2A receptor binding domain. Scaffolds as described herein can stably support an adenosine A2A receptor binding domain. The adenosine A2A receptor binding domain may be designed based on surface interactions of an adenosine A2A receptor ligand and adenosine A2A receptor. Also provided herein are methods and compositions relating to G protein-coupled receptor (GPCR) binding libraries for adenosine A2B receptor (ADORA2B) comprising nucleic acids encoding for a scaffold comprising an adenosine A2B receptor binding domain. Scaffolds as described herein can stably support an adenosine A2B receptor binding domain. The adenosine A2B receptor binding domain may be designed based on surface interactions of an adenosine A2B receptor ligand and adenosine A2B receptor. Libraries as described herein may be further variegated to provide for variant libraries comprising nucleic acids each encoding for a predetermined variant of at least one predetermined reference nucleic acid sequence. Further described herein are protein libraries that may be generated when the nucleic acid libraries are translated. In some instances, nucleic acid libraries as described herein are transferred into cells to generate a cell library. Also provided herein are downstream applications for the libraries synthesized using methods described herein. Downstream applications include identification of variant nucleic acids or protein sequences with enhanced biologically relevant functions, e.g., improved stability, affinity, binding, functional activity, and for the treatment or prevention of a disease state associated with adenosine A2A receptor signaling, adenosine A2B receptor signaling, or both adenosine A2A receptor signaling and adenosine A2B receptor signaling.

Methods, compositions, and systems described herein for the optimization of adenosine A2A receptor immunoglobulins or antibodies, adenosine A2B receptor immunoglobulins or antibodies, or both comprise a ratio-variant approach that mirror the natural diversity of antibody sequences. In some instances, libraries of optimized adenosine A2A receptor immunoglobulins or antibodies comprise variant adenosine A2A receptor immunoglobulin or antibody sequences. In some instances, the variant adenosine A2A receptor immunoglobulin or antibody sequences are designed comprising variant CDR regions. In some instances, the variant adenosine A2A receptor immunoglobulin or antibody sequences comprising variant CDR regions are generated by shuffling the natural CDR sequences in a llama, humanized, or chimeric framework. In some instances, libraries of optimized adenosine A2B receptor immunoglobulins or antibodies comprise variant adenosine A2B receptor immunoglobulin or antibody sequences. In some instances, the variant adenosine A2B receptor immunoglobulin or antibody sequences are designed comprising variant CDR regions. In some instances, the variant adenosine A2B receptor immunoglobulin or antibody sequences comprising variant CDR regions are generated by shuffling the natural CDR sequences in a llama, humanized, or chimeric framework In some instances, such libraries are synthesized, cloned into expression vectors, and translation products (antibodies) evaluated for activity. In some instances, fragments of sequences are synthesized and subsequently assembled. In some instances, expression vectors are used to display and enrich desired antibodies, such as phage display. In some instances, the phage vector is a Fab phagemid vector. Selection pressures used during enrichment in some instances includes binding affinity, toxicity, immunological tolerance, stability, or other factor. Such expression vectors allow antibodies with specific properties to be selected (“panning”), and subsequent propagation or amplification of such sequences enriches the library with these sequences. Panning rounds can be repeated any number of times, such as 1, 2, 3, 4, 5, 6, 7, or more than 7 rounds. In some instances, each round of panning involves a number of washes. In some instances, each round of panning involves at least or about 1, 2, 3, 4, 5, 6, 7, 8, 9, 10, 11, 12, 13, 14, 15, 16, or more than 16 washes.

Described herein are methods and systems of in-silico library design. Libraries as described herein, in some instances, are designed based on a database comprising a variety of antibody sequences. In some instances, the database comprises a plurality of variant antibody sequences against various targets. In some instances, the database comprises at least 100, 500, 1000, 1500, 2000, 2500, 3000, 3500, 4000, 4500, 5000, or more than 5000 antibody sequences. An exemplary database is an iCAN database. In some instances, the database comprises naïve and memory B-cell receptor sequences. In some instances, the naïve and memory B-cell receptor sequences are human, mouse, or primate sequences. In some instances, the naïve and memory B-cell receptor sequences are human sequences. In some instances, the database is analyzed for position specific variation. In some instances, antibodies described herein comprise position specific variations in CDR regions. In some instances, the CDR regions comprise multiple sites for variation.

### Scaffold Libraries

Provided herein are libraries comprising nucleic acids encoding for a scaffold, wherein sequences for adenosine A2A receptor binding domains are placed in the scaffold. Scaffolds described herein allow for improved stability for a range of adenosine A2A receptor binding domain encoding sequences when inserted into the scaffold, as compared to an unmodified scaffold. Exemplary scaffolds include, but are not limited to, a protein, a peptide, an immunoglobulin, derivatives thereof, or combinations thereof. In some instances, the scaffold is an immunoglobulin. Scaffolds as described herein comprise improved functional activity, structural stability, expression, specificity, or a combination thereof. In some instances, scaffolds comprise long regions for supporting an adenosine A2A receptor binding domain.

Provided herein are libraries comprising nucleic acids encoding for a scaffold, wherein sequences for adenosine A2B receptor binding domains are placed in the scaffold. Scaffolds described herein allow for improved stability for a range of adenosine A2B receptor binding domain encoding sequences when inserted into the scaffold, as compared to an unmodified scaffold. Exemplary scaffolds include, but are not limited to, a protein, a peptide, an immunoglobulin, derivatives thereof, or combinations thereof. In some instances, the scaffold is an immunoglobulin. Scaffolds as described herein comprise improved functional activity, structural stability, expression, specificity, or a combination thereof. In some instances, scaffolds comprise long regions for supporting an adenosine A2B receptor binding domain.

Provided herein are libraries comprising nucleic acids encoding for a scaffold, wherein the scaffold is an immunoglobulin. In some instances, the immunoglobulin is an antibody. As used herein, the term antibody will be understood to include proteins having the characteristic two-armed, Y-shape of a typical antibody molecule as well as one or more fragments of an antibody that retain the ability to specifically bind to an antigen. Exemplary antibodies include, but are not limited to, a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv) (including fragments in which the VL and VH are joined using recombinant methods by a synthetic or natural linker that enables them to be made as a single protein chain in which the VL and VH regions pair to form monovalent molecules, including single chain Fab and scFab), a single chain antibody, a Fab fragment (including monovalent fragments comprising the VL, VH, CL, and CHI domains), a F(ab′)2 fragment (including bivalent fragments comprising two Fab fragments linked by a disulfide bridge at the hinge region), a Fd fragment (including fragments comprising the VH and CHI fragment), a Fv fragment (including fragments comprising the VL and VH domains of a single arm of an antibody), a single-domain antibody (dAb or sdAb) (including fragments comprising a VH domain), an isolated complementarity determining region (CDR), a diabody (including fragments comprising bivalent dimers such as two VL and VH domains bound to each other and recognizing two different antigens), a fragment comprised of only a single monomeric variable domain, disulfide-linked Fvs (sdFv), an intrabody, an anti-idiotypic (anti-Id) antibody, or ab antigen-binding fragments thereof. In some instances, the libraries disclosed herein comprise nucleic acids encoding for a scaffold, wherein the scaffold is a Fv antibody, including Fv antibodies comprised of the minimum antibody fragment which contains a complete antigen-recognition and antigen-binding site. In some embodiments, the Fv antibody consists of a dimer of one heavy chain and one light chain variable domain in tight, non-covalent association, and the three hypervariable regions of each variable domain interact to define an antigen-binding site on the surface of the VH-VL dimer. In some embodiments, the six hypervariable regions confer antigen-binding specificity to the antibody. In some embodiments, a single variable domain (or half of an Fv comprising only three hypervariable regions specific for an antigen, including single domain antibodies isolated from camelid animals comprising one heavy chain variable domain or variable region of a heavy chain such as VHH antibodies or nanobodies) has the ability to recognize and bind antigen. In some instances, the libraries disclosed herein comprise nucleic acids encoding for a scaffold, wherein the scaffold is a single-chain Fv or scFv, including antibody fragments comprising a VH, a VL, or both a VH and VL domain, wherein both domains are present in a single polypeptide chain. In some embodiments, the Fv polypeptide further comprises a polypeptide linker between the VH and VL domains allowing the scFv to form the desired structure for antigen binding. In some instances, a scFv is linked to the Fc fragment or a VHH is linked to the Fc fragment (including minibodies). In some instances, the antibody comprises immunoglobulin molecules and immunologically active fragments of immunoglobulin molecules, e.g., molecules that contain an antigen binding site. Immunoglobulin molecules are of any type (e.g., IgG, IgE, IgM, IgD, IgA and IgY), class (e.g., IgG 1, IgG 2, IgG 3, IgG 4, IgA 1 and IgA 2) or subclass.

In some embodiments, libraries comprise immunoglobulins that are adapted to the species of an intended therapeutic target. Generally, these methods include “mammalization” and comprises methods for transferring donor antigen-binding information to a less immunogenic mammal antibody acceptor to generate useful therapeutic treatments. In some instances, the mammal is mouse, rat, equine, sheep, cow, primate (e.g., chimpanzee, baboon, gorilla, orangutan, monkey), dog, cat, pig, donkey, rabbit, and human. In some instances, provided herein are libraries and methods for felinization and caninization of antibodies.

“Humanized” forms of non-human antibodies can be chimeric antibodies that contain minimal sequence derived from the non-human antibody. A humanized antibody is generally a human antibody (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. In some instances, these modifications are made to further refine antibody performance.

“Caninization” can comprise a method for transferring non-canine antigen-binding information from a donor antibody to a less immunogenic canine antibody acceptor to generate treatments useful as therapeutics in dogs. In some instances, caninized forms of non-canine antibodies provided herein are chimeric antibodies that contain minimal sequence derived from non-canine antibodies. In some instances, caninized antibodies are canine antibody sequences (“acceptor” or “recipient” antibody) in which hypervariable region residues of the recipient are replaced by hypervariable region residues from a non-canine species (“donor” antibody) such as mouse, rat, rabbit, cat, dogs, goat, chicken, bovine, horse, llama, camel, dromedaries, sharks, non-human primates, human, humanized, recombinant sequence, or an engineered sequence having the desired properties. In some instances, framework region (FR) residues of the canine antibody are replaced by corresponding non-canine FR residues. In some instances, caninized antibodies include residues that are not found in the recipient antibody or in the donor antibody. In some instances, these modifications are made to further refine antibody performance. The caninized antibody may also comprise at least a portion of an immunoglobulin constant region (Fc) of a canine antibody.

“Felinization” can comprise a method for transferring non-feline antigen-binding information from a donor antibody to a less immunogenic feline antibody acceptor to generate treatments useful as therapeutics in cats. In some instances, felinized forms of non-feline antibodies provided herein are chimeric antibodies that contain minimal sequence derived from non-feline antibodies. In some instances, felinized antibodies are feline antibody sequences (“acceptor” or “recipient” antibody) in which hypervariable region residues of the recipient are replaced by hypervariable region residues from a non-feline species (“donor” antibody) such as mouse, rat, rabbit, cat, dogs, goat, chicken, bovine, horse, llama, camel, dromedaries, sharks, non-human primates, human, humanized, recombinant sequence, or an engineered sequence having the desired properties. In some instances, framework region (FR) residues of the feline antibody are replaced by corresponding non-feline FR residues. In some instances, felinized antibodies include residues that are not found in the recipient antibody or in the donor antibody. In some instances, these modifications are made to further refine antibody performance. The felinized antibody may also comprise at least a portion of an immunoglobulin constant region (Fc) of a felinize antibody.

Provided herein are libraries comprising nucleic acids encoding for a scaffold, wherein the scaffold is a non-immunoglobulin. In some instances, the scaffold is a non-immunoglobulin binding domain. For example, the scaffold is an antibody mimetic. Exemplary antibody mimetics include, but are not limited to, anticalins, affilins, affibody molecules, affimers, affitins, alphabodies, avimers, atrimers, DARPins, fynomers, Kunitz domain-based proteins, monobodies, anticalins, knottins, armadillo repeat protein-based proteins, and bicyclic peptides.

Libraries described herein comprising nucleic acids encoding for a scaffold, wherein the scaffold is an immunoglobulin, comprise variations in at least one region of the immunoglobulin. Exemplary regions of the antibody for variation include, but are not limited to, a complementarity-determining region (CDR), a variable domain, or a constant domain. In some instances, the CDR is CDR1, CDR2, or CDR3. In some instances, the CDR is a heavy domain including, but not limited to, CDRH1, CDRH2, and CDRH3. In some instances, the CDR is a light domain including, but not limited to, CDRL1, CDRL2, and CDRL3. In some instances, the variable domain is variable domain, light chain (VL) or variable domain, heavy chain (VH). In some instances, the VL domain comprises kappa or lambda chains. In some instances, the constant domain is constant domain, light chain (CL) or constant domain, heavy chain (CH).

Methods described herein provide for synthesis of libraries comprising nucleic acids encoding for a scaffold, wherein each nucleic acid encodes for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. In some instances, the scaffold library comprises varied nucleic acids collectively encoding variations at multiple positions. In some instances, the variant library comprises sequences encoding for variation of at least a single codon of a CDRH1, CDRH2, CDRH3, CDRL1, CDRL2, CDRL3, VL, or VH domain. In some instances, the variant library comprises sequences encoding for variation of multiple codons of a CDRH1, CDRH2, CDRH3, CDRL1, CDRL2, CDRL3, VL, or VH domain. In some instances, the variant library comprises sequences encoding for variation of multiple codons of framework element 1 (FW1), framework element 2 (FW2), framework element 3 (FW3), or framework element 4 (FW4). An exemplary number of codons for variation include, but are not limited to, at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons.

In some instances, the at least one region of the immunoglobulin for variation is from heavy chain V-gene family, heavy chain D-gene family, heavy chain J-gene family, light chain V-gene family, or light chain J-gene family. In some instances, the light chain V-gene family comprises immunoglobulin kappa (IGK) gene or immunoglobulin lambda (IGL). Exemplary genes include, but are not limited to, IGHV1-18, IGHV1-69, IGHV1-8, IGHV3-21, IGHV3-23, IGHV3-30/33rn, IGHV3-28, IGHV1-69, IGHV3-74, IGHV4-39, IGHV4-59/61, IGKV1-39, IGKV1-9, IGKV2-28, IGKV3-11, IGKV3-15, IGKV3-20, IGKV4-1, IGLV1-51, IGLV2-14, IGLV1-40, and IGLV3-1. In some instances, the gene is IGHV1-69, IGHV3-30, IGHV3-23, IGHV3, IGHV1-46, IGHV3-7, IGHV1, or IGHV1-8. In some instances, the gene is IGHV1-69 and IGHV3-30. In some instances, the gene is IGHJ3, IGHJ6, IGHJ, IGHJ4, IGHJ5, IGHJ2, or IGH1. In some instances, the gene is IGHJ3, IGHJ6, IGHJ, or IGHJ4.

Provided herein are libraries comprising nucleic acids encoding for immunoglobulin scaffolds, wherein the libraries are synthesized with various numbers of fragments. In some instances, the fragments comprise the CDRH1, CDRH2, CDRH3, CDRL1, CDRL2, CDRL3, VL, or VH domain. In some instances, the fragments comprise framework element 1 (FW1), framework element 2 (FW2), framework element 3 (FW3), or framework element 4 (FW4). In some instances, the scaffold libraries are synthesized with at least or about 2 fragments, 3 fragments, 4 fragments, 5 fragments, or more than 5 fragments. The length of each of the nucleic acid fragments or average length of the nucleic acids synthesized may be at least or about 50, 75, 100, 125, 150, 175, 200, 225, 250, 275, 300, 325, 350, 375, 400, 425, 450, 475, 500, 525, 550, 575, 600, or more than 600 base pairs. In some instances, the length is about 50 to 600, 75 to 575, 100 to 550, 125 to 525, 150 to 500, 175 to 475, 200 to 450, 225 to 425, 250 to 400, 275 to 375, or 300 to 350 base pairs.

Libraries comprising nucleic acids encoding for immunoglobulin scaffolds as described herein comprise various lengths of amino acids when translated. In some instances, the length of each of the amino acid fragments or average length of the amino acid synthesized may be at least or about 15, 20, 25, 30, 35, 40, 45, 50, 55, 60, 65, 70, 75, 80, 85, 90, 95, 100, 105, 110, 115, 120, 125, 130, 135, 140, 145, 150, or more than 150 amino acids. In some instances, the length of the amino acid is about 15 to 150, 20 to 145, 25 to 140, 30 to 135, 35 to 130, 40 to 125, 45 to 120, 50 to 115, 55 to 110, 60 to 110, 65 to 105, 70 to 100, or 75 to 95 amino acids. In some instances, the length of the amino acid is about 22 amino acids to about 75 amino acids. In some instances, the immunoglobulin scaffolds comprise at least or about 100, 200, 300, 400, 500, 600, 700, 800, 900, 1000, 2000, 3000, 4000, 5000, or more than 5000 amino acids.

A number of variant sequences for the at least one region of the immunoglobulin for variation are de novo synthesized using methods as described herein. In some instances, a number of variant sequences is de novo synthesized for CDRH1, CDRH2, CDRH3, CDRL1, CDRL2, CDRL3, VL, VH, or combinations thereof. In some instances, a number of variant sequences is de novo synthesized for framework element 1 (FW1), framework element 2 (FW2), framework element 3 (FW3), or framework element 4 (FW4). The number of variant sequences may be at least or about 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, or more than 500 sequences. In some instances, the number of variant sequences is at least or about 500, 600, 700, 800, 900, 1000, 2000, 3000, 4000, 5000, 6000, 7000, 8000, or more than 8000 sequences. In some instances, the number of variant sequences is about 10 to 500, 25 to 475, 50 to 450, 75 to 425, 100 to 400, 125 to 375, 150 to 350, 175 to 325, 200 to 300, 225 to 375, 250 to 350, or 275 to 325 sequences.

Variant sequences for the at least one region of the immunoglobulin, in some instances, vary in length or sequence. In some instances, the at least one region that is de novo synthesized is for CDRH1, CDRH2, CDRH3, CDRL1, CDRL2, CDRL3, VL, VH, or combinations thereof. In some instances, the at least one region that is de novo synthesized is for framework element 1 (FW1), framework element 2 (FW2), framework element 3 (FW3), or framework element 4 (FW4). In some instances, the variant sequence comprises at least or about 1, 2, 3, 4, 5, 6, 7, 8, 9, 10, 15, 20, 25, 30, 35, 40, 45, 50, or more than 50 variant nucleotides or amino acids as compared to wild-type. In some instances, the variant sequence comprises at least or about 1, 2, 3, 4, 5, 6, 7, 8, 9, 10, 15, 20, 25, 30, 35, 40, 45, or 50 additional nucleotides or amino acids as compared to wild-type. In some instances, the variant sequence comprises at least or about 1, 2, 3, 4, 5, 6, 7, 8, 9, 10, 15, 20, 25, 30, 35, 40, 45, or 50 less nucleotides or amino acids as compared to wild-type. In some instances, the libraries comprise at least or about 10¹, 10², 10³, 10⁴, 10⁵, 10⁶, 10⁷, 10⁸, 10⁹, 10¹⁰, or more than 10¹⁰ variants.

Following synthesis of scaffold libraries, scaffold libraries may be used for screening and analysis. For example, scaffold libraries are assayed for library displayability and panning. In some instances, displayability is assayed using a selectable tag. Exemplary tags include, but are not limited to, a radioactive label, a fluorescent label, an enzyme, a chemiluminescent tag, a colorimetric tag, an affinity tag or other labels or tags that are known in the art. In some instances, the tag is histidine, polyhistidine, myc, hemagglutinin (HA), or FLAG. In some instances, scaffold libraries are assayed by sequencing using various methods including, but not limited to, single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis.

In some instances, the scaffold libraries are assayed for functional activity, structural stability (e.g., thermal stable or pH stable), expression, specificity, or a combination thereof. In some instances, the scaffold libraries are assayed for scaffolds capable of folding. In some instances, a region of the antibody is assayed for functional activity, structural stability, expression, specificity, folding, or a combination thereof. For example, a VH region or VL region is assayed for functional activity, structural stability, expression, specificity, folding, or a combination thereof.

### Adenosine A2A Receptor Libraries

Provided herein are adenosine A2A receptor binding libraries comprising nucleic acids encoding for scaffolds comprising sequences for adenosine A2A receptor binding domains. In some instances, the scaffolds are immunoglobulins. In some instances, the scaffolds comprising sequences for adenosine A2A receptor binding domains are determined by interactions between the adenosine A2A receptor binding domains and the adenosine A2A receptor.

Provided herein are libraries comprising nucleic acids encoding scaffolds comprising adenosine A2A receptor binding domains, wherein the adenosine A2A receptor binding domains are designed based on surface interactions on adenosine A2A receptor. In some instances, the adenosine A2A receptor binding domain comprises a sequence as defined by SEQ ID NO: 1. In some instances, the adenosine A2A receptor binding domains interact with the amino- or carboxy-terminus of the adenosine A2A receptor. In some instances, the adenosine A2A receptor binding domains interact with at least one transmembrane domain including, but not limited to, transmembrane domain 1 (TM1), transmembrane domain 2 (TM2), transmembrane domain 3 (TM3), transmembrane domain 4 (TM4), transmembrane domain 5 (TM5), transmembrane domain 6 (TM6), and transmembrane domain 7 (TM7). In some instances, the adenosine A2A receptor binding domains interact with an intracellular surface of the adenosine A2A receptor. For example, the adenosine A2A receptor binding domains interact with at least one intracellular loop including, but not limited to, intracellular loop 1 (ICL1), intracellular loop 2 (ICL2), and intracellular loop 3 (ICL3). In some instances, the adenosine A2A receptor binding domains interact with an extracellular surface of the adenosine A2A receptor For example, the adenosine A2A receptor binding domains interact with at least one extracellular domain (ECD) or extracellular loop (ECL) of the adenosine A2A receptor. The extracellular loops include, but are not limited to, extracellular loop 1 (ECL1), extracellular loop 2 (ECL2), and extracellular loop 3 (ECL3).

Described herein are adenosine A2A receptor binding domains, wherein the adenosine A2A receptor binding domains are designed based on surface interactions between an adenosine A2A receptor ligand and the adenosine A2A receptor. In some instances, the ligand is a peptide. In some instances, the ligand is an adenosine A2A receptor agonist. In some instances, the ligand is an adenosine A2A receptor antagonist. In some instances, the ligand is an adenosine A2A receptor allosteric modulator. In some instances, the allosteric modulator is a negative allosteric modulator. In some instances, the allosteric modulator is a positive allosteric modulator. Exemplary ligands of the adenosine A2A receptor include, but are not limited to DU172, PSB36, ZM241385, XAC, caffeine, T4G, T4E, 6DY, 6DZ, 6DX, 6DV, 8D1b, theophylline, UK-432097, adenosine, NECA, and CGS21680.

Sequences of adenosine A2A receptor binding domains based on surface interactions between an adenosine A2A receptor ligand and the adenosine A2A receptor are analyzed using various methods. For example, multispecies computational analysis is performed. In some instances, a structure analysis is performed. In some instances, a sequence analysis is performed. Sequence analysis can be performed using a database known in the art. Non-limiting examples of databases include, but are not limited to, NCBI BLAST (blast.ncbi.nlm.nih.gov/Blast.cgi), UCSC Genome Browser (genome.ucsc.edu/), UniProt (uniprot.org/), and IUPHAR/BPS Guide to PHARMACOLOGY (guidetopharmacology.org/).

Described herein are adenosine A2A receptor binding domains designed based on sequence analysis among various organisms. For example, sequence analysis is performed to identify homologous sequences in different organisms. Exemplary organisms include, but are not limited to, mouse, rat, equine, sheep, cow, primate (e.g., chimpanzee, baboon, gorilla, orangutan, monkey), dog, cat, pig, donkey, rabbit, fish, fly, and human.

Following identification of adenosine A2A receptor binding domains, libraries comprising nucleic acids encoding for the adenosine A2A receptor binding domains may be generated. In some instances, libraries of adenosine A2A receptor binding domains comprise sequences of adenosine A2A receptor binding domains designed based on conformational ligand interactions, peptide ligand interactions, small molecule ligand interactions, extracellular domains of adenosine A2A receptor, or antibodies that target adenosine A2A receptor. In some instances, libraries of adenosine A2A receptor binding domains comprise sequences of adenosine A2A receptor binding domains designed based on peptide ligand interactions. In some instances, the ligand is a not an antibody ligand. Libraries of adenosine A2A receptor binding domains may be translated to generate protein libraries. In some instances, libraries of adenosine A2A receptor binding domains are translated to generate peptide libraries, immunoglobulin libraries, derivatives thereof, or combinations thereof. In some instances, libraries of adenosine A2A receptor binding domains are translated to generate protein libraries that are further modified to generate peptidomimetic libraries. In some instances, libraries of adenosine A2A receptor binding domains are translated to generate protein libraries that are used to generate small molecules.

Methods described herein provide for synthesis of libraries of adenosine A2A receptor binding domains comprising nucleic acids each encoding for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. In some instances, the libraries of adenosine A2A receptor binding domains comprise varied nucleic acids collectively encoding variations at multiple positions. In some instances, the variant library comprises sequences encoding for variation of at least a single codon in an adenosine A2A receptor binding domain. In some instances, the variant library comprises sequences encoding for variation of multiple codons in an adenosine A2A receptor binding domain. An exemplary number of codons for variation include, but are not limited to, at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons.

Methods described herein provide for synthesis of libraries comprising nucleic acids encoding for the adenosine A2A receptor binding domains, wherein the libraries comprise sequences encoding for variation of length of the adenosine A2A receptor binding domains. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons less as compared to a predetermined reference sequence. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 200, 225, 250, 275, 300, or more than 300 codons more as compared to a predetermined reference sequence.

Following identification of adenosine A2A receptor binding domains, the adenosine A2A receptor binding domains may be placed in scaffolds as described herein. In some instances, the scaffolds are immunoglobulins. In some instances, the adenosine A2A receptor binding domains are placed in the CDRH3 region. Adenosine A2A receptor binding domains that may be placed in scaffolds can also be referred to as a motif. Scaffolds comprising adenosine A2A receptor binding domains may be designed based on binding, specificity, stability, expression, folding, or downstream activity. In some instances, the scaffolds comprising adenosine A2A receptor binding domains enable contact with the adenosine A2A receptor. In some instances, the scaffolds comprising adenosine A2A receptor binding domains enables high affinity binding with the adenosine A2A receptor. An exemplary amino acid sequence of adenosine A2A receptor binding domain is described in Table 1.

TABLE 1
Adenosine A2A receptor binding domain
amino acid sequences

| SEQ | | |
|---|---|---|
| ID | | |
| NO | GPCR | Amino Acid Sequence |
| 1 | Adenosine | MPIMGSSVYITVELAIAVLAILGNVLVCWAVWL |
| | A2A | NSNLQNVTNYFVVSLAAADIAVGVLAIPFAITI |
| | receptor | STGFCAACHGCLFIACFVLVLTQSSIFSLLAIA |
| | | IDRYIAIRIPLRYNGLVTGTRAKGIIAICWVLS |
| | | FAIGLTPMLGWNNCGQPKEGKNHSQGCGEGQVA |
| | | CLFEDVVPMNYMVYFNFFACVLVPLLLMLGVYL |
| | | RIFLAARRQLKQMESQPLPGERARSTLQKEVHA |
| | | AKSLAIIVGLFALCWLPLHIINCFTFFCPDCSH |
| | | APLWLMYLAIVLSHTNSVVNPFIYAYRIREFRQ |
| | | TFRKIIRSHVLRQQEPFKAAGTSARVLAAHGSD |
| | | GEQVSLRLNGHPPGVWANGSAPHPERRPNGYAL |
| | | GLVSGGSAQESQGNTGLPDVELLSHELKGVCPE |
| | | PPGLDDPLAQDGAGVS |

Provided herein are scaffolds or immunoglobulins comprising adenosine A2A receptor binding domains, wherein the sequences of the adenosine A2A receptor binding domains support interaction with adenosine A2A receptor. The sequence may be homologous or identical to a sequence of an adenosine A2A receptor ligand. In some instances, the adenosine A2A receptor binding domain sequence comprises at least or about 70%, 80%, 85%, 90%, 91%, 92%, 93%, 94%, 95%, 96%, 97%, 98%, 99%, or 100% sequence identity to SEQ ID NO: 1. In some instances, the adenosine A2A receptor binding domain sequence comprises at least or about 95% homology to SEQ ID NO: 1. In some instances, the adenosine A2A receptor binding domain sequence comprises at least or about 97% homology to SEQ ID NO: 1. In some instances, the adenosine A2A receptor binding domain sequence comprises at least or about 99% homology to SEQ ID NO: 1. In some instances, the adenosine A2A receptor binding domain sequence comprises at least or about 100% homology to SEQ ID NO: 1. In some instances, the adenosine A2A receptor binding domain sequence comprises at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, or more than 400 amino acids of SEQ ID NO: 1.

Provided herein are antibodies or immunoglobulins, wherein the antibody or immunoglobulin comprises a sequence at least or about 70%, 80%, 85%, 90%, 91%, 92%, 93%, 94%, 95%, 96%, 97%, 98%, 99%, or 100% sequence identity to any one of SEQ ID NOs: 540-717. In some instances, the antibody or immunoglobulin sequence comprises at least or about 95% sequence identity to any one of SEQ ID NOs: 540-717. In some instances, the antibody or immunoglobulin sequence comprises at least or about 97% sequence identity to any one of SEQ ID NOs: 540-717. In some instances, the antibody or immunoglobulin sequence comprises at least or about 99% sequence identity to any one of SEQ ID NOs: 540-717. In some instances, the antibody or immunoglobulin sequence comprises at least or about 100% sequence identity to any one SEQ ID NOs: 540-717. In some instances, the antibody or immunoglobulin sequence comprises at least a portion having at least or about 1, 2, 3, 4, 5, 6, 7, 8, 9, 10, 12, 14, 16, 18, 20, 30, 40, 50, 60, 70, 80, 90, 100, 110, or more than 110 amino acids of any one of SEQ ID NOs: 540-717.

In some embodiments, the antibody or immunoglobulin sequence comprises complementarity determining regions (CDRs) comprising a sequence as set forth in Tables 15-16. In some embodiments, the antibody or immunoglobulin sequence comprises complementarity determining regions (CDRs) comprising at least or about 70%, 80%, 85%, 90%, 91%, 92%, 93%, 94%, 95%, 96%, 97%, 98%, 99%, or 100% sequence identity to any one of SEQ ID NOs: 6-539. In some instances, the antibody or immunoglobulin sequence comprises complementarity determining regions (CDRs) comprising at least or about 95% homology to any one of SEQ ID NOs: 6-539. In some instances, the antibody or immunoglobulin sequence comprises complementarity determining regions (CDRs) comprising at least or about 97% homology to any one of SEQ ID NOs: 6-539. In some instances, the antibody or immunoglobulin sequence comprises complementarity determining regions (CDRs) comprising at least or about 99% homology to any one of SEQ ID NOs: 6-539. In some instances, the antibody or immunoglobulin sequence comprises complementarity determining regions (CDRs) comprising at least or about 100% homology to any one of SEQ ID NOs: 6-539. In some instances, the antibody or immunoglobulin sequence comprises complementarity determining regions (CDRs) comprising at least a portion having at least or about 3, 4, 5, 6, 7, 8, 9, 10, 12, 14, 16, or more than 16 amino acids of any one of SEQ ID NOs: 6-539.

In some embodiments, the antibody or immunoglobulin sequence comprises a CDR1 comprising at least or about 70%, 80%, 85%, 90%, 91%, 92%, 93%, 94%, 95%, 96%, 97%, 98%, 99%, or 100% sequence identity to any one of SEQ ID NOs: 6-94 or 273-361. In some instances, the antibody or immunoglobulin sequence comprises CDR1 comprising at least or about 95% homology of any one of SEQ ID NOs: 6-94 and 273-361. In some instances, the antibody or immunoglobulin sequence comprises CDR1 comprising at least or about 97% homology to any one of SEQ ID NOs: 6-94 or 273-361. In some instances, the antibody or immunoglobulin sequence comprises CDR 1 comprising at least or about 99% homology to any one of SEQ ID NOs: 6-94 or 273-361. In some instances, the antibody or immunoglobulin sequence comprises CDR1 comprising at least or about 100% homology to any one of SEQ ID NOs: 6-270 or 273-537. In some instances, the antibody or immunoglobulin sequence comprises CDR1 comprising at least a portion having at least or about 3, 4, 5, 6, 7, 8, 9, 10, 12, 14, 16, or more than 16 amino acids of any one of SEQ ID NOs: 6-94 or 273-361.

In some embodiments, the antibody or immunoglobulin sequence comprises a CDR2 comprising at least or about 70%, 80%, 85%, 90%, 91%, 92%, 93%, 94%, 95%, 96%, 97%, 98%, 99%, or 100% sequence identity to any one of SEQ ID NOs: 95-183 and 362-450. In some instances, the antibody or immunoglobulin sequence comprises CDR2 comprising at least or about 95% homology to any one of SEQ ID NOs: 95-183 and 362-450. In some instances, the antibody or immunoglobulin sequence comprises CDR2 comprising at least or about 97% homology to any one of SEQ ID NOs: 795-183 and 362-450. In some instances, the antibody or immunoglobulin sequence comprises CDR2 comprising at least or about 99% homology to any one of SEQ ID NOs: 95-183 and 362-450. In some instances, the antibody or immunoglobulin sequence comprises CDR2 comprising at least or about 100% homology to any one of SEQ ID NOs: 95-183 and 362-450. In some instances, the antibody or immunoglobulin sequence comprises CDR2 comprising at least a portion having at least or about 3, 4, 5, 6, 7, 8, 9, 10, 12, 14, 16, or more than 16 amino acids of any one of SEQ ID NOs: 95-183 and 362-450.

In some embodiments, the antibody or immunoglobulin sequence comprises a CDR3 comprising at least or about 70%, 80%, 85%, 90%, 91%, 92%, 93%, 94%, 95%, 96%, 97%, 98%, 99%, or 100% sequence identity to any one of SEQ ID NOs: 184-272 and 451-539. In some instances, the antibody or immunoglobulin sequence comprises CDR3 comprising at least or about 95% homology to any one of SEQ ID NOs: 184-272 and 451-539. In some instances, the antibody or immunoglobulin sequence comprises CDR3 comprising at least or about 97% homology to any one of SEQ ID NOs: 184-272 and 451-539. In some instances, the antibody or immunoglobulin sequence comprises CDR3 comprising at least or about 99% homology to any one of SEQ ID NOs: 184-272 and 451-539. In some instances, the antibody or immunoglobulin sequence comprises CDR3 comprising at least or about 100% homology to any one of SEQ ID NOs: 184-272 and 451-539. In some instances, the antibody or immunoglobulin sequence comprises CDR3 comprising at least a portion having at least or about 3, 4, 5, 6, 7, 8, 9, 10, 12, 14, 16, or more than 16 amino acids of any one of SEQ ID NOs: 184-272 and 451-539.

In some embodiments, the antibody or immunoglobulin sequence comprises a CDRH1 comprising at least or about 70%, 80%, 85%, 90%, 91%, 92%, 93%, 94%, 95%, 96%, 97%, 98%, 99%, or 100% sequence identity to any one of SEQ ID NOs: 6-94; a CDRH2 comprising at least or about 70%, 80%, 85%, 90%, 91%, 92%, 93%, 94%, 95%, 96%, 97%, 98%, 99%, or 100% sequence identity to any one of SEQ ID NOs: 95-183; and a CDRH3 comprising at least or about 70%, 80%, 85%, 90%, 91%, 92%, 93%, 94%, 95%, 96%, 97%, 98%, 99%, or 100% sequence identity to any one of SEQ ID NOs: 184-272. In some instances, the antibody or immunoglobulin sequence comprises CDRH1 comprising at least or about 95%, 97%, 99%, or 100% homology to any one of SEQ ID NOs: 6-94; a CDRH2 comprising at least or about 95%, 97%, 99%, or 100% homology to any one of SEQ ID NOs: 95-183; and a CDRH3 comprising at least or about 95%, 97%, 99%, or 100% homology to any one of SEQ ID NOs: 184-272. In some instances, the antibody or immunoglobulin sequence comprises CDRH1 comprising at least a portion having at least or about 3, 4, 5, 6, 7, 8, 9, 10, 12, 14, 16, or more than 16 amino acids of SEQ ID NO: 6-94; a CDRH2 comprising at least a portion having at least or about 3, 4, 5, 6, 7, 8, 9, 10, 12, 14, 16, or more than 16 amino acids of SEQ ID NO: 95-183; and a CDRH3 comprising at least a portion having at least or about 3, 4, 5, 6, 7, 8, 9, 10, 12, 14, 16, or more than 16 amino acids of SEQ ID NO: 184-272.

In some embodiments, the antibody or immunoglobulin sequence comprises a CDRL1 comprising at least or about 70%, 80%, 85%, 90%, 91%, 92%, 93%, 94%, 95%, 96%, 97%, 98%, 99%, or 100% sequence identity to SEQ ID NO: 273-361; a CDRL2 comprising at least or about 70%, 80%, 85%, 90%, 91%, 92%, 93%, 94%, 95%, 96%, 97%, 98%, 99%, or 100% sequence identity to SEQ ID NO: 362-450; and a CDRL3 comprising at least or about 70%, 80%, 85%, 90%, 91%, 92%, 93%, 94%, 95%, 96%, 97%, 98%, 99%, or 100% sequence identity to SEQ ID NO: 451-539. In some instances, the antibody or immunoglobulin sequence comprises CDRL1 comprising at least or about 95%, 97%, 99%, or 100% homology to SEQ ID NO: 273-361; a CDRL2 comprising at least or about 95%, 97%, 99%, or 100% homology to SEQ ID NO: 362-450; and a CDRL3 comprising at least or about 95%, 97%, 99%, or 100% homology to SEQ ID NO: 451-539. In some instances, the antibody or immunoglobulin sequence comprises CDRL1 comprising at least a portion having at least or about 3, 4, 5, 6, 7, 8, 9, 10, 12, 14, 16, or more than 16 amino acids of SEQ ID NO: 273-361; a CDRL2 comprising at least a portion having at least or about 3, 4, 5, 6, 7, 8, 9, 10, 12, 14, 16, or more than 16 amino acids of SEQ ID NO: 362-450; and a CDRL3 comprising at least a portion having at least or about 3, 4, 5, 6, 7, 8, 9, 10, 12, 14, 16, or more than 16 amino acids of SEQ ID NO: 451-539.

In some embodiments, the antibody or immunoglobulin sequence comprises a CDRH1 comprising at least or about 70%, 80%, 85%, 90%, 91%, 92%, 93%, 94%, 95%, 96%, 97%, 98%, 99%, or 100% sequence identity to any one of SEQ ID NOs: 6-94; a CDRH2 comprising at least or about 70%, 80%, 85%, 90%, 91%, 92%, 93%, 94%, 95%, 96%, 97%, 98%, 99%, or 100% sequence identity to any one of SEQ ID NOs: 95-183; a CDRH3 comprising at least or about 70%, 80%, 85%, 90%, 91%, 92%, 93%, 94%, 95%, 96%, 97%, 98%, 99%, or 100% sequence identity to any one of SEQ ID NOs: 184-272, a CDRL1 comprising at least or about 70%, 80%, 85%, 90%, 91%, 92%, 93%, 94%, 95%, 96%, 97%, 98%, 99%, or 100% sequence identity to any one of SEQ ID NOs: 273-362; a CDRL2 comprising at least or about 70%, 80%, 85%, 90%, 91%, 92%, 93%, 94%, 95%, 96%, 97%, 98%, 99%, or 100% sequence identity to any one of SEQ ID NOs: 362-450; and a CDRL3 comprising at least or about 70%, 80%, 85%, 90%, 91%, 92%, 93%, 94%, 95%, 96%, 97%, 98%, 99%, or 100% sequence identity to any one of SEQ ID NOs: 451-539. In some instances, the antibody or immunoglobulin sequence comprises CDRH1 comprising at least or about 95%, 97%, 99%, or 100% homology to any one of SEQ ID NOs: 6-94; a CDRH2 comprising at least or about 95%, 97%, 99%, or 100% homology to any one of SEQ ID NOs: 95-183; a CDRH3 comprising at least or about 95%, 97%, 99%, or 100% homology to any one of SEQ ID NOs: 184-272; a CDRL1 comprising at least or about 95%, 97%, 99%, or 100% homology to any one of SEQ ID NOs: 273-362; a CDRL2 comprising at least or about 95%, 97%, 99%, or 100% homology to any one of SEQ ID NOs: 362-450; and a CDRL3 comprising at least or about 95%, 97%, 99%, or 100% homology to any one of SEQ ID NOs: 451-539. In some instances, the antibody or immunoglobulin sequence comprises CDRH1 comprising at least a portion having at least or about 3, 4, 5, 6, 7, 8, 9, 10, 12, 14, 16, or more than 16 amino acids of any one of SEQ ID NOs: 6-94; a CDRH2 comprising at least a portion having at least or about 3, 4, 5, 6, 7, 8, 9, 10, 12, 14, 16, or more than 16 amino acids of any one of SEQ ID NOs: 95-183; a CDRH3 comprising at least a portion having at least or about 3, 4, 5, 6, 7, 8, 9, 10, 12, 14, 16, or more than 16 amino acids of any one of SEQ ID NOs: 184-272; a CDRL1 comprising at least a portion having at least or about 3, 4, 5, 6, 7, 8, 9, 10, 12, 14, 16, or more than 16 amino acids of any one of SEQ ID NOs: 273-362; a CDRL2 comprising at least a portion having at least or about 3, 4, 5, 6, 7, 8, 9, 10, 12, 14, 16, or more than 16 amino acids of any one of SEQ ID NOs: 362-450; and a CDRL3 comprising at least a portion having at least or about 3, 4, 5, 6, 7, 8, 9, 10, 12, 14, 16, or more than 16 amino acids of any one of SEQ ID NOs: 451-539.

Described herein, in some embodiments, are antibodies or immunoglobulins that bind to the adenosine A2A receptor. In some instances, the adenosine A2A receptor antibody or immunoglobulin sequence comprises a heavy chain variable domain comprising at least or about 70%, 80%, 85%, 90%, 91%, 92%, 93%, 94%, 95%, 96%, 97%, 98%, 99%, or 100% sequence identity to any one of SEQ ID NOs: 540-628. In some instances, the adenosine A2A receptor antibody or immunoglobulin sequence comprises a heavy chain variable domain comprising at least or about 95% sequence identity to any one of SEQ ID NOs: 540-628. In some instances, the adenosine A2A receptor antibody or immunoglobulin sequence comprises a heavy chain variable domain comprising at least or about 97% sequence identity to any one of SEQ ID NOs: 540-628. In some instances, the adenosine A2A receptor antibody or immunoglobulin sequence comprises a heavy chain variable domain comprising at least or about 99% sequence identity to any one of SEQ ID NOs: 540-628. In some instances, the adenosine A2A receptor antibody or immunoglobulin sequence comprises a heavy chain variable domain comprising at least or about 100% sequence identity to any one of SEQ ID NOs: 540-628. In some instances, the adenosine A2A receptor antibody or immunoglobulin sequence comprises a heavy chain variable domain comprising at least a portion having at least or about 1, 2, 3, 4, 5, 6, 7, 8, 9, 10, 12, 14, 16, 18, 20, 30, 40, 50, 60, 70, 80, 90, 100, 110, or more than 110 amino acids of SEQ ID NOs: 540-628.

In some instances, the adenosine A2A receptor antibody or immunoglobulin sequence comprises a light chain variable domain comprising at least or about 70%, 80%, 85%, 90%, 91%, 92%, 93%, 94%, 95%, 96%, 97%, 98%, 99%, or 100% sequence identity to any one of SEQ ID NOs: 629-717. In some instances, the adenosine A2A receptor antibody or immunoglobulin sequence comprises a light chain variable domain comprising at least or about 95% sequence identity to any one of SEQ ID NOs: 629-717. In some instances, the adenosine A2A receptor antibody or immunoglobulin sequence comprises a light chain variable domain comprising at least or about 97% sequence identity to any one of SEQ ID NOs: 629-717. In some instances, the adenosine A2A receptor antibody or immunoglobulin sequence comprises a light chain variable domain comprising at least or about 99% sequence identity to any one of SEQ ID NOs: 629-717. In some instances, the adenosine A2A receptor antibody or immunoglobulin sequence comprises a light chain variable domain comprising at least or about 100% sequence identity to any one of SEQ ID NOs: 629-717. In some instances, the adenosine A2A receptor antibody or immunoglobulin sequence comprises a light chain variable domain comprising at least a portion having at least or about 1, 2, 3, 4, 5, 6, 7, 8, 9, 10, 12, 14, 16, 18, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, or more than 400 amino acids of SEQ ID NOs: 629-717.

In some embodiments, the immunoglobulin heavy chain comprises an amino acid sequence at least about 90% identical to that set forth in SEQ ID NO: 540; and the immunoglobulin light chain comprises an amino acid sequence at least about 90% identical to that set forth in SEQ ID NO: 629. In some embodiments, the immunoglobulin heavy chain comprises an amino acid sequence at least about 90% identical to that set forth in SEQ ID NO: 541; and the immunoglobulin light chain comprises an amino acid sequence at least about 90% identical to that set forth in SEQ ID NO: 630. In some embodiments, the immunoglobulin heavy chain comprises an amino acid sequence at least about 90% identical to that set forth in SEQ ID NO: 542; and the immunoglobulin light chain comprises an amino acid sequence at least about 90% identical to that set forth in SEQ ID NO: 631. In some embodiments, the immunoglobulin heavy chain comprises an amino acid sequence at least about 90% identical to that set forth in SEQ ID NO: 543; and the immunoglobulin light chain comprises an amino acid sequence at least about 90% identical to that set forth in SEQ ID NO: 632. In some embodiments, the immunoglobulin heavy chain comprises an amino acid sequence at least about 90% identical to that set forth in SEQ ID NO: 544; and the immunoglobulin light chain comprises an amino acid sequence at least about 90% identical to that set forth in SEQ ID NO: 633. In some embodiments, the immunoglobulin heavy chain comprises an amino acid sequence at least about 90% identical to that set forth in SEQ ID NO: 545; and the immunoglobulin light chain comprises an amino acid sequence at least about 90% identical to that set forth in SEQ ID NO: 634. In some embodiments, the immunoglobulin heavy chain comprises an amino acid sequence at least about 90% identical to that set forth in SEQ ID NO: 546; and the immunoglobulin light chain comprises an amino acid sequence at least about 90% identical to that set forth in SEQ ID NO: 635. In some embodiments, the immunoglobulin heavy chain comprises an amino acid sequence at least about 90% identical to that set forth in SEQ ID NO: 547; and the immunoglobulin light chain comprises an amino acid sequence at least about 90% identical to that set forth in SEQ ID NO: 636. In some embodiments, the immunoglobulin heavy chain comprises an amino acid sequence at least about 90% identical to that set forth in SEQ ID NO: 548; and the immunoglobulin light chain comprises an amino acid sequence at least about 90% identical to that set forth in SEQ ID NO: 637. In some embodiments, the immunoglobulin heavy chain comprises an amino acid sequence at least about 90% identical to that set forth in SEQ ID NO: 549; and the immunoglobulin light chain comprises an amino acid sequence at least about 90% identical to that set forth in SEQ ID NO: 638. In some embodiments, the immunoglobulin heavy chain comprises an amino acid sequence at least about 90% identical to that set forth in SEQ ID NO: 550; and the immunoglobulin light chain comprises an amino acid sequence at least about 90% identical to that set forth in SEQ ID NO: 639. In some embodiments, the immunoglobulin heavy chain comprises an amino acid sequence at least about 90% identical to that set forth in SEQ ID NO: 551; and the immunoglobulin light chain comprises an amino acid sequence at least about 90% identical to that set forth in SEQ ID NO: 640. In some embodiments, the immunoglobulin heavy chain comprises an amino acid sequence at least about 90% identical to that set forth in SEQ ID NO: 552; and the immunoglobulin light chain comprises an amino acid sequence at least about 90% identical to that set forth in SEQ ID NO: 641. In some embodiments, the immunoglobulin heavy chain comprises an amino acid sequence at least about 90% identical to that set forth in SEQ ID NO: 553; and the immunoglobulin light chain comprises an amino acid sequence at least about 90% identical to that set forth in SEQ ID NO: 642. In some embodiments, the immunoglobulin heavy chain comprises an amino acid sequence at least about 90% identical to that set forth in SEQ ID NO: 554; and the immunoglobulin light chain comprises an amino acid sequence at least about 90% identical to that set forth in SEQ ID NO: 643. In some embodiments, the immunoglobulin heavy chain comprises an amino acid sequence at least about 90% identical to that set forth in SEQ ID NO: 555; and the immunoglobulin light chain comprises an amino acid sequence at least about 90% identical to that set forth in SEQ ID NO: 644. In some embodiments, the immunoglobulin heavy chain comprises an amino acid sequence at least about 90% identical to that set forth in SEQ ID NO: 556; and the immunoglobulin light chain comprises an amino acid sequence at least about 90% identical to that set forth in SEQ ID NO: 645. In some embodiments, the immunoglobulin heavy chain comprises an amino acid sequence at least about 90% identical to that set forth in SEQ ID NO: 557; and the immunoglobulin light chain comprises an amino acid sequence at least about 90% identical to that set forth in SEQ ID NO: 646. In some embodiments, the immunoglobulin heavy chain comprises an amino acid sequence at least about 90% identical to that set forth in SEQ ID NO: 558; and the immunoglobulin light chain comprises an amino acid sequence at least about 90% identical to that set forth in SEQ ID NO: 647. In some embodiments, the immunoglobulin heavy chain comprises an amino acid sequence at least about 90% identical to that set forth in SEQ ID NO: 559; and the immunoglobulin light chain comprises an amino acid sequence at least about 90% identical to that set forth in SEQ ID NO: 648. In some embodiments, the immunoglobulin heavy chain comprises an amino acid sequence at least about 90% identical to that set forth in SEQ ID NO: 560; and the immunoglobulin light chain comprises an amino acid sequence at least about 90% identical to that set forth in SEQ ID NO: 649. In some embodiments, the immunoglobulin heavy chain comprises an amino acid sequence at least about 90% identical to that set forth in SEQ ID NO: 561; and the immunoglobulin light chain comprises an amino acid sequence at least about 90% identical to that set forth in SEQ ID NO: 650. In some embodiments, the immunoglobulin heavy chain comprises an amino acid sequence at least about 90% identical to that set forth in SEQ ID NO: 562; and the immunoglobulin light chain comprises an amino acid sequence at least about 90% identical to that set forth in SEQ ID NO: 651. In some embodiments, the immunoglobulin heavy chain comprises an amino acid sequence at least about 90% identical to that set forth in SEQ ID NO: 563; and the immunoglobulin light chain comprises an amino acid sequence at least about 90% identical to that set forth in SEQ ID NO: 652. In some embodiments, the immunoglobulin heavy chain comprises an amino acid sequence at least about 90% identical to that set forth in SEQ ID NO: 564; and the immunoglobulin light chain comprises an amino acid sequence at least about 90% identical to that set forth in SEQ ID NO: 653. In some embodiments, the immunoglobulin heavy chain comprises an amino acid sequence at least about 90% identical to that set forth in SEQ ID NO: 565; and the immunoglobulin light chain comprises an amino acid sequence at least about 90% identical to that set forth in SEQ ID NO: 654. In some embodiments, the immunoglobulin heavy chain comprises an amino acid sequence at least about 90% identical to that set forth in SEQ ID NO: 566; and the immunoglobulin light chain comprises an amino acid sequence at least about 90% identical to that set forth in SEQ ID NO: 655. In some embodiments, the immunoglobulin heavy chain comprises an amino acid sequence at least about 90% identical to that set forth in SEQ ID NO: 567; and the immunoglobulin light chain comprises an amino acid sequence at least about 90% identical to that set forth in SEQ ID NO: 656. In some embodiments, the immunoglobulin heavy chain comprises an amino acid sequence at least about 90% identical to that set forth in SEQ ID NO: 568; and the immunoglobulin light chain comprises an amino acid sequence at least about 90% identical to that set forth in SEQ ID NO: 657. In some embodiments, the immunoglobulin heavy chain comprises an amino acid sequence at least about 90% identical to that set forth in SEQ ID NO: 569; and the immunoglobulin light chain comprises an amino acid sequence at least about 90% identical to that set forth in SEQ ID NO: 658. In some embodiments, the immunoglobulin heavy chain comprises an amino acid sequence at least about 90% identical to that set forth in SEQ ID NO: 570; and the immunoglobulin light chain comprises an amino acid sequence at least about 90% identical to that set forth in SEQ ID NO: 659. In some embodiments, the immunoglobulin heavy chain comprises an amino acid sequence at least about 90% identical to that set forth in SEQ ID NO: 571; and the immunoglobulin light chain comprises an amino acid sequence at least about 90% identical to that set forth in SEQ ID NO: 660. In some embodiments, the immunoglobulin heavy chain comprises an amino acid sequence at least about 90% identical to that set forth in SEQ ID NO: 572; and the immunoglobulin light chain comprises an amino acid sequence at least about 90% identical to that set forth in SEQ ID NO: 661. In some embodiments, the immunoglobulin heavy chain comprises an amino acid sequence at least about 90% identical to that set forth in SEQ ID NO: 573; and the immunoglobulin light chain comprises an amino acid sequence at least about 90% identical to that set forth in SEQ ID NO: 662. In some embodiments, the immunoglobulin heavy chain comprises an amino acid sequence at least about 90% identical to that set forth in SEQ ID NO: 574; and the immunoglobulin light chain comprises an amino acid sequence at least about 90% identical to that set forth in SEQ ID NO: 663. In some embodiments, the immunoglobulin heavy chain comprises an amino acid sequence at least about 90% identical to that set forth in SEQ ID NO: 575; and the immunoglobulin light chain comprises an amino acid sequence at least about 90% identical to that set forth in SEQ ID NO: 664. In some embodiments, the immunoglobulin heavy chain comprises an amino acid sequence at least about 90% identical to that set forth in SEQ ID NO: 576; and the immunoglobulin light chain comprises an amino acid sequence at least about 90% identical to that set forth in SEQ ID NO: 665. In some embodiments, the immunoglobulin heavy chain comprises an amino acid sequence at least about 90% identical to that set forth in SEQ ID NO: 577; and the immunoglobulin light chain comprises an amino acid sequence at least about 90% identical to that set forth in SEQ ID NO: 666. In some embodiments, the immunoglobulin heavy chain comprises an amino acid sequence at least about 90% identical to that set forth in SEQ ID NO: 578; and the immunoglobulin light chain comprises an amino acid sequence at least about 90% identical to that set forth in SEQ ID NO: 667. In some embodiments, the immunoglobulin heavy chain comprises an amino acid sequence at least about 90% identical to that set forth in SEQ ID NO: 579; and the immunoglobulin light chain comprises an amino acid sequence at least about 90% identical to that set forth in SEQ ID NO: 668. In some embodiments, the immunoglobulin heavy chain comprises an amino acid sequence at least about 90% identical to that set forth in SEQ ID NO: 580; and the immunoglobulin light chain comprises an amino acid sequence at least about 90% identical to that set forth in SEQ ID NO: 669. In some embodiments, the immunoglobulin heavy chain comprises an amino acid sequence at least about 90% identical to that set forth in SEQ ID NO: 581; and the immunoglobulin light chain comprises an amino acid sequence at least about 90% identical to that set forth in SEQ ID NO: 670. In some embodiments, the immunoglobulin heavy chain comprises an amino acid sequence at least about 90% identical to that set forth in SEQ ID NO: 582; and the immunoglobulin light chain comprises an amino acid sequence at least about 90% identical to that set forth in SEQ ID NO: 671. In some embodiments, the immunoglobulin heavy chain comprises an amino acid sequence at least about 90% identical to that set forth in SEQ ID NO: 583; and the immunoglobulin light chain comprises an amino acid sequence at least about 90% identical to that set forth in SEQ ID NO: 672. In some embodiments, the immunoglobulin heavy chain comprises an amino acid sequence at least about 90% identical to that set forth in SEQ ID NO: 584; and the immunoglobulin light chain comprises an amino acid sequence at least about 90% identical to that set forth in SEQ ID NO: 673. In some embodiments, the immunoglobulin heavy chain comprises an amino acid sequence at least about 90% identical to that set forth in SEQ ID NO: 585; and the immunoglobulin light chain comprises an amino acid sequence at least about 90% identical to that set forth in SEQ ID NO: 674. In some embodiments, the immunoglobulin heavy chain comprises an amino acid sequence at least about 90% identical to that set forth in SEQ ID NO: 586; and the immunoglobulin light chain comprises an amino acid sequence at least about 90% identical to that set forth in SEQ ID NO: 675. In some embodiments, the immunoglobulin heavy chain comprises an amino acid sequence at least about 90% identical to that set forth in SEQ ID NO: 587; and the immunoglobulin light chain comprises an amino acid sequence at least about 90% identical to that set forth in SEQ ID NO: 676. In some embodiments, the immunoglobulin heavy chain comprises an amino acid sequence at least about 90% identical to that set forth in SEQ ID NO: 588; and the immunoglobulin light chain comprises an amino acid sequence at least about 90% identical to that set forth in SEQ ID NO: 677. In some embodiments, the immunoglobulin heavy chain comprises an amino acid sequence at least about 90% identical to that set forth in SEQ ID NO: 589; and the immunoglobulin light chain comprises an amino acid sequence at least about 90% identical to that set forth in SEQ ID NO: 678. In some embodiments, the immunoglobulin heavy chain comprises an amino acid sequence at least about 90% identical to that set forth in SEQ ID NO: 590; and the immunoglobulin light chain comprises an amino acid sequence at least about 90% identical to that set forth in SEQ ID NO: 679. In some embodiments, the immunoglobulin heavy chain comprises an amino acid sequence at least about 90% identical to that set forth in SEQ ID NO: 591; and the immunoglobulin light chain comprises an amino acid sequence at least about 90% identical to that set forth in SEQ ID NO: 680. In some embodiments, the immunoglobulin heavy chain comprises an amino acid sequence at least about 90% identical to that set forth in SEQ ID NO: 592; and the immunoglobulin light chain comprises an amino acid sequence at least about 90% identical to that set forth in SEQ ID NO: 681. In some embodiments, the immunoglobulin heavy chain comprises an amino acid sequence at least about 90% identical to that set forth in SEQ ID NO: 593; and the immunoglobulin light chain comprises an amino acid sequence at least about 90% identical to that set forth in SEQ ID NO: 682. In some embodiments, the immunoglobulin heavy chain comprises an amino acid sequence at least about 90% identical to that set forth in SEQ ID NO: 594; and the immunoglobulin light chain comprises an amino acid sequence at least about 90% identical to that set forth in SEQ ID NO: 683. In some embodiments, the immunoglobulin heavy chain comprises an amino acid sequence at least about 90% identical to that set forth in SEQ ID NO: 595; and the immunoglobulin light chain comprises an amino acid sequence at least about 90% identical to that set forth in SEQ ID NO: 684. In some embodiments, the immunoglobulin heavy chain comprises an amino acid sequence at least about 90% identical to that set forth in SEQ ID NO: 596; and the immunoglobulin light chain comprises an amino acid sequence at least about 90% identical to that set forth in SEQ ID NO: 685. In some embodiments, the immunoglobulin heavy chain comprises an amino acid sequence at least about 90% identical to that set forth in SEQ ID NO: 597; and the immunoglobulin light chain comprises an amino acid sequence at least about 90% identical to that set forth in SEQ ID NO: 686. In some embodiments, the immunoglobulin heavy chain comprises an amino acid sequence at least about 90% identical to that set forth in SEQ ID NO: 598; and the immunoglobulin light chain comprises an amino acid sequence at least about 90% identical to that set forth in SEQ ID NO: 687. In some embodiments, the immunoglobulin heavy chain comprises an amino acid sequence at least about 90% identical to that set forth in SEQ ID NO: 599; and the immunoglobulin light chain comprises an amino acid sequence at least about 90% identical to that set forth in SEQ ID NO: 688. In some embodiments, the immunoglobulin heavy chain comprises an amino acid sequence at least about 90% identical to that set forth in SEQ ID NO: 600; and the immunoglobulin light chain comprises an amino acid sequence at least about 90% identical to that set forth in SEQ ID NO: 689. In some embodiments, the immunoglobulin heavy chain comprises an amino acid sequence at least about 90% identical to that set forth in SEQ ID NO: 601; and the immunoglobulin light chain comprises an amino acid sequence at least about 90% identical to that set forth in SEQ ID NO: 690. In some embodiments, the immunoglobulin heavy chain comprises an amino acid sequence at least about 90% identical to that set forth in SEQ ID NO: 602; and the immunoglobulin light chain comprises an amino acid sequence at least about 90% identical to that set forth in SEQ ID NO: 691. In some embodiments, the immunoglobulin heavy chain comprises an amino acid sequence at least about 90% identical to that set forth in SEQ ID NO: 603; and the immunoglobulin light chain comprises an amino acid sequence at least about 90% identical to that set forth in SEQ ID NO: 692. In some embodiments, the immunoglobulin heavy chain comprises an amino acid sequence at least about 90% identical to that set forth in SEQ ID NO: 604; and the immunoglobulin light chain comprises an amino acid sequence at least about 90% identical to that set forth in SEQ ID NO: 693. In some embodiments, the immunoglobulin heavy chain comprises an amino acid sequence at least about 90% identical to that set forth in SEQ ID NO: 605; and the immunoglobulin light chain comprises an amino acid sequence at least about 90% identical to that set forth in SEQ ID NO: 694. In some embodiments, the immunoglobulin heavy chain comprises an amino acid sequence at least about 90% identical to that set forth in SEQ ID NO: 606; and the immunoglobulin light chain comprises an amino acid sequence at least about 90% identical to that set forth in SEQ ID NO: 695. In some embodiments, the immunoglobulin heavy chain comprises an amino acid sequence at least about 90% identical to that set forth in SEQ ID NO: 607; and the immunoglobulin light chain comprises an amino acid sequence at least about 90% identical to that set forth in SEQ ID NO: 696. In some embodiments, the immunoglobulin heavy chain comprises an amino acid sequence at least about 90% identical to that set forth in SEQ ID NO: 608; and the immunoglobulin light chain comprises an amino acid sequence at least about 90% identical to that set forth in SEQ ID NO: 697. In some embodiments, the immunoglobulin heavy chain comprises an amino acid sequence at least about 90% identical to that set forth in SEQ ID NO: 609; and the immunoglobulin light chain comprises an amino acid sequence at least about 90% identical to that set forth in SEQ ID NO: 698. In some embodiments, the immunoglobulin heavy chain comprises an amino acid sequence at least about 90% identical to that set forth in SEQ ID NO: 610; and the immunoglobulin light chain comprises an amino acid sequence at least about 90% identical to that set forth in SEQ ID NO: 699. In some embodiments, the immunoglobulin heavy chain comprises an amino acid sequence at least about 90% identical to that set forth in SEQ ID NO: 611; and the immunoglobulin light chain comprises an amino acid sequence at least about 90% identical to that set forth in SEQ ID NO: 700. In some embodiments, the immunoglobulin heavy chain comprises an amino acid sequence at least about 90% identical to that set forth in SEQ ID NO: 612; and the immunoglobulin light chain comprises an amino acid sequence at least about 90% identical to that set forth in SEQ ID NO: 701. In some embodiments, the immunoglobulin heavy chain comprises an amino acid sequence at least about 90% identical to that set forth in SEQ ID NO: 613; and the immunoglobulin light chain comprises an amino acid sequence at least about 90% identical to that set forth in SEQ ID NO: 702. In some embodiments, the immunoglobulin heavy chain comprises an amino acid sequence at least about 90% identical to that set forth in SEQ ID NO: 614; and the immunoglobulin light chain comprises an amino acid sequence at least about 90% identical to that set forth in SEQ ID NO: 703. In some embodiments, the immunoglobulin heavy chain comprises an amino acid sequence at least about 90% identical to that set forth in SEQ ID NO: 615; and the immunoglobulin light chain comprises an amino acid sequence at least about 90% identical to that set forth in SEQ ID NO: 704. In some embodiments, the immunoglobulin heavy chain comprises an amino acid sequence at least about 90% identical to that set forth in SEQ ID NO: 616; and the immunoglobulin light chain comprises an amino acid sequence at least about 90% identical to that set forth in SEQ ID NO: 705. In some embodiments, the immunoglobulin heavy chain comprises an amino acid sequence at least about 90% identical to that set forth in SEQ ID NO: 617; and the immunoglobulin light chain comprises an amino acid sequence at least about 90% identical to that set forth in SEQ ID NO: 706. In some embodiments, the immunoglobulin heavy chain comprises an amino acid sequence at least about 90% identical to that set forth in SEQ ID NO: 618; and the immunoglobulin light chain comprises an amino acid sequence at least about 90% identical to that set forth in SEQ ID NO: 707. In some embodiments, the immunoglobulin heavy chain comprises an amino acid sequence at least about 90% identical to that set forth in SEQ ID NO: 619; and the immunoglobulin light chain comprises an amino acid sequence at least about 90% identical to that set forth in SEQ ID NO: 708. In some embodiments, the immunoglobulin heavy chain comprises an amino acid sequence at least about 90% identical to that set forth in SEQ ID NO: 620; and the immunoglobulin light chain comprises an amino acid sequence at least about 90% identical to that set forth in SEQ ID NO: 709. In some embodiments, the immunoglobulin heavy chain comprises an amino acid sequence at least about 90% identical to that set forth in SEQ ID NO: 621; and the immunoglobulin light chain comprises an amino acid sequence at least about 90% identical to that set forth in SEQ ID NO: 710. In some embodiments, the immunoglobulin heavy chain comprises an amino acid sequence at least about 90% identical to that set forth in SEQ ID NO: 622; and the immunoglobulin light chain comprises an amino acid sequence at least about 90% identical to that set forth in SEQ ID NO: 711. In some embodiments, the immunoglobulin heavy chain comprises an amino acid sequence at least about 90% identical to that set forth in SEQ ID NO: 623; and the immunoglobulin light chain comprises an amino acid sequence at least about 90% identical to that set forth in SEQ ID NO: 712. In some embodiments, the immunoglobulin heavy chain comprises an amino acid sequence at least about 90% identical to that set forth in SEQ ID NO: 624; and the immunoglobulin light chain comprises an amino acid sequence at least about 90% identical to that set forth in SEQ ID NO: 713. In some embodiments, the immunoglobulin heavy chain comprises an amino acid sequence at least about 90% identical to that set forth in SEQ ID NO: 625; and the immunoglobulin light chain comprises an amino acid sequence at least about 90% identical to that set forth in SEQ ID NO: 714. In some embodiments, the immunoglobulin heavy chain comprises an amino acid sequence at least about 90% identical to that set forth in SEQ ID NO: 626; and the immunoglobulin light chain comprises an amino acid sequence at least about 90% identical to that set forth in SEQ ID NO: 715. In some embodiments, the immunoglobulin heavy chain comprises an amino acid sequence at least about 90% identical to that set forth in SEQ ID NO: 627; and the immunoglobulin light chain comprises an amino acid sequence at least about 90% identical to that set forth in SEQ ID NO: 716. In some embodiments, the immunoglobulin heavy chain comprises an amino acid sequence at least about 90% identical to that set forth in SEQ ID NO: 628; and the immunoglobulin light chain comprises an amino acid sequence at least about 90% identical to that set forth in SEQ ID NO: 717.

Provided herein are adenosine A2A receptor binding libraries comprising nucleic acids encoding for scaffolds or immunoglobulins comprising adenosine A2A receptor binding domains comprise variation in domain type, domain length, or residue variation. In some instances, the domain is a region in the scaffold comprising the adenosine A2A receptor binding domains. For example, the region is the VH, CDRH3, or VL domain. In some instances, the domain is the adenosine A2A receptor binding domain.

Methods described herein provide for synthesis of an adenosine A2A receptor binding library of nucleic acids each encoding for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. In some instances, the adenosine A2A receptor binding library comprises varied nucleic acids collectively encoding variations at multiple positions. In some instances, the variant library comprises sequences encoding for variation of at least a single codon of a VH, CDRH3, or VL domain. In some instances, the variant library comprises sequences encoding for variation of at least a single codon in an adenosine A2A receptor binding domain. For example, at least one single codon of an adenosine A2A receptor binding domain as listed in Table 1 is varied. In some instances, the variant library comprises sequences encoding for variation of multiple codons of a VH, CDRH3, or VL domain. In some instances, the variant library comprises sequences encoding for variation of multiple codons in an adenosine A2A receptor binding domain. An exemplary number of codons for variation include, but are not limited to, at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons.

Methods described herein provide for synthesis of an adenosine A2A receptor binding library of nucleic acids each encoding for a predetermined variant of at least one predetermined reference nucleic acid sequence, wherein the adenosine A2A receptor binding library comprises sequences encoding for variation of length of a domain. In some instances, the domain is VH, CDRH3, or VL domain. In some instances, the domain is the adenosine A2A receptor binding domain. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons less as compared to a predetermined reference sequence. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 200, 225, 250, 275, 300, or more than 300 codons more as compared to a predetermined reference sequence.

Provided herein are adenosine A2A receptor binding libraries comprising nucleic acids encoding for scaffolds comprising adenosine A2A receptor binding domains, wherein the adenosine A2A receptor binding libraries are synthesized with various numbers of fragments. In some instances, the fragments comprise the VH, CDRH3, or VL domain. In some instances, the adenosine A2A receptor binding libraries are synthesized with at least or about 2 fragments, 3 fragments, 4 fragments, 5 fragments, or more than 5 fragments. The length of each of the nucleic acid fragments or average length of the nucleic acids synthesized may be at least or about 50, 75, 100, 125, 150, 175, 200, 225, 250, 275, 300, 325, 350, 375, 400, 425, 450, 475, 500, 525, 550, 575, 600, or more than 600 base pairs. In some instances, the length is about 50 to 600, 75 to 575, 100 to 550, 125 to 525, 150 to 500, 175 to 475, 200 to 450, 225 to 425, 250 to 400, 275 to 375, or 300 to 350 base pairs.

Adenosine A2A receptor binding libraries comprising nucleic acids encoding for scaffolds comprising adenosine A2A receptor binding domains as described herein comprise various lengths of amino acids when translated. In some instances, the length of each of the amino acid fragments or average length of the amino acid synthesized may be at least or about 15, 20, 25, 30, 35, 40, 45, 50, 55, 60, 65, 70, 75, 80, 85, 90, 95, 100, 105, 110, 115, 120, 125, 130, 135, 140, 145, 150, or more than 150 amino acids. In some instances, the length of the amino acid is about 15 to 150, 20 to 145, 25 to 140, 30 to 135, 35 to 130, 40 to 125, 45 to 120, 50 to 115, 55 to 110, 60 to 110, 65 to 105, 70 to 100, or 75 to 95 amino acids. In some instances, the length of the amino acid is about 22 to about 75 amino acids.

Adenosine A2A receptor binding libraries comprising de novo synthesized variant sequences encoding for scaffolds comprising adenosine A2A receptor binding domains comprise a number of variant sequences. In some instances, a number of variant sequences is de novo synthesized for a CDRH1, CDRH2, CDRH3, CDRL1, CDRL2, CDRL3, VL, VH, or a combination thereof. In some instances, a number of variant sequences is de novo synthesized for framework element 1 (FW1), framework element 2 (FW2), framework element 3 (FW3), or framework element 4 (FW4). In some instances, a number of variant sequences is de novo synthesized for an adenosine A2A receptor binding domain. For example, the number of variant sequences is about 1 to about 10 sequences for the VH domain, about 108 sequences for the adenosine A2A receptor binding domain, and about 1 to about 44 sequences for the VK domain. The number of variant sequences may be at least or about 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, or more than 500 sequences. In some instances, the number of variant sequences is about 10 to 300, 25 to 275, 50 to 250, 75 to 225, 100 to 200, or 125 to 150 sequences.

Adenosine A2A receptor binding libraries comprising de novo synthesized variant sequences encoding for scaffolds comprising adenosine A2A receptor binding domains comprise improved diversity. For example, variants are generated by placing adenosine A2A receptor binding domain variants in immunoglobulin scaffold variants comprising N-terminal CDRH3 variations and C-terminal CDRH3 variations. In some instances, variants include affinity maturation variants. Alternatively or in combination, variants include variants in other regions of the immunoglobulin including, but not limited to, CDRH1, CDRH2, CDRL1, CDRL2, and CDRL3. In some instances, the number of variants of the adenosine A2A receptor binding libraries is least or about 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ non-identical sequences. For example, a library comprising about 10 variant sequences for a VH region, about 237 variant sequences for a CDRH3 region, and about 43 variant sequences for a VL and CDRL3 region comprises 105 non-identical sequences (10×237×43).

Provided herein are libraries comprising nucleic acids encoding for an adenosine A2A receptor antibody comprising variation in at least one region of the antibody, wherein the region is the CDR region. In some instances, the adenosine A2A receptor antibody is a single domain antibody comprising one heavy chain variable domain such as a VHH antibody. In some instances, the VHH antibody comprises variation in one or more CDR regions. In some instances, libraries described herein comprise at least or about 1, 10, 20, 30, 40, 50, 60, 70, 80, 90, 100, 200, 300, 400, 500, 600, 700, 800, 900, 1000, 1200, 1400, 1600, 1800, 2000, 2400, 2600, 2800, 3000, or more than 3000 sequences of a CDR1, CDR2, or CDR3. In some instances, libraries described herein comprise at least or about 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences of a CDR1, CDR2, or CDR3. For example, the libraries comprise at least 2000 sequences of a CDR1, at least 1200 sequences for CDR2, and at least 1600 sequences for CDR3. In some instances, each sequence is non-identical.

In some instances, the CDR1, CDR2, or CDR3 is of a variable domain, light chain (VL). CDR1, CDR2, or CDR3 of a variable domain, light chain (VL) can be referred to as CDRL1, CDRL2, or CDRL3, respectively. In some instances, libraries described herein comprise at least or about 1, 2, 3, 4, 5, 6, 7, 8, 9, 10, 20, 30, 40, 50, 60, 70, 80, 90, 100, 200, 300, 400, 500, 600, 700, 800, 900, 1000, 1200, 1400, 1600, 1800, 2000, 2400, 2600, 2800, 3000, or more than 3000 sequences of a CDR1, CDR2, or CDR3 of the VL. In some instances, libraries described herein comprise at least or about 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences of a CDR1, CDR2, or CDR3 of the VL. For example, the libraries comprise at least 20 sequences of a CDR1 of the VL, at least 4 sequences of a CDR2 of the VL, and at least 140 sequences of a CDR3 of the VL. In some instances, the libraries comprise at least 2 sequences of a CDR1 of the VL, at least 1 sequence of CDR2 of the VL, and at least 3000 sequences of a CDR3 of the VL. In some instances, the VL is IGKV1-39, IGKV1-9, IGKV2-28, IGKV3-11, IGKV3-15, IGKV3-20, IGKV4-1, IGLV1-51, IGLV2-14, IGLV1-40, or IGLV3-1. In some instances, the VL is IGKV2-28. In some instances, the VL is IGLV1-51.

In some instances, the CDR1, CDR2, or CDR3 is of a variable domain, heavy chain (VH). CDR1, CDR2, or CDR3 of a variable domain, heavy chain (VH) can be referred to as CDRH1, CDRH2, or CDRH3, respectively. In some instances, libraries described herein comprise at least or about 1, 2, 3, 4, 5, 6, 7, 8, 9, 10, 20, 30, 40, 50, 60, 70, 80, 90, 100, 200, 300, 400, 500, 600, 700, 800, 900, 1000, 1200, 1400, 1600, 1800, 2000, 2400, 2600, 2800, 3000, or more than 3000 sequences of a CDR1, CDR2, or CDR3 of the VH. In some instances, libraries described herein comprise at least or about 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences of a CDR1, CDR2, or CDR3 of the VH. For example, the libraries comprise at least 30 sequences of a CDR1 of the VH, at least 570 sequences of a CDR2 of the VH, and at least 108 sequences of a CDR3 of the VH. In some instances, the libraries comprise at least 30 sequences of a CDR1 of the VH, at least 860 sequences of a CDR2 of the VH, and at least 107 sequences of a CDR3 of the VH. In some instances, the VH is IGHV1-18, IGHV1-69, IGHV1-8 IGHV3-21, IGHV3-23, IGHV3-30/33rn, IGHV3-28, IGHV3-74, IGHV4-39, or IGHV4-59/61. In some instances, the VH is IGHV1-69, IGHV3-30, IGHV3-23, IGHV3, IGHV1-46, IGHV3-7, IGHV1, or IGHV1-8. In some instances, the VH is IGHV1-69 and IGHV3-30. In some instances, the VH is IGHV3-23.

Libraries as described herein, in some embodiments, comprise varying lengths of a CDRL1, CDRL2, CDRL3, CDRH1, CDRH2, or CDRH3. In some instances, the length of the CDRL1, CDRL2, CDRL3, CDRH1, CDRH2, or CDRH3 comprises at least or about 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or more than 90 amino acids in length. For example, the CDRH3 comprises at least or about 12, 15, 16, 17, 20, 21, or 23 amino acids in length. In some instances, the CDRL1, CDRL2, CDRL3, CDRH1, CDRH2, or CDRH3 comprises a range of about 1 to about 10, about 5 to about 15, about 10 to about 20, or about 15 to about 30 amino acids in length.

Libraries comprising nucleic acids encoding for antibodies having variant CDR sequences as described herein comprise various lengths of amino acids when translated. In some instances, the length of each of the amino acid fragments or average length of the amino acid synthesized may be at least or about 15, 20, 25, 30, 35, 40, 45, 50, 55, 60, 65, 70, 75, 80, 85, 90, 95, 100, 105, 110, 115, 120, 125, 130, 135, 140, 145, 150, or more than 150 amino acids. In some instances, the length of the amino acid is about 15 to 150, 20 to 145, 25 to 140, 30 to 135, 35 to 130, 40 to 125, 45 to 120, 50 to 115, 55 to 110, 60 to 110, 65 to 105, 70 to 100, or 75 to 95 amino acids. In some instances, the length of the amino acid is about 22 amino acids to about 75 amino acids. In some instances, the antibodies comprise at least or about 100, 200, 300, 400, 500, 600, 700, 800, 900, 1000, 2000, 3000, 4000, 5000, or more than 5000 amino acids.

Ratios of the lengths of a CDRL1, CDRL2, CDRL3, CDRH1, CDRH2, or CDRH3 may vary in libraries described herein. In some instances, a CDRL1, CDRL2, CDRL3, CDRH1, CDRH2, or CDRH3 comprising at least or about 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or more than 90 amino acids in length comprises about 10%, 20%, 30%, 40%, 50%, 60%, 70%, 80%, 90%, or more than 90% of the library. For example, a CDRH3 comprising about 23 amino acids in length is present in the library at 40%, a CDRH3 comprising about 21 amino acids in length is present in the library at 30%, a CDRH3 comprising about 17 amino acids in length is present in the library at 20%, and a CDRH3 comprising about 12 amino acids in length is present in the library at 10%. In some instances, a CDRH3 comprising about 20 amino acids in length is present in the library at 40%, a CDRH3 comprising about 16 amino acids in length is present in the library at 30%, a CDRH3 comprising about 15 amino acids in length is present in the library at 20%, and a CDRH3 comprising about 12 amino acids in length is present in the library at 10%.

Libraries as described herein encoding for a VHH antibody comprise variant CDR sequences that are shuffled to generate a library with a theoretical diversity of at least or about 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences. In some instances, the library has a final library diversity of at least or about 10⁷, 10⁸, 10⁹, 10¹⁰ 10¹¹, 10¹², 10¹³, 10¹⁴, 10¹⁵, 10¹⁶, 10¹⁷, 10¹⁸, 10¹⁹, 10²⁰, or more than 10²⁰ sequences.

Provided herein are adenosine A2A receptor binding libraries encoding for an immunoglobulin. In some instances, the adenosine A2A receptor immunoglobulin is an antibody. In some instances, the adenosine A2A receptor immunoglobulin is a VHH antibody. In some instances, the adenosine A2A receptor immunoglobulin comprises a binding affinity (e.g., K_{D})) to adenosine A2A receptor of less than 1 nM, less than 1.2 nM, less than 2 nM, less than 5 nM, less than 10 nM, less than 11 nm, less than 13.5 nM, less than 15 nM, less than 20 nM, less than 25 nM, or less than 30 nM. In some instances, the adenosine A2A receptor immunoglobulin comprises a K_{D} of less than 1 nM. In some instances, the adenosine A2A receptor immunoglobulin comprises a K_{D} of less than 1.2 nM. In some instances, the adenosine A2A receptor immunoglobulin comprises a K_{D} of less than 2 nM. In some instances, the adenosine A2A receptor immunoglobulin comprises a K_{D} of less than 5 nM. In some instances, the adenosine A2A receptor immunoglobulin comprises a K_{D} of less than 10 nM. In some instances, the adenosine A2A receptor immunoglobulin comprises a K_{D} of less than 13.5 nM. In some instances, the adenosine A2A receptor immunoglobulin comprises a K_{D} of less than 15 nM. In some instances, the adenosine A2A receptor immunoglobulin comprises a K_{D} of less than 20 nM. In some instances, the adenosine A2A receptor immunoglobulin comprises a K_{D} of less than 25 nM. In some instances, the adenosine A2A receptor immunoglobulin comprises a K_{D} of less than 30 nM.

In some instances, the adenosine A2A receptor immunoglobulin is an adenosine A2A receptor agonist. In some instances, the adenosine A2A receptor immunoglobulin is an adenosine A2A receptor antagonist. In some instances, the adenosine A2A receptor immunoglobulin is an adenosine A2A receptor allosteric modulator. In some instances, the allosteric modulator is a negative allosteric modulator. In some instances, the allosteric modulator is a positive allosteric modulator. In some instances, the adenosine A2A receptor immunoglobulin results in agonistic, antagonistic, or allosteric effects at a concentration of at least or about 1 nM, 2 nM, 4 nM, 6 nM, 8 nM, 10 nM, 20 nM, 30 nM, 40 nM, 50 nM, 60 nM, 70 nM, 80 nM, 90 nM, 100 nM, 120 nM, 140 nM, 160 nM, 180 nM, 200 nM, 300 nM, 400 nM, 500 nM, 600 nM, 700 nM, 800 nM, 900 nM, 1000 nM, or more than 1000 nM. In some instances, the adenosine A2A receptor immunoglobulin is a negative allosteric modulator. In some instances, the adenosine A2A receptor immunoglobulin is a negative allosteric modulator at a concentration of at least or about 0.001, 0.005, 0.01, 0.05, 0.1, 0.5, 1 nM, 2 nM, 4 nM, 6 nM, 8 nM, 10 nM, 20 nM, 30 nM, 40 nM, 50 nM, 60 nM, 70 nM, 80 nM, 90 nM, 100 nM, or more than 100 nM. In some instances, the adenosine A2A receptor immunoglobulin is a negative allosteric modulator at a concentration in a range of about 0.001 to about 100, 0.01 to about 90, about 0.1 to about 80, 1 to about 50, about 10 to about 40 nM, or about 1 to about 10 nM. In some instances, the adenosine A2A receptor immunoglobulin comprises an EC50 or IC50 of at least or about 0.001, 0.0025, 0.005, 0.01, 0.025, 0.05, 0.06, 0.07, 0.08, 0.9, 0.1, 0.5, 1, 2, 3, 4, 5, 6, or more than 6 nM. In some instances, the adenosine A2A receptor immunoglobulin comprises an EC50 or IC50 of at least or about 1 nM, 2 nM, 4 nM, 6 nM, 8 nM, 10 nM, 20 nM, 30 nM, 40 nM, 50 nM, 60 nM, 70 nM, 80 nM, 90 nM, 100 nM, or more than 100 nM.

Adenosine A2A receptor immunoglobulins as described herein may comprise improved properties. In some instances, the adenosine A2A receptor immunoglobulins are monomeric. In some instances, the adenosine A2A receptor immunoglobulins are not prone to aggregation. In some instances, at least or about 70%, 75%, 80%, 85%, 90%, 95%, or 99% of the adenosine A2A receptor immunoglobulins are monomeric. In some instances, the adenosine A2A receptor immunoglobulins are thermostable. In some instances, the adenosine A2A receptor immunoglobulins result in reduced non-specific binding.

Following synthesis of adenosine A2A receptor binding libraries comprising nucleic acids encoding scaffolds comprising adenosine A2A receptor binding domains, libraries may be used for screening and analysis. For example, libraries are assayed for library displayability and panning. In some instances, displayability is assayed using a selectable tag. Exemplary tags include, but are not limited to, a radioactive label, a fluorescent label, an enzyme, a chemiluminescent tag, a colorimetric tag, an affinity tag or other labels or tags that are known in the art. In some instances, the tag is histidine, polyhistidine, myc, hemagglutinin (HA), or FLAG. The adenosine A2A receptor binding libraries may comprise nucleic acids encoding scaffolds comprising adenosine A2A receptor binding domains with multiple tags such as GFP, FLAG, and Lucy as well as a DNA barcode. In some instances, libraries are assayed by sequencing using various methods including, but not limited to, single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis.

### Adenosine A2B Receptor Libraries

Provided herein are adenosine A2B receptor binding libraries comprising nucleic acids encoding for scaffolds comprising sequences for adenosine A2B receptor binding domains. In some instances, the scaffolds are immunoglobulins. In some instances, the scaffolds comprising sequences for adenosine A2B receptor binding domains are determined by interactions between the adenosine A2B receptor binding domains and the adenosine A2B receptor.

Provided herein are libraries comprising nucleic acids encoding scaffolds comprising adenosine A2B receptor binding domains, wherein the adenosine A2B receptor binding domains are designed based on surface interactions on adenosine A2B receptor. In some instances, the adenosine A2B receptor binding domains interact with the amino- or carboxy-terminus of the adenosine A2B receptor. In some instances, the adenosine A2B receptor binding domains interact with at least one transmembrane domain including, but not limited to, transmembrane domain 1 (TM1), transmembrane domain 2 (TM2), transmembrane domain 3 (TM3), transmembrane domain 4 (TM4), transmembrane domain 5 (TM5), transmembrane domain 6 (TM6), and transmembrane domain 7 (TM7). In some instances, the adenosine A2B receptor binding domains interact with an intracellular surface of the adenosine A2B receptor. For example, the adenosine A2B receptor binding domains interact with at least one intracellular loop including, but not limited to, intracellular loop 1 (ICL1), intracellular loop 2 (ICL2), and intracellular loop 3 (ICL3). In some instances, the adenosine A2B receptor binding domains interact with an extracellular surface of the adenosine A2B receptor For example, the adenosine A2B receptor binding domains interact with at least one extracellular domain (ECD) or extracellular loop (ECL) of the adenosine A2B receptor. The extracellular loops include, but are not limited to, extracellular loop 1 (ECL1), extracellular loop 2 (ECL2), and extracellular loop 3 (ECL3).

Described herein are adenosine A2B receptor binding domains, wherein the adenosine A2B receptor binding domains are designed based on surface interactions between an adenosine A2B receptor ligand and the adenosine A2B receptor. In some instances, the ligand is a peptide. In some instances, the ligand is an adenosine A2B receptor agonist. In some instances, the ligand is an adenosine A2B receptor antagonist. In some instances, the ligand is an adenosine A2B receptor allosteric modulator. In some instances, the allosteric modulator is a negative allosteric modulator. In some instances, the allosteric modulator is a positive allosteric modulator. Exemplary ligands of the adenosine A2B receptor include, but are not limited to DU172, PSB36, ZM241385, XAC, caffeine, T4G, T4E, 6DY, 6DZ, 6DX, 6DV, 8D1b, theophylline, UK-432097, adenosine, NECA, and CGS21680.

Sequences of adenosine A2B receptor binding domains based on surface interactions between an adenosine A2B receptor ligand and the adenosine A2B receptor are analyzed using various methods. For example, multispecies computational analysis is performed. In some instances, a structure analysis is performed. In some instances, a sequence analysis is performed. Sequence analysis can be performed using a database known in the art. Non-limiting examples of databases include, but are not limited to, NCBI BLAST (blast.ncbi.nlm.nih.gov/Blast.cgi), UCSC Genome Browser (genome.ucsc.edu/), UniProt (uniprot.org/), and IUPHAR/BPS Guide to PHARMACOLOGY (guidetopharmacology.org/).

Described herein are adenosine A2B receptor binding domains designed based on sequence analysis among various organisms. For example, sequence analysis is performed to identify homologous sequences in different organisms. Exemplary organisms include, but are not limited to, mouse, rat, equine, sheep, cow, primate (e.g., chimpanzee, baboon, gorilla, orangutan, monkey), dog, cat, pig, donkey, rabbit, fish, fly, and human.

Following identification of adenosine A2B receptor binding domains, libraries comprising nucleic acids encoding for the adenosine A2B receptor binding domains may be generated. In some instances, libraries of adenosine A2B receptor binding domains comprise sequences of adenosine A2B receptor binding domains designed based on conformational ligand interactions, peptide ligand interactions, small molecule ligand interactions, extracellular domains of adenosine A2B receptor, or antibodies that target adenosine A2B receptor. In some instances, libraries of adenosine A2B receptor binding domains comprise sequences of adenosine A2B receptor binding domains designed based on peptide ligand interactions. In some instances, the ligand is a not an antibody ligand. Libraries of adenosine A2B receptor binding domains may be translated to generate protein libraries. In some instances, libraries of adenosine A2B receptor binding domains are translated to generate peptide libraries, immunoglobulin libraries, derivatives thereof, or combinations thereof. In some instances, libraries of adenosine A2B receptor binding domains are translated to generate protein libraries that are further modified to generate peptidomimetic libraries. In some instances, libraries of adenosine A2B receptor binding domains are translated to generate protein libraries that are used to generate small molecules.

Methods described herein provide for synthesis of libraries of adenosine A2B receptor binding domains comprising nucleic acids each encoding for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. In some instances, the libraries of adenosine A2B receptor binding domains comprise varied nucleic acids collectively encoding variations at multiple positions. In some instances, the variant library comprises sequences encoding for variation of at least a single codon in an adenosine A2B receptor binding domain. In some instances, the variant library comprises sequences encoding for variation of multiple codons in an adenosine A2B receptor binding domain. An exemplary number of codons for variation include, but are not limited to, at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons.

Methods described herein provide for synthesis of libraries comprising nucleic acids encoding for the adenosine A2B receptor binding domains, wherein the libraries comprise sequences encoding for variation of length of the adenosine A2B receptor binding domains. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons less as compared to a predetermined reference sequence. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 200, 225, 250, 275, 300, or more than 300 codons more as compared to a predetermined reference sequence.

Following identification of adenosine A2B receptor binding domains, the adenosine A2B receptor binding domains may be placed in scaffolds as described herein. In some instances, the scaffolds are immunoglobulins. In some instances, the adenosine A2B receptor binding domains are placed in the CDRH3 region. Adenosine A2B receptor binding domains that may be placed in scaffolds can also be referred to as a motif. Scaffolds comprising adenosine A2B receptor binding domains may be designed based on binding, specificity, stability, expression, folding, or downstream activity. In some instances, the scaffolds comprising adenosine A2B receptor binding domains enable contact with the adenosine A2B receptor. In some instances, the scaffolds comprising adenosine A2B receptor binding domains enables high affinity binding with the adenosine A2B receptor.

Described herein, in some embodiments, are antibodies or immunoglobulins that bind to the adenosine A2B receptor.

Provided herein are adenosine A2B receptor binding libraries comprising nucleic acids encoding for scaffolds or immunoglobulins comprising adenosine A2B receptor binding domains comprise variation in domain type, domain length, or residue variation. In some instances, the domain is a region in the scaffold comprising the adenosine A2B receptor binding domains. For example, the region is the VH, CDRH3, or VL domain. In some instances, the domain is the adenosine A2B receptor binding domain.

Methods described herein provide for synthesis of an adenosine A2B receptor binding library of nucleic acids each encoding for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. In some instances, the adenosine A2B receptor binding library comprises varied nucleic acids collectively encoding variations at multiple positions. In some instances, the variant library comprises sequences encoding for variation of at least a single codon of a VH, CDRH3, or VL domain. In some instances, the variant library comprises sequences encoding for variation of at least a single codon in an adenosine A2B receptor binding domain. In some instances, the variant library comprises sequences encoding for variation of multiple codons of a VH, CDRH3, or VL domain. In some instances, the variant library comprises sequences encoding for variation of multiple codons in an adenosine A2B receptor binding domain. An exemplary number of codons for variation include, but are not limited to, at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons.

Methods described herein provide for synthesis of an adenosine A2B receptor binding library of nucleic acids each encoding for a predetermined variant of at least one predetermined reference nucleic acid sequence, wherein the adenosine A2B receptor binding library comprises sequences encoding for variation of length of a domain. In some instances, the domain is VH, CDRH3, or VL domain. In some instances, the domain is the adenosine A2B receptor binding domain. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons less as compared to a predetermined reference sequence. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 200, 225, 250, 275, 300, or more than 300 codons more as compared to a predetermined reference sequence.

Provided herein are adenosine A2B receptor binding libraries comprising nucleic acids encoding for scaffolds comprising adenosine A2B receptor binding domains, wherein the adenosine A2B receptor binding libraries are synthesized with various numbers of fragments. In some instances, the fragments comprise the VH, CDRH3, or VL domain. In some instances, the adenosine A2B receptor binding libraries are synthesized with at least or about 2 fragments, 3 fragments, 4 fragments, 5 fragments, or more than 5 fragments. The length of each of the nucleic acid fragments or average length of the nucleic acids synthesized may be at least or about 50, 75, 100, 125, 150, 175, 200, 225, 250, 275, 300, 325, 350, 375, 400, 425, 450, 475, 500, 525, 550, 575, 600, or more than 600 base pairs. In some instances, the length is about 50 to 600, 75 to 575, 100 to 550, 125 to 525, 150 to 500, 175 to 475, 200 to 450, 225 to 425, 250 to 400, 275 to 375, or 300 to 350 base pairs.

Adenosine A2B receptor binding libraries comprising nucleic acids encoding for scaffolds comprising adenosine A2B receptor binding domains as described herein comprise various lengths of amino acids when translated. In some instances, the length of each of the amino acid fragments or average length of the amino acid synthesized may be at least or about 15, 20, 25, 30, 35, 40, 45, 50, 55, 60, 65, 70, 75, 80, 85, 90, 95, 100, 105, 110, 115, 120, 125, 130, 135, 140, 145, 150, or more than 150 amino acids. In some instances, the length of the amino acid is about 15 to 150, 20 to 145, 25 to 140, 30 to 135, 35 to 130, 40 to 125, 45 to 120, 50 to 115, 55 to 110, 60 to 110, 65 to 105, 70 to 100, or 75 to 95 amino acids. In some instances, the length of the amino acid is about 22 to about 75 amino acids.

Adenosine A2B receptor binding libraries comprising de novo synthesized variant sequences encoding for scaffolds comprising adenosine A2B receptor binding domains comprise a number of variant sequences. In some instances, a number of variant sequences is de novo synthesized for a CDRH1, CDRH2, CDRH3, CDRL1, CDRL2, CDRL3, VL, VH, or a combination thereof. In some instances, a number of variant sequences is de novo synthesized for framework element 1 (FW1), framework element 2 (FW2), framework element 3 (FW3), or framework element 4 (FW4). In some instances, a number of variant sequences is de novo synthesized for an adenosine A2B receptor binding domain. For example, the number of variant sequences is about 1 to about 10 sequences for the VH domain, about 108 sequences for the adenosine A2B receptor binding domain, and about 1 to about 44 sequences for the VK domain. The number of variant sequences may be at least or about 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, or more than 500 sequences. In some instances, the number of variant sequences is about 10 to 300, 25 to 275, 50 to 250, 75 to 225, 100 to 200, or 125 to 150 sequences.

Adenosine A2B receptor binding libraries comprising de novo synthesized variant sequences encoding for scaffolds comprising adenosine A2B receptor binding domains comprise improved diversity. For example, variants are generated by placing adenosine A2B receptor binding domain variants in immunoglobulin scaffold variants comprising N-terminal CDRH3 variations and C-terminal CDRH3 variations. In some instances, variants include affinity maturation variants. Alternatively or in combination, variants include variants in other regions of the immunoglobulin including, but not limited to, CDRH1, CDRH2, CDRL1, CDRL2, and CDRL3. In some instances, the number of variants of the adenosine A2B receptor binding libraries is least or about 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ non-identical sequences. For example, a library comprising about 10 variant sequences for a VH region, about 237 variant sequences for a CDRH3 region, and about 43 variant sequences for a VL and CDRL3 region comprises 105 non-identical sequences (10×237×43).

Provided herein are libraries comprising nucleic acids encoding for an adenosine A2B receptor antibody comprising variation in at least one region of the antibody, wherein the region is the CDR region. In some instances, the adenosine A2B receptor antibody is a single domain antibody comprising one heavy chain variable domain such as a VHH antibody. In some instances, the VHH antibody comprises variation in one or more CDR regions. In some instances, libraries described herein comprise at least or about 1, 10, 20, 30, 40, 50, 60, 70, 80, 90, 100, 200, 300, 400, 500, 600, 700, 800, 900, 1000, 1200, 1400, 1600, 1800, 2000, 2400, 2600, 2800, 3000, or more than 3000 sequences of a CDR1, CDR2, or CDR3. In some instances, libraries described herein comprise at least or about 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences of a CDR1, CDR2, or CDR3. For example, the libraries comprise at least 2000 sequences of a CDR1, at least 1200 sequences for CDR2, and at least 1600 sequences for CDR3. In some instances, each sequence is non-identical.

In some instances, the CDR1, CDR2, or CDR3 is of a variable domain, light chain (VL). CDR1, CDR2, or CDR3 of a variable domain, light chain (VL) can be referred to as CDRL1, CDRL2, or CDRL3, respectively. In some instances, libraries described herein comprise at least or about 1, 2, 3, 4, 5, 6, 7, 8, 9, 10, 20, 30, 40, 50, 60, 70, 80, 90, 100, 200, 300, 400, 500, 600, 700, 800, 900, 1000, 1200, 1400, 1600, 1800, 2000, 2400, 2600, 2800, 3000, or more than 3000 sequences of a CDR1, CDR2, or CDR3 of the VL. In some instances, libraries described herein comprise at least or about 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences of a CDR1, CDR2, or CDR3 of the VL. For example, the libraries comprise at least 20 sequences of a CDR1 of the VL, at least 4 sequences of a CDR2 of the VL, and at least 140 sequences of a CDR3 of the VL. In some instances, the libraries comprise at least 2 sequences of a CDR1 of the VL, at least 1 sequence of CDR2 of the VL, and at least 3000 sequences of a CDR3 of the VL. In some instances, the VL is IGKV1-39, IGKV1-9, IGKV2-28, IGKV3-11, IGKV3-15, IGKV3-20, IGKV4-1, IGLV1-51, IGLV2-14, IGLV1-40, or IGLV3-1. In some instances, the VL is IGKV2-28. In some instances, the VL is IGLV1-51.

In some instances, the CDR1, CDR2, or CDR3 is of a variable domain, heavy chain (VH). CDR1, CDR2, or CDR3 of a variable domain, heavy chain (VH) can be referred to as CDRH1, CDRH2, or CDRH3, respectively. In some instances, libraries described herein comprise at least or about 1, 2, 3, 4, 5, 6, 7, 8, 9, 10, 20, 30, 40, 50, 60, 70, 80, 90, 100, 200, 300, 400, 500, 600, 700, 800, 900, 1000, 1200, 1400, 1600, 1800, 2000, 2400, 2600, 2800, 3000, or more than 3000 sequences of a CDR1, CDR2, or CDR3 of the VH. In some instances, libraries described herein comprise at least or about 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences of a CDR1, CDR2, or CDR3 of the VH. For example, the libraries comprise at least 30 sequences of a CDR1 of the VH, at least 570 sequences of a CDR2 of the VH, and at least 108 sequences of a CDR3 of the VH. In some instances, the libraries comprise at least 30 sequences of a CDR1 of the VH, at least 860 sequences of a CDR2 of the VH, and at least 107 sequences of a CDR3 of the VH. In some instances, the VH is IGHV1-18, IGHV1-69, IGHV1-8 IGHV3-21, IGHV3-23, IGHV3-30/33rn, IGHV3-28, IGHV3-74, IGHV4-39, or IGHV4-59/61. In some instances, the VH is IGHV1-69, IGHV3-30, IGHV3-23, IGHV3, IGHV1-46, IGHV3-7, IGHV1, or IGHV1-8. In some instances, the VH is IGHV1-69 and IGHV3-30. In some instances, the VH is IGHV3-23.

Libraries as described herein, in some embodiments, comprise varying lengths of a CDRL1, CDRL2, CDRL3, CDRH1, CDRH2, or CDRH3. In some instances, the length of the CDRL1, CDRL2, CDRL3, CDRH1, CDRH2, or CDRH3 comprises at least or about 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or more than 90 amino acids in length. For example, the CDRH3 comprises at least or about 12, 15, 16, 17, 20, 21, or 23 amino acids in length. In some instances, the CDRL1, CDRL2, CDRL3, CDRH1, CDRH2, or CDRH3 comprises a range of about 1 to about 10, about 5 to about 15, about 10 to about 20, or about 15 to about 30 amino acids in length.

Libraries comprising nucleic acids encoding for antibodies having variant CDR sequences as described herein comprise various lengths of amino acids when translated. In some instances, the length of each of the amino acid fragments or average length of the amino acid synthesized may be at least or about 15, 20, 25, 30, 35, 40, 45, 50, 55, 60, 65, 70, 75, 80, 85, 90, 95, 100, 105, 110, 115, 120, 125, 130, 135, 140, 145, 150, or more than 150 amino acids. In some instances, the length of the amino acid is about 15 to 150, 20 to 145, 25 to 140, 30 to 135, 35 to 130, 40 to 125, 45 to 120, 50 to 115, 55 to 110, 60 to 110, 65 to 105, 70 to 100, or 75 to 95 amino acids. In some instances, the length of the amino acid is about 22 amino acids to about 75 amino acids. In some instances, the antibodies comprise at least or about 100, 200, 300, 400, 500, 600, 700, 800, 900, 1000, 2000, 3000, 4000, 5000, or more than 5000 amino acids.

Ratios of the lengths of a CDRL1, CDRL2, CDRL3, CDRH1, CDRH2, or CDRH3 may vary in libraries described herein. In some instances, a CDRL1, CDRL2, CDRL3, CDRH1, CDRH2, or CDRH3 comprising at least or about 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or more than 90 amino acids in length comprises about 10%, 20%, 30%, 40%, 50%, 60%, 70%, 80%, 90%, or more than 90% of the library. For example, a CDRH3 comprising about 23 amino acids in length is present in the library at 40%, a CDRH3 comprising about 21 amino acids in length is present in the library at 30%, a CDRH3 comprising about 17 amino acids in length is present in the library at 20%, and a CDRH3 comprising about 12 amino acids in length is present in the library at 10%. In some instances, a CDRH3 comprising about 20 amino acids in length is present in the library at 40%, a CDRH3 comprising about 16 amino acids in length is present in the library at 30%, a CDRH3 comprising about 15 amino acids in length is present in the library at 20%, and a CDRH3 comprising about 12 amino acids in length is present in the library at 10%.

Libraries as described herein encoding for a VHH antibody comprise variant CDR sequences that are shuffled to generate a library with a theoretical diversity of at least or about 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences.

In some instances, the library has a final library diversity of at least or about 10⁷, 10⁸, 10⁹, 10¹⁰ 10¹¹, 10¹², 10¹³, 10¹⁴, 10¹⁵, 10¹⁶, 10¹⁷, 10¹⁸, 10¹⁹, 10²⁰, or more than 10²⁰ sequences.

Provided herein are adenosine A2B receptor binding libraries encoding for an immunoglobulin. In some instances, the adenosine A2B receptor immunoglobulin is an antibody. In some instances, the adenosine A2B receptor immunoglobulin is a VHH antibody. In some instances, the adenosine A2B receptor immunoglobulin comprises a binding affinity (e.g., K_{D}) to adenosine A2A receptor of less than 1 nM, less than 1.2 nM, less than 2 nM, less than 5 nM, less than 10 nM, less than 11 nm, less than 13.5 nM, less than 15 nM, less than 20 nM, less than 25 nM, or less than 30 nM. In some instances, the adenosine A2B receptor immunoglobulin comprises a K_{D} of less than 1 nM. In some instances, the adenosine A2B receptor immunoglobulin comprises a K_{D}) of less than 1.2 nM. In some instances, the adenosine A2B receptor immunoglobulin comprises a K_{D} of less than 2 nM. In some instances, the adenosine A2B receptor immunoglobulin comprises a K_{D} of less than 5 nM. In some instances, the adenosine A2B receptor immunoglobulin comprises a K_{D} of less than 10 nM. In some instances, the adenosine A2B receptor immunoglobulin comprises a K_{D} of less than 13.5 nM. In some instances, the adenosine A2B receptor immunoglobulin comprises a K_{D} of less than 15 nM. In some instances, the adenosine A2B receptor immunoglobulin comprises a K_{D} of less than 20 nM. In some instances, the adenosine A2B receptor immunoglobulin comprises a K_{D} of less than 25 nM. In some instances, the adenosine A2B receptor immunoglobulin comprises a K_{D} of less than 30 nM.

In some instances, the adenosine A2B receptor immunoglobulin is an adenosine A2B receptor agonist. In some instances, the adenosine A2B receptor immunoglobulin is an adenosine A2B receptor antagonist. In some instances, the adenosine A2B receptor immunoglobulin is an adenosine A2B receptor allosteric modulator. In some instances, the allosteric modulator is a negative allosteric modulator. In some instances, the allosteric modulator is a positive allosteric modulator. In some instances, the adenosine A2B receptor immunoglobulin results in agonistic, antagonistic, or allosteric effects at a concentration of at least or about 1 nM, 2 nM, 4 nM, 6 nM, 8 nM, 10 nM, 20 nM, 30 nM, 40 nM, 50 nM, 60 nM, 70 nM, 80 nM, 90 nM, 100 nM, 120 nM, 140 nM, 160 nM, 180 nM, 200 nM, 300 nM, 400 nM, 500 nM, 600 nM, 700 nM, 800 nM, 900 nM, 1000 nM, or more than 1000 nM. In some instances, the adenosine A2B receptor immunoglobulin is a negative allosteric modulator. In some instances, the adenosine A2B receptor immunoglobulin is a negative allosteric modulator at a concentration of at least or about 0.001, 0.005, 0.01, 0.05, 0.1, 0.5, 1 nM, 2 nM, 4 nM, 6 nM, 8 nM, 10 nM, 20 nM, 30 nM, 40 nM, 50 nM, 60 nM, 70 nM, 80 nM, 90 nM, 100 nM, or more than 100 nM. In some instances, the adenosine A2B receptor immunoglobulin is a negative allosteric modulator at a concentration in a range of about 0.001 to about 100, 0.01 to about 90, about 0.1 to about 80, 1 to about 50, about 10 to about 40 nM, or about 1 to about 10 nM. In some instances, the adenosine A2B receptor immunoglobulin comprises an EC50 or IC50 of at least or about 0.001, 0.0025, 0.005, 0.01, 0.025, 0.05, 0.06, 0.07, 0.08, 0.9, 0.1, 0.5, 1, 2, 3, 4, 5, 6, or more than 6 nM. In some instances, the adenosine A2B receptor immunoglobulin comprises an EC50 or IC50 of at least or about 1 nM, 2 nM, 4 nM, 6 nM, 8 nM, 10 nM, 20 nM, 30 nM, 40 nM, 50 nM, 60 nM, 70 nM, 80 nM, 90 nM, 100 nM, or more than 100 nM.

Adenosine A2B receptor immunoglobulins as described herein may comprise improved properties. In some instances, the adenosine A2B receptor immunoglobulins are monomeric. In some instances, the adenosine A2B receptor immunoglobulins are not prone to aggregation. In some instances, at least or about 70%, 75%, 80%, 85%, 90%, 95%, or 99% of the adenosine A2B receptor immunoglobulins are monomeric. In some instances, the adenosine A2B receptor immunoglobulins are thermostable. In some instances, the adenosine A2B receptor immunoglobulins result in reduced non-specific binding.

Following synthesis of adenosine A2B receptor binding libraries comprising nucleic acids encoding scaffolds comprising adenosine A2B receptor binding domains, libraries may be used for screening and analysis. For example, libraries are assayed for library displayability and panning. In some instances, displayability is assayed using a selectable tag. Exemplary tags include, but are not limited to, a radioactive label, a fluorescent label, an enzyme, a chemiluminescent tag, a colorimetric tag, an affinity tag or other labels or tags that are known in the art. In some instances, the tag is histidine, polyhistidine, myc, hemagglutinin (HA), or FLAG. The adenosine A2B receptor binding libraries may comprise nucleic acids encoding scaffolds comprising adenosine A2B receptor binding domains with multiple tags such as GFP, FLAG, and Lucy as well as a DNA barcode. In some instances, libraries are assayed by sequencing using various methods including, but not limited to, single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis.

### Expression Systems

Provided herein are libraries comprising nucleic acids encoding for scaffolds comprising adenosine A2A receptor binding domains, adenosine A2B receptor binding domains, or a combination thereof, wherein the libraries have improved specificity, stability, expression, folding, or downstream activity. In some instances, libraries described herein are used for screening and analysis.

Provided herein are libraries comprising nucleic acids encoding for scaffolds comprising adenosine A2A receptor binding domains, adenosine A2B receptor binding domains, or a combination thereof, wherein the nucleic acid libraries are used for screening and analysis. In some instances, screening and analysis comprises in vitro, in vivo, or ex vivo assays. Cells for screening include primary cells taken from living subjects or cell lines. Cells may be from prokaryotes (e.g., bacteria and fungi) or eukaryotes (e.g., animals and plants). Exemplary animal cells include, without limitation, those from a mouse, rabbit, primate, and insect. In some instances, cells for screening include a cell line including, but not limited to, Chinese Hamster Ovary (CHO) cell line, human embryonic kidney (HEK) cell line, or baby hamster kidney (BHK) cell line. In some instances, nucleic acid libraries described herein may also be delivered to a multicellular organism. Exemplary multicellular organisms include, without limitation, a plant, a mouse, rabbit, primate, and insect.

Nucleic acid libraries or protein libraries encoded thereof described herein may be screened for various pharmacological or pharmacokinetic properties. In some instances, the libraries are screened using in vitro assays, in vivo assays, or ex vivo assays. For example, in vitro pharmacological or pharmacokinetic properties that are screened include, but are not limited to, binding affinity, binding specificity, and binding avidity. Exemplary in vivo pharmacological or pharmacokinetic properties of libraries described herein that are screened include, but are not limited to, therapeutic efficacy, activity, preclinical toxicity properties, clinical efficacy properties, clinical toxicity properties, immunogenicity, potency, and clinical safety properties.

Pharmacological or pharmacokinetic properties that may be screened include, but are not limited to, cell binding affinity and cell activity. For example, cell binding affinity assays or cell activity assays are performed to determine agonistic, antagonistic, or allosteric effects of libraries described herein. In some instances, the cell activity assay is a cAMP assay. In some instances, libraries as described herein are compared to cell binding or cell activity of ligands of adenosine A2A receptor, adenosine A2B receptor, or both adenosine A2A receptor and adenosine A2B receptor.

Libraries as described herein may be screened in cell-based assays or in non-cell-based assays. Examples of non-cell-based assays include, but are not limited to, using viral particles, using in vitro translation proteins, and using protealiposomes with adenosine A2A receptor, adenosine A2B receptor, or both adenosine A2A receptor and adenosine A2B receptor.

Nucleic acid libraries as described herein may be screened by sequencing. In some instances, next generation sequence is used to determine sequence enrichment of adenosine A2A receptor binding variants, adenosine A2B receptor binding variants, or a combination thereof. In some instances, V gene distribution, J gene distribution, V gene family, CDR3 counts per length, or a combination thereof is determined. In some instances, clonal frequency, clonal accumulation, lineage accumulation, or a combination thereof is determined. In some instances, number of sequences, sequences with VH clones, clones, clones greater than 1, clonotypes, clonotypes greater than 1, lineages, simpsons, or a combination thereof is determined. In some instances, a percentage of non-identical CDR3s is determined. For example, the percentage of non-identical CDR3s is calculated as the number of non-identical CDR3s in a sample divided by the total number of sequences that had a CDR3 in the sample.

Provided herein are nucleic acid libraries, wherein the nucleic acid libraries may be expressed in a vector. Expression vectors for inserting nucleic acid libraries disclosed herein may comprise eukaryotic or prokaryotic expression vectors. Exemplary expression vectors include, without limitation, mammalian expression vectors: pSF-CMV-NEO-NH2-PPT-3×FLAG, pSF-CMV-NEO-COOH-3×FLAG, pSF-CMV-PURO-NH2-GST-TEV, pSF-OXB20-COOH-TEV-FLAG (R)-6His, pCEP4 pDEST27, pSF-CMV-Ub-KrYFP, pSF-CMV-FMDV-daGFP, pEF1a-mCherry-N1 Vector, pEF1a-tdTomato Vector, pSF-CMV-FMDV-Hygro, pSF-CMV-PGK-Puro, pMCP-tag (m), and pSF-CMV-PURO-NH2-CMYC; bacterial expression vectors: pSF-OXB20-BetaGal, pSF-OXB20-Fluc, pSF-OXB20, and pSF-Tac; plant expression vectors: pRI 101-AN DNA and pCambia2301; and yeast expression vectors: pTYB21 and pKLAC2, and insect vectors: pAc5.1/V5-His A and pDEST8. In some instances, the vector is pcDNA3 or pcDNA3.1.

Described herein are nucleic acid libraries that are expressed in a vector to generate a construct comprising a scaffold comprising sequences of adenosine A2A receptor binding domains, adenosine A2B receptor domains, or a combination thereof. In some instances, a size of the construct varies. In some instances, the construct comprises at least or about 500, 600, 700, 800, 900, 1000, 1100, 1300, 1400, 1500, 1600, 1700, 1800, 2000, 2400, 2600, 2800, 3000, 3200, 3400, 3600, 3800, 4000, 4200, 4400, 4600, 4800, 5000, 6000, 7000, 8000, 9000, 10000, or more than 10000 bases. In some instances, a the construct comprises a range of about 300 to 1,000, 300 to 2,000, 300 to 3,000, 300 to 4,000, 300 to 5,000, 300 to 6,000, 300 to 7,000, 300 to 8,000, 300 to 9,000, 300 to 10,000, 1,000 to 2,000, 1,000 to 3,000, 1,000 to 4,000, 1,000 to 5,000, 1,000 to 6,000, 1,000 to 7,000, 1,000 to 8,000, 1,000 to 9,000, 1,000 to 10,000, 2,000 to 3,000, 2,000 to 4,000, 2,000 to 5,000, 2,000 to 6,000, 2,000 to 7,000, 2,000 to 8,000, 2,000 to 9,000, 2,000 to 10,000, 3,000 to 4,000, 3,000 to 5,000, 3,000 to 6,000, 3,000 to 7,000, 3,000 to 8,000, 3,000 to 9,000, 3,000 to 10,000, 4,000 to 5,000, 4,000 to 6,000, 4,000 to 7,000, 4,000 to 8,000, 4,000 to 9,000, 4,000 to 10,000, 5,000 to 6,000, 5,000 to 7,000, 5,000 to 8,000, 5,000 to 9,000, 5,000 to 10,000, 6,000 to 7,000, 6,000 to 8,000, 6,000 to 9,000, 6,000 to 10,000, 7,000 to 8,000, 7,000 to 9,000, 7,000 to 10,000, 8,000 to 9,000, 8,000 to 10,000, or 9,000 to 10,000 bases.

Provided herein are libraries comprising nucleic acids encoding for scaffolds comprising adenosine A2A receptor binding domains, adenosine A2B receptor domains, or a combination thereof, wherein the nucleic acid libraries are expressed in a cell. In some instances, the libraries are synthesized to express a reporter gene. Exemplary reporter genes include, but are not limited to, acetohydroxyacid synthase (AHAS), alkaline phosphatase (AP), beta galactosidase (LacZ), beta glucoronidase (GUS), chloramphenicol acetyltransferase (CAT), green fluorescent protein (GFP), red fluorescent protein (RFP), yellow fluorescent protein (YFP), cyan fluorescent protein (CFP), cerulean fluorescent protein, citrine fluorescent protein, orange fluorescent protein, cherry fluorescent protein, turquoise fluorescent protein, blue fluorescent protein, horseradish peroxidase (HRP), luciferase (Luc), nopaline synthase (NOS), octopine synthase (OCS), luciferase, and derivatives thereof. Methods to determine modulation of a reporter gene are well known in the art, and include, but are not limited to, fluorometric methods (e.g. fluorescence spectroscopy, Fluorescence Activated Cell Sorting (FACS), fluorescence microscopy), and antibiotic resistance determination.

### Diseases and Disorders

Provided herein are adenosine A2A receptor binding libraries comprising nucleic acids encoding for scaffolds comprising adenosine A2A receptor binding domains that may have therapeutic effects. Also provided herein are adenosine A2B receptor binding libraries comprising nucleic acids encoding for scaffolds comprising adenosine A2B receptor binding domains that may have therapeutic effects. In some instances, the adenosine A2A receptor binding libraries and adenosine A2B receptor libraries result in protein when translated that is used to treat a disease or disorder. In some instances, the protein is an immunoglobulin. In some instances, the protein is a peptidomimetic. Exemplary diseases include, but are not limited to, cancer, inflammatory diseases or disorders, a metabolic disease or disorder, a cardiovascular disease or disorder, a respiratory disease or disorder, pain, a digestive disease or disorder, a reproductive disease or disorder, an endocrine disease or disorder, or a neurological disease or disorder. In some instances, an inhibitor of adenosine A2A receptor, adenosine A2B receptor, or a combination thereof as described herein is used for treatment of a disease or disorder of the central nervous system, kidney, intestine, lung, hair, skin, bone, or cartilage. In some instances, an inhibitor of adenosine A2A receptor, adenosine A2B receptor, or a combination thereof as described herein is used for sleep regulation, angiogenesis, or modulation of the immune system.

In some instances, the A2AR immunoglobulins, A2BR immunoglobulins, or a combination thereof described herein are used for treating a neurological disease or disorder. In some instances, the neurological disease or disorder is a neurodegenerative disease or disorder. In some instances, the neurological disease or disorder is Parkinson's disease, Alzheimer's disease, or multiple sclerosis.

In some instances, the A2AR immunoglobulins, A2BR immunoglobulins, or a combination thereof described herein are used for treating cancer. In some instances, the cancer is a solid cancer or a hematologic cancer. In some instances, the A2AR immunoglobulins, A2BR immunoglobulins, or a combination thereof described herein are used as a monotherapy for treating cancer. In some instances, the A2AR immunoglobulins, A2BR immunoglobulins, or a combination thereof described herein are used in combination with other therapeutic agents for treating cancer. In some embodiments, the cancer is lung, colorectal, or prostate cancer. In some instances, the A2AR immunoglobulins, A2BR immunoglobulins, or a combination thereof described herein enhance tumor vaccines, checkpoint blockade and adoptive T cell therapy. In some instances, the A2AR immunoglobulin, A2BR immunoglobulins, or a combination thereof targets immune cells and blocks immunosuppression in order to treat cancer.

In some instances, the A2AR immunoglobulins, A2BR immunoglobulins, or a combination thereof described herein reduce tumor size by at least or about 10%, 15%, 20%, 25%, 30%, 40%, 45%, 50%, 55%, 60%, 65%, 70%, 75%, 80%, 85%, 90%, 95%, or more than 95%. In some instances, the A2AR immunoglobulins, A2BR immunoglobulins, or a combination thereof described herein reduce tumor size by at least or about 10%, 15%, 20%, 25%, 30%, 40%, 45%, 50%, 55%, 60%, 65%, 70%, 75%, 80%, 85%, 90%, 95%, or more than 95% compared to a comparator antibody (e.g., pembrolizumab or nivolumab) or a control. In some instances, the control is no treatment or placebo.

In some instances, the A2AR immunoglobulins, A2BR immunoglobulins, or a combination thereof described herein increase cell number of cells of the lymphoid or myeloid compartment. In some instances, the A2AR immunoglobulins, A2BR immunoglobulins, or a combination thereof described herein increase tumor infiltrating lymphocytes (TIL) CD45+ cells, total T-cells, CD4+ cells, CD8+ cells, regulatory T-cells (Tregs), M1 tumor associated macrophages (TAM), or combinations thereof by at least or about 10%, 15%, 20%, 25%, 30%, 40%, 45%, 50%, 55%, 60%, 65%, 70%, 75%, 80%, 85%, 90%, 95%, or more than 95%. In some instances, the A2AR immunoglobulins, A2BR immunoglobulins, or a combination thereof described herein increase tumor infiltrating lymphocytes (TIL) CD45+ cells, total T-cells, CD4+ cells, CD8+ cells, regulatory T-cells (Tregs), M1 tumor associated macrophages (TAM), or combinations thereof by at least or about 10%, 15%, 20%, 25%, 30%, 40%, 45%, 50%, 55%, 60%, 65%, 70%, 75%, 80%, 85%, 90%, 95%, or more than 95% compared to a comparator antibody (e.g., pembrolizumab or nivolumab) or a control. In some instances, the control is no treatment or placebo.

In some instances, the A2AR immunoglobulins, A2BR immunoglobulins, or a combination thereof described herein increase cytokine expression. In some embodiments, the cytokine is interferon gamma, interleukin 2, interleukin 4, interleukin 6, interleukin 8, interleukin 10, or TNF alpha. In some embodiments, the cytokine is interleukin 1B, interleukin 1Ra, GM-CSF, interleukin 2, interleukin 7, interleukin 15, interleukin 6, interleukin 6, interleukin 10, interferon gamma, or TNF alpha. In some instances, the A2AR immunoglobulins, A2BR immunoglobulins, or a combination thereof described herein increase cytokine expression by at least or about 10%, 15%, 20%, 25%, 30%, 40%, 45%, 50%, 55%, 60%, 65%, 70%, 75%, 80%, 85%, 90%, 95%, or more than 95%. In some instances, the A2AR immunoglobulins, A2BR immunoglobulins, or a combination thereof described herein increase cytokine expression by at least or about 10%, 15%, 20%, 25%, 30%, 40%, 45%, 50%, 55%, 60%, 65%, 70%, 75%, 80%, 85%, 90%, 95%, or more than 95% compared to a comparator antibody (e.g., pembrolizumab or nivolumab) or a control. In some instances, the control is no treatment or placebo.

In some instances, the subject is a mammal. In some instances, the subject is a mouse, rabbit, dog, or human. Subjects treated by methods described herein may be infants, adults, or children. Pharmaceutical compositions comprising antibodies or antibody fragments as described herein may be administered intravenously or subcutaneously.

### Variant Libraries

### Codon Variation

Variant nucleic acid libraries described herein may comprise a plurality of nucleic acids, wherein each nucleic acid encodes for a variant codon sequence compared to a reference nucleic acid sequence. In some instances, each nucleic acid of a first nucleic acid population contains a variant at a single variant site. In some instances, the first nucleic acid population contains a plurality of variants at a single variant site such that the first nucleic acid population contains more than one variant at the same variant site. The first nucleic acid population may comprise nucleic acids collectively encoding multiple codon variants at the same variant site. The first nucleic acid population may comprise nucleic acids collectively encoding up to 19 or more codons at the same position. The first nucleic acid population may comprise nucleic acids collectively encoding up to 60 variant triplets at the same position, or the first nucleic acid population may comprise nucleic acids collectively encoding up to 61 different triplets of codons at the same position. Each variant may encode for a codon that results in a different amino acid during translation. Table 2 provides a listing of each codon possible (and the representative amino acid) for a variant site.

TABLE 2
List of codons and amino acids

| | One | Three | | | | | | |
|---|---|---|---|---|---|---|---|---|
| | letter | letter | | | | | | |
| Amino Acids | code | code | Codons | | | | | |
| Alanine | A | Ala | GCA | GCC | GCG | GCT | | |
| Cysteine | C | Cys | TGC | TGT | | | | |
| Aspartic acid | D | Asp | GAC | GAT | | | | |
| Glutamic acid | E | Glu | GAA | GAG | | | | |
| Phenylalanine | F | Phe | TTC | TTT | | | | |
| Glycine | G | Gly | GGA | GGC | GGG | GGT | | |
| Histidine | H | His | CAC | CAT | | | | |
| Isoleucine | I | Iso | ATA | ATC | ATT | | | |
| Lysine | K | Lys | AAA | AAG | | | | |
| Leucine | L | Leu | TTA | TTG | CTA | CTC | CTG | CTT |
| Methionine | M | Met | ATG | | | | | |
| Asparagine | N | Asn | AAC | AAT | | | | |
| Proline | P | Pro | CCA | CCC | CCG | CCT | | |
| Glutamine | Q | Gln | CAA | CAG | | | | |
| Arginine | R | Arg | AGA | AGG | CGA | CGC | CGG | CGT |
| Serine | S | Ser | AGC | AGT | TCA | TCC | TCG | TCT |

| | | | | | | |
|---|---|---|---|---|---|---|
| Threonine | T | Thr | ACA | ACC | ACG | ACT |
| Valine | V | Val | GTA | GTC | GTG | GTT |

| Tryptophan | W | Trp | TGG | |
|---|---|---|---|---|
| Tyrosine | Y | Tyr | TAC | TAT |

A nucleic acid population may comprise varied nucleic acids collectively encoding up to 20 codon variations at multiple positions. In such cases, each nucleic acid in the population comprises variation for codons at more than one position in the same nucleic acid. In some instances, each nucleic acid in the population comprises variation for codons at 1, 2, 3, 4, 5, 6, 7, 8, 9, 10, 11, 12, 13, 14, 15, 16, 17, 18, 19, 20 or more codons in a single nucleic acid. In some instances, each variant long nucleic acid comprises variation for codons at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more codons in a single long nucleic acid. In some instances, the variant nucleic acid population comprises variation for codons at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more codons in a single nucleic acid. In some instances, the variant nucleic acid population comprises variation for codons in at least about 10, 20, 30, 40, 50, 60, 70, 80, 90, 100 or more codons in a single long nucleic acid.

### Highly Parallel Nucleic Acid Synthesis

Provided herein is a platform approach utilizing miniaturization, parallelization, and vertical integration of the end-to-end process from polynucleotide synthesis to gene assembly within nanowells on silicon to create a revolutionary synthesis platform. Devices described herein provide, with the same footprint as a 96-well plate, a silicon synthesis platform is capable of increasing throughput by a factor of up to 1,000 or more compared to traditional synthesis methods, with production of up to approximately 1,000,000 or more polynucleotides, or 10,000 or more genes in a single highly-parallelized run.

With the advent of next-generation sequencing, high resolution genomic data has become an important factor for studies that delve into the biological roles of various genes in both normal biology and disease pathogenesis. At the core of this research is the central dogma of molecular biology and the concept of “residue-by-residue transfer of sequential information.” Genomic information encoded in the DNA is transcribed into a message that is then translated into the protein that is the active product within a given biological pathway.

Another exciting area of study is on the discovery, development and manufacturing of therapeutic molecules focused on a highly-specific cellular target. High diversity DNA sequence libraries are at the core of development pipelines for targeted therapeutics. Gene mutants are used to express proteins in a design, build, and test protein engineering cycle that ideally culminates in an optimized gene for high expression of a protein with high affinity for its therapeutic target. As an example, consider the binding pocket of a receptor. The ability to test all sequence permutations of all residues within the binding pocket simultaneously will allow for a thorough exploration, increasing chances of success. Saturation mutagenesis, in which a researcher attempts to generate all possible mutations at a specific site within the receptor, represents one approach to this development challenge. Though costly and time and labor-intensive, it enables each variant to be introduced into each position. In contrast, combinatorial mutagenesis, where a few selected positions or short stretch of DNA may be modified extensively, generates an incomplete repertoire of variants with biased representation.

To accelerate the drug development pipeline, a library with the desired variants available at the intended frequency in the right position available for testing—in other words, a precision library, enables reduced costs as well as turnaround time for screening. Provided herein are methods for synthesizing nucleic acid synthetic variant libraries which provide for precise introduction of each intended variant at the desired frequency. To the end user, this translates to the ability to not only thoroughly sample sequence space but also be able to query these hypotheses in an efficient manner, reducing cost and screening time. Genome-wide editing can elucidate important pathways, libraries where each variant and sequence permutation can be tested for optimal functionality, and thousands of genes can be used to reconstruct entire pathways and genomes to re-engineer biological systems for drug discovery.

In a first example, a drug itself can be optimized using methods described herein. For example, to improve a specified function of an antibody, a variant polynucleotide library encoding for a portion of the antibody is designed and synthesized. A variant nucleic acid library for the antibody can then be generated by processes described herein (e.g., PCR mutagenesis followed by insertion into a vector). The antibody is then expressed in a production cell line and screened for enhanced activity. Example screens include examining modulation in binding affinity to an antigen, stability, or effector function (e.g., ADCC, complement, or apoptosis). Exemplary regions to optimize the antibody include, without limitation, the Fc region, Fab region, variable region of the Fab region, constant region of the Fab region, variable domain of the heavy chain or light chain (V_{H} or V_{L}), and specific complementarity-determining regions (CDRs) of V_{H} or V_{L}.

Nucleic acid libraries synthesized by methods described herein may be expressed in various cells associated with a disease state. Cells associated with a disease state include cell lines, tissue samples, primary cells from a subject, cultured cells expanded from a subject, or cells in a model system. Exemplary model systems include, without limitation, plant and animal models of a disease state.

To identify a variant molecule associated with prevention, reduction or treatment of a disease state, a variant nucleic acid library described herein is expressed in a cell associated with a disease state, or one in which a cell a disease state can be induced. In some instances, an agent is used to induce a disease state in cells. Exemplary tools for disease state induction include, without limitation, a Cre/Lox recombination system, LPS inflammation induction, and streptozotocin to induce hypoglycemia. The cells associated with a disease state may be cells from a model system or cultured cells, as well as cells from a subject having a particular disease condition. Exemplary disease conditions include a bacterial, fungal, viral, autoimmune, or proliferative disorder (e.g., cancer). In some instances, the variant nucleic acid library is expressed in the model system, cell line, or primary cells derived from a subject, and screened for changes in at least one cellular activity. Exemplary cellular activities include, without limitation, proliferation, cycle progression, cell death, adhesion, migration, reproduction, cell signaling, energy production, oxygen utilization, metabolic activity, and aging, response to free radical damage, or any combination thereof.

### Substrates

Devices used as a surface for polynucleotide synthesis may be in the form of substrates which include, without limitation, homogenous array surfaces, patterned array surfaces, channels, beads, gels, and the like. Provided herein are substrates comprising a plurality of clusters, wherein each cluster comprises a plurality of loci that support the attachment and synthesis of polynucleotides. In some instances, substrates comprise a homogenous array surface. For example, the homogenous array surface is a homogenous plate. The term “locus” as used herein refers to a discrete region on a structure which provides support for polynucleotides encoding for a single predetermined sequence to extend from the surface. In some instances, a locus is on a two dimensional surface, e.g., a substantially planar surface. In some instances, a locus is on a three-dimensional surface, e.g., a well, microwell, channel, or post. In some instances, a surface of a locus comprises a material that is actively functionalized to attach to at least one nucleotide for polynucleotide synthesis, or preferably, a population of identical nucleotides for synthesis of a population of polynucleotides. In some instances, polynucleotide refers to a population of polynucleotides encoding for the same nucleic acid sequence. In some cases, a surface of a substrate is inclusive of one or a plurality of surfaces of a substrate. The average error rates for polynucleotides synthesized within a library described here using the systems and methods provided are often less than 1 in 1000, less than about 1 in 2000, less than about 1 in 3000 or less often without error correction.

Provided herein are surfaces that support the parallel synthesis of a plurality of polynucleotides having different predetermined sequences at addressable locations on a common support. In some instances, a substrate provides support for the synthesis of more than 50, 100, 200, 400, 600, 800, 1000, 1200, 1400, 1600, 1800, 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical polynucleotides. In some cases, the surfaces provide support for the synthesis of more than 50, 100, 200, 400, 600, 800, 1000, 1200, 1400, 1600, 1800, 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more polynucleotides encoding for distinct sequences. In some instances, at least a portion of the polynucleotides have an identical sequence or are configured to be synthesized with an identical sequence. In some instances, the substrate provides a surface environment for the growth of polynucleotides having at least 80, 90, 100, 120, 150, 175, 200, 225, 250, 275, 300, 325, 350, 375, 400, 425, 450, 475, 500 bases or more.

Provided herein are methods for polynucleotide synthesis on distinct loci of a substrate, wherein each locus supports the synthesis of a population of polynucleotides. In some cases, each locus supports the synthesis of a population of polynucleotides having a different sequence than a population of polynucleotides grown on another locus. In some instances, each polynucleotide sequence is synthesized with 1, 2, 3, 4, 5, 6, 7, 8, 9 or more redundancy across different loci within the same cluster of loci on a surface for polynucleotide synthesis. In some instances, the loci of a substrate are located within a plurality of clusters. In some instances, a substrate comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some instances, a substrate comprises more than 2,000; 5,000; 10,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct loci. In some instances, a substrate comprises about 10,000 distinct loci. The amount of loci within a single cluster is varied in different instances. In some cases, each cluster includes 1, 2, 3, 4, 5, 6, 7, 8, 9, 10, 20, 30, 40, 50, 60, 70, 80, 90, 100, 120, 130, 150, 200, 300, 400, 500 or more loci. In some instances, each cluster includes about 50-500 loci. In some instances, each cluster includes about 100-200 loci. In some instances, each cluster includes about 100-150 loci. In some instances, each cluster includes about 109, 121, 130 or 137 loci. In some instances, each cluster includes about 19, 20, 61, 64 or more loci. Alternatively or in combination, polynucleotide synthesis occurs on a homogenous array surface.

In some instances, the number of distinct polynucleotides synthesized on a substrate is dependent on the number of distinct loci available in the substrate. In some instances, the density of loci within a cluster or surface of a substrate is at least or about 1, 10, 25, 50, 65, 75, 100, 130, 150, 175, 200, 300, 400, 500, 1,000 or more loci per mm². In some cases, a substrate comprises 10-500, 25-400, 50-500, 100-500, 150-500, 10-250, 50-250, 10-200, or 50-200 mm². In some instances, the distance between the centers of two adjacent loci within a cluster or surface is from about 10-500, from about 10-200, or from about 10-100 um. In some instances, the distance between two centers of adjacent loci is greater than about 10, 20, 30, 40, 50, 60, 70, 80, 90 or 100 μm. In some instances, the distance between the centers of two adjacent loci is less than about 200, 150, 100, 80, 70, 60, 50, 40, 30, 20 or 10 um. In some instances, each locus has a width of about 0.5, 1, 2, 3, 4, 5, 6, 7, 8, 9, 10, 20, 30, 40, 50, 60, 70, 80, 90 or 100 um. In some cases, each locus has a width of about 0.5-100, 0.5-50, 10-75, or 0.5-50 um.

In some instances, the density of clusters within a substrate is at least or about 1 cluster per 100 mm², 1 cluster per 10 mm², 1 cluster per 5 mm², 1 cluster per 4 mm², 1 cluster per 3 mm², 1 cluster per 2 mm², 1 cluster per 1 mm², 2 clusters per 1 mm², 3 clusters per 1 mm², 4 clusters per 1 mm², 5 clusters per 1 mm², 10 clusters per 1 mm², 50 clusters per 1 mm² or more. In some instances, a substrate comprises from about 1 cluster per 10 mm² to about 10 clusters per 1 mm². In some instances, the distance between the centers of two adjacent clusters is at least or about 50, 100, 200, 500, 1000, 2000, or 5000 um. In some cases, the distance between the centers of two adjacent clusters is between about 50-100, 50-200, 50-300, 50-500, and 100-2000 um. In some cases, the distance between the centers of two adjacent clusters is between about 0.05-50, 0.05-10, 0.05-5, 0.05-4, 0.05-3, 0.05-2, 0.1-10, 0.2-10, 0.3-10, 0.4-10, 0.5-10, 0.5-5, or 0.5-2 mm. In some cases, each cluster has a cross section of about 0.5 to about 2, about 0.5 to about 1, or about 1 to about 2 mm. In some cases, each cluster has a cross section of about 0.5, 0.6, 0.7, 0.8, 0.9, 1, 1.1, 1.2, 1.3, 1.4, 1.5, 1.6, 1.7, 1.8, 1.9 or 2 mm. In some cases, each cluster has an interior cross section of about 0.5, 0.6, 0.7, 0.8, 0.9, 1, 1.1, 1.15, 1.2, 1.3, 1.4, 1.5, 1.6, 1.7, 1.8, 1.9 or 2 mm.

In some instances, a substrate is about the size of a standard 96 well plate, for example between about 100 and about 200 mm by between about 50 and about 150 mm. In some instances, a substrate has a diameter less than or equal to about 1000, 500, 450, 400, 300, 250, 200, 150, 100 or 50 mm. In some instances, the diameter of a substrate is between about 25-1000, 25-800, 25-600, 25-500, 25-400, 25-300, or 25-200 mm. In some instances, a substrate has a planar surface area of at least about 100; 200; 500; 1,000; 2,000; 5,000; 10,000; 12,000; 15,000; 20,000; 30,000; 40,000; 50,000 mm² or more. In some instances, the thickness of a substrate is between about 50-2000, 50-1000, 100-1000, 200-1000, or 250-1000 mm.

### Surface Materials

Substrates, devices, and reactors provided herein are fabricated from any variety of materials suitable for the methods, compositions, and systems described herein. In certain instances, substrate materials are fabricated to exhibit a low level of nucleotide binding. In some instances, substrate materials are modified to generate distinct surfaces that exhibit a high level of nucleotide binding. In some instances, substrate materials are transparent to visible and/or UV light. In some instances, substrate materials are sufficiently conductive, e.g., are able to form uniform electric fields across all or a portion of a substrate. In some instances, conductive materials are connected to an electric ground. In some instances, the substrate is heat conductive or insulated. In some instances, the materials are chemical resistant and heat resistant to support chemical or biochemical reactions, for example polynucleotide synthesis reaction processes. In some instances, a substrate comprises flexible materials. For flexible materials, materials can include, without limitation: nylon, both modified and unmodified, nitrocellulose, polypropylene, and the like. In some instances, a substrate comprises rigid materials. For rigid materials, materials can include, without limitation: glass; fuse silica; silicon, plastics (for example polytetrafluoroethylene, polypropylene, polystyrene, polycarbonate, and blends thereof, and the like); metals (for example, gold, platinum, and the like). The substrate, solid support or reactors can be fabricated from a material selected from the group consisting of silicon, polystyrene, agarose, dextran, cellulosic polymers, polyacrylamides, polydimethylsiloxane (PDMS), and glass. The substrates/solid supports or the microstructures, reactors therein may be manufactured with a combination of materials listed herein or any other suitable material known in the art.

### Surface Architecture

Provided herein are substrates for the methods, compositions, and systems described herein, wherein the substrates have a surface architecture suitable for the methods, compositions, and systems described herein. In some instances, a substrate comprises raised and/or lowered features. One benefit of having such features is an increase in surface area to support polynucleotide synthesis. In some instances, a substrate having raised and/or lowered features is referred to as a three-dimensional substrate. In some cases, a three-dimensional substrate comprises one or more channels. In some cases, one or more loci comprise a channel. In some cases, the channels are accessible to reagent deposition via a deposition device such as a material deposition device. In some cases, reagents and/or fluids collect in a larger well in fluid communication one or more channels. For example, a substrate comprises a plurality of channels corresponding to a plurality of loci with a cluster, and the plurality of channels are in fluid communication with one well of the cluster. In some methods, a library of polynucleotides is synthesized in a plurality of loci of a cluster.

Provided herein are substrates for the methods, compositions, and systems described herein, wherein the substrates are configured for polynucleotide synthesis. In some instances, the structure is configured to allow for controlled flow and mass transfer paths for polynucleotide synthesis on a surface. In some instances, the configuration of a substrate allows for the controlled and even distribution of mass transfer paths, chemical exposure times, and/or wash efficacy during polynucleotide synthesis. In some instances, the configuration of a substrate allows for increased sweep efficiency, for example by providing sufficient volume for a growing polynucleotide such that the excluded volume by the growing polynucleotide does not take up more than 50, 45, 40, 35, 30, 25, 20, 15, 14, 13, 12, 11, 10, 9, 8, 7, 6, 5, 4, 3, 2, 1%, or less of the initially available volume that is available or suitable for growing the polynucleotide. In some instances, a three-dimensional structure allows for managed flow of fluid to allow for the rapid exchange of chemical exposure.

Provided herein are substrates for the methods, compositions, and systems described herein, wherein the substrates comprise structures suitable for the methods, compositions, and systems described herein. In some instances, segregation is achieved by physical structure. In some instances, segregation is achieved by differential functionalization of the surface generating active and passive regions for polynucleotide synthesis. In some instances, differential functionalization is achieved by alternating the hydrophobicity across the substrate surface, thereby creating water contact angle effects that cause beading or wetting of the deposited reagents. Employing larger structures can decrease splashing and cross-contamination of distinct polynucleotide synthesis locations with reagents of the neighboring spots. In some cases, a device, such as a material deposition device, is used to deposit reagents to distinct polynucleotide synthesis locations. Substrates having three-dimensional features are configured in a manner that allows for the synthesis of a large number of polynucleotides (e.g., more than about 10,000) with a low error rate (e.g., less than about 1:500, 1:1000, 1:1500, 1:2,000, 1:3,000, 1:5,000, or 1:10,000). In some cases, a substrate comprises features with a density of about or greater than about 1, 5, 10, 20, 30, 40, 50, 60, 70, 80, 100, 110, 120, 130, 140, 150, 160, 170, 180, 190, 200, 300, 400 or 500 features per mm².

A well of a substrate may have the same or different width, height, and/or volume as another well of the substrate. A channel of a substrate may have the same or different width, height, and/or volume as another channel of the substrate. In some instances, the diameter of a cluster or the diameter of a well comprising a cluster, or both, is between about 0.05-50, 0.05-10, 0.05-5, 0.05-4, 0.05-3, 0.05-2, 0.05-1, 0.05-0.5, 0.05-0.1, 0.1-10, 0.2-10, 0.3-10, 0.4-10, 0.5-10, 0.5-5, or 0.5-2 mm. In some instances, the diameter of a cluster or well or both is less than or about 5, 4, 3, 2, 1, 0.5, 0.1, 0.09, 0.08, 0.07, 0.06, or 0.05 mm. In some instances, the diameter of a cluster or well or both is between about 1.0 and 1.3 mm. In some instances, the diameter of a cluster or well, or both is about 1.150 mm. In some instances, the diameter of a cluster or well, or both is about 0.08 mm. The diameter of a cluster refers to clusters within a two-dimensional or three-dimensional substrate.

In some instances, the height of a well is from about 20-1000, 50-1000, 100-1000, 200-1000, 300-1000, 400-1000, or 500-1000 μm. In some cases, the height of a well is less than about 1000, 900, 800, 700, or 600 um.

In some instances, a substrate comprises a plurality of channels corresponding to a plurality of loci within a cluster, wherein the height or depth of a channel is 5-500, 5-400, 5-300, 5-200, 5-100, 5-50, or 10-50 um. In some cases, the height of a channel is less than 100, 80, 60, 40, or 20 um.

In some instances, the diameter of a channel, locus (e.g., in a substantially planar substrate) or both channel and locus (e.g., in a three-dimensional substrate wherein a locus corresponds to a channel) is from about 1-1000, 1-500, 1-200, 1-100, 5-100, or 10-100 um, for example, about 90, 80, 70, 60, 50, 40, 30, 20 or 10 um. In some instances, the diameter of a channel, locus, or both channel and locus is less than about 100, 90, 80, 70, 60, 50, 40, 30, 20 or 10 um. In some instances, the distance between the center of two adjacent channels, loci, or channels and loci is from about 1-500, 1-200, 1-100, 5-200, 5-100, 5-50, or 5-30, for example, about 20 um.

### Surface Modifications

Provided herein are methods for polynucleotide synthesis on a surface, wherein the surface comprises various surface modifications. In some instances, the surface modifications are employed for the chemical and/or physical alteration of a surface by an additive or subtractive process to change one or more chemical and/or physical properties of a substrate surface or a selected site or region of a substrate surface. For example, surface modifications include, without limitation, (1) changing the wetting properties of a surface, (2) functionalizing a surface, i.e., providing, modifying or substituting surface functional groups, (3) defunctionalizing a surface, i.e., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface.

In some cases, the addition of a chemical layer on top of a surface (referred to as adhesion promoter) facilitates structured patterning of loci on a surface of a substrate. Exemplary surfaces for application of adhesion promotion include, without limitation, glass, silicon, silicon dioxide and silicon nitride. In some cases, the adhesion promoter is a chemical with a high surface energy. In some instances, a second chemical layer is deposited on a surface of a substrate. In some cases, the second chemical layer has a low surface energy. In some cases, surface energy of a chemical layer coated on a surface supports localization of droplets on the surface. Depending on the patterning arrangement selected, the proximity of loci and/or area of fluid contact at the loci are alterable.

In some instances, a substrate surface, or resolved loci, onto which nucleic acids or other moieties are deposited, e.g., for polynucleotide synthesis, are smooth or substantially planar (e.g., two-dimensional) or have irregularities, such as raised or lowered features (e.g., three-dimensional features). In some instances, a substrate surface is modified with one or more different layers of compounds. Such modification layers of interest include, without limitation, inorganic and organic layers such as metals, metal oxides, polymers, small organic molecules and the like.

In some instances, resolved loci of a substrate are functionalized with one or more moieties that increase and/or decrease surface energy. In some cases, a moiety is chemically inert. In some cases, a moiety is configured to support a desired chemical reaction, for example, one or more processes in a polynucleotide synthesis reaction. The surface energy, or hydrophobicity, of a surface is a factor for determining the affinity of a nucleotide to attach onto the surface. In some instances, a method for substrate functionalization comprises: (a) providing a substrate having a surface that comprises silicon dioxide; and (b) silanizing the surface using, a suitable silanizing agent described herein or otherwise known in the art, for example, an organofunctional alkoxysilane molecule. Methods and functionalizing agents are described in U.S. Pat. No. 5,474,796, which is herein incorporated by reference in its entirety.

In some instances, a substrate surface is functionalized by contact with a derivatizing composition that contains a mixture of silanes, under reaction conditions effective to couple the silanes to the substrate surface, typically via reactive hydrophilic moieties present on the substrate surface. Silanization generally covers a surface through self-assembly with organofunctional alkoxysilane molecules. A variety of siloxane functionalizing reagents can further be used as currently known in the art, e.g., for lowering or increasing surface energy. The organofunctional alkoxysilanes are classified according to their organic functions.

### Polynucleotide Synthesis

Methods of the current disclosure for polynucleotide synthesis may include processes involving phosphoramidite chemistry. In some instances, polynucleotide synthesis comprises coupling a base with phosphoramidite. Polynucleotide synthesis may comprise coupling a base by deposition of phosphoramidite under coupling conditions, wherein the same base is optionally deposited with phosphoramidite more than once, i.e., double coupling. Polynucleotide synthesis may comprise capping of unreacted sites. In some instances, capping is optional. Polynucleotide synthesis may also comprise oxidation or an oxidation step or oxidation steps. Polynucleotide synthesis may comprise deblocking, detritylation, and sulfurization. In some instances, polynucleotide synthesis comprises either oxidation or sulfurization. In some instances, between one or each step during a polynucleotide synthesis reaction, the device is washed, for example, using tetrazole or acetonitrile. Time frames for any one step in a phosphoramidite synthesis method may be less than about 2 min, 1 min, 50 sec, 40 sec, 30 sec, 20 sec and 10 sec.

Polynucleotide synthesis using a phosphoramidite method may comprise a subsequent addition of a phosphoramidite building block (e.g., nucleoside phosphoramidite) to a growing polynucleotide chain for the formation of a phosphite triester linkage. Phosphoramidite polynucleotide synthesis proceeds in 3′ to 5′ direction. Phosphoramidite polynucleotide synthesis allows for the controlled addition of one nucleotide to a growing nucleic acid chain per synthesis cycle. In some instances, each synthesis cycle comprises a coupling step. Phosphoramidite coupling involves the formation of a phosphite triester linkage between an activated nucleoside phosphoramidite and a nucleoside bound to the substrate, for example, via a linker. In some instances, the nucleoside phosphoramidite is provided to the device activated. In some instances, the nucleoside phosphoramidite is provided to the device with an activator. In some instances, nucleoside phosphoramidites are provided to the device in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition of a nucleoside phosphoramidite, the device is optionally washed. In some instances, the coupling step is repeated one or more additional times, optionally with a wash step between nucleoside phosphoramidite additions to the substrate. In some instances, a polynucleotide synthesis method used herein comprises 1, 2, 3 or more sequential coupling steps. Prior to coupling, in many cases, the nucleoside bound to the device is de-protected by removal of a protecting group, where the protecting group functions to prevent polymerization. A common protecting group is 4,4′-dimethoxytrityl (DMT).

Following coupling, phosphoramidite polynucleotide synthesis methods optionally comprise a capping step. In a capping step, the growing polynucleotide is treated with a capping agent. A capping step is useful to block unreacted substrate-bound 5′-OH groups after coupling from further chain elongation, preventing the formation of polynucleotides with internal base deletions. Further, phosphoramidites activated with 1H-tetrazole may react, to a small extent, with the O6 position of guanosine. Without being bound by theory, upon oxidation with I₂/water, this side product, possibly via O6-N7 migration, may undergo depurination. The apurinic sites may end up being cleaved in the course of the final deprotection of the polynucleotide thus reducing the yield of the full-length product. The O6 modifications may be removed by treatment with the capping reagent prior to oxidation with I₂/water. In some instances, inclusion of a capping step during polynucleotide synthesis decreases the error rate as compared to synthesis without capping. As an example, the capping step comprises treating the substrate-bound polynucleotide with a mixture of acetic anhydride and 1-methylimidazole. Following a capping step, the device is optionally washed.

In some instances, following addition of a nucleoside phosphoramidite, and optionally after capping and one or more wash steps, the device bound growing nucleic acid is oxidized. The oxidation step comprises the phosphite triester is oxidized into a tetracoordinated phosphate triester, a protected precursor of the naturally occurring phosphate diester internucleoside linkage. In some instances, oxidation of the growing polynucleotide is achieved by treatment with iodine and water, optionally in the presence of a weak base (e.g., pyridine, lutidine, collidine). Oxidation may be carried out under anhydrous conditions using, e.g. tert-Butyl hydroperoxide or (1S)-(+)-(10-camphorsulfonyl)-oxaziridine (CSO). In some methods, a capping step is performed following oxidation. A second capping step allows for device drying, as residual water from oxidation that may persist can inhibit subsequent coupling. Following oxidation, the device and growing polynucleotide is optionally washed. In some instances, the step of oxidation is substituted with a sulfurization step to obtain polynucleotide phosphorothioates, wherein any capping steps can be performed after the sulfurization. Many reagents are capable of the efficient sulfur transfer, including but not limited to 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

In order for a subsequent cycle of nucleoside incorporation to occur through coupling, the protected 5′ end of the device bound growing polynucleotide is removed so that the primary hydroxyl group is reactive with a next nucleoside phosphoramidite. In some instances, the protecting group is DMT and deblocking occurs with trichloroacetic acid in dichloromethane. Conducting detritylation for an extended time or with stronger than recommended solutions of acids may lead to increased depurination of solid support-bound polynucleotide and thus reduces the yield of the desired full-length product. Methods and compositions of the disclosure described herein provide for controlled deblocking conditions limiting undesired depurination reactions. In some instances, the device bound polynucleotide is washed after deblocking. In some instances, efficient washing after deblocking contributes to synthesized polynucleotides having a low error rate.

Methods for the synthesis of polynucleotides typically involve an iterating sequence of the following steps: application of a protected monomer to an actively functionalized surface (e.g., locus) to link with either the activated surface, a linker or with a previously deprotected monomer; deprotection of the applied monomer so that it is reactive with a subsequently applied protected monomer; and application of another protected monomer for linking. One or more intermediate steps include oxidation or sulfurization. In some instances, one or more wash steps precede or follow one or all of the steps.

Methods for phosphoramidite-based polynucleotide synthesis comprise a series of chemical steps. In some instances, one or more steps of a synthesis method involve reagent cycling, where one or more steps of the method comprise application to the device of a reagent useful for the step. For example, reagents are cycled by a series of liquid deposition and vacuum drying steps. For substrates comprising three-dimensional features such as wells, microwells, channels and the like, reagents are optionally passed through one or more regions of the device via the wells and/or channels.

Methods and systems described herein relate to polynucleotide synthesis devices for the synthesis of polynucleotides. The synthesis may be in parallel. For example, at least or about at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 100, 150, 200, 250, 300, 350, 400, 450, 500, 550, 600, 650, 700, 750, 800, 850, 900, 1000, 10000, 50000, 75000, 100000 or more polynucleotides can be synthesized in parallel. The total number polynucleotides that may be synthesized in parallel may be from 2-100000, 3-50000, 4-10000, 5-1000, 6-900, 7-850, 8-800, 9-750, 10-700, 11-650, 12-600, 13-550, 14-500, 15-450, 16-400, 17-350, 18-300, 19-250, 20-200, 21-150,22-100, 23-50, 24-45, 25-40, 30-35. Those of skill in the art appreciate that the total number of polynucleotides synthesized in parallel may fall within any range bound by any of these values, for example 25-100. The total number of polynucleotides synthesized in parallel may fall within any range defined by any of the values serving as endpoints of the range. Total molar mass of polynucleotides synthesized within the device or the molar mass of each of the polynucleotides may be at least or at least about 10, 20, 30, 40, 50, 100, 250, 500, 750, 1000, 2000, 3000, 4000, 5000, 6000, 7000, 8000, 9000, 10000, 25000, 50000, 75000, 100000 picomoles, or more. The length of each of the polynucleotides or average length of the polynucleotides within the device may be at least or about at least 10, 15, 20, 25, 30, 35, 40, 45, 50, 100, 150, 200, 300, 400, 500 nucleotides, or more. The length of each of the polynucleotides or average length of the polynucleotides within the device may be at most or about at most 500, 400, 300, 200, 150, 100, 50, 45, 35, 30, 25, 20, 19, 18, 17, 16, 15, 14, 13, 12, 11, 10 nucleotides, or less. The length of each of the polynucleotides or average length of the polynucleotides within the device may fall from 10-500, 9-400, 11-300, 12-200, 13-150, 14-100, 15-50, 16-45, 17-40, 18-35, 19-25. Those of skill in the art appreciate that the length of each of the polynucleotides or average length of the polynucleotides within the device may fall within any range bound by any of these values, for example 100-300. The length of each of the polynucleotides or average length of the polynucleotides within the device may fall within any range defined by any of the values serving as endpoints of the range.

Methods for polynucleotide synthesis on a surface provided herein allow for synthesis at a fast rate. As an example, at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 100, 125, 150, 175, 200 nucleotides per hour, or more are synthesized. Nucleotides include adenine, guanine, thymine, cytosine, uridine building blocks, or analogs/modified versions thereof. In some instances, libraries of polynucleotides are synthesized in parallel on substrate. For example, a device comprising about or at least about 100; 1,000; 10,000; 30,000; 75,000; 100,000; 1,000,000; 2,000,000; 3,000,000; 4,000,000; or 5,000,000 resolved loci is able to support the synthesis of at least the same number of distinct polynucleotides, wherein polynucleotide encoding a distinct sequence is synthesized on a resolved locus. In some instances, a library of polynucleotides is synthesized on a device with low error rates described herein in less than about three months, two months, one month, three weeks, 15, 14, 13, 12, 11, 10, 9, 8, 7, 6, 5, 4, 3, 2 days, 24 hours or less. In some instances, larger nucleic acids assembled from a polynucleotide library synthesized with low error rate using the substrates and methods described herein are prepared in less than about three months, two months, one month, three weeks, 15, 14, 13, 12, 11, 10, 9, 8, 7, 6, 5, 4, 3, 2 days, 24 hours or less.

In some instances, methods described herein provide for generation of a library of nucleic acids comprising variant nucleic acids differing at a plurality of codon sites. In some instances, a nucleic acid may have 1 site, 2 sites, 3 sites, 4 sites, 5 sites, 6 sites, 7 sites, 8 sites, 9 sites, 10 sites, 11 sites, 12 sites, 13 sites, 14 sites, 15 sites, 16 sites, 17 sites 18 sites, 19 sites, 20 sites, 30 sites, 40 sites, 50 sites, or more of variant codon sites.

In some instances, the one or more sites of variant codon sites may be adjacent. In some instances, the one or more sites of variant codon sites may not be adjacent and separated by 1, 2, 3, 4, 5, 6, 7, 8, 9, 10, or more codons.

In some instances, a nucleic acid may comprise multiple sites of variant codon sites, wherein all the variant codon sites are adjacent to one another, forming a stretch of variant codon sites. In some instances, a nucleic acid may comprise multiple sites of variant codon sites, wherein none the variant codon sites are adjacent to one another. In some instances, a nucleic acid may comprise multiple sites of variant codon sites, wherein some the variant codon sites are adjacent to one another, forming a stretch of variant codon sites, and some of the variant codon sites are not adjacent to one another.

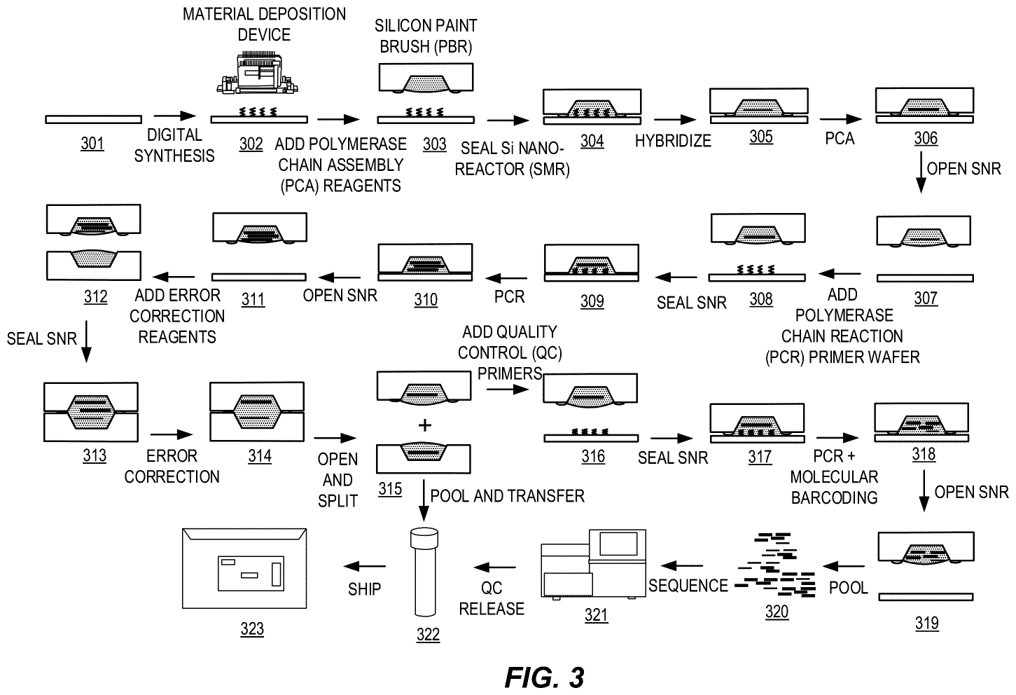

Referring to the Figures, FIG. 3 illustrates an exemplary process workflow for synthesis of nucleic acids (e.g., genes) from shorter nucleic acids. The workflow is divided generally into phases: (1) de novo synthesis of a single stranded nucleic acid library, (2) joining nucleic acids to form larger fragments, (3) error correction, (4) quality control, and (5) shipment. Prior to de novo synthesis, an intended nucleic acid sequence or group of nucleic acid sequences is preselected. For example, a group of genes is preselected for generation.

Once large nucleic acids for generation are selected, a predetermined library of nucleic acids is designed for de novo synthesis. Various suitable methods are known for generating high density polynucleotide arrays. In the workflow example, a device surface layer is provided. In the example, chemistry of the surface is altered in order to improve the polynucleotide synthesis process. Areas of low surface energy are generated to repel liquid while areas of high surface energy are generated to attract liquids. The surface itself may be in the form of a planar surface or contain variations in shape, such as protrusions or microwells which increase surface area. In the workflow example, high surface energy molecules selected serve a dual function of supporting DNA chemistry, as disclosed in International Patent Application Publication WO/2015/021080, which is herein incorporated by reference in its entirety.

In situ preparation of polynucleotide arrays is generated on a solid support and utilizes single nucleotide extension process to extend multiple oligomers in parallel. A deposition device, such as a material deposition device, is designed to release reagents in a step wise fashion such that multiple polynucleotides extend, in parallel, one residue at a time to generate oligomers with a predetermined nucleic acid sequence 302. In some instances, polynucleotides are cleaved from the surface at this stage. Cleavage includes gas cleavage, e.g., with ammonia or methylamine.

The generated polynucleotide libraries are placed in a reaction chamber. In this exemplary workflow, the reaction chamber (also referred to as “nanoreactor”) is a silicon coated well, containing PCR reagents and lowered onto the polynucleotide library 303. Prior to or after the sealing 304 of the polynucleotides, a reagent is added to release the polynucleotides from the substrate. In the exemplary workflow, the polynucleotides are released subsequent to sealing of the nanoreactor 305. Once released, fragments of single stranded polynucleotides hybridize in order to span an entire long range sequence of DNA. Partial hybridization 305 is possible because each synthesized polynucleotide is designed to have a small portion overlapping with at least one other polynucleotide in the pool.

After hybridization, a PCA reaction is commenced. During the polymerase cycles, the polynucleotides anneal to complementary fragments and gaps are filled in by a polymerase. Each cycle increases the length of various fragments randomly depending on which polynucleotides find each other. Complementarity amongst the fragments allows for forming a complete large span of double stranded DNA 306.

After PCA is complete, the nanoreactor is separated from the device 307 and positioned for interaction with a device having primers for PCR 308. After sealing, the nanoreactor is subject to PCR 309 and the larger nucleic acids are amplified. After PCR 310, the nanochamber is opened 311, error correction reagents are added 312, the chamber is sealed 313 and an error correction reaction occurs to remove mismatched base pairs and/or strands with poor complementarity from the double stranded PCR amplification products 314. The nanoreactor is opened and separated 315. Error corrected product is next subject to additional processing steps, such as PCR and molecular bar coding, and then packaged 322 for shipment 323.

In some instances, quality control measures are taken. After error correction, quality control steps include for example interaction with a wafer having sequencing primers for amplification of the error corrected product 316, sealing the wafer to a chamber containing error corrected amplification product 317, and performing an additional round of amplification 318. The nanoreactor is opened 319 and the products are pooled 320 and sequenced 321. After an acceptable quality control determination is made, the packaged product 322 is approved for shipment 323.

In some instances, a nucleic acid generated by a workflow such as that in FIG. 3 is subject to mutagenesis using overlapping primers disclosed herein. In some instances, a library of primers are generated by in situ preparation on a solid support and utilize single nucleotide extension process to extend multiple oligomers in parallel. A deposition device, such as a material deposition device, is designed to release reagents in a step wise fashion such that multiple polynucleotides extend, in parallel, one residue at a time to generate oligomers with a predetermined nucleic acid sequence 302.

### Computer Systems

Any of the systems described herein, may be operably linked to a computer and may be automated through a computer either locally or remotely. In various instances, the methods and systems of the disclosure may further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the disclosure. The computer systems may be programmed to interface between the user specified base sequence and the position of a material deposition device to deliver the correct reagents to specified regions of the substrate.

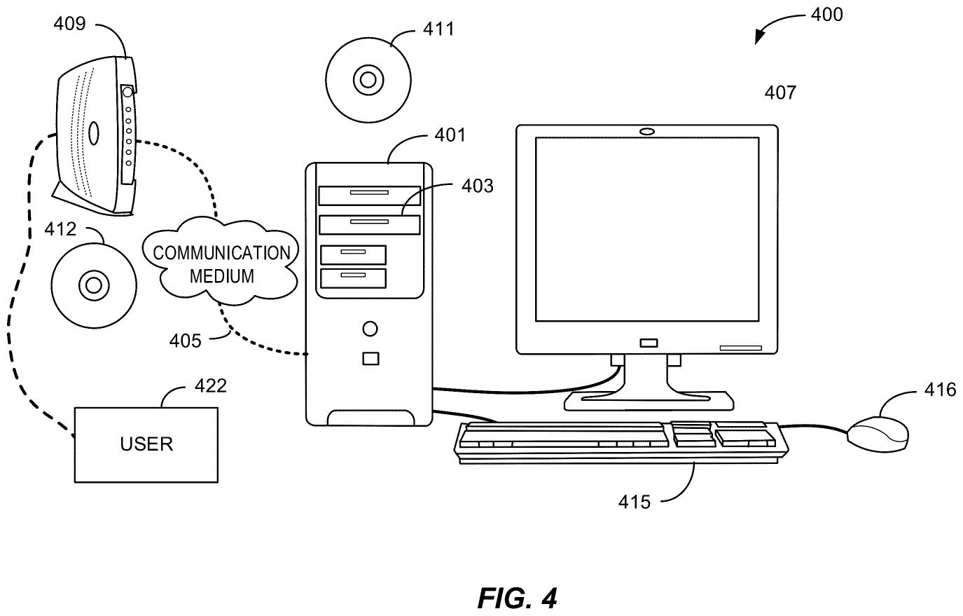

The computer system 400 illustrated in FIG. 4 may be understood as a logical apparatus that can read instructions from media 411 and/or a network port 405, which can optionally be connected to server 409 having fixed media 412. The system, such as shown in FIG. 4 can include a CPU 401, disk drives 403, optional input devices such as keyboard 415 and/or mouse 416 and optional monitor 407. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 422 as illustrated in FIG. 4.

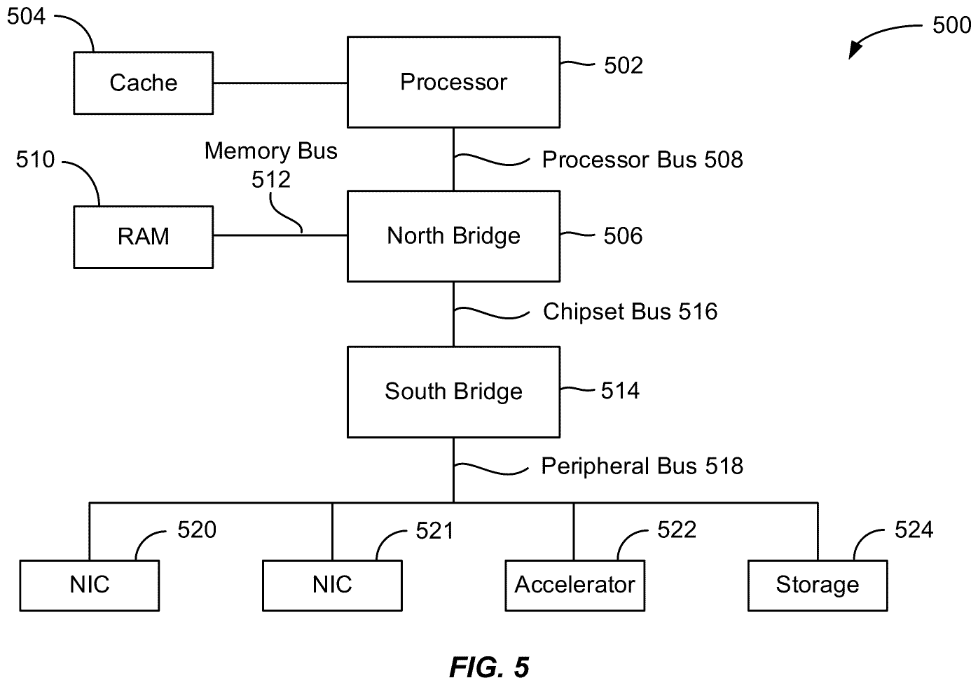

As illustrated in FIG. 5, a high speed cache 504 can be connected to, or incorporated in, the processor 502 to provide a high speed memory for instructions or data that have been recently, or are frequently, used by processor 502. The processor 502 is connected to a north bridge 506 by a processor bus 508. The north bridge 506 is connected to random access memory (RAM) 510 by a memory bus 512 and manages access to the RAM 510 by the processor 502. The north bridge 506 is also connected to a south bridge 514 by a chipset bus 516. The south bridge 514 is, in turn, connected to a peripheral bus 518. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 518. In some alternative architectures, the functionality of the north bridge can be incorporated into the processor instead of using a separate north bridge chip. In some instances, system 500 can include an accelerator card 522 attached to the peripheral bus 518. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 524 and can be loaded into RAM 510 and/or cache 504 for use by the processor. The system 500 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example instances of the present disclosure. In this example, system 500 also includes network interface cards (NICs) 520 and 521 connected to the peripheral bus for providing network interfaces to external storage, such as Network Attached Storage (NAS) and other computer systems that can be used for distributed parallel processing.

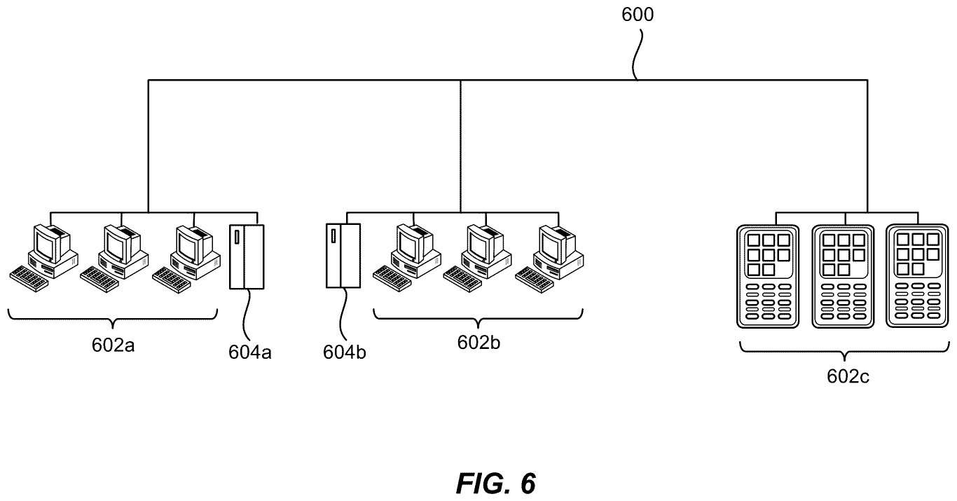

FIG. 6 is a diagram showing a network 600 with a plurality of computer systems 602a, and 602b, a plurality of cell phones and personal data assistants 602c, and Network Attached Storage (NAS) 604a, and 604b. In example instances, systems 602a, 602b, and 602c can manage data storage and optimize data access for data stored in Network Attached Storage (NAS) 604a and 604b. A mathematical model can be used for the data and be evaluated using distributed parallel processing across computer systems 602a, and 602b, and cell phone and personal data assistant systems 602c. Computer systems 602a, and 602b, and cell phone and personal data assistant systems 602c can also provide parallel processing for adaptive data restructuring of the data stored in Network Attached Storage (NAS) 604a and 604b. FIG. 6 illustrates an example only, and a wide variety of other computer architectures and systems can be used in conjunction with the various instances of the present disclosure. For example, a blade server can be used to provide parallel processing. Processor blades can be connected through a back plane to provide parallel processing. Storage can also be connected to the back plane or as Network Attached Storage (NAS) through a separate network interface. In some example instances, processors can maintain separate memory spaces and transmit data through network interfaces, back plane or other connectors for parallel processing by other processors. In other instances, some or all of the processors can use a shared virtual address memory space.

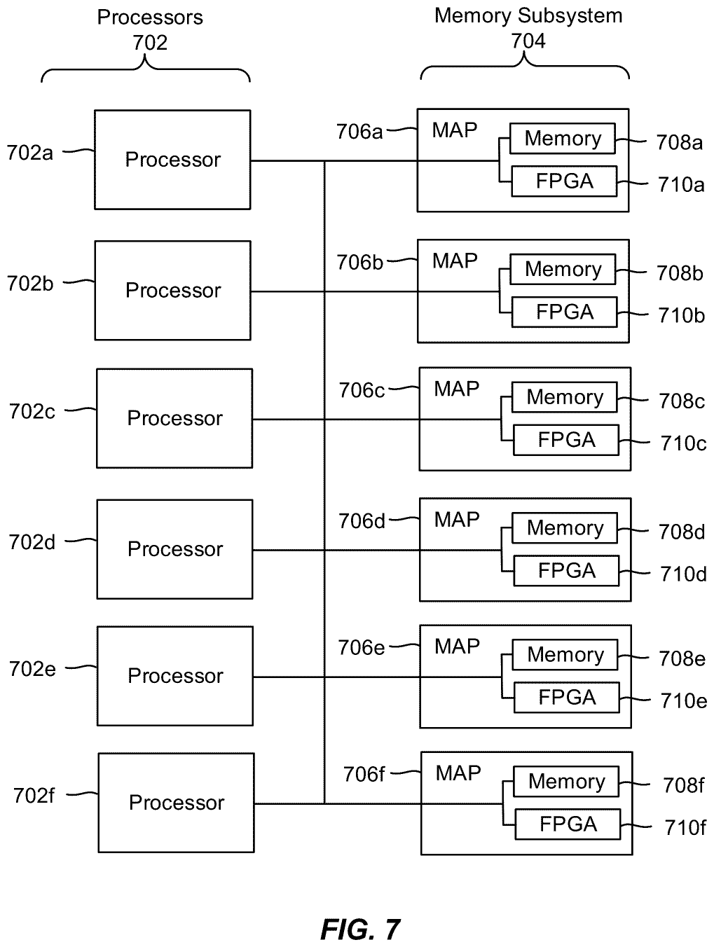

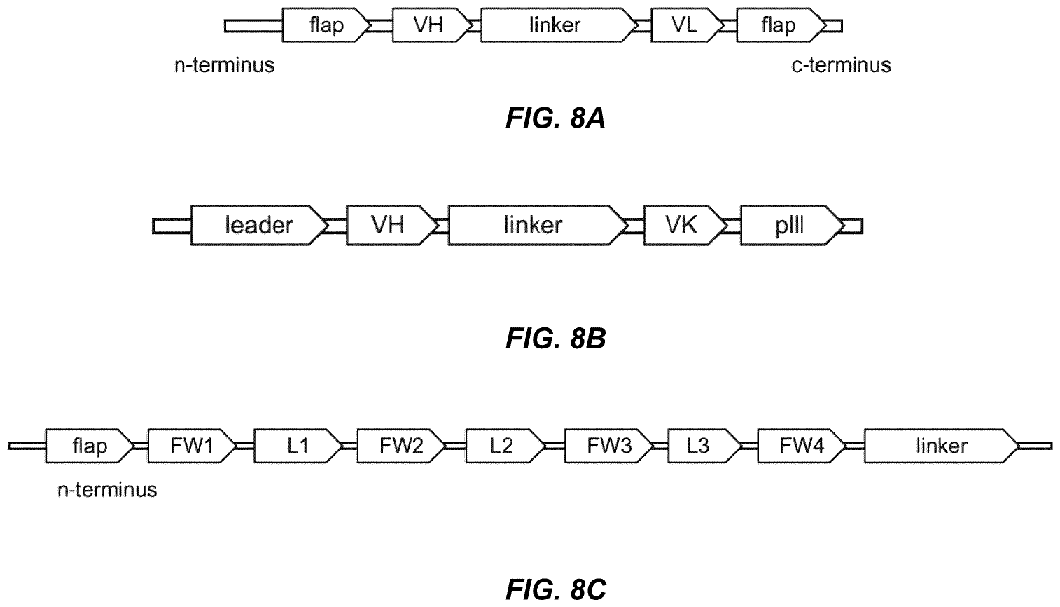

FIG. 7 is a block diagram of a multiprocessor computer system 700 using a shared virtual address memory space in accordance with an example instance. The system includes a plurality of processors 702a-f that can access a shared memory subsystem 704. The system incorporates a plurality of programmable hardware memory algorithm processors (MAPs) 706a-f in the memory subsystem 704. Each MAP 706a-f can comprise a memory 708a-f and one or more field programmable gate arrays (FPGAs) 710a-f. The MAP provides a configurable functional unit and particular algorithms or portions of algorithms can be provided to the FPGAs 710a-f for processing in close coordination with a respective processor. For example, the MAPs can be used to evaluate algebraic expressions regarding the data model and to perform adaptive data restructuring in example instances. In this example, each MAP is globally accessible by all of the processors for these purposes. In one configuration, each MAP can use Direct Memory Access (DMA) to access an associated memory 708a-f, allowing it to execute tasks independently of, and asynchronously from the respective microprocessor 702a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example instances, including systems using any combination of general processors, co-processors, FPGAs and other programmable logic devices, system on chips (SOCs), application specific integrated circuits (ASICs), and other processing and logic elements. In some instances, all or part of the computer system can be implemented in software or hardware. Any variety of data storage media can be used in connection with example instances, including random access memory, hard drives, flash memory, tape drives, disk arrays, Network Attached Storage (NAS) and other local or distributed data storage devices and systems.

In example instances, the computer system can be implemented using software modules executing on any of the above or other computer architectures and systems. In other instances, the functions of the system can be implemented partially or completely in firmware, programmable logic devices such as field programmable gate arrays (FPGAs) as referenced in FIG. 5, system on chips (SOCs), application specific integrated circuits (ASICs), or other processing and logic elements. For example, the Set Processor and Optimizer can be implemented with hardware acceleration through the use of a hardware accelerator card, such as accelerator card 522 illustrated in FIG. 5.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Functionalization of a Device Surface

A device was functionalized to support the attachment and synthesis of a library of polynucleotides. The device surface was first wet cleaned using a piranha solution comprising 90% H₂SO₄ and 10% H₂O₂ for 20 minutes. The device was rinsed in several beakers with DI water, held under a DI water gooseneck faucet for 5 min, and dried with N₂. The device was subsequently soaked in NH₄OH (1:100; 3 mL: 300 mL) for 5 min, rinsed with DI water using a handgun, soaked in three successive beakers with DI water for 1 min each, and then rinsed again with DI water using the handgun. The device was then plasma cleaned by exposing the device surface to O₂. A SAMCO PC-300 instrument was used to plasma etch O₂ at 250 watts for 1 min in downstream mode.

The cleaned device surface was actively functionalized with a solution comprising N-(3-triethoxysilylpropyl)-4-hydroxybutyramide using a YES-1224P vapor deposition oven system with the following parameters: 0.5 to 1 torr, 60 min, 70° C., 135° C. vaporizer. The device surface was resist coated using a Brewer Science 200X spin coater. SPR™ 3612 photoresist was spin coated on the device at 2500 rpm for 40 sec. The device was pre-baked for 30 min at 90° C. on a Brewer hot plate. The device was subjected to photolithography using a Karl Suss MA6 mask aligner instrument. The device was exposed for 2.2 sec and developed for 1 min in MSF 26A. Remaining developer was rinsed with the handgun and the device soaked in water for 5 min. The device was baked for 30 min at 100° C. in the oven, followed by visual inspection for lithography defects using a Nikon L200. A descum process was used to remove residual resist using the SAMCO PC-300 instrument to O₂ plasma etch at 250 watts for 1 min.

The device surface was passively functionalized with a 100 μL solution of perfluorooctyltrichlorosilane mixed with 10 μL light mineral oil. The device was placed in a chamber, pumped for 10 min, and then the valve was closed to the pump and left to stand for 10 min. The chamber was vented to air. The device was resist stripped by performing two soaks for 5 min in 500 mL NMP at 70° C. with ultrasonication at maximum power (9 on Crest system). The device was then soaked for 5 min in 500 mL isopropanol at room temperature with ultrasonication at maximum power. The device was dipped in 300 mL of 200 proof ethanol and blown dry with N₂. The functionalized surface was activated to serve as a support for polynucleotide synthesis.

### Example 2: Synthesis of a 50-Mer Sequence on an Oligonucleotide Synthesis Device

A two dimensional oligonucleotide synthesis device was assembled into a flowcell, which was connected to a flowcell (Applied Biosystems (ABI394 DNA Synthesizer”). The two-dimensional oligonucleotide synthesis device was uniformly functionalized with N-(3-TRIETHOXYSILYLPROPYL)-4-HYDROXYBUTYRAMIDE (Gelest) was used to synthesize an exemplary polynucleotide of 50 bp (“50-mer polynucleotide”) using polynucleotide synthesis methods described herein.

The sequence of the 50-mer was as described in SEQ ID NO.: 2. 5′AGACAATCAACCATTIGGGGTGGACAGCCTTGACCTCTAGACTTCGGCAT ##TTTTTTT TTT3′ (SEQ ID NO.: 2), where #denotes Thymidine-succinyl hexamide CED phosphoramidite (CLP-2244 from ChemGenes), which is a cleavable linker enabling the release of oligos from the surface during deprotection.

The synthesis was done using standard DNA synthesis chemistry (coupling, capping, oxidation, and deblocking) according to the protocol in Table 3 and an ABI synthesizer.

TABLE 3
Synthesis protocols

| General DNA Synthesis | Table 3 | |
|---|---|---|
| Process Name | Process Step | Time (sec) |
| WASH (Acetonitrile Wash | Acetonitrile System Flush | 4 |
| Flow) | Acetonitrile to Flowcell | 23 |
| | N2 System Flush | 4 |
| | Acetonitrile System Flush | 4 |
| DNA BASE ADDITION | Activator Manifold Flush | 2 |
| (Phosphoramidite + | Activator to Flowcell | 6 |
| Activator Flow) | Activator + | 6 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Incubate for 25 sec | 25 |
| WASH (Acetonitrile Wash | Acetonitrile System Flush | 4 |
| Flow) | Acetonitrile to Flowcell | 15 |
| | N2 System Flush | 4 |
| | Acetonitrile System Flush | 4 |
| DNA BASE ADDITION | Activator Manifold Flush | 2 |
| (Phosphoramidite + | Activator to Flowcell | 5 |
| Activator Flow) | Activator + | 18 |
| | Phosphoramidite to | |
| | Flowcell | |
| | Incubate for 25 sec | 25 |
| WASH (Acetonitrile Wash | Acetonitrile System Flush | 4 |
| Flow) | Acetonitrile to Flowcell | 15 |
| | N2 System Flush | 4 |
| | Acetonitrile System Flush | 4 |
| CAPPING (CapA + B, 1:1, | CapA + B to Flowcell | 15 |
| Flow) | | |
| WASH (Acetonitrile Wash | Acetonitrile System Flush | 4 |
| Flow) | Acetonitrile to Flowcell | 15 |
| | Acetonitrile System Flush | 4 |
| OXIDATION (Oxidizer | Oxidizer to Flowcell | 18 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 15 |
| | Acetonitrile System Flush | 4 |
| | Acetonitrile to Flowcell | 15 |
| | N2 System Flush | 4 |
| | Acetonitrile System Flush | 4 |
| | Acetonitrile to Flowcell | 23 |
| | N2 System Flush | 4 |
| | Acetonitrile System Flush | 4 |
| DEBLOCKING (Deblock | Deblock to Flowcell | 36 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 18 |
| | N2 System Flush | 4.13 |
| | Acetonitrile System Flush | 4.13 |
| | Acetonitrile to Flowcell | 15 |

The phosphoramidite/activator combination was delivered similar to the delivery of bulk reagents through the flowcell. No drying steps were performed as the environment stays “wet” with reagent the entire time.

The flow restrictor was removed from the ABI 394 synthesizer to enable faster flow. Without flow restrictor, flow rates for amidites (0.1M in ACN), Activator, (0.25M Benzoylthiotetrazole (“BTT”; 30-3070-xx from GlenResearch) in ACN), and Ox (0.02M 12 in 20% pyridine, 10% water, and 70% THF) were roughly ˜100 uL/sec, for acetonitrile (“ACN”) and capping reagents (1:1 mix of CapA and CapB, wherein CapA is acetic anhydride in THF/Pyridine and CapB is 16% 1-methylimidizole in THF), roughly ˜200 uL/sec, and for Deblock (3% dichloroacetic acid in toluene), roughly ˜300 uL/sec (compared to ˜50 uL/sec for all reagents with flow restrictor). The time to completely push out Oxidizer was observed, the timing for chemical flow times was adjusted accordingly and an extra ACN wash was introduced between different chemicals. After polynucleotide synthesis, the chip was deprotected in gaseous ammonia overnight at 75 psi. Five drops of water were applied to the surface to recover polynucleotides. The recovered polynucleotides were then analyzed on a BioAnalyzer small RNA chip.

### Example 3: Synthesis of a 100-Mer Sequence on an Oligonucleotide Synthesis Device

The same process as described in Example 2 for the synthesis of the 50-mer sequence was used for the synthesis of a 100-mer polynucleotide (“100-mer polynucleotide”; 5′ CGGGATCCTTATCGTCATCGTCGTACAGATCCCGACCCATTTGCTGTCCACCAGTCATG CTAGCCATACCATGATGATGATGATGATGAGAACCCCGCAT ##TTTTTTTTTT3′, where #denotes Thymidine-succinyl hexamide CED phosphoramidite (CLP-2244 from ChemGenes); SEQ ID NO.: 3) on two different silicon chips, the first one uniformly functionalized with N-(3-TRIETHOXYSILYLPROPYL)-4-HYDROXYBUTYRAMIDE and the second one functionalized with 5/95 mix of 11-acetoxyundecyltriethoxysilane and n-decyltriethoxysilane, and the polynucleotides extracted from the surface were analyzed on a BioAnalyzer instrument.

All ten samples from the two chips were further PCR amplified using a forward (5′ATGCGGGGTTCTCATCATC3′; SEQ ID NO.: 4) and a reverse (5′CGGGATCCTTATCGTCATCG3′; SEQ ID NO.: 5) primer in a 50 μL PCR mix (25 μL NEB Q5 mastermix, 2.5 μL 10 uM Forward primer, 2.5 μL 10 uM Reverse primer, 1 uL polynucleotide extracted from the surface, and water up to 50 μL) using the following thermalcycling program:
98° C., 30 sec
98° C., 10 sec; 63° C., 10 sec; 72° C., 10 sec; repeat 12 cycles
72° C., 2 min

The PCR products were also run on a BioAnalyzer, demonstrating sharp peaks at the 100-mer position. Next, the PCR amplified samples were cloned, and Sanger sequenced. Table 4 summarizes the results from the Sanger sequencing for samples taken from spots 1-5 from chip 1 and for samples taken from spots 6-10 from chip 2.

TABLE 4
Sequencing results

| Spot | Error rate | Cycle efficiency |
|---|---|---|
| 1 | 1/763 bp | 99.87% |
| 2 | 1/824 bp | 99.88% |
| 3 | 1/780 bp | 99.87% |
| 4 | 1/429 bp | 99.77% |
| 5 | 1/1525 bp | 99.93% |
| 6 | 1/1615 bp | 99.94% |
| 7 | 1/531 bp | 99.81% |
| 8 | 1/1769 bp | 99.94% |
| 9 | 1/854 bp | 99.88% |
| 10 | 1/1451 bp | 99.93% |

Thus, the high quality and uniformity of the synthesized polynucleotides were repeated on two chips with different surface chemistries. Overall, 89% of the 100-mers that were sequenced were perfect sequences with no errors, corresponding to 233 out of 262.

Table 5 summarizes error characteristics for the sequences obtained from the polynucleotide samples from spots 1-10.

TABLE 5
Error characteristics

| | Sample ID/Spot no. | | | | | | | | | |
|---|---|---|---|---|---|---|---|---|---|---|
| | OSA_ | OSA_ | OSA_ | OSA_ | OSA_ | OSA_ | OSA_ | OSA_ | OSA_ | OSA_ |
| | 0046/1 | 0047/2 | 0048/3 | 0049/4 | 0050/5 | 0051/6 | 0052/7 | 0053/8 | 0054/9 | 0055/10 |
| Total | 32 | 32 | 32 | 32 | 32 | 32 | 32 | 32 | 32 | 32 |
| Sequences | | | | | | | | | | |
| Sequencing | 25 of | 27 of | 26 of | 21 of | 25 of | 29 of | 27 of | 29 of | 28 of | 25 of |
| Quality | 28 | 27 | 30 | 23 | 26 | 30 | 31 | 31 | 29 | 28 |
| Oligo | 23 of | 25 of | 22 of | 18 of | 24 of | 25 of | 22 of | 28 of | 26 of | 20 of |
| Quality | 25 | 27 | 26 | 21 | 25 | 29 | 27 | 29 | 28 | 25 |
| ROI | 2500 | 2698 | 2561 | 2122 | 2499 | 2666 | 2625 | 2899 | 2798 | 2348 |
| Match | | | | | | | | | | |
| Count | | | | | | | | | | |
| ROI | 2 | 2 | 1 | 3 | 1 | 0 | 2 | 1 | 2 | 1 |
| Mutation | | | | | | | | | | |
| ROI Multi | 0 | 0 | 0 | 0 | 0 | 0 | 0 | 0 | 0 | 0 |
| Base | | | | | | | | | | |
| Deletion | | | | | | | | | | |
| ROI Small | 1 | 0 | 0 | 0 | 0 | 0 | 0 | 0 | 0 | 0 |
| Insertion | | | | | | | | | | |
| ROI | 0 | 0 | 0 | 0 | 0 | 0 | 0 | 0 | 0 | 0 |
| Single | | | | | | | | | | |
| Base | | | | | | | | | | |
| Deletion | | | | | | | | | | |
| Large | 0 | 0 | 1 | 0 | 0 | 1 | 1 | 0 | 0 | 0 |
| Deletion | | | | | | | | | | |
| Count | | | | | | | | | | |
| Mutation: | 2 | 2 | 1 | 2 | 1 | 0 | 2 | 1 | 2 | 1 |
| G >A | | | | | | | | | | |
| Mutation: | 0 | 0 | 0 | 1 | 0 | 0 | 0 | 0 | 0 | 0 |
| T >C | | | | | | | | | | |
| ROI Error | 3 | 2 | 2 | 3 | 1 | 1 | 3 | 1 | 2 | 1 |
| Count | | | | | | | | | | |
| ROI Error | Err: ~1 | Err: ~1 | Err: ~1 | Err: ~1 | Err: ~1 | Err: ~1 | Err: ~1 | Err: ~1 | Err: ~1 | Err: ~1 |
| Rate | in 834 | in 1350 | in 1282 | in 708 | in 2500 | in 2667 | in 876 | in 2900 | in 1400 | in 2349 |
| ROI | MP Err: | MP Err: | MP Err: | MP Err: | MP Err: | MP Err: | MP Err: | MP Err: | MP Err: | MP Err: |
| Minus | ~1 in | ~1 in | ~1 in | ~1 in | ~1 in | ~1 in | ~1 in | ~1 in | ~1 in | ~1 in |
| Primer | 763 | 824 | 780 | 429 | 1525 | 1615 | 531 | 1769 | 854 | 1451 |
| Error Rate | | | | | | | | | | |

### Example 4: Design of Antibody Scaffolds

To generate scaffolds, structural analysis, repertoire sequencing analysis of the heavy chain, and specific analysis of heterodimer high-throughput sequencing datasets were performed. Each heavy chain was associated with each light chain scaffold. Each heavy chain scaffold was assigned 5 different long CDRH3 loop options. Each light chain scaffold was assigned 5 different L3 scaffolds. The heavy chain CDRH3 stems were chosen from the frequently observed long H3 loop stems (10 amino acids on the N-terminus and the C-terminus) found both across individuals and across V-gene segments. The light chain scaffold L3s were chosen from heterodimers comprising long H3s. Direct heterodimers based on information from the Protein Data Bank (PDB) and deep sequencing datasets were used in which CDR H1, H2, L1, L2, L3, and CDRH3 stems were fixed. The various scaffolds were then formatted for display on phage to assess for expression.

### Structural Analysis

About 2,017 antibody structures were analyzed from which 22 structures with long CDRH3s of at least 25 amino acids in length were observed. The heavy chains included the following: IGHV1-69, IGHV3-30, IGHV4-49, and IGHV3-21. The light chains identified included the following: IGLV3-21, IGKV3-11, IGKV2-28, IGKV1-5, IGLV1-51, IGLV1-44, and IGKV1-13. In the analysis, four heterodimer combinations were observed multiple times including: IGHV4-59/61-IGLV3-21, IGHV3-21-IGKV2-28, IGHV1-69-IGKV3-11, and IGHV1-69-IGKV1-5. An analysis of sequences and structures identified intra-CDRH3 disulfide bonds in a few structures with packing of bulky side chains such as tyrosine in the stem providing support for long H3 stability. Secondary structures including beta-turn-beta sheets and a “hammerhead” subdomain were also observed.

### Repertoire Analysis

A repertoire analysis was performed on 1,083,875 IgM+/CD27-naïve B cell receptor (BCR) sequences and 1,433,011 CD27+ sequences obtained by unbiased 5′RACE from 12 healthy controls. The 12 healthy controls comprised equal numbers of male and female and were made up of 4 Caucasian, 4 Asian, and 4 Hispanic individuals. The repertoire analysis demonstrated that less than 1% of the human repertoire comprises BCRs with CDRH3s longer than 21 amino acids. A V-gene bias was observed in the long CDR3 subrepertoire, with IGHV1-69, IGHV4-34, IGHV1-18, and IGHV1-8 showing preferential enrichment in BCRs with long H3 loops. A bias against long loops was observed for IGHV3-23, IGHV4-59/61, IGHV5-51, IGHV3-48, IGHV3-53/66, IGHV3-15, IGHV3-74, IGHV3-73, IGHV3-72, and IGHV2-70. The IGHV4-34 scaffold was demonstrated to be autoreactive and had a short half-life.

Viable N-terminal and C-terminal CDRH3 scaffold variation for long loops were also designed based on 5′RACE reference repertoire. About 81,065 CDRH3s of amino acid length 22 amino acids or greater were observed. By comparing across V-gene scaffolds, scaffold-specific H3 stem variation was avoided as to allow the scaffold diversity to be cloned into multiple scaffold references.

### Heterodimer Analysis

Heterodimer analysis was performed on scaffolds. Variant sequences and lengths of the scaffolds were assayed.

### Structural Analysis

Structural analysis was performed using GPCR scaffolds of variant sequences and lengths were assayed.

### Example 5: Generation of GPCR Antibody Libraries

Based on GPCR-ligand interaction surfaces and scaffold arrangements, libraries were designed and de novo synthesized. See Example 4. 10 variant sequences were designed for the variable domain, heavy chain, 237 variant sequences were designed for the heavy chain complementarity determining region 3, and 44 variant sequences were designed for the variable domain, light chain. The fragments were synthesized as three fragments following similar methods as described in Examples 1-3.

Following de novo synthesis, 10 variant sequences were generated for the variable domain, heavy chain, 236 variant sequences were generated for the heavy chain complementarity determining region 3, and 43 variant sequences were designed for a region comprising the variable domain, light chain and CDRL3 and of which 9 variants for variable domain, light chain were designed. This resulted in a library with about 105 diversity (10×236×43). This was confirmed using next generation sequencing (NGS) with 16 million reads.

The various light and heavy chains were then tested for expression and protein folding. The 10 variant sequences for variable domain, heavy chain included the following: IGHV1-18, IGHV1-69, IGHV1-8 IGHV3-21, IGHV3-23, IGHV3-30/33rn, IGHV3-28, IGHV3-74, IGHV4-39, and IGHV4-59/61. Of the 10 variant sequences, IGHV1-18, IGHV1-69, and IGHV3-30/33rn exhibited improved characteristics such as improved thermostability. 9 variant sequences for variable domain, light chain included the following: IGKV1-39, IGKV1-9, IGKV2-28, IGKV3-11, IGKV3-15, IGKV3-20, IGKV4-1, IGLV1-51, and IGLV2-14. Of the 9 variant sequences, IGKV1-39, IGKV3-15, IGLV1-51, and IGLV2-14 exhibited improved characteristics such as improved thermostability.

### Example 6: GPCR Libraries

This example describes the generation of GPCR libraries.

### Materials and Method

### Stable Cell Line and Phage Library Generation

The full length human GLP-1R gene (UniProt—P43220) with an N-terminal FLAG tag and C-terminal GFP tag cloned into pCDNA3.1 (+) vector (ThermoFisher) was transfected into suspension Chinese Hamster Ovary (CHO) cells to generate the stable cell line expressing GLP-1R. Target expression was confirmed by FACS. Cells expressing>80% of GLP-1R by GFP were then directly used for cell-based selections.

Germline heavy chain IGHV1-69, IGHV3-30 and germline light chain IGKV1-39, IGKV3-15, IGLV1-51, IGLV2-14 framework combinations were used in the GPCR-focused phage-displayed library, and all six CDR diversities were encoded by oligo pools synthesized similar to Examples 1-3 above. The CDRs were also screened to ensure they did not contain manufacturability liabilities, cryptic splice sites, or commonly used nucleotide restriction sites. The heavy chain variable region (VH) and light chain variable region (VL) were linked by (G4S)3 linker (SEQ ID NO: 718). The resulting scFv (VH-linker-VL) gene library was cloned into a pADL 22-2c (Antibody Design Labs) phage display vector by NotI restriction digestion and electroporated into TG1 electro-competent E. coli cells. (Lucigen). The final library has a diversity of 1.1×10¹⁰ size which was verified by NGS.

Panning and Screening Strategy Used to Isolate Agonist GLP-1R scFy Clones

Before panning on GLP-1R expressing CHO cells, phage particles were blocked with 5% BSA/PBS and depleted for non-specific binders on CHO parent cells. For CHO parent cell depletion, the input phage aliquot was rotated at 14 rpm/min with 1×10⁸ CHO parent cells for 1 hour at room temperature (RT). The cells were then pelleted by centrifuging at 1,200 rpm for 10 mins in a tabletop Eppendorf centrifuge 5920RS/4×1000 rotor to deplete the non-specific CHO cell binders. The phage supernatant, depleted of CHO cell binders, was then transferred to 1×10⁸ GLP-1R expressing CHO cells. The phage supernatant and GLP-1R expressing CHO cells were rotated at 14 rpm/min for 1 hour at RT to select for GLP-1R binders. After incubation, the cells were washed several times with 1×PBS/0.5% Tween to remove non-binding clones. To elute the phage bound to the GLP-1R cells, the cells were incubated with trypsin in PBS buffer for 30 minutes at 37° C. The cells were pelleted by centrifuging at 1,200 rpm for 10 mins. The output supernatant enriched in GLP-1R binding clones was amplified in TG1 E. coli cells to use as input phage for the next round of selection. This selection strategy was repeated for five rounds. Every round was depleted against the CHO parent background. Amplified output phage from a round was used as the input phage for the subsequent round, and the stringency of washes were increased in each subsequent round of selections with more washes. After five rounds of selection, 500 clones from each of round 4 and round 5 were Sanger sequenced to identify unique clones.

### Next-Generation Sequencing Analysis

The phagemid DNA was miniprepped from the output bacterial stocks of all panning rounds. The variable heavy chain (VH) was PCR amplified from the phagemid DNA using the Forward Primer ACAGAATTCATTAAAGAGGAGAAATTAACC (SEQ ID NO: 719) and reverse primer TGAACCGCCTCCACCGCTAG (SEQ ID NO: 720). The PCR product was directly used for library preparation using the KAPA HyperPlus Library Preparation Kit (Kapa Biosystems, product #KK8514). To add diversity in the library, the samples were spiked with 15% PhiX Control purchased from Illumina, Inc. (product #FC-110-3001). The library was then loaded onto Illumina's 600 cycle MiSeq Reagent Kit v3 (Illumina, product #MS-102-3003) and run on the MiSeq instrument.

### Reformatting and High Throughput (HT) IgG Purification

Expi293 cells were transfected using Expifectamine (ThermoFisher, A14524) with the heavy chain and light chain DNA at a 2:1 ratio and supernatants were harvested 4 days post-transfection before cell viability dropped below 80%. Purifications were undertaken using either King Fisher (ThermoFisher) with protein A magnetic beads or Phynexus protein A column tips (Hamilton). For large scale production of IgG clones that were evaluated in in vivo mouse studies an Akta HPLC purification system (GE) was used.

IgG characterization and quality control. The purified IgGs for the positive GLP-1R binders (hits) were subjected to characterization for their purity by LabChip GXII Touch HT Protein Express high-sensitivity assay. Dithiothreitol (DTT) was used to reduce the IgG into VH and VL. IgG concentrations were measured using Lunatic (UnChain). IgG for in vivo mouse studies were further characterized by HPLC and tested for endotoxin levels (Endosafe® nexgen-PTS™ Endotoxin Testing, Charles River), with less than 5 EU per kg dosing.

### Binding Assays and Flow Cytometry

GLP-1R IgG clones were tested in a binding assay coupled to flow cytometry analysis as follows: FLAG-GLP-1R-GFP expressing CHO cells (CHO-GLP-1R) and CHO-parent cells were incubated with 100 nM IgG for 1 h on ice, washed three times and incubated with Alexa 647 conjugated goat-anti-human antibody (1:200) (Jackson ImmunoResearch Laboratories, 109-605-044) for 30 min on ice, followed by three washes, centrifuging to pellet the cells between each washing step. All incubations and washes were in buffer containing PBS+1% BSA. For titrations, IgG was serially diluted 1:3 starting from 100 nM down to 0.046 nM. Cells were analyzed by flow cytometry and hits (a hit is an IgG that specifically binds to CHO-GLP-1R) were identified by measuring the GFP signal against the Alexa 647 signal. Flow cytometry data of binding assays with 100 nM IgG are presented as dot plots. Analyses of binding assays with IgG titrations are presented as binding curves plotting IgG concentrations against MFI (mean fluorescence intensity).

### Ligand Competition Assay

Ligand competition assays involved co-incubating the primary IgG with 1 μM GLP-1 (7-36). For each data point, IgG (600 nM) was prepared in Flow buffer (PBS+1% BSA) and diluted 1:3 down for 8 titration points. Peptide GLP-1 7-36 (2 μM) was prepared similarly with the Flow buffer (PBS+1% BSA). Each well contained 100,000 cells to which 50 μL of IgG and 50 μL of peptide (=plus) or buffer alone without peptide (=minus) were added. Cells and IgG/peptide mix were incubated for 1 hr on ice, and after washing, secondary antibody (goat anti-human APC, Jackson ImmunoResearch Laboratories, product #109-605-044) diluted 1:200 in PBS+1% BSA was added. This was incubated on ice for 30 mins (50 μL per well), before washing and resuspending in 60 μL buffer. Finally, the assay read-out was measured on an Intellicyt® IQue3 Screener at a rate of 4 seconds per well.

### Results

### Design of GPCR-Focused Antibody Library is Based on GPOR Binding Motifs and GPOR Antibodies

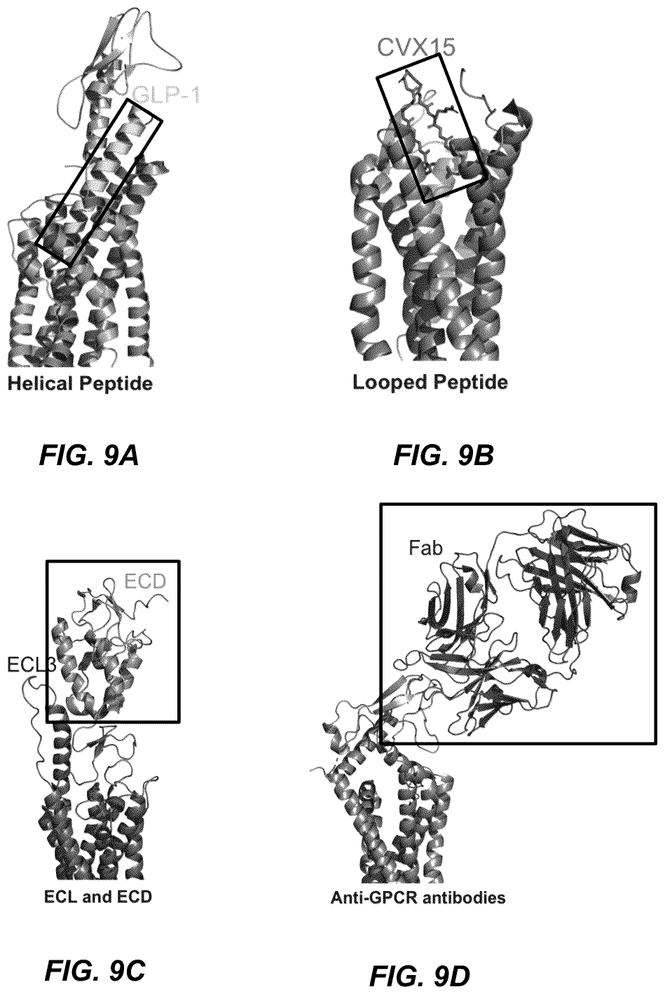

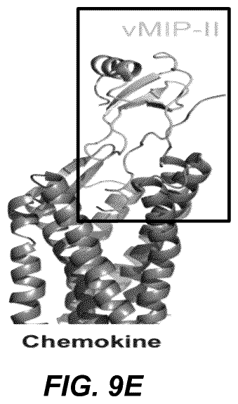

All known GPCR interactions, which include interactions of GPCRs with ligands, peptides, antibodies, endogenous extracellular loops and small molecules were analyzed to map the GPCR binding molecular determinants. Crystal structures of almost 150 peptides, ligand or antibodies bound to ECDs of around 50 GPCRs (gpcrdb.org) were used to identify GPCR binding motifs. Over 1000 GPCR binding motifs were extracted from this analysis. In addition, by analysis of all solved structures of GPCRs (zhanglab.ccmb.med.umich.edu/GPCR-EXP/), over 2000 binding motifs from endogenous extracellular loops of GPCRs were identified. Finally, by analysis of structures of over 100 small molecule ligands bound to GPCR, a reduced amino acid library of 5 amino acids (Tyr, Phe, His, Pro and Gly) that may be able to recapitulate many of the structural contacts of these ligands was identified. A sub-library with this reduced amino acid diversity was placed within a CxxxxxC motif. In total, over 5000 GPCR binding motifs were identified (FIGS. 9A-9E). These binding motifs were placed in one of five different stem regions:

(SEQ ID NO: 721)
CARDLRELECEEWTxxxxxSRGPCVDPRGVAGSFDVW,
(SEQ ID NO: 722)
CARDMYYDFxxxxxEVVPADDAFDIW,
(SEQ ID NO: 723)
CARDGRGSLPRPKGGPxxxxxYDSSEDSGGAFDIW,
(SEQ ID NO: 724)
CARANQHFxxxxxGYHYYGMDVW,
(SEQ ID NO: 725)
CAKHMSMQxxxxxRADLVGDAFDVW.

These stem regions were selected from structural antibodies with ultra-long HCDR3s. Antibody germlines were specifically chosen to tolerate these ultra-long HCDR3s. Structure and sequence analysis of human antibodies with longer than 21 amino acids revealed a V-gene bias in antibodies with long CDR3s. Finally, the germline IGHV (IGHV1-69 and IGHV3-30), IGKV (IGKV1-39 and IGKV3-15) and IGLV (IGLV1-51 and IGLV2-14) genes were chosen based on this analysis.

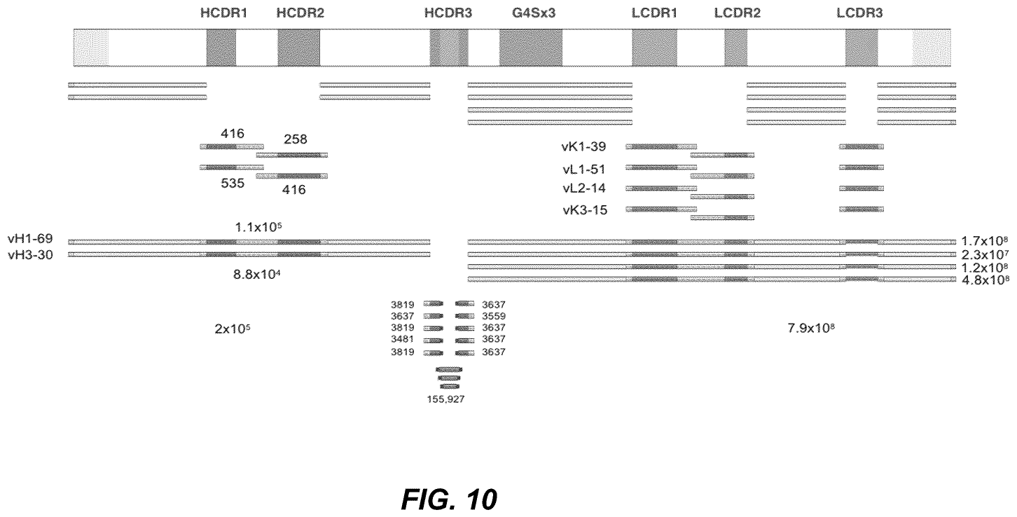

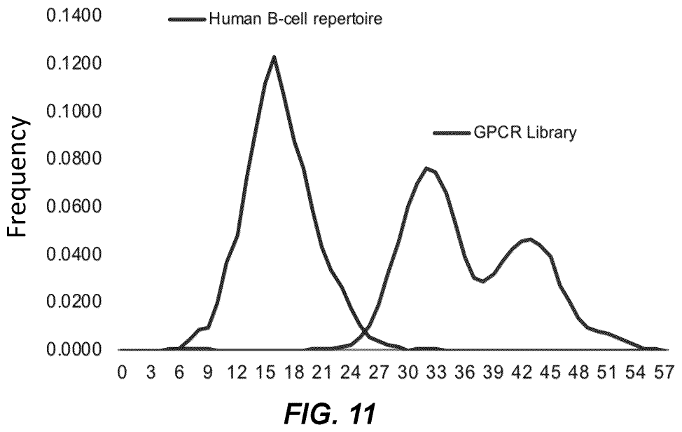

In addition to HCDR3 diversity, limited diversity was also introduced in the other 5 CDRs. There were 416 HCDR1 and 258 HCDR2 variants in the IGHV1-69 domain; 535 HCDR1 and 416 HCDR2 variants in the IGHV3-30 domain; 490 LCDR1, 420 LCDR2 and 824 LCDR3 variants in the IGKV1-39 domain; 490 LCDR1, 265 LCDR2 and 907 LCDR3 variants in the IGKV3-15 domain; 184 LCDR1, 151 LCDR2 and 824 LCDR3 variants in the IGLV1-51 domain; 967 LCDR1, 535 LCDR2 and 922 LCDR3 variants in the IGLV2-14 domain (FIG. 10). These CDR variants were selected by comparing the germline CDRs with the near-germline space of single, double and triple mutations observed in the CDRs within the V-gene repertoire of at least two out of 12 human donors. All CDRs have were pre-screened to remove manufacturability liabilities, cryptic splice sites or nucleotide restriction sites. The CDRs were synthesized as an oligo pool and incorporated into the selected antibody scaffolds. The heavy chain (VH) and light chain (VL) genes were linked by (G₄S)₃ linker (SEQ ID NO: 718). The resulting scFv (VH-linker-VL) gene pool was cloned into a phagemid display vector at the N-terminal of the M13 gene-3 minor coat protein. The final size of the GPCR library is 1×10¹⁰ in a scFv format. Next-generation sequencing (NGS) was performed on the final phage library to analyze the HCDR3 length distribution in the library for comparison with the HCDR3 length distribution in B-cell populations from three healthy adult donors. The HCDR3 sequences from the three healthy donors used were derived from a publicly available database with over 37 million B-cell receptor sequences³¹. The HCDR3 length in the GPCR library is much longer than the HCDR3 length observed in B-cell repertoire sequences. On average, the median HCDR3 length in the GPCR library (which shows a biphasic pattern of distribution) is two or three times longer (33 to 44 amino acids) than the median lengths observed in natural B-cell repertoire sequences (15 to 17 amino acids) (FIG. 11). The biphasic length distribution of HCDR3 in the GPCR library is mainly caused by the two groups of stems (8aa, 9aaxxxxx10aa, 12aa) and (14aa, 16aa xxxxx18aa, 14aa) used to present the motifs within HCDR3.

### Example 7: VHH Libraries

Synthetic VHH libraries were developed. For the ‘VHH Ratio’ library with tailored CDR diversity, 2391 VHH sequences (iCAN database) were aligned using Clustal Omega to determine the consensus at each position and the framework was derived from the consensus at each position. The CDRs of all of the 2391 sequences were analyzed for position-specific variation, and this diversity was introduced in the library design. For the ‘VHH Shuffle’ library with shuffled CDR diversity, the iCAN database was scanned for unique CDRs in the nanobody sequences. 1239 unique CDR1's, 1600 unique CDR2's, and 1608 unique CDR3's were identified and the framework was derived from the consensus at each framework position amongst the 2391 sequences in the iCAN database. Each of the unique CDR's was individually synthesized and shuffled in the consensus framework to generate a library with theoretical diversity of 3.2×10⁹. The library was then cloned in the phagemid vector using restriction enzyme digest. For the ‘VHH hShuffle’ library (a synthetic “human” VHH library with shuffled CDR diversity), the iCAN database was scanned for unique CDRs in the nanobody sequences. 1239 unique CDR1's, 1600 unique CDR2's, and 1608 unique CDR3's were identified and framework 1, 3, and 4 was derived from the human germline DP-47 framework. Framework 2 was derived from the consensus at each framework position amongst the 2391 sequences in the iCAN database. Each of the unique CDR's was individually synthesized and shuffled in the partially humanized framework using the NUGE tool to generate a library with theoretical diversity of 3.2×10⁹. The library was then cloned in the phagemid vector using the NUGE tool.

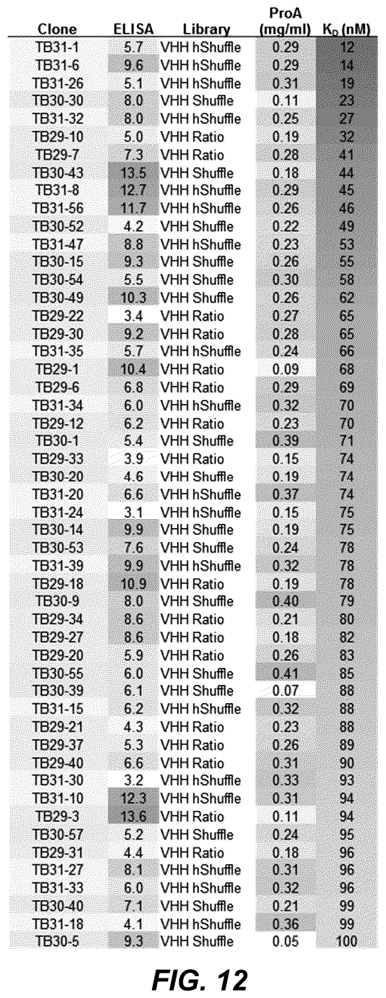

The Carterra SPR system was used to assess binding affinity and affinity distribution for VHH-Fc variants. VHH-Fc demonstrate a range of affinities for TIGIT, with a low end of 12 nM K_{D} and a high end of 1685 nM K_{D} (data not shown). FIG. 12 provides specific values for the VHH-Fc clones for ELISA, Protein A (mg/ml), and K_{D}) (nM).

### Example 8. Hyperimmune Immunoglobulin Library for A2A Receptor

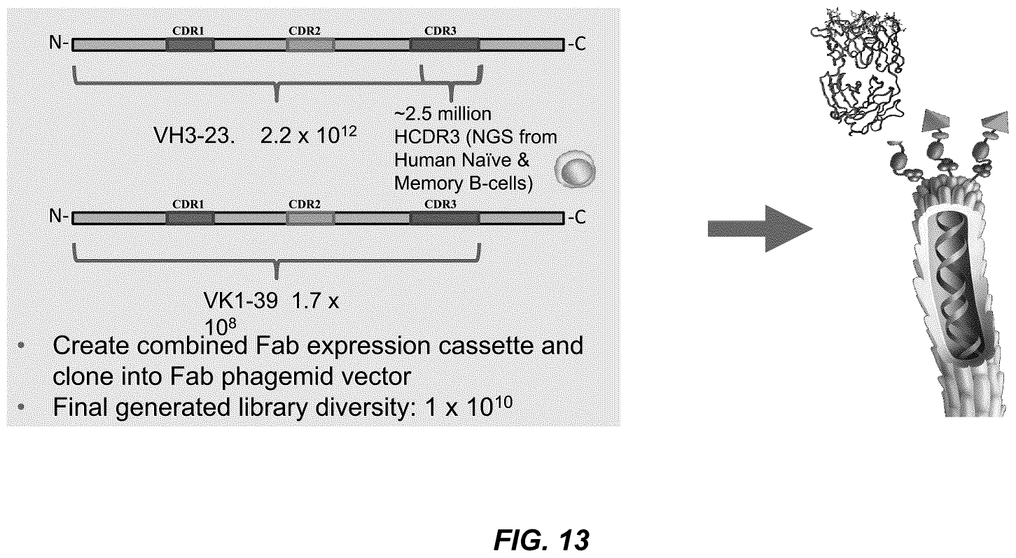

A hyperimmune immunoglobulin (IgG) library was created using similar methods as described in Example 7. Briefly, the hyperimmune IgG library was generated from analysis of databases of human naïve and memory B-cell receptor sequences consisting of more than 37 million unique IgH sequences from each of 3 healthy donors. More than two million CDRH3 sequences were gathered from the analysis and individually constructed using methods similar to Examples 1-3. The CDRH3 sequences were incorporated into the VHH hShuffle library described in Example 9. The final library diversity was determined to be 1.3×10¹⁰. A schematic of the design can be seen in FIG. 13.

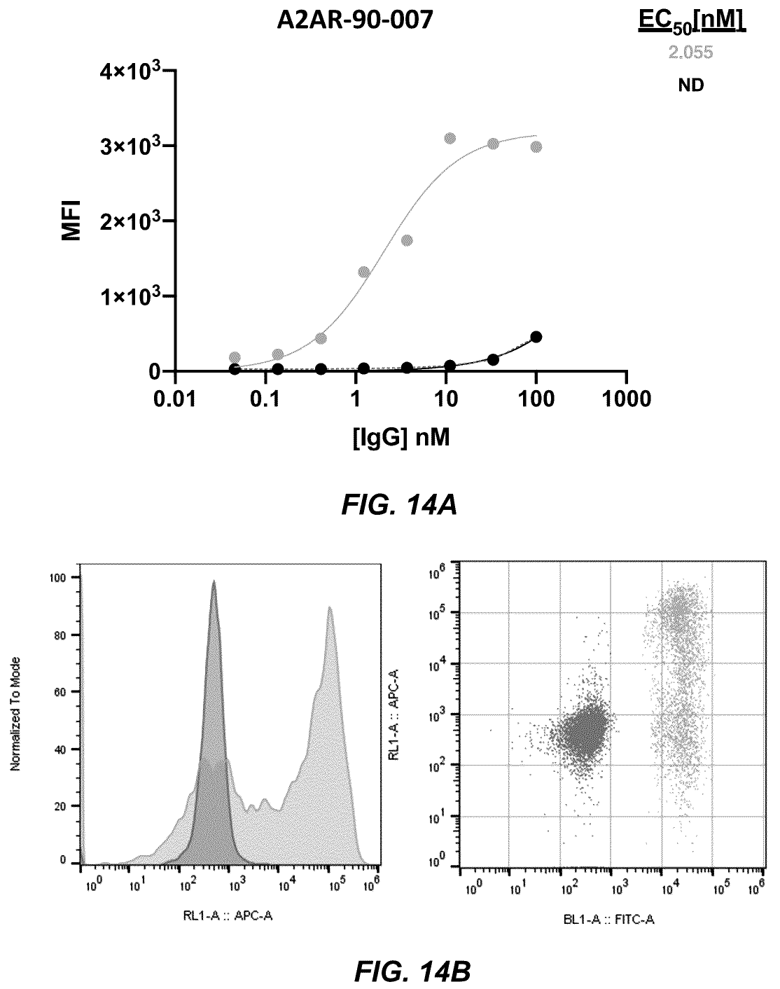

73 out of 88 unique clones had a target cell MFI values 2 fold over parental cells. 15 out of 88 unique Clones with target cell MFI values 20 fold over parental cells. Data for adenosine A2A receptor variant A2AR-90-007 is seen in FIGS. 14A-14B.

This Example shows generation of a VHH library for the A2AR with high affinity and K_{D}) values in the sub-nanomolar range.

### Example 9. GPCR Libraries with Varied CDR's

A GPCR library was created using a CDR randomization scheme.

Briefly, GPCR libraries were designed based on GPCR antibody sequences. Over sixty different GPCR antibodies were analyzed and sequences from these GPCRs were modified using a CDR randomization scheme.

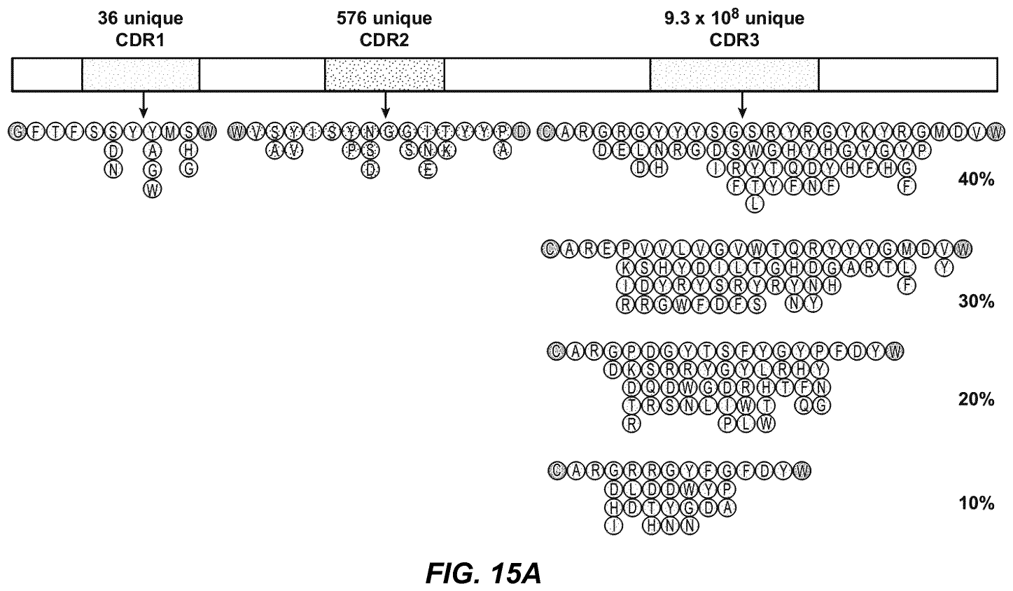

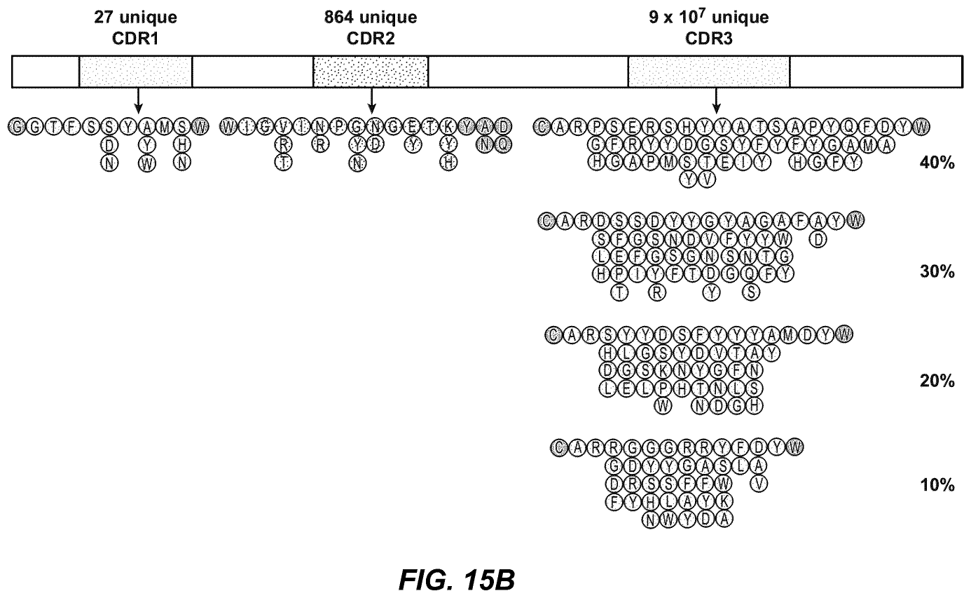

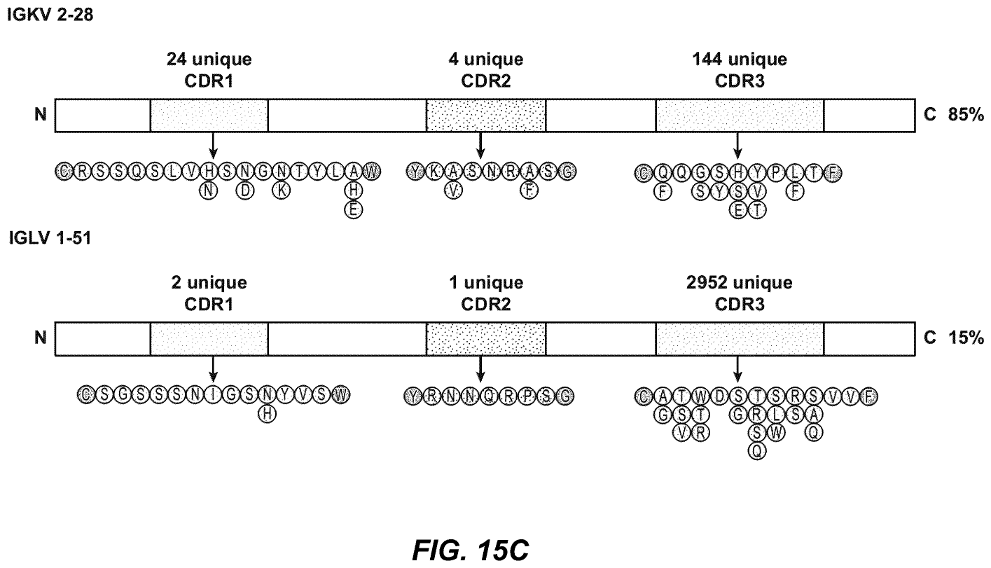

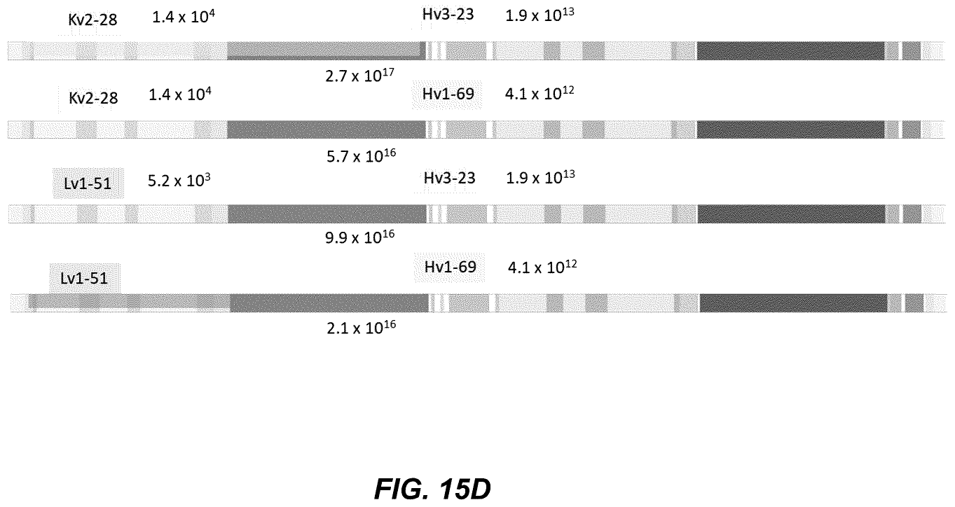

The heavy chain IGHV3-23 design is seen in FIG. 15A. As seen in FIG. 15A, IGHV3-23 CDRH3's had four distinctive lengths: 23 amino acids, 21 amino acids, 17 amino acids, and 12 amino acids, with each length having its residue diversity. The ratio for the four lengths were the following: 40% for the CDRH3 23 amino acids in length, 30% for the CDRH3 21 amino acids in length, 20% for the CDRH3 17 amino acids in length, and 10% for the CDRH3 12 amino acids in length. The CDRH3 diversity was determined to be 9.3×10⁸, and the full heavy chain IGHV3-23 diversity was 1.9×10¹³.

The heavy chain IGHV1-69 design is seen in FIG. 15B. As seen in FIG. 15B, IGHV1-69 CDRH3's had four distinctive lengths: 20 amino acids, 16 amino acids, 15 amino acids, and 12 amino acids, with each length having its residue diversity. The ratio for the four lengths were the following: 40% for the CDRH3 20 amino acids in length, 30% for the CDRH3 16 amino acids in length, 20% for the CDRH3 15 amino acids in length, and 10% for the CDRH3 12 amino acids in length. The CDRH3 diversity was determined to be 9×10⁷, and the full heavy chain IGHV-69 diversity is 4.1×10¹².

The light chains IGKV 2-28 and IGLV 1-51 design is seen in FIG. 15C. Antibody light chain CDR sequences were analyzed for position-specific variation. Two light chain frameworks were selected with fixed CDR lengths. The theoretical diversities were determined to be 13800 and 5180 for kappa and light chains, respectively.

The final theoretical diversity was determined to be 4.7×10¹⁷ and the final, generated Fab library had a diversity of 6×10⁹. See FIG. 15D.

### Example 10. Adenosine A2A Receptor Libraries with Varied CDR's

An adenosine A2A receptor library is created using a CDR randomization scheme similarly described in Example 9.

Briefly, adenosine A2A receptor libraries are designed based on GPCR antibody sequences. Over sixty different GPCR antibodies are analyzed and sequences from these GPCRs are modified using a CDR randomization scheme. Adenosine A2A receptor variant IgGs designed using the CDR randomization scheme are purified and are assayed to determine cell-based affinity measurements and for functional analysis.

### Example 11. A2A Variant Immunoglobulins

A2AR variant immunoglobulins generated were assayed in various functional assays.

First, A2AR immunoglobulin scFv phage libraries were panned on cells and immobilized A2a proteins, and screened. The output phage numbers from each round of selection are seen in Tables 7-8.

TABLE 7

| Target | Library | Round 1 | Round 2 | Round 3 | Round 4 | Round 5 |
|---|---|---|---|---|---|---|
| HEK293- | Mouse | 2.7 ×10⁶ | 4.1 ×10⁷ | 5.0 ×10⁷ | 5.0 ×10⁷ | 1.2 ×10⁸ |
| A2a Cells | Immune | | | | | |
| A2a protein | Humanized | 4.1 ×10⁶ | 8.0 ×10⁷ | 2.3 ×10⁸ | 1.2 ×10⁷ | 5.8 ×10⁷ |
| | Synthetic | | | | | |
| A2a protein + | Humanized | 5.2 ×10⁶ | 4.5 ×10⁷ | 1.3 ×10⁸ | 3.0 ×10⁷ | 6.7 ×10⁷ |
| ZM241385 | Synthetic | | | | | |
| A2a protein | Mouse | 4.3 ×10⁷ | 5.8 ×10⁷ | 3.0 ×10⁷ | 4.8 ×10⁷ | 3.2 ×10⁷ |
| | Immune | | | | | |
| A2a protein + | Mouse | 2.4 ×10⁷ | 3.7 ×10⁷ | 1.9 ×10⁸ | 6.0 ×10⁷ | 6.0 ×10⁶ |
| ZM241385 | Immune | | | | | |

TABLE 8

| Target | Library | Round 1 | Round 2 | Round 3 | Round 4 | Round 5 |
|---|---|---|---|---|---|---|
| HEK293- | Immune | 1.3 ×10⁶ | 3.1 ×10⁷ | 5.0 ×10⁷ | 5.0 ×10⁷ | 1.3 ×10⁸ |
| A2a Cells | | | | | | |
| A2a protein | Synthetic | 5.2 ×10⁶ | 3.7 ×10⁷ | 1.5 ×10⁸ | 1.2 ×10⁷ | 4.9 ×10⁷ |
| A2a protein + | Synthetic | 6.7 ×10⁶ | 2.9 ×10⁷ | 6.0 ×10⁷ | 2.0 ×10⁷ | 6.0 ×10⁷ |
| ZM241385 | | | | | | |
| A2a protein | Immune | 8.0 ×10⁶ | 2.0 ×10⁷ | 9.0 ×10⁷ | 2.3 ×10⁷ | 2.8 ×10⁷ |
| A2a protein + | Immune | 6.0 ×10⁶ | 1.7 ×10⁷ | 1.3 ×10⁸ | 4.6 ×10⁷ | 1.9 ×10⁷ |
| ZM241385 | | | | | | |

### Example 12. Screening Antibody Binding

Selected A2AR immunoglobulins from the groups listed in Tables 15-18 were assayed for binding to the targets as listed in the tables.

### HEK293-A2a Cells

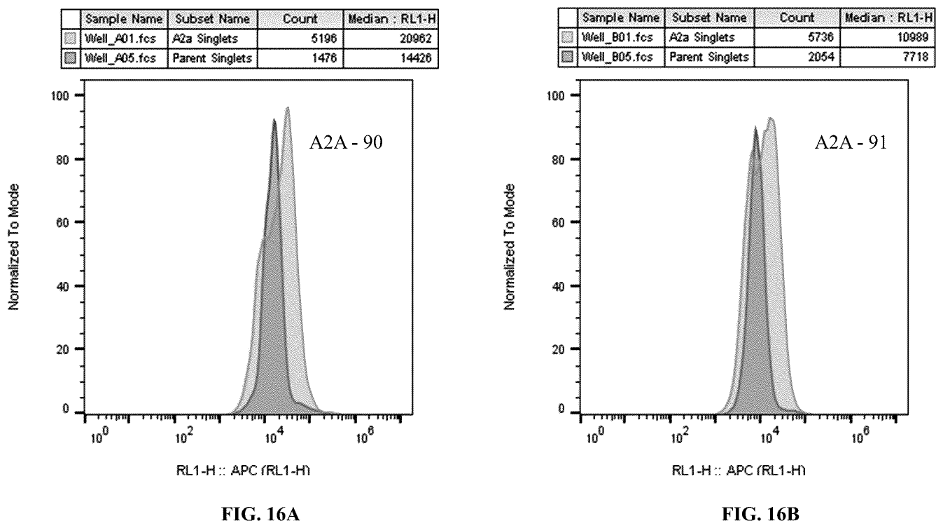

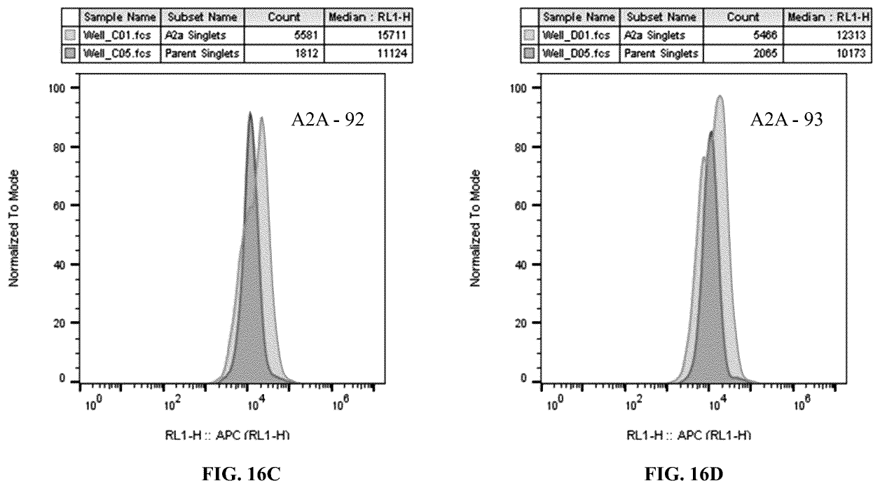

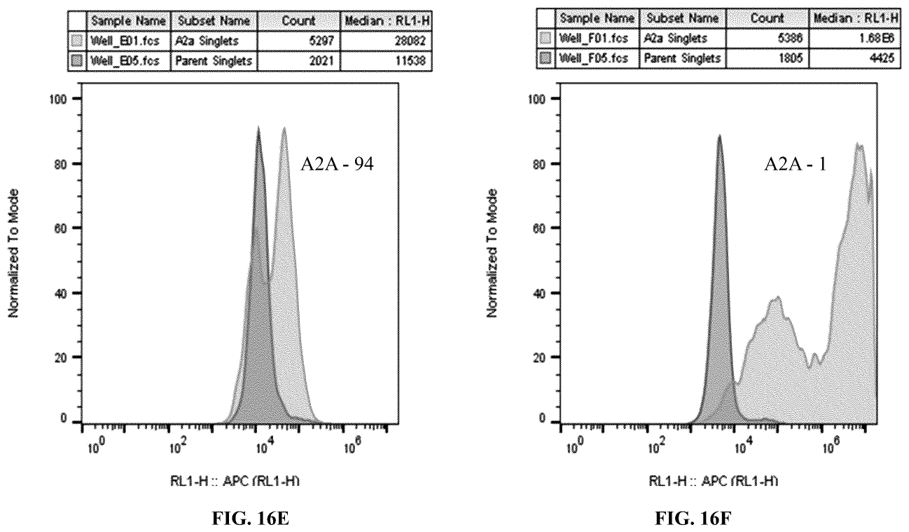

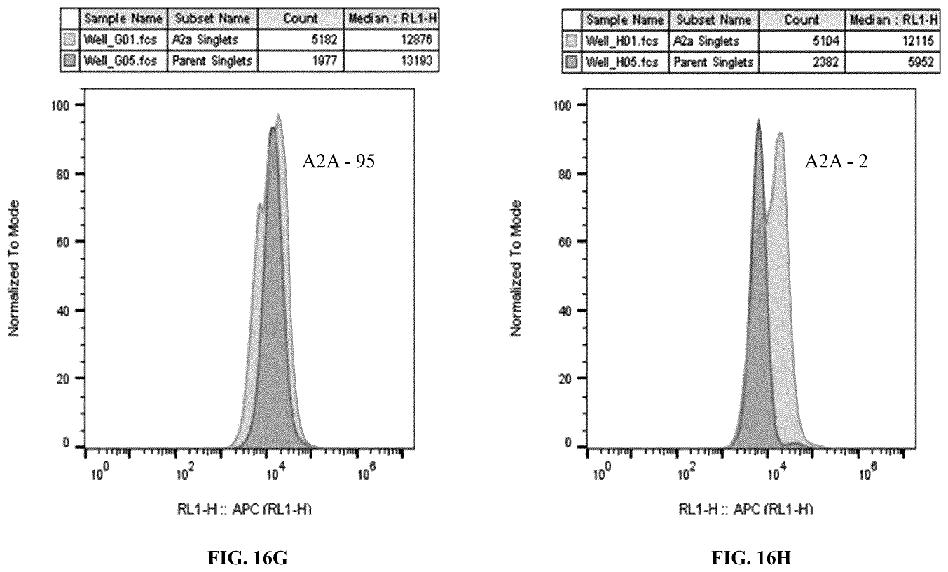

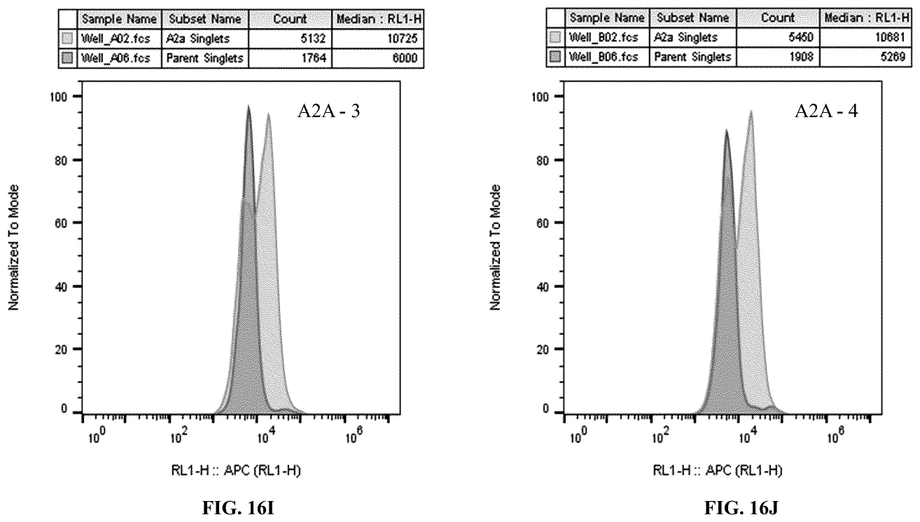

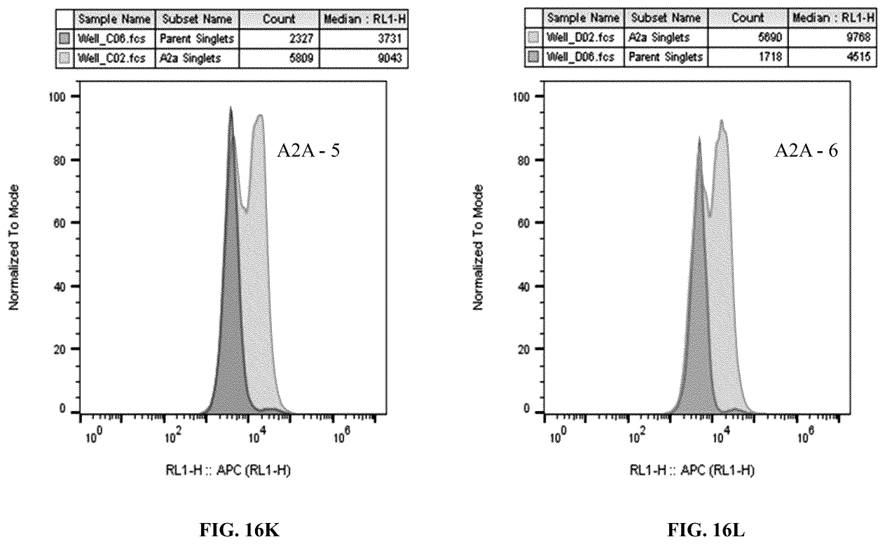

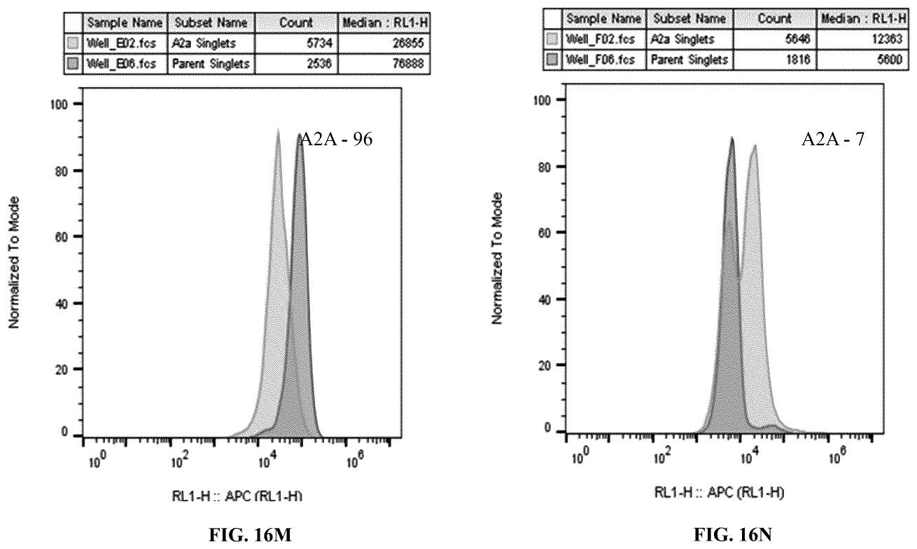

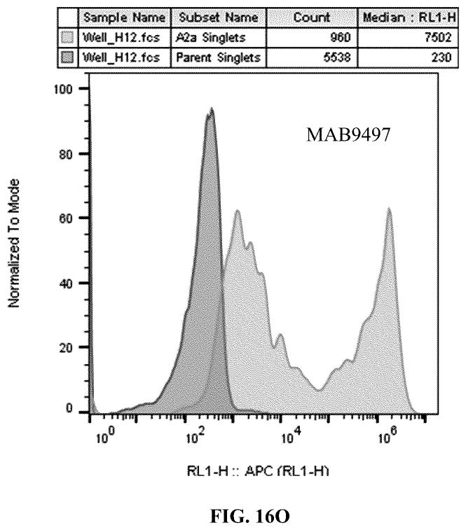

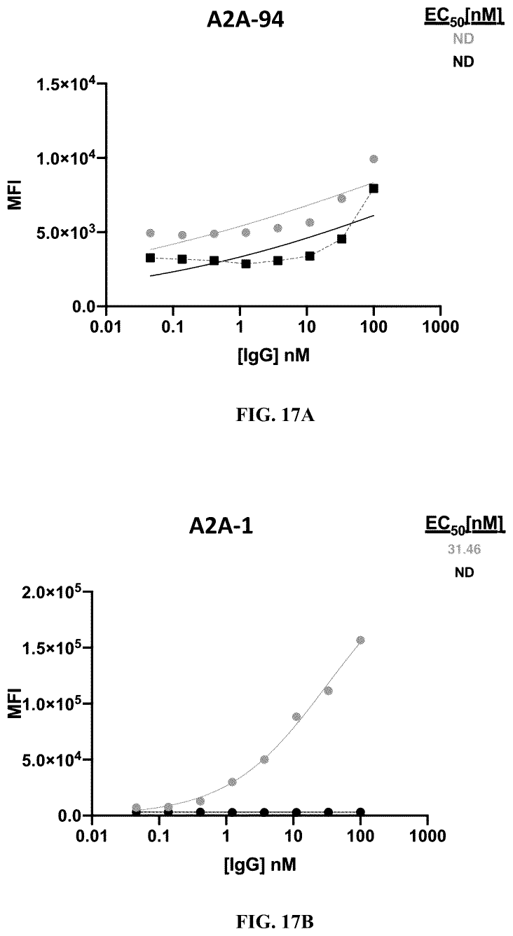

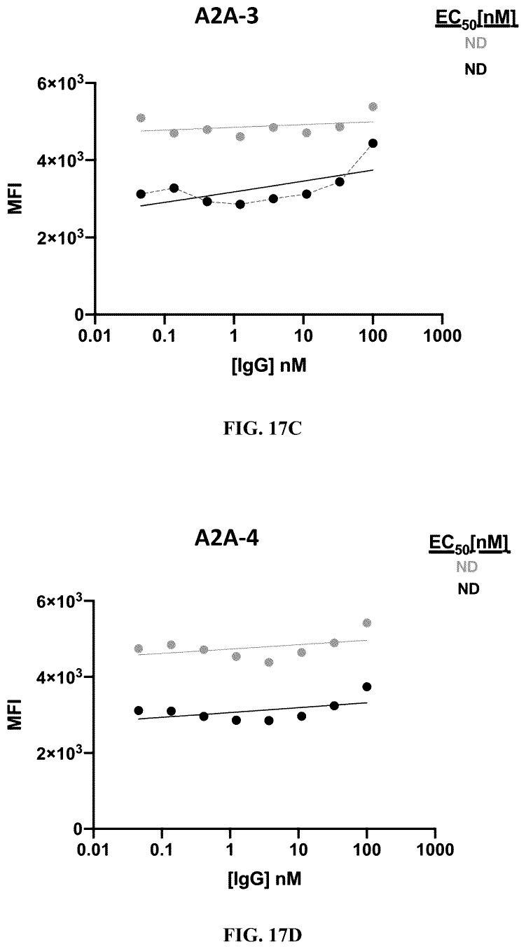

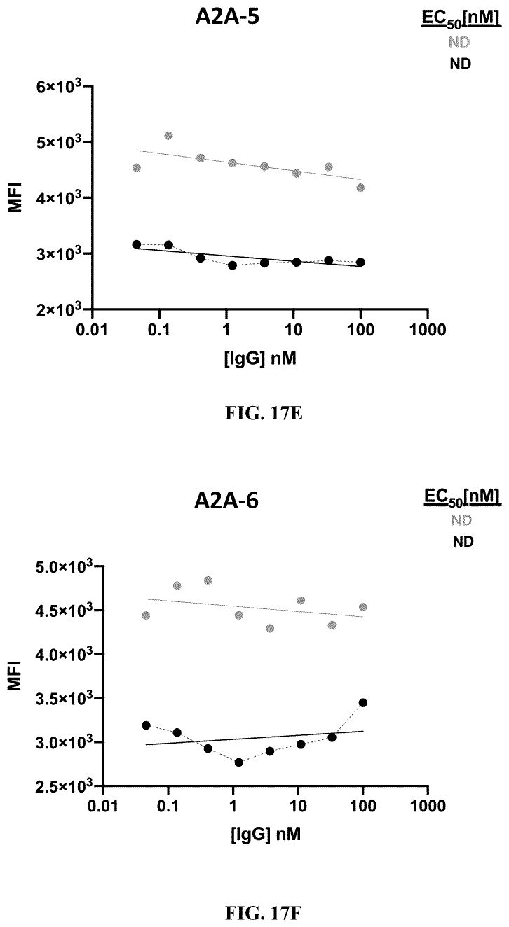

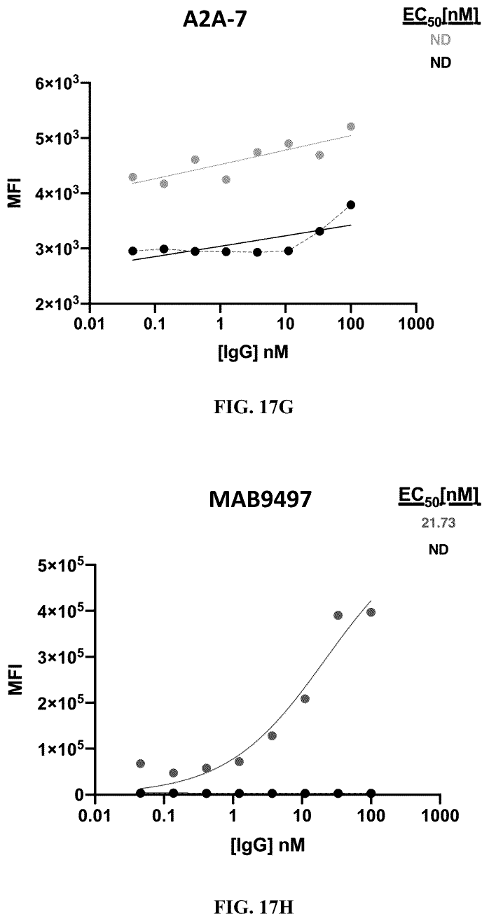

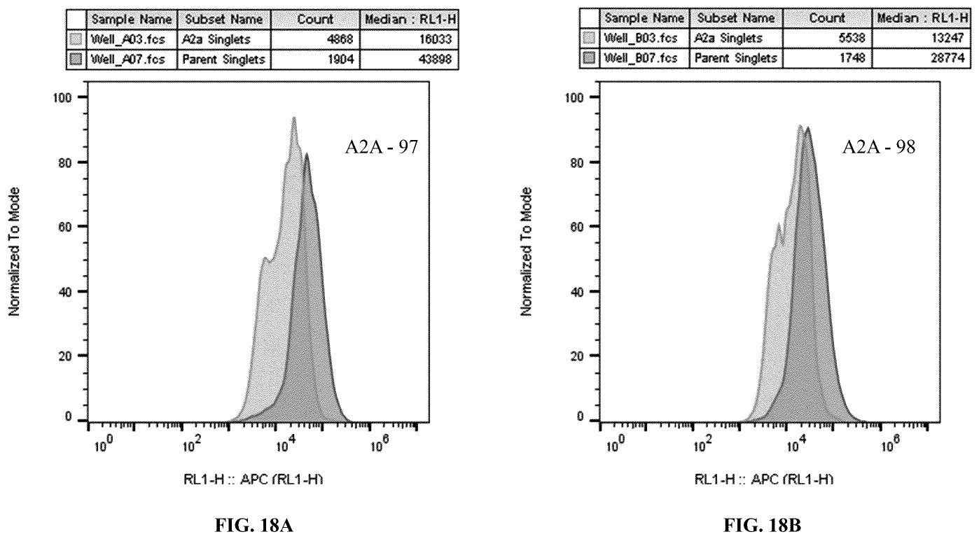

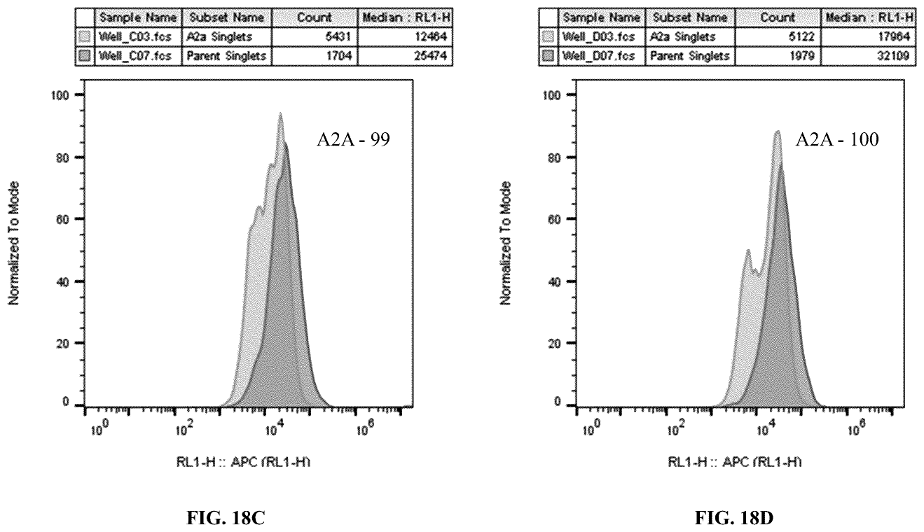

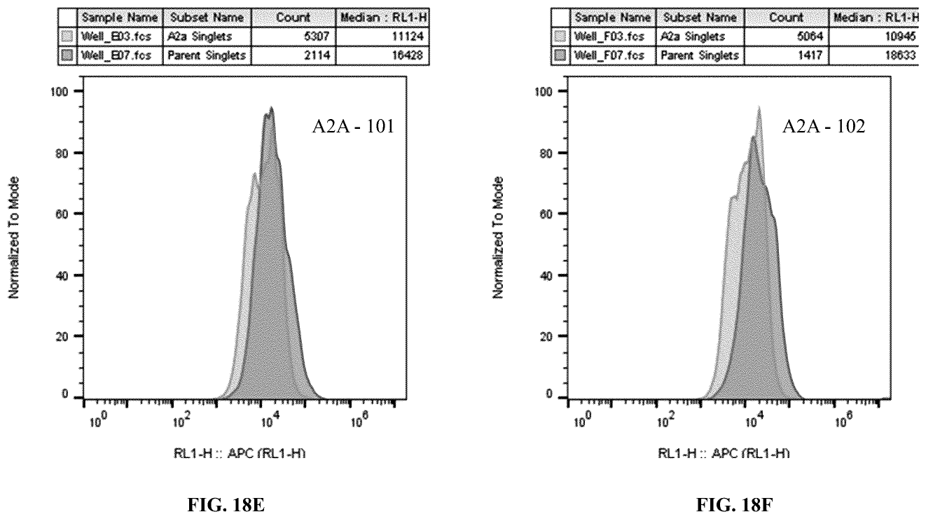

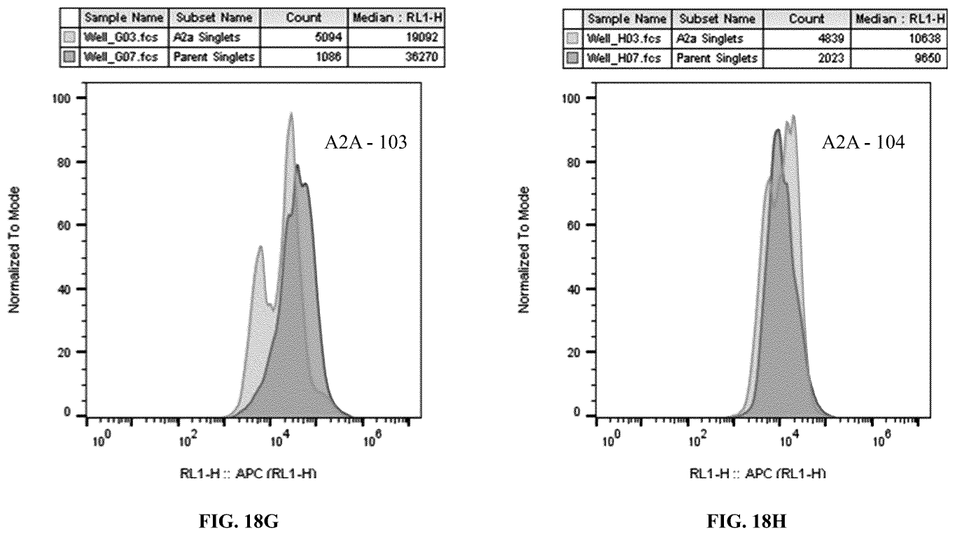

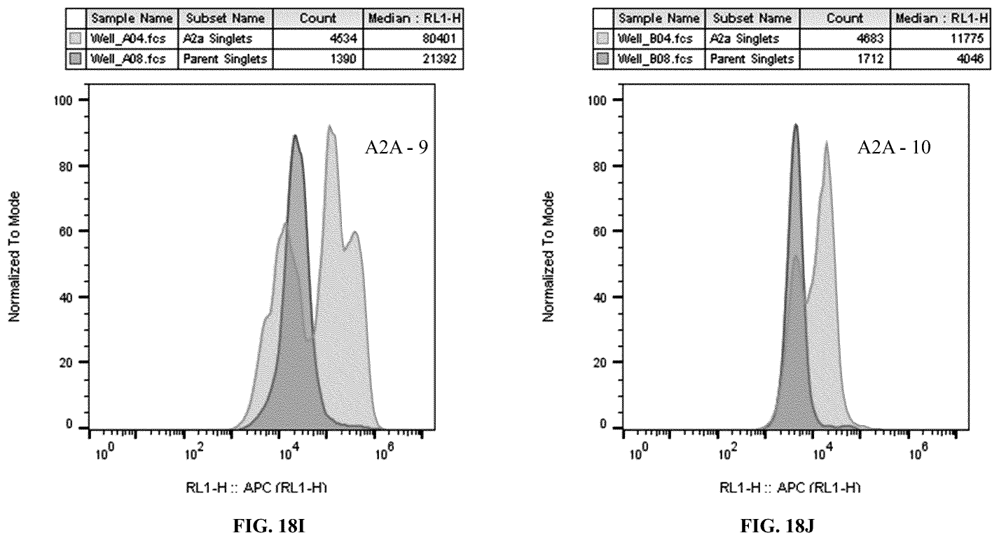

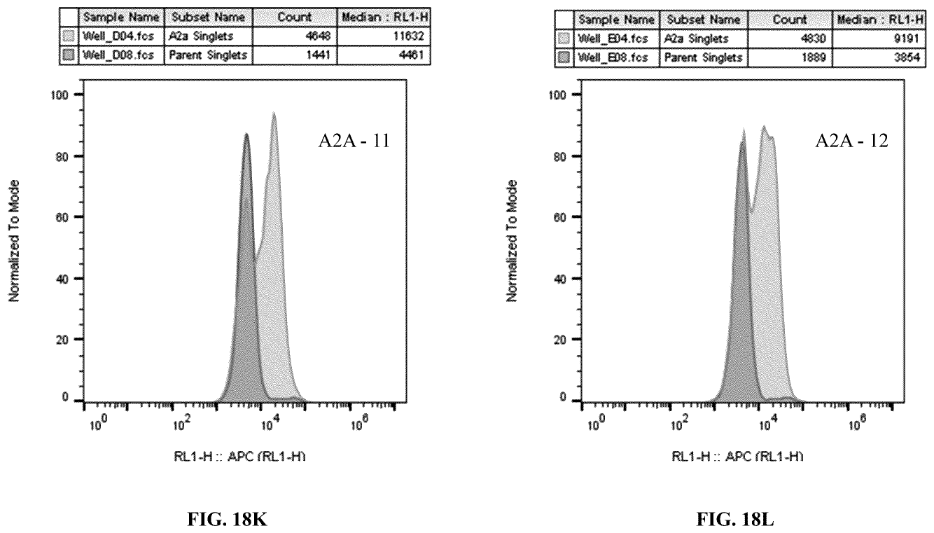

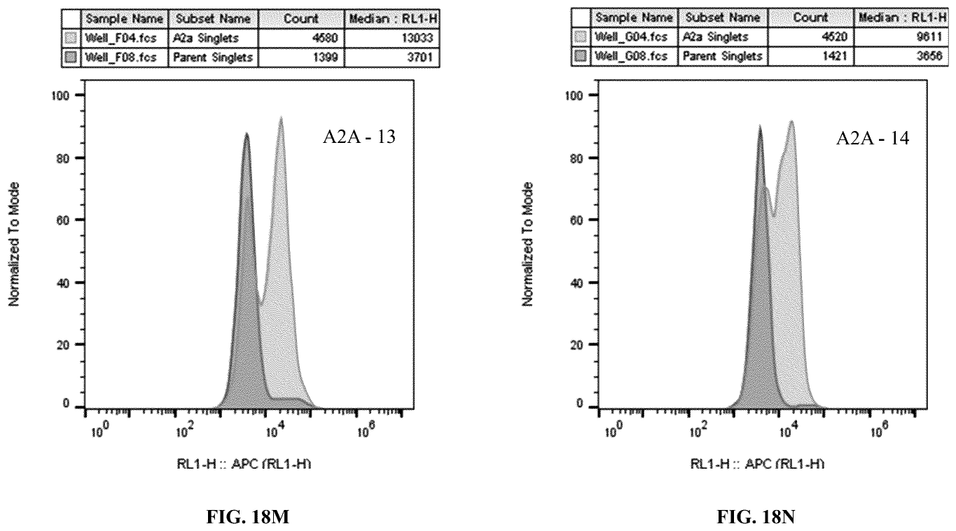

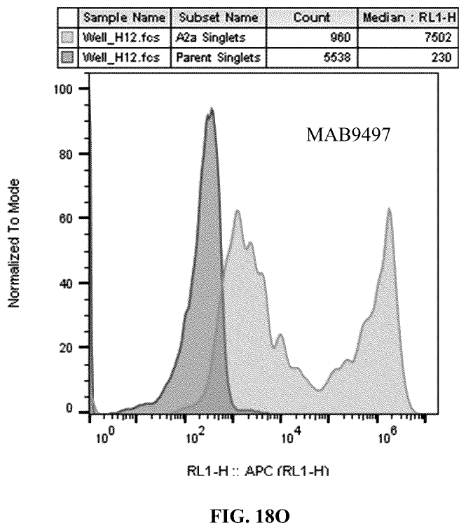

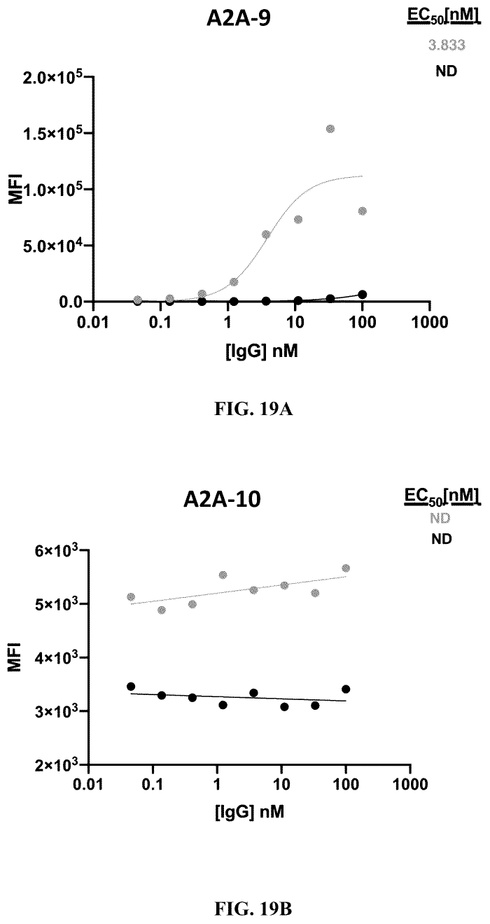

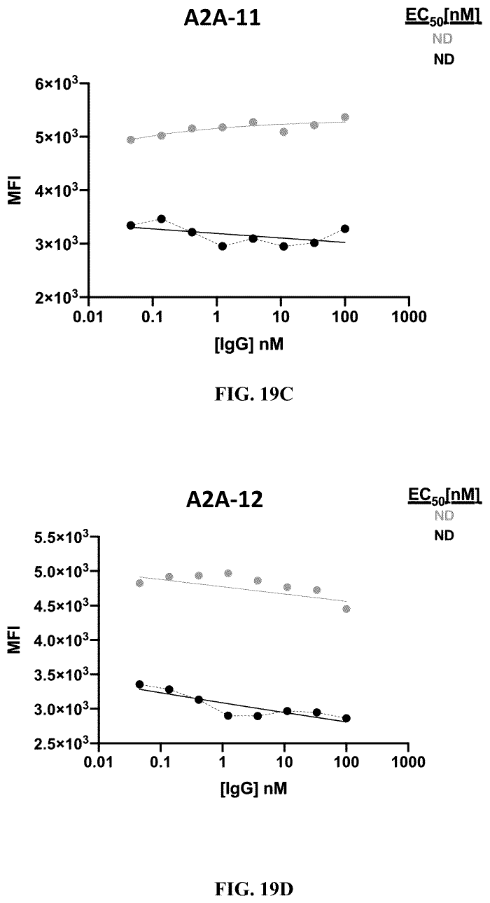

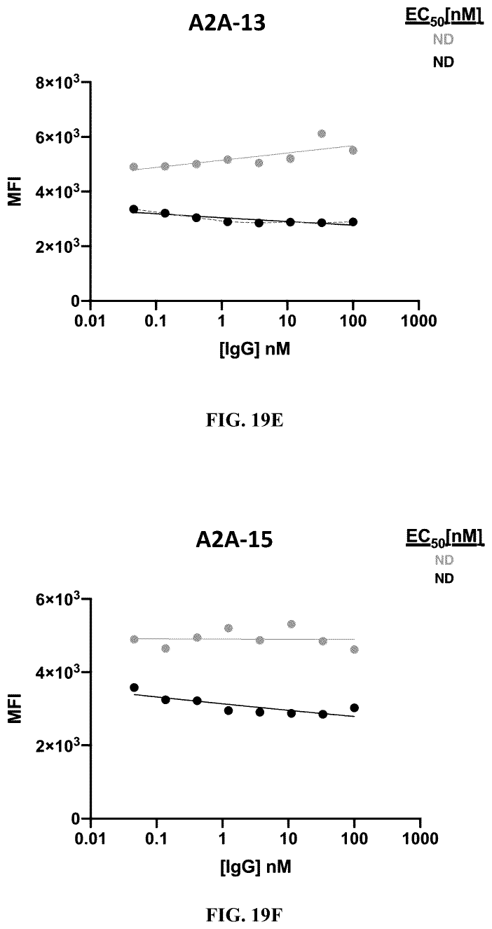

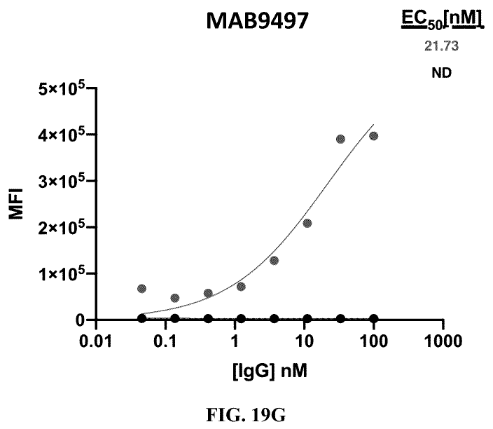

Flow cytometry data showing binding to HEK293-A2a cells using immunoglobulins from variant libraries were generated using 100 nM IgG and compared to detected binding in parent cells. Binding using variants from an immune library are shown in FIGS. 16A-16N. A control is shown in FIG. 16O, showing cell binding with Human Adenosine A2aR monoclonal (MAB9497). Selected variants were assessed for binding at concentrations titrated from 100 nM. Resulting curves are show in FIGS. 17A-17H. Binding curves are plotted with IgG concentration vs. MFI (mean fluorescence intensity). Binding using variants from a mouse immune library are shown in FIGS. 18A-18N. A control is shown in FIG. 18O, showing cell binding with Human Adenosine A2aR monoclonal (MAB9497). Selected variants were assessed for binding at concentrations titrated from 100 nM. Resulting curves are show in FIGS. 19A-19G. Binding curves are plotted with IgG concentration vs. MFI (mean fluorescence intensity).

### Protein Bindings

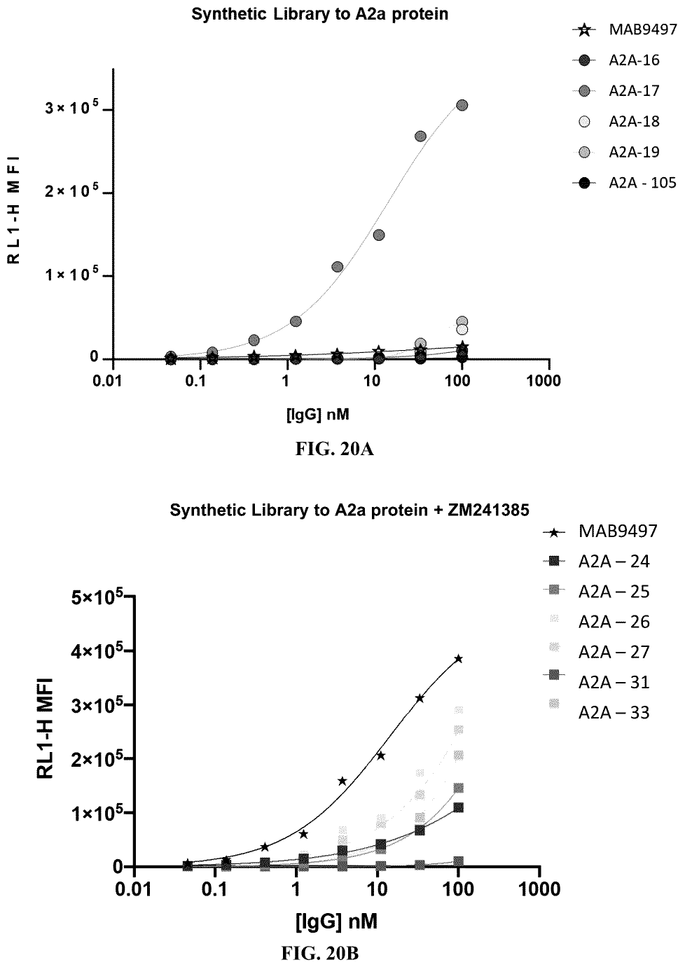

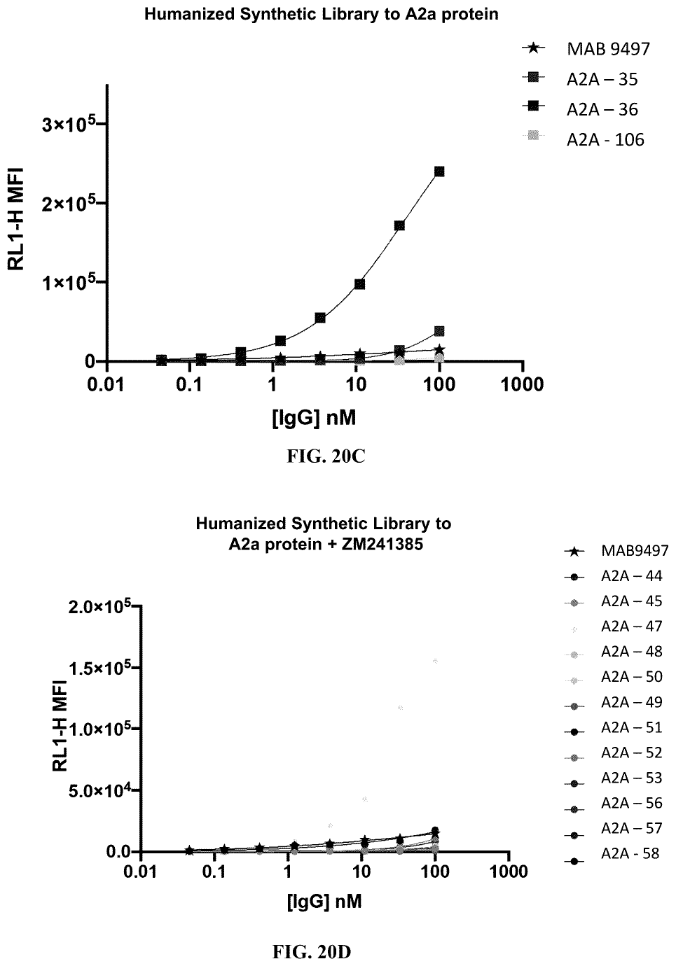

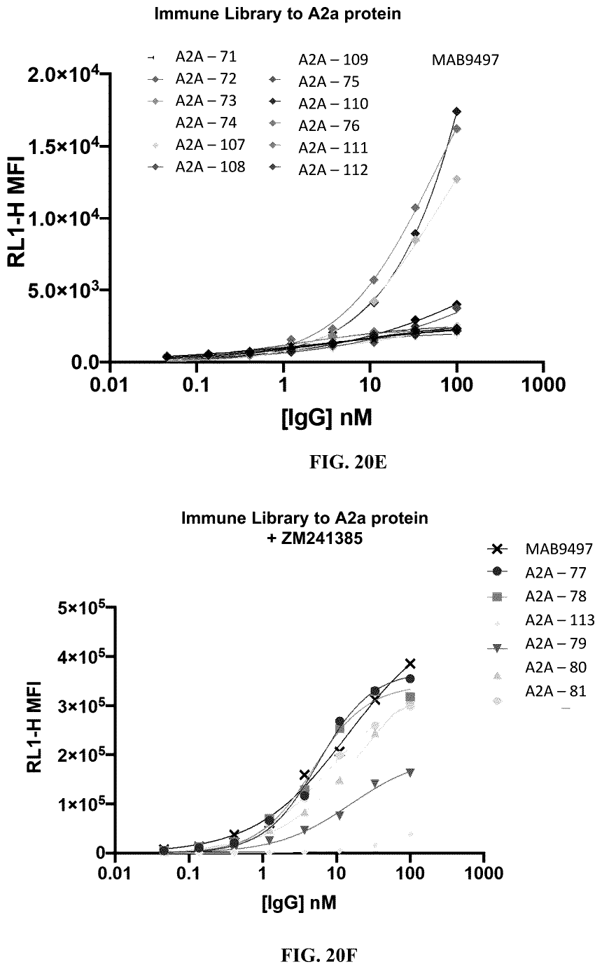

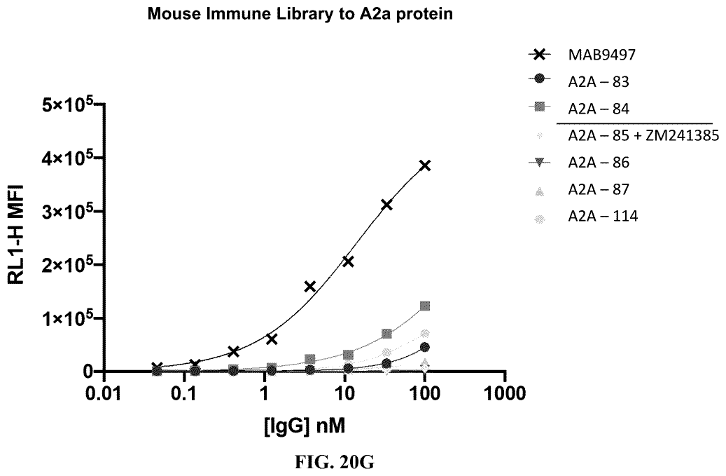

Purified A2a immunoglobulins from Tables 15-18 were assayed for binding in a titration from 100 nM. Results of selected variants are shown in FIGS. 20A-20G.

### Example 13. Agonist Response in LANCE® CAMP Assay

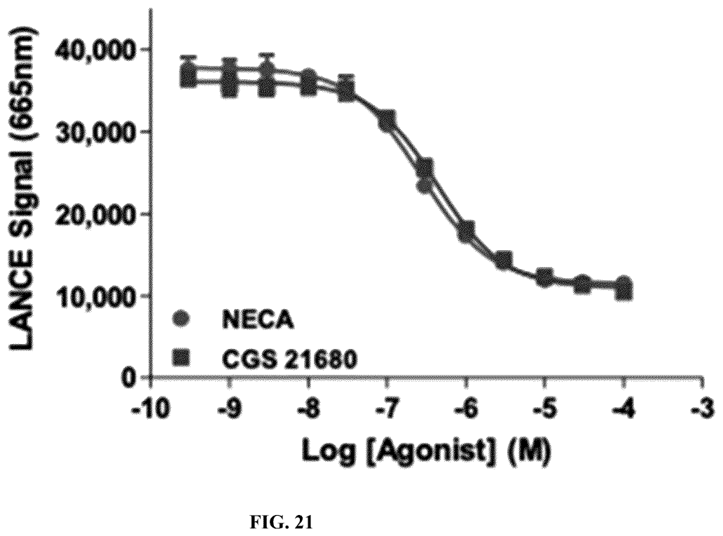

An agonist dose-response assay was performed using a LANCER CAMP assay in 384-well format using 2500 cells/well according to manufacturer's instructions. Cell stimulation with NECA and CGS 21680 was performed for 30 min at room temperature. Readings were taken on a En Vision plate reader in Laser mode. Data is shown in FIG. 21. The Z′-factor was calculated for NECA with at least 16 background and 16 maximal signal points (Z′=0.80). Calculated EC₅₀ (M) for NECA=2.7×10⁻⁷ and for CGS 21680=4.3×10⁻⁷.

### Example 14. Antagonist Response in LANCE® CAMP Assay

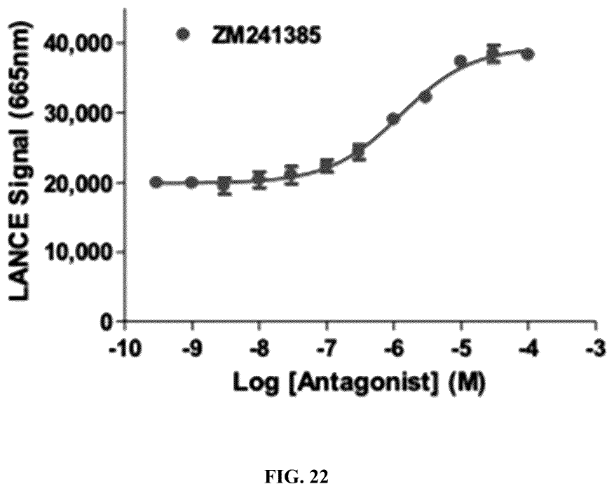

An antagonist dose-response assay was performed using a LANCER CAMP assay in 384-well format using 2500 cells/well and 1 μM NECA (reference agonist) according to manufacturer's instruction. Cell stimulation with ZM241385 was performed for 30 min at room temperature. Readings were taken on a EnVision plate reader in Laser mode. Data is shown in FIG. 22. Calculated IC₅₀ (M) for ZM241385=1.25×10⁻⁵.

### Example 15. A2A CAMP Antagonist Titration

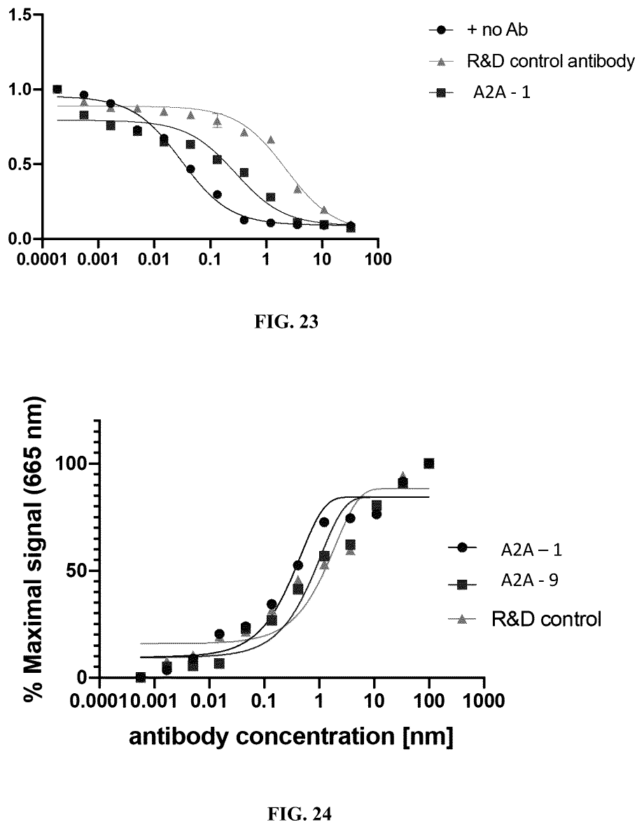

Cells were plated at 3000/well and pre-incubated with fixed 100 nM IgG for 1 hr at room temperature, followed by stimulation with NECA titration for 30 min at room temperature according to manufacturer's instructions. Buffer was PBS+0.1% BSA+0.5 mM IBMX. Results shown in FIG. 23. Absolute IC50 is shown in Table 9, indicating A2A-1 is a negative allosteric modulator.

TABLE 9

| | +no Ab | A2A − 1 | R&D control antibody |
|---|---|---|---|
| IC50 | 0.03040 | 0.2816 | 2.253 |

### Example 16. LANCER Allosteric cAMP Assay

A2A-1 and A2A-9 were assayed for allosteric modulation. Cells were pre-incubated with titrated IgG for 1 hr at room temperature, followed by stimulation with fixed NECA concentration. Results are shown in FIG. 24. IC50 values are shown in Table 10, indicating A2A-1 is a negative allosteric modulator.

TABLE 10

| | A2A − 1 | A2A − 9 | R&D control antibody |
|---|---|---|---|
| Absolute IC50 | 1.833 | 4.106 | 9.432 |

### Example 17. cAMP Allosteric A2A Perkin Elmer

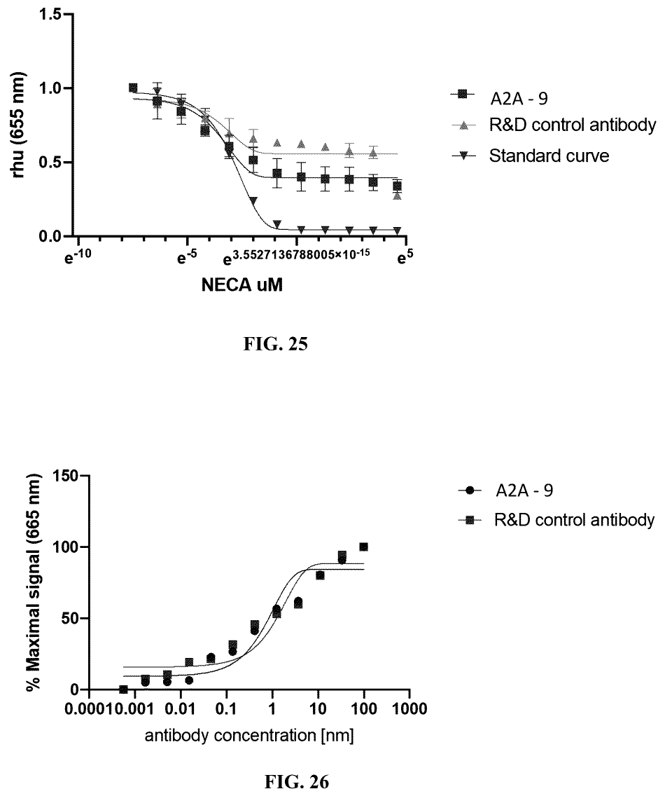

A2A-9 was assayed as described in Example 15. Resulting response curves are shown in FIG. 25. Calculated IC50 for A2A-9 is shown in Table 11.

TABLE 11

| | A2A − 9 | R&D control antibody | No antibody |
|---|---|---|---|
| Absolute IC50 | ~0.4513 | ~0.5126 | ~0.2556 |

### Example 18. A2A CAMP Antagonist Titration

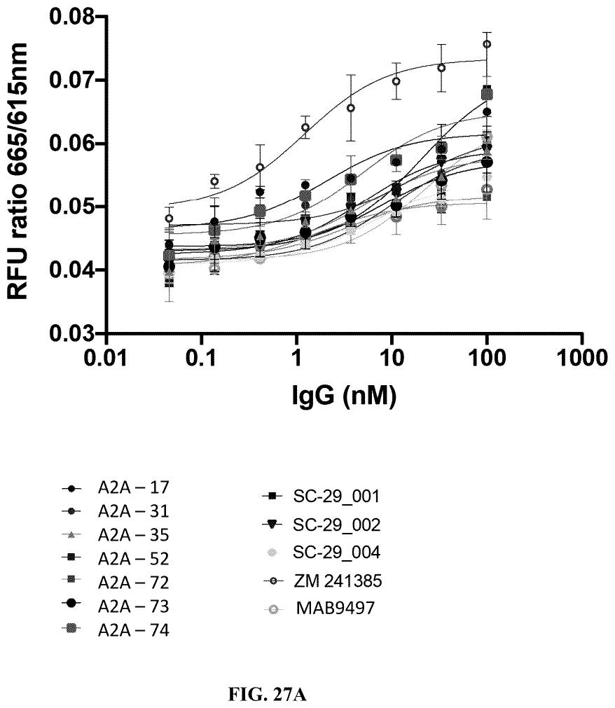

A2A-9 was assayed as described in Example 16. Resulting response curves are shown in FIG. 26. Calculated IC50 values are shown in Table 12. Results indicate A2A-9 is an antagonist.

TABLE 12

| | A2A − 9 | R&D control antibody |
|---|---|---|
| Absolute IC50 | 4.106 | 9.432 |

### Example 19. A2A Antagonistic CAMP Assay

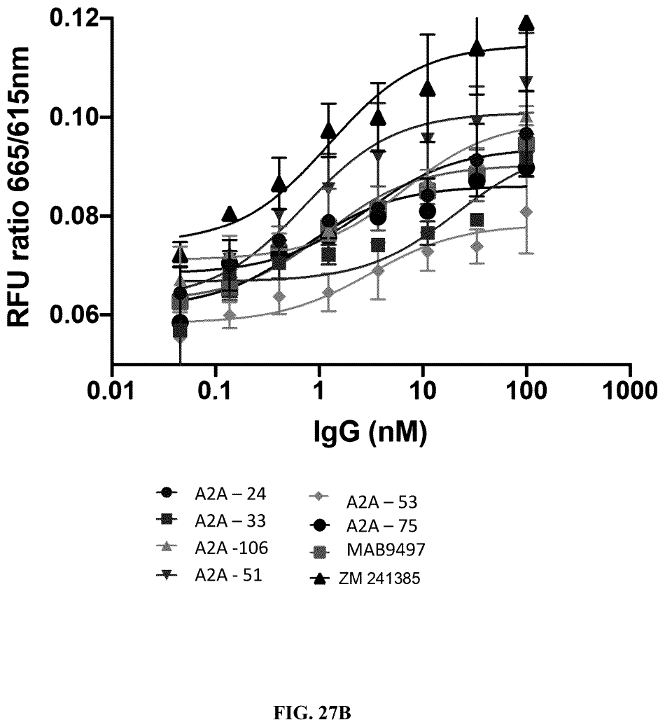

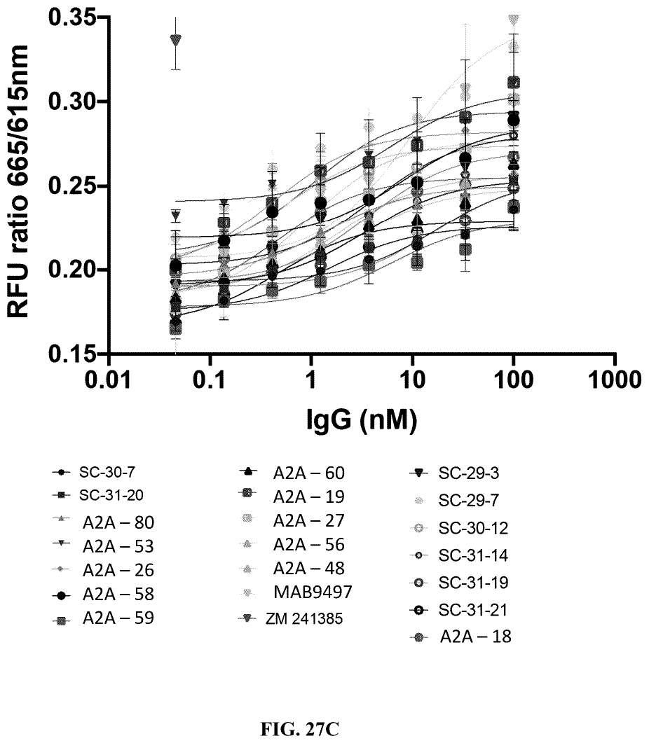

Selected variants were assayed for binding to target. Immunoglobulins were titrated in triplicate and incubated on cells for 1 hour, followed by incubation with 0.5 μM NECA for 30 minutes. Binding curves showing relative fluorescent units (RFU) ratio at 665 nm/615 nm versus nM IgG on a log scale are shown in FIGS. 27A-27C. Final binding studies found functional antibodies in the generated libraries as listed in Table 13 and Table 14.

TABLE 13

| Target | Library | Reformatted | Functional |
|---|---|---|---|
| HEK293-A2a Cells | Mouse Immune | 14 | |
| A2a protein | Humanized Synthetic | 95 | 2 |
| A2a protein + ZM241385 | Humanized Synthetic | 95 | 3 |
| A2a protein | Mouse Immune | 12 | 1 |
| A2a protein + ZM241385 | Mouse Immune | 22 | 0 |

TABLE 14

| Target | Library | Reformatted | Functional |
|---|---|---|---|
| HEK293-A2a Cells | Immune | 14 | |
| A2a protein | Synthetic | 95 | 2 |
| A2a protein + ZM241385 | Synthetic | 95 | 5 |
| A2a protein | Immune | 29 | 4 |
| A2a protein + ZM241385 | Immune | 10 | 5 |

### Example 20. A2AR Cell Functional cAMP Assays

Allosteric and Antagonistic cAMP Assays were Performed Using A2A Cell Lines

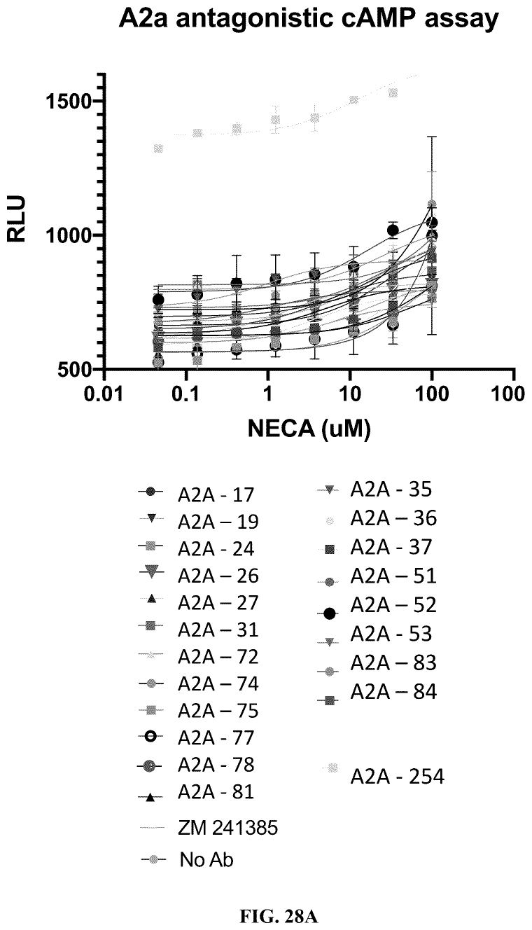

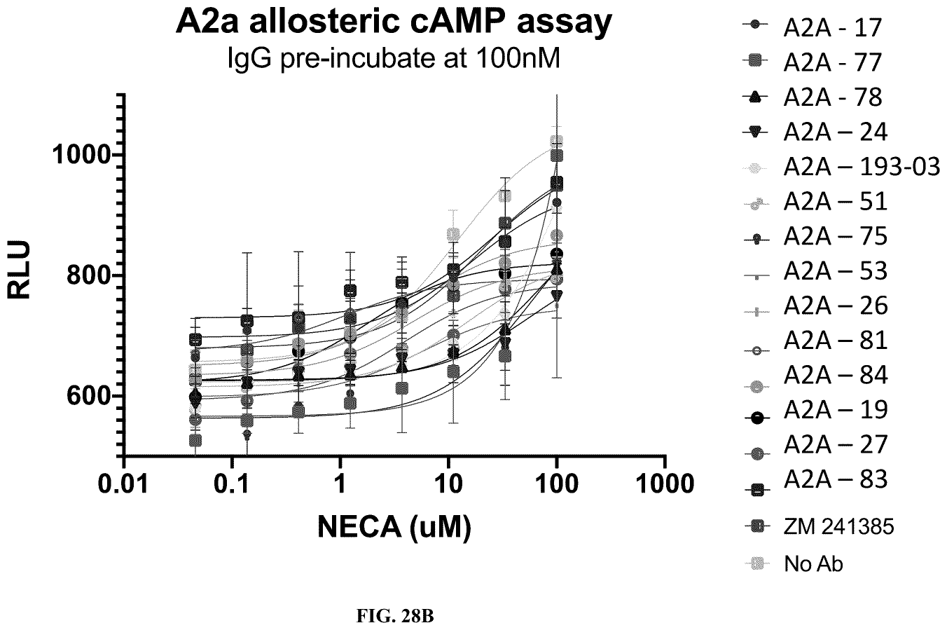

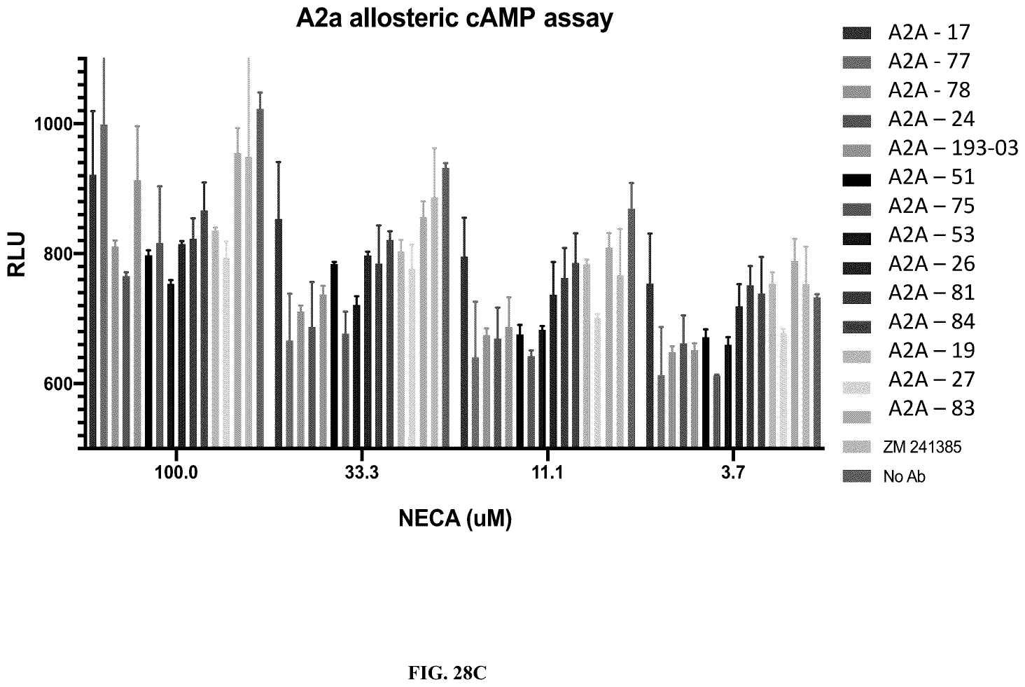

Briefly, cells were pre-incubated with anti-A2AR antibody at 100 nM followed by NECA stimulation 3× titration from 100 uM. Data from a functional allosteric cAMP assay is seen in FIGS. 28A-28C. ZM241385 functioned as an antagonist. “No Ab” functioned as agonist only.

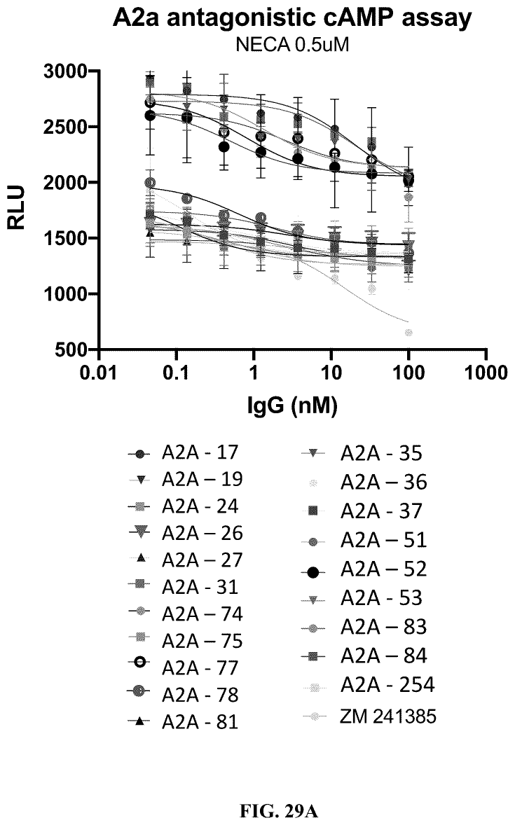

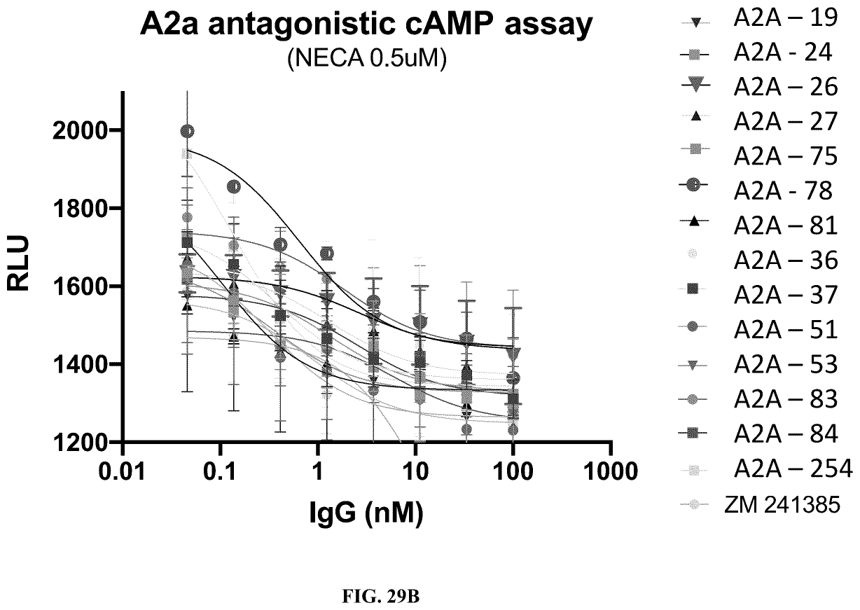

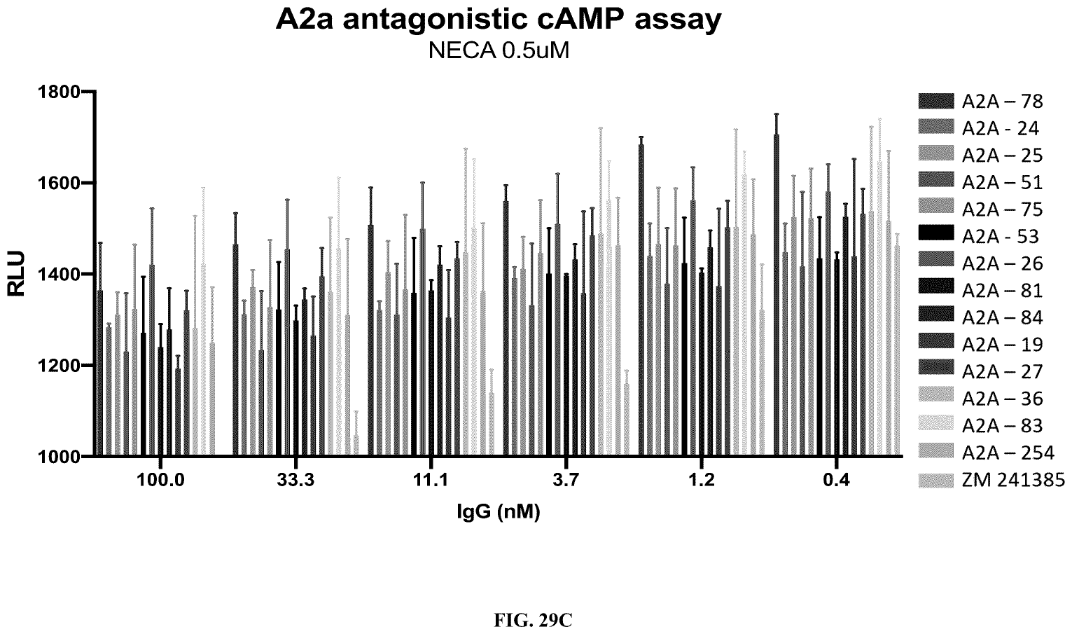

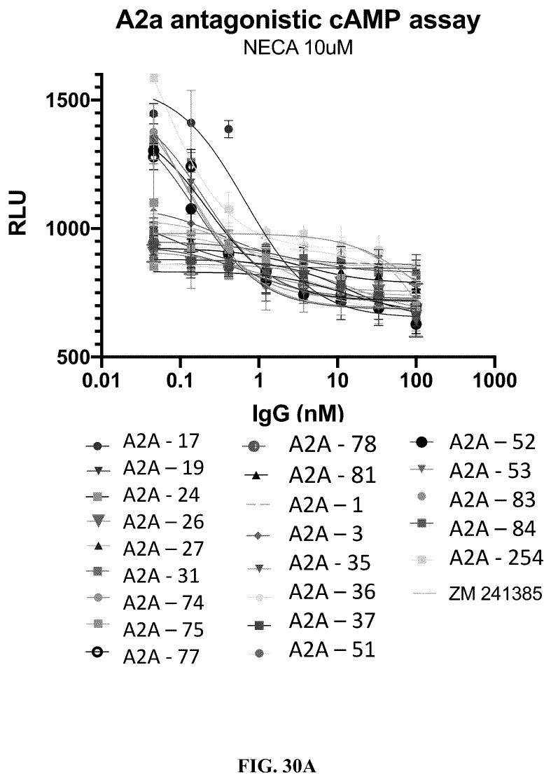

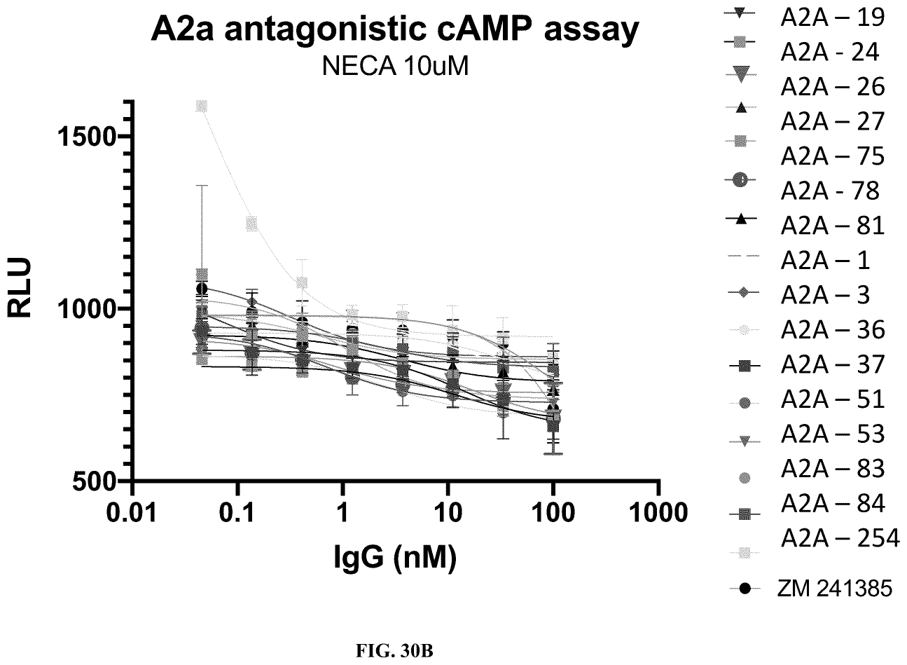

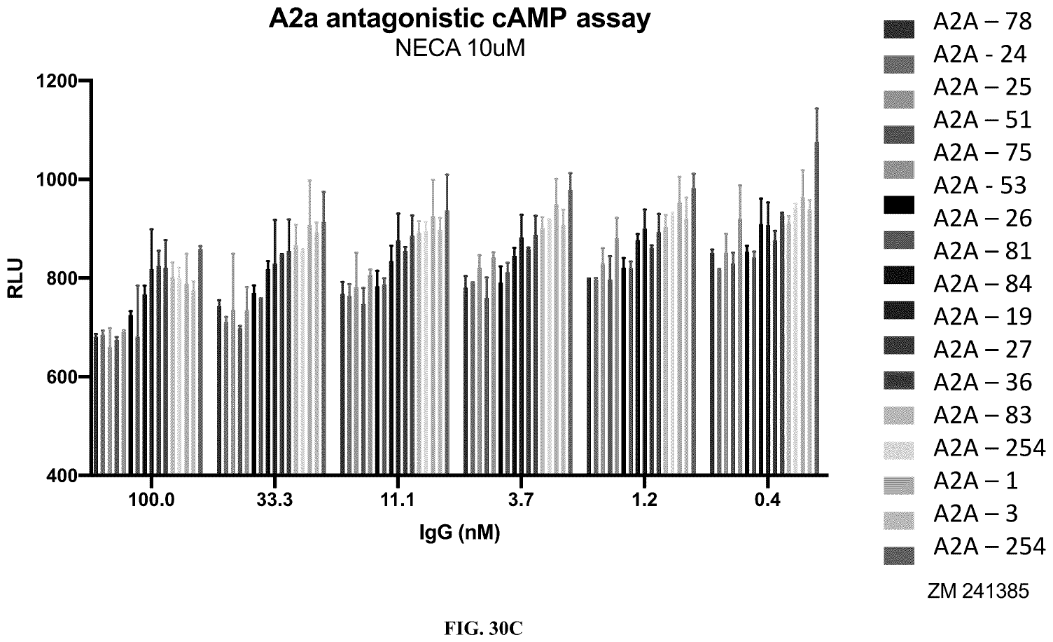

For a functional antagonistic cAMP assays, cells were pre-incubated with anti-A2AR antibody 3× titration from 100 nM followed by NECA stimulation at 0.5 uM. Data is seen in FIGS. 29A-29C. Cells were also pre-incubated with anti-A2AR antibody 3× titration from 100 nM followed by NECA stimulation at 10 uM. Data is seen in FIGS. 30A-30C.

Based on the data, for NECA titration, IgG titration (NECA 0.5 uM), and IgG titration (NECA 10 uM), A2AR variant A2A-17, A2A-19, A2A-24, A2A-26, and A2A-27 exhibited improved function in cAMP assays.

### Example 21. T Cell Activation Assays

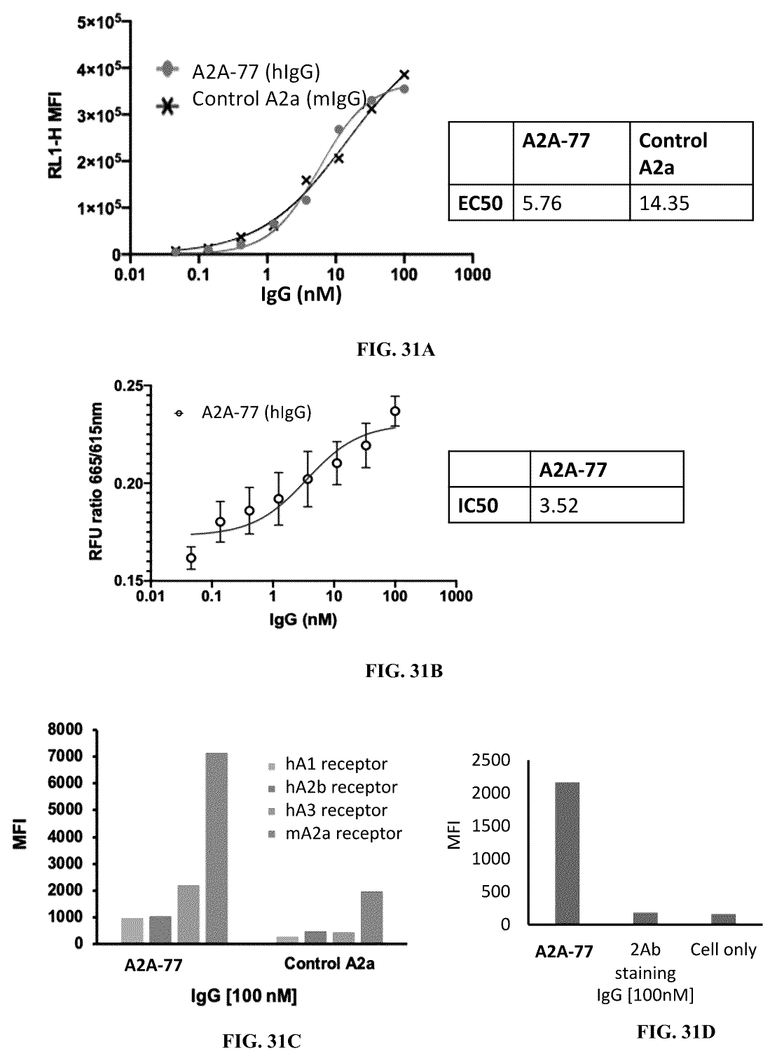

Variant A2A-77 developed according to the previous examples was found to be a high affinity binder for hA2a (FIG. 31A). A2A-77 was determined to be a functional antagonist in vitro (FIG. 31B) and had high specificity in vitro (FIG. 31C). A2A-77 was found to bind to cynomolgus PBMC with cells expressing A2AR, including T cell, NK cell, dendritic cell, and macrophage (FIG. 31D). Further studies were performed to determine effects in T-cell activation assays.

Briefly, 2×10⁵ PBMCs per well were incubated with antagonist ZM-241385 or A2aR immunoglobulins that were titrated from 100 nM for 30 minutes at 37° C., followed by treatment with A2AR agonist NECA at 1 μM for 30 minutes at 37° C. The cells were then activated by magnetic beads coating with anti-CD3E/CD28 antibodies. Three days after incubation, the supernatant was collected for detecting IFN-γ release and evaluating T cell activation. ZM-241385 is potent and used as a selective small molecule A2A antagonist control.

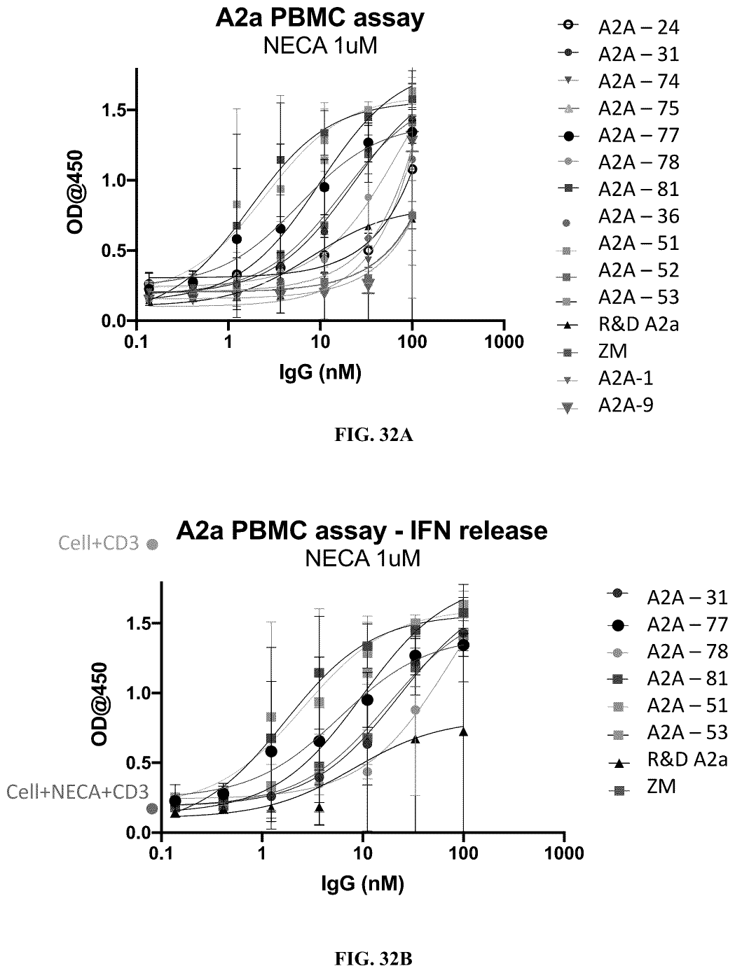

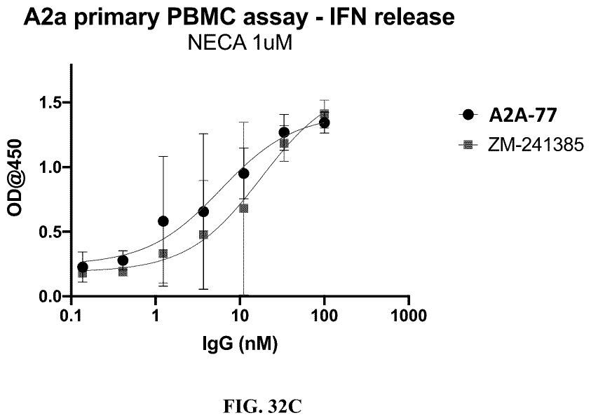

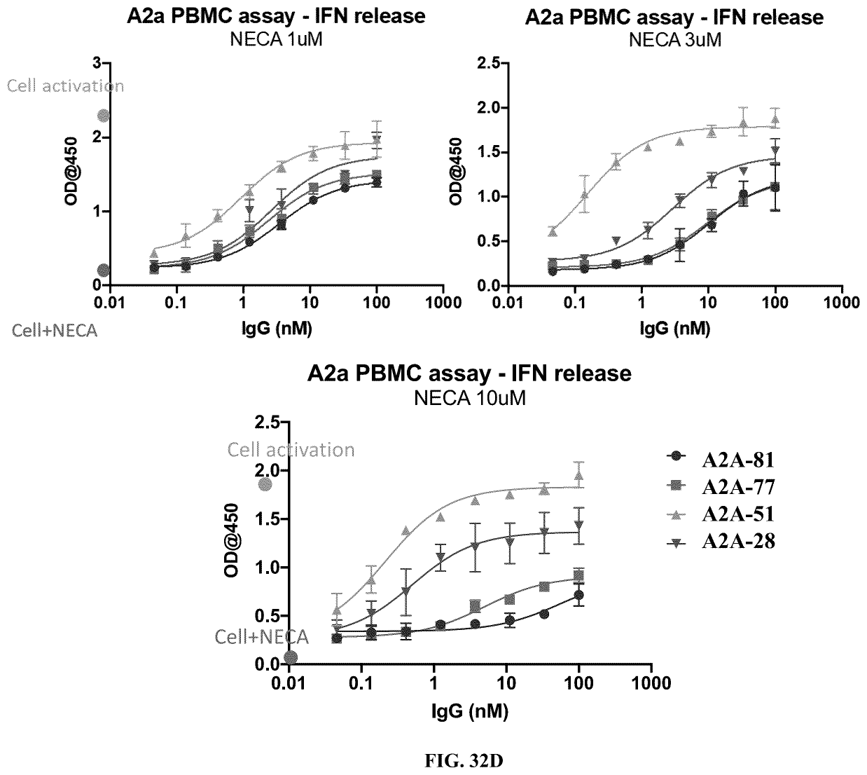

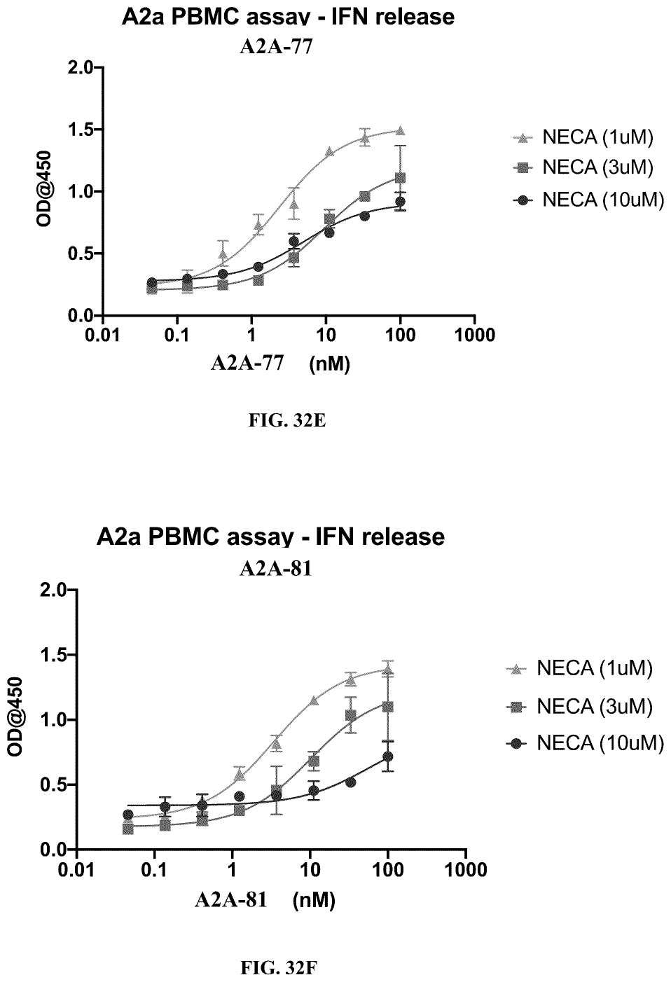

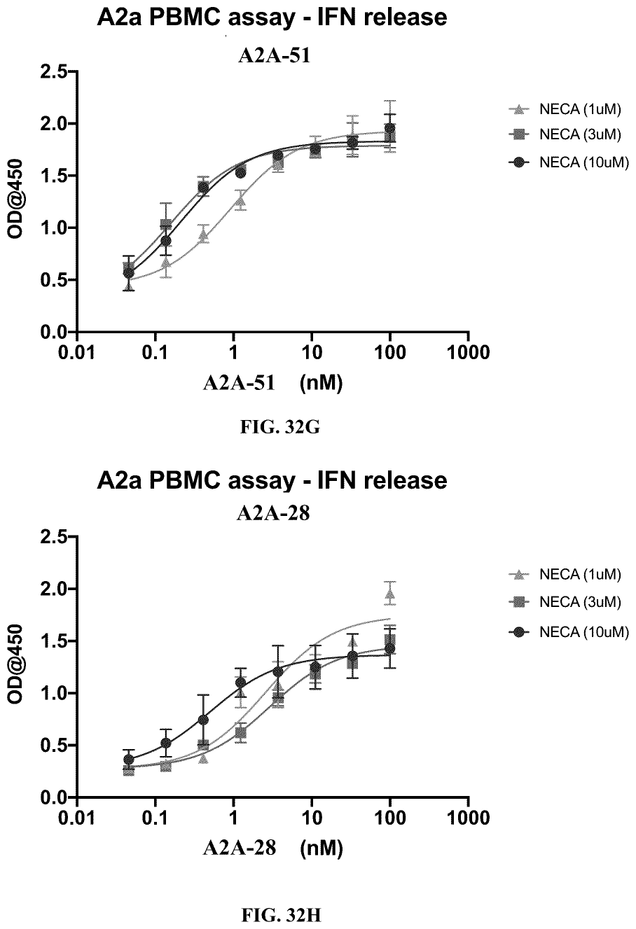

Data is seen in FIGS. 32A-32H. As seen in FIGS. 32A-32B, T cell activation was observed with variants A2A-81, A2A-51, A2A-53, A2A-77, A2A-31, and A2A-78. A2A-77 was further observed to have an IC₅₀ of 5.92 nM (FIG. 32C). Data in FIGS. 32D-32G demonstrate while using more NECA to suppress T cell activation, A2A-77 and A2A-81 cannot restore T cell activation as in low NECA. A2A-51 still works well in high NECA.

This Example shows that A2A-77 is a functional antagonist of A2AR to block immunosuppression.

### Example 22. Exemplary Sequences

TABLE 15
Variable Heavy Chain CDRs

| | SEQ | | SEQ | | SEQ | |
|---|---|---|---|---|---|---|
| A2AR | ID | CDR1 | ID | CDR2 | ID | CDR3 |
| Variant | NO | Sequence | NO | Sequence | NO | Sequence |
| A2A-1 | 6 | GGSISSSN | 95 | YPSGN | 184 | DEGY |
| A2A-2 | 7 | GYTFTGY | 96 | NPNSGG | 185 | GGPFDY |
| A2A-3 | 8 | GYTFTGY | 97 | NPNSGG | 186 | VYSYGFDY |
| A2A-4 | 9 | GFTFSDY | 98 | SSSGST | 187 | DNWAFDL |
| A2A-5 | 10 | GFTFSSY | 99 | SSSSSY | 188 | TWYSSSPFDY |
| A2A-6 | 11 | GFTFSNY | 100 | SSSSSY | 189 | DSGSYYDWFDP |
| A2A-7 | 12 | GFTFSSY | 101 | SGSGGS | 190 | YSNYFDY |
| A2A-8 | 13 | GYSITSGY | 102 | SYDGS | 191 | VHHYYGSSYFDY |
| A2A-9 | 14 | GYSITSGY | 103 | RYDGS | 192 | VHHYYGSSYFDY |
| A2A-10 | 15 | GYSITSGY | 104 | SYDGS | 193 | DPYYYGSSYFDY |
| A2A-11 | 16 | GFTFSDY | 105 | NYDGSS | 194 | EYYYGSSSFAY |
| A2A-12 | 17 | GFTFNDY | 106 | NYDGSS | 195 | EYYYGSSSFAY |
| A2A-13 | 18 | GFTFSDF | 107 | SSGSST | 196 | REFAY |
| A2A-14 | 19 | GFTFSDY | 108 | SSGSGT | 197 | PNYHGSSPFAY |
| A2A-15 | 20 | GFTFSTY | 109 | SGSGGS | 198 | ARGKWRWRLGRRYDY |
| A2A-16 | 21 | GFTFNNY | 110 | SGSGGD | 199 | ARGYWRWRLLRRYDY |
| A2A-17 | 22 | GFNIGNT | 111 | NPNYGT | 200 | DYGSSSFDY |
| A2A-18 | 23 | GFSFSGY | 112 | SGSGGS | 201 | ARGYPRWRLGRRYDY |
| A2A-19 | 24 | GFTFSGY | 113 | SGSGAS | 202 | ARGYKRWRLGRRYDY |
| A2A-20 | 25 | GFAFSNY | 114 | YPKSGS | 203 | LYGYDLHWYFDV |
| A2A-21 | 26 | GGSISSGGY | 115 | NPNSGN | 204 | DEVAAAGLFDY |
| A2A-22 | 27 | GYTFTEY | 116 | HPSSGS | 205 | HEVEYYGPSSSWFAY |
| A2A-23 | 28 | GFTFSTY | 117 | SGSGGS | 206 | ARGKWRWRLGRRYDY |
| A2A-24 | 29 | GFNIGNT | 118 | NPNYGT | 207 | DYGSSSFDY |
| A2A-25 | 30 | GFTFGNY | 119 | DPANGD | 208 | EGDNSNYYAMDY |
| A2A-26 | 31 | GFTFSTY | 120 | SGSAGS | 209 | ARGHWRWRLGRRYDY |
| A2A-27 | 32 | GFTFSSY | 121 | SGSGGS | 210 | ARGYWRWRLWRRYDY |
| A2A-28 | 33 | GFTFSSQ | 122 | SGSGVS | 211 | ARGRWRWRLGRRYDY |
| A2A-29 | 34 | GYSFTGY | 123 | YPGSGN | 212 | EDDYGWYFGV |
| A2A-30 | 35 | GYRLTGY | 124 | DPASGD | 213 | HEDPIYYGNYVFAY |
| A2A-31 | 36 | GYLFTDY | 125 | YPGTG | 214 | LYYGSSWERYFDV |
| A2A-32 | 37 | GFTFIDY | 126 | NPNYGT | 215 | QGSNYGGYFDV |
| A2A-33 | 38 | GFPFSSY | 127 | SGSGGR | 216 | ARGYWRWRLGRRADY |
| A2A-34 | 39 | GFNFNTY | 128 | YPGNSD | 217 | VIYYYGSSDYTLDY |
| A2A-35 | 40 | GFTFSTY | 129 | SGSGGS | 218 | ARGKWRWRLGRRYDY |
| A2A-36 | 41 | GFNIGNT | 130 | NPNYGT | 219 | DYGSSSFDY |
| A2A-37 | 42 | GYTFTSY | 131 | NHDGSN | 220 | SMITRFAY |
| A2A-38 | 43 | GFSLTSY | 132 | DPETDD | 221 | YYYGSSAFAY |
| A2A-39 | 44 | GFTFSNY | 133 | NPNNGG | 222 | AYYSNYGVMYF |
| A2A-40 | 45 | GFNFRSY | 134 | SGGGGS | 223 | ARGGWRWRLGRRYDY |
| A2A-41 | 46 | GFSLSIY | 135 | SPGSGS | 224 | PYYYGSSRYYAMDY |
| A2A-42 | 47 | GYTFTSY | 136 | SSGGDS | 225 | DYYGSSWHFDV |
| A2A-43 | 48 | GFTFSSY | 137 | SDGGSY | 226 | YIWYYGSSWSWYFDA |
| A2A-44 | 49 | GFTFSAY | 138 | GTAGD | 227 | GYNWIFDL |
| A2A-45 | 50 | GYSFTGY | 139 | LPGSGG | 228 | GNYDAMDY |
| A2A-46 | 51 | GGYISSSN | 140 | EQDGSE | 229 | GEYSRLWYFDL |
| A2A-47 | 52 | GTFTDY | 141 | LPGSGG | 230 | PYDYDFDY |
| A2A-48 | 53 | GYTFTSS | 142 | YPRDGS | 231 | TVVADWYFDV |
| A2A-49 | 54 | GYTFNDD | 143 | NPNNGA | 232 | KGDGGSYAAMDY |
| A2A-50 | 55 | GYSFTGY | 144 | YPKDGS | 233 | TVVADWYFDV |
| A2A-51 | 56 | GYTFNDY | 145 | NPNNGA | 234 | NYGSSYYALDY |
| A2A-52 | 57 | GYTFNDY | 146 | NPNNGG | 235 | QGSNYGGYFDV |
| A2A-53 | 58 | GFNIIDD | 147 | TDTGEP | 236 | DYIYAMDY |
| A2A-54 | 59 | GYTFTDY | 148 | DPANGD | 237 | GDYGSSYAMDY |
| A2A-55 | 60 | GYEFSSS | 149 | YPGTGN | 238 | YYYGSSAFAY |
| A2A-56 | 61 | GFTFSSY | 150 | DPGTGG | 239 | IYYDYSAMDY |
| A2A-57 | 62 | GFIFSDF | 151 | DPEDG | 240 | DYYGSSYLDY |
| A2A-58 | 63 | GFNIKDY | 152 | NPNNGG | 241 | DYYGSFHRRWYFDV |
| A2A-59 | 64 | GYTFTDY | 153 | NINNGG | 242 | DYHGSSFYWYFDV |
| A2A-60 | 65 | GYTFTEY | 154 | NFDGSS | 243 | YYDSSYYAMDY |
| A2A-61 | 66 | GFTFSTY | 155 | YPGDTD | 244 | GIAVAGTFDY |
| A2A-62 | 67 | GYTFTNY | 156 | NPNNGG | 245 | HALLWYYYAMDY |
| A2A-63 | 68 | GFTFSDH | 157 | NPNSGI | 246 | VSYSGSLHY |
| A2A-64 | 69 | GFTFDDY | 158 | NTNTGN | 247 | SNWNYFDY |
| A2A-65 | 70 | GSAFSAS | 159 | DPDNGD | 248 | PRDSGPSFAS |
| A2A-66 | 71 | GFTFSSY | 160 | YPKDGS | 249 | SRGYYYGSSYGYYDV |
| A2A-67 | 72 | GHTITSY | 161 | LPGSGT | 250 | NWGFAY |
| A2A-68 | 73 | GYTFSGY | 162 | DPSDSF | 251 | DYGSSYEFTY |
| A2A-69 | 74 | GGYISSSN | 163 | KTKTDGGT | 252 | GYSGSVDY |
| A2A-70 | 75 | GSNIKDY | 164 | SDGGS | 253 | DATGTFAY |
| A2A-71 | 76 | GGSISSSN | 165 | YHSGS | 254 | EVVSGMIGTVFDY |
| A2A-72 | 77 | GFTISTY | 166 | GTAGD | 255 | GYNWIFDY |
| A2A-73 | 78 | GFTVSTY | 167 | GTAGD | 256 | GYNWIFDF |
| A2A-74 | 79 | GFTFTTY | 168 | GTAGD | 257 | GYNWIFDF |
| A2A-75 | 80 | GGSISSSN | 169 | YHSGN | 258 | EVVSGMIGTIFDY |
| A2A-76 | 81 | GFTFSSY | 170 | GTAGD | 259 | GYNWIFDF |
| A2A-77 | 82 | GGSISSSN | 171 | YHSGN | 260 | EVVSGMIGTIFDY |
| A2A-78 | 83 | GFTFSAY | 172 | GTAGD | 261 | GYNWVFDL |
| A2A-79 | 84 | GFTFDDY | 173 | TWNGDR | 262 | DGLTGIFDY |
| A2A-80 | 85 | GFTISTY | 174 | GTAGD | 263 | GYNWIFDY |
| A2A-81 | 86 | GGSISSSN | 175 | YHSGS | 264 | EVVSGLYGTIFDY |
| A2A-82 | 87 | GYSITSGY | 176 | SYGGS | 265 | DYDYFDY |
| A2A-83 | 88 | GYAFSSY | 177 | YPGDGD | 266 | GAY |
| A2A-84 | 89 | GYTFTEY | 178 | SGGGSY | 267 | PNYSGSSPFAY |
| A2A-85 | 90 | GFSLTAY | 179 | WTGGG | 268 | SRGYYYGSSYGYFDV |
| A2A-86 | 91 | GYSITSD | 180 | NYSGS | 269 | KLDWDGYFDV |
| A2A-87 | 92 | GFNIKNT | 181 | DPANGN | 270 | GSPYGYDGHYVMDY |
| A2A-88 | 93 | GFTFRTY | 182 | SAEGSN | 271 | DGRGSLPRPKGG |
| | | | | | | FIGALSFHWPFG |
| | | | | | | RWLGGSYGTYDS |
| | | | | | | SEDSGGAFDI |
| A2A-89 | 94 | GFTFNNY | 183 | SYGGSD | 272 | DGRGSLPRPKGG |
| | | | | | | FIGDLSFHWPFG |
| | | | | | | RWLGKSYGTYDS |
| | | | | | | SEDSGGAFDI |

TABLE 16
Variable Light Chain CDRs

| A2AR | SEQ | | SEQ | | SEQ | |
|---|---|---|---|---|---|---|
| Variant | ID NO | CDR1 Sequence | ID NO | CDR2 Sequence | ID NO | CDR3 Sequence |
| A2A-1 | 273 | RSSQSLVYSDGNTYLN | 362 | KVSNRDS | 451 | MQGTHWPRT |
| A2A-2 | 274 | KASQDIDDDMN | 363 | EATTLVP | 452 | LQHDNFPMYT |
| A2A-3 | 275 | KSSQSVLYSSNNKNYLA | 364 | WASTRES | 453 | QQYYSTPYT |
| A2A-4 | 276 | RASQSVSSNLA | 365 | GASTRAT | 454 | QQYYSTPLT |
| A2A-5 | 277 | KASQDIDDDMN | 366 | EATTLVP | 455 | LQHDNFPWT |
| A2A-6 | 278 | RASQGISSWLA | 367 | AASSLQS | 456 | QQTNSFPRT |
| A2A-7 | 279 | KASQDVDDDMN | 368 | EATTLVP | 457 | LQHDNFPWT |
| A2A-8 | 280 | KASQNVGTNVA | 369 | SASYRYS | 458 | QRFNNYPLT |
| A2A-9 | 281 | KASQNVGSSVA | 370 | STSYRYS | 459 | QQYNSYPLT |
| A2A-10 | 282 | RASQSISDYLH | 371 | YASQSIS | 460 | QNGHSFPLT |
| A2A-11 | 283 | KASRNVGTNVA | 372 | SASYRYS | 461 | QQYNSYPLT |
| A2A-12 | 284 | RASQSISDYLH | 373 | YASQSIS | 462 | QNGHSFPHT |
| A2A-13 | 285 | KASQNVGTNVA | 374 | SASYRYS | 463 | QQYNIYPLT |
| A2A-14 | 286 | RASQSISNYLH | 375 | YASQSIS | 464 | QNGHSFPLT |
| A2A-15 | 287 | RASQSIGRYLN | 376 | AASSLHS | 465 | QQSYVTPWT |
| A2A-16 | 288 | RASQSIGTYLN | 377 | GASTLHS | 466 | QQSYSAPWT |
| A2A-17 | 289 | KASQSVRNDVV | 378 | RGNTLRP | 467 | QQYYGIPLT |
| A2A-18 | 290 | RASQSVTTYLN | 379 | SASSLQS | 468 | QQTYATPWT |
| A2A-19 | 291 | RASQSISDYLN | 380 | TASTLQS | 469 | EQSYSTPWT |
| A2A-20 | 292 | KASHSVDYDGDNYMN | 381 | WASTRLT | 470 | LQHIEYPFT |
| A2A-21 | 293 | KSSQSVLYSSNNKNYFA | 382 | DAPNRAT | 471 | QQGYTTPYT |
| A2A-22 | 294 | RASQDIGRSLS | 383 | DASRFIS | 472 | QWSNSWPYT |
| A2A-23 | 295 | RASQSIGRYLN | 384 | AASSLHS | 473 | QQSYVTPWT |
| A2A-24 | 296 | KASQSVRNDVV | 385 | RGNTLRP | 474 | QQYYGIPLT |
| A2A-25 | 297 | KASQSVDYDGDSYMN | 386 | RANRLVD | 475 | QNGHSFPLT |
| A2A-26 | 298 | RASQTISRYLN | 387 | SASTLQS | 476 | QQSYSTPHT |
| A2A-27 | 299 | RASQSIGSYLN | 388 | GASNLQS | 477 | QQGYSAPRT |
| A2A-28 | 300 | RASRSISSYLN | 389 | AASSLPS | 478 | QQSYSTPRT |
| A2A-29 | 301 | KVSQDVRTAVA | 390 | DTSYLAS | 479 | QQSYSWSLT |
| A2A-30 | 302 | GGGNDIGSSMY | 391 | WMSNLAS | 480 | QQYSTYPFA |
| A2A-31 | 303 | RASQSISDYLN | 392 | GASPRES | 481 | QQDNIWPYT |
| A2A-32 | 304 | GGGNDIGSSMY | 393 | DASRFIS | 482 | QQSNEDPPFT |
| A2A-33 | 305 | RASESVDSFGNNFMN | 394 | HTSRLNS | 483 | QQNNEVPRT |
| A2A-34 | 306 | RASSSVTYIH | 395 | AVSRLDS | 484 | HQSNEDPYT |
| A2A-35 | 307 | RASQSIGRYLN | 396 | AASSLHS | 485 | QQSYVTPWT |
| A2A-36 | 308 | KASQSVRNDVV | 397 | RGNTLRP | 486 | QQYYGIPLT |
| A2A-37 | 309 | KASHSVDYDGDNYMN | 398 | DASRFIS | 487 | LRYASYRT |
| A2A-38 | 310 | RASESVNSYGNSFMH | 399 | DASRFIS | 488 | LQYGESPLT |
| A2A-39 | 311 | RSSKSLLHSSGNAYVY | 400 | YTSKPNS | 489 | QHHYGIPLT |
| A2A-40 | 312 | RASQSIGTYLN | 401 | AASSLES | 490 | QQTYNTPWT |
| A2A-41 | 313 | RASSRVSSSYLY | 402 | ATYSLDY | 491 | LQHGERPLT |
| A2A-42 | 314 | GASQSIGTIIH | 403 | DTSYLAS | 492 | QQGNTRPWT |
| A2A-43 | 315 | RASENIYVPLN | 404 | DASRFIS | 493 | QQYNSFPLYT |
| A2A-44 | 316 | RASQSVSSSYLA | 405 | GASSRAT | 494 | QQYGSSPIT |
| A2A-45 | 317 | KSSQSLLYSGEKTYPY | 406 | WASTRLT | 495 | QQSNEDSWT |
| A2A-46 | 318 | QSSQDIFNYLE | 407 | TASNLDT | 496 | QQGYSTPPEIT |
| A2A-47 | 319 | RSTRNILSNMP | 408 | NANTLAE | 497 | LQHWNYPYM |
| A2A-48 | 320 | RASQDISNNLH | 409 | EISGWLS | 498 | QQSNSWSLLT |
| A2A-49 | 321 | SASQSMSNNLH | 410 | LASNLGY | 499 | RQNGHSFPLT |
| A2A-50 | 322 | RASQDISNNLH | 411 | WASTRLT | 500 | QQWSDYPFT |
| A2A-51 | 323 | SASSSLSYMH | 412 | GASPRES | 501 | RQMSSYPPT |
| A2A-52 | 324 | SASSSVSYMN | 413 | EISGWLS | 502 | LRYASYRT |
| A2A-53 | 325 | KASQNMGSNVA | 414 | SASHRSS | 503 | QQWNYPRIT |
| A2A-54 | 326 | KASQNGGTNVD | 415 | EISGWLS | 504 | QHYYSWPPT |
| A2A-55 | 327 | RASENIYVPLN | 416 | LASYRFT | 505 | QQINGWPYT |
| A2A-56 | 328 | KASQNMGSNVA | 417 | AATRLAD | 506 | RQHYSSPPT |
| A2A-57 | 329 | KASQNGGTNVD | 418 | VASNQGT | 507 | QQYYTYPLT |
| A2A-58 | 330 | KASQGVDTNVA | 419 | SSSIS | 508 | AQNRELPFT |
| A2A-59 | 331 | KASQDVGTAIT | 420 | SASKRNT | 509 | LHHYGTPYT |
| A2A-60 | 332 | KASQDVGTSVA | 421 | PASYRSS | 510 | QQGSSNPLT |
| A2A-61 | 333 | RASQVIDDDIN | 422 | LGSNRAP | 511 | HQSYTTPHT |
| A2A-62 | 334 | RASQEISGYLT | 423 | SASHRSS | 512 | QQWDNNPYT |
| A2A-63 | 335 | RASQSISRYLN | 424 | KASSLER | 513 | LQPNSYPWT |
| A2A-64 | 336 | RASQGISSWLD | 425 | TPFSLQS | 514 | QHYDDLPLT |
| A2A-65 | 337 | KASQNMGSNVA | 426 | EASTRFS | 515 | QQYSSYPLR |
| A2A-66 | 338 | RASQGILGYLN | 427 | STSNLLL | 516 | RQLSSNPLT |
| A2A-67 | 339 | RASESVDNYGISFMS | 428 | DASRFIS | 517 | QQINSWPLT |
| A2A-68 | 340 | KSSQSLLYSGEKTYPY | 429 | EASNRYT | 518 | QQWSSYPPIA |
| A2A-69 | 341 | RASQGLRHDLG | 430 | WASNRES | 519 | QKYSSTPYT |
| A2A-70 | 342 | HASESVSVAGTSLLH | 431 | AASNRES | 520 | QHWSSFPLT |
| A2A-71 | 343 | RVSQGISNYLN | 432 | AASSLQS | 521 | QQSYSTPLT |
| A2A-72 | 344 | RASQSVSSNLA | 433 | GASSRAT | 522 | QQYGSSPPT |
| A2A-73 | 345 | RASQSVSSNLA | 434 | GASSRAT | 523 | QQYGSSPLT |
| A2A-74 | 346 | RASQSVSSSYLA | 435 | GASSRAT | 524 | QQYYSTPLT |
| A2A-75 | 347 | RASQSISSYLN | 436 | AASSLQS | 525 | QQANSFPIT |
| A2A-76 | 348 | RASQSVSSNLA | 437 | DASNRAT | 526 | QQYGSSPLT |
| A2A-77 | 349 | RASQRISSYLN | 438 | AASSLQS | 527 | QQSYSTPLT |
| A2A-78 | 350 | RAIQSVSSSYLA | 439 | GASSRAT | 528 | QQYGSSPLT |
| A2A-79 | 351 | RASQSVSSYLA | 440 | GASSRAT | 529 | QQYGNSYT |
| A2A-80 | 352 | RASQSVSSNLA | 441 | GASTRAT | 530 | QQYGSSPPT |
| A2A-81 | 353 | RASQSISSYLN | 442 | AASSLQS | 531 | QQSYSTPIT |
| A2A-82 | 354 | KASQSVSNDVA | 443 | YASNRYT | 532 | QQDYRSPLT |
| A2A-83 | 355 | KASQNVGTNVA | 444 | SASYRYS | 533 | QQYNSYPLT |
| A2A-84 | 356 | SASSSVSYMY | 445 | DTSNLAS | 534 | QQWNSNPLT |
| A2A-85 | 357 | RASQSISDYLH | 446 | YASQSIS | 535 | QNGHSFPLT |
| A2A-86 | 358 | HASQNINVWLN | 447 | KASNLHT | 536 | QQGQSYPLT |
| A2A-87 | 359 | KASQNVGSNVA | 448 | SASYRYS | 537 | QQYNSYPLT |
| A2A-88 | 360 | SGISSNIGNNYVS | 449 | DNNKRASG | 538 | GTWDTSLSAGV |
| A2A-89 | 361 | SGSSSNIGNHYVS | 450 | DNTKRPSG | 539 | GTWDASLSTWV |

TABLE 17
Variable Heavy Chain Sequences

| A2AR Variant | SEQ ID NO | Sequence |
|---|---|---|
| A2A-1 | 540 | QVQLQESGPGLVKPSGTLSLTCAVSGGSISSSNWWSWVRQPPG |
| | | KGLEWIGEIYPSGNTYYNPSLKSRVTISVDKSKNQFSLKLNSVT |
| | | AADTAVYYCARDEGYWGQGTLVTVSS |
| A2A-2 | 541 | EVQLLESGAEVKKPGASVKVSCKASGYTFTGYYMHWVRQAP |
| | | GQGLEWMGWINPNSGGTNYAQKFQGRVTMTRDTSISTAYME |
| | | LSSLRSEDTAVYYCARGGPFDYWGQGTMVTVSS |
| A2A-3 | 542 | EVQLLESGAEVKKPGASVKASCKASGYTFTGYYMHWVRQAP |
| | | GQGLEWMGWINPNSGGTNYAQKFQGRVTMTRDTSISTAYME |
| | | LSRLRSDDTAVYYCARVYSYGFDYWGQGTLVTVSS |
| A2A-4 | 543 | AGQLQESGGGLVKPGGSLRPSCAASGFTFSDYYMSWIRQAPG |
| | | KGLEWVSYISSSGSTIYYADSVKGRFTISRDNAKNSLYLQMNSL |
| | | RAEDTAVYYCARDNWAFDLWGQGTLVTVSS |
| A2A-5 | 544 | GGALVQSGGGLVQPGGSLRLSCAASGFTFSSYSMNWVRQATG |
| | | KGLEWVSSISSSSSYIYYADSVKGRFTISRDNAKNSLHLQMNSL |
| | | RDEDTAVYYCARTWYSSSPFDYWGQGTLVTVSS |
| A2A-6 | 545 | EVQLLESGGGLVKPGGSPRLSCAASGFTFSNYSMNWVRQAPG |
| | | KGLEWVSSISSSSSYIYYADSVNGRFTISRDYAKNSLYLQMNSL |
| | | RAEDTAVCYCARDSGSYYDWFDPWGQGTLVTVSS |
| A2A-7 | 546 | QVQLVQSGGGLVQPGGSLRLSCAASGFTFSSYAMSWVRRAPG |
| | | KGLEWVSAISGSGGSTYYADSVKGRFTISRDNSKNTLYLQMNS |
| | | LRAEDTAVYYCAKYSNYFDYWGQGTLVTVSS |
| A2A-8 | 547 | EVQLQQPGPGLVKPSLSLSHTCSVTGYSITSGYYWNWIRQFPG |
| | | KKLEWMGYISYDGSNNYNPSLKNRTSITRDTSKNQFFLKLSSV |
| | | TTDDTATYYCARVHHYYGSSYFDYWGQGTTLTVSS |
| A2A-9 | 548 | EVQLQQSGPGLVKPSQSLSLTCSVTGYSITSGYYWNWIRQFPG |
| | | KKLEWMGYIRYDGSNNYNPSLKNRISITRDTSKNQFFLKLNSV |
| | | TTDDTATYYCARVHHYYGSSYFDYWGQGTTLTVSS |
| A2A-10 | 549 | EVQLQQSGPGLVKPSQSLSLTCSVTGYSITSGYYWNWIRQFPG |
| | | NKLEWMGYISYDGSNNYNPSLKNRISITRDTSKNQFFLKLNSV |
| | | TTEDTATYYCARDPYYYGSSYFDYWGQGTTLTVSS |
| A2A-11 | 550 | EVKLVESEGGLVQPGSSMKLSCTASGFTFSDYYMAWVRQVPE |
| | | KGLEWVANINYDGSSTYYLDSLKSRFTISRDNAKNILYLQMSS |
| | | LKSEDIATYYCAREYYYGSSSFAYWGQGTTLTVSS |
| A2A-12 | 551 | EVNPVESEGGLVQPGSSMKLSCTASGFTFNDYYMAWVRQVPE |
| | | KGLEWVANINYDGSSTYYLDSLKSRFIISRDNAKNILYLQMSSL |
| | | KSEDTATYYCAREYYYGSSSFAYWGQGTLVTVSA |
| A2A-13 | 552 | GGEVVESGGGLVKPGGSLKLSCAASGFTFSDFGMHWVRQAPE |
| | | KGLEWVAYISSGSSTIYYADTVKGRFTISRDNAKNTLFLQMTSL |
| | | RSEDTAMYYCARREFAYWGQGTLVTVSA |
| A2A-14 | 553 | EVKLEESGGGLVKPGGSLKLSCAVSGFTFSDYGMHWVRQAPE |
| | | KGLEWVAYISSGSGTIYYEDTVKGRFTISRDNAKNTLFLQMTS |
| | | LRSEGTAIYYCARPNYHGSSPFAYWGQGTLVTVSA |
| A2A-15 | 554 | EVQLVESGGGLVKPGGSLRLSCAASGFTFSTYGMSWVRQAPG |
| | | KGLEWVSGISGSGGSTNYADSVKGRFTISRDNSKNTLYLQMNS |
| | | LRAEDTAVYYCAAARGKWRWRLGRRYDYWGQGTLVTVSS |
| A2A-16 | 555 | EVQLVESGGGLVKPGGSLRLSCAASGFTFNNYAMNWVRQAPG |
| | | KGLEWVSSISGSGGDTYYADSVKGRFTISRDNSKNTLYLQMNS |
| | | LRAEDTAVYYCAAARGYWRWRLLRRYDYWGQGTLVTVSS |
| A2A-17 | 556 | EVQLVESGGGLVKPGGSLRLSCAASGFNIGNTYMHWFRQAPG |
| | | KGLEWVGVINPNYGTTRYNDSVKGRFTISRDNSKNTLYLQMN |
| | | SLRAEDTAVYYCARDYGSSSFDYWGQGTLVTVSS |
| A2A-18 | 557 | EVQLVESGGGLVKPGGSLRLSCAASGFSFSGYAMSWVRQAPG |
| | | KGLEWVSVISGSGGSTNYADSVKGRFTISRDNSKNTLYLQMNS |
| | | LRAEDTAVYYCAAARGYPRWRLGRRYDYWGQGTLVTVSS |
| A2A-19 | 558 | EVQLVESGGGLVKPGGSLRLSCAASGFTFSGYAMNWVRQAPG |
| | | KGLEWVSTISGSGASTYYADSVKGRFTISRDNSKNTLYLQMNS |
| | | LRAEDTAVYYCAAARGYKRWRLGRRYDYWGQGTLVTVSS |
| A2A-20 | 559 | EVQLVESGGGLVKPGGSLRLSCAASGFAFSNYWMNWVRQAP |
| | | GKGLEWVGWFYPKSGSIKYNDSVKGRFTISRDNSKNTLYLQM |
| | | NSLRAEDTAVYYCTGLYGYDLHWYFDVWGQGTLVTVSS |
| A2A-21 | 560 | QVQLVQSGAEVKKPGASVKVSCKASGGSISSGGYYWNWVRQ |
| | | ATGQGLEWMGWMNPNSGNRGSAQKFQGRVTMTRDTSTSTV |
| | | YMELSSLRSEDTAVYYCARDEVAAAGLFDYWGQGTLVTVSS |
| A2A-22 | 561 | EVQLVESGGGLVKPGGSLRLSCAASGYTFTEYITHWVRQAPGK |
| | | GLEWVGMIHPSSGSISYNDSVKGRFTISRDNSKNTLYLQMNSL |
| | | RAEDTAVYYCARHEVEYYGPSSSWFAYWGQGTLVTVSS |
| A2A-23 | 562 | EVQLVESGGGLVKPGGSLRLSCAASGFTFSTYGMSWVRQAPG |
| | | KGLEWVSGISGSGGSTNYADSVKGRFTISRDNSKNTLYLQMNS |
| | | LRAEDTAVYYCAAARGKWRWRLGRRYDYWGQGTLVTVSS |
| A2A-24 | 563 | EVQLVESGGGLVKPGGSLRLSCAASGFNIGNTYMHWFRQAPG |
| | | KGLEWVGVINPNYGTTRYNDSVKGRFTISRDNSKNTLYLQMN |
| | | SLRAEDTAVYYCARDYGSSSFDYWGQGTLVTVSS |
| A2A-25 | 564 | EVQLVESGGGLVKPGGSLRLSCAASGFTFGNYWMNWVRQAP |
| | | GKGLEWVGRIDPANGDTKYADSVKGRFTISRDNSKNTLYLQM |
| | | NSLRAEDTAVYYCAREGDNSNYYAMDYWGQGTLVTVSS |
| A2A-26 | 565 | EVQLLESGGGLVQPGGSLRLSCAASGFTFSTYAMGWVRQAPG |
| | | KGLEWVSGISGSAGSTYYADSVKGRFTISRDNSKNTLYLQMNS |
| | | LRAEDTAVYYCAAARGHWRWRLGRRYDYWGQGTLVTVSS |
| A2A-27 | 566 | EVQLVESGGGLVKPGGSLRLSCAASGFTFSSYAMAWVRQAPG |
| | | KGLEWVSAISGSGGSTYFADSVKGRFTISRDNSKNTLYLQMNS |
| | | LRAEDTAVYYCAAARGYWRWRLWRRYDYWGQGTLVTVSS |
| A2A-28 | 567 | EVQLVESGGGLVKPGGSLRLSCAASGFTFSSQAMSWVRQAPG |
| | | KGLEWVSSISGSGVSTYYADSVKGRFTISRDNSKNTLYLQMNS |
| | | LRAEDTAVYYCAAARGRWRWRLGRRYDYWGQGTLVTVSS |
| A2A-29 | 568 | EVQLVESGGGLVKPGGSLRLSCAASGYSFTGYDISWVRQAPGK |
| | | GLEWVARIYPGSGNTYYDDSVKGRFTISRDNSKNTLYLQMNSL |
| | | RAEDTAVYYCAREDDYGWYFGVWGQGTLVTVSS |
| A2A-30 | 569 | EVQLVESGGGLVKPGGSLRLSCAASGYRLTGYWIEWVRQAPG |
| | | KGLEWVGRIDPASGDTTYADSVKGRFTISRDNSKNTLYLQMNS |
| | | LRAEDTAVYYCARHEDPIYYGNYVFAYWGQGTLVTVSS |
| A2A-31 | 570 | EVQLVESGGGLVKPGGSLRLSCAASGYLFTDYNMNWVRQAP |
| | | GKGLEWVGWIYPGTGNTYNDSVKGRFTISRDNSKNTLYLQMN |
| | | SLRAEDTAVYYCTALYYGSSWERYFDVWGQGTLVTVSS |
| A2A-32 | 571 | EVQLVESGGGLVKPGGSLRLSCAASGFTFIDYGMHWVRQAPG |
| | | KGLEWVGVINPNYGTTRYNDSVKGRFTISRDNSKNTLYLQMN |
| | | SLRAEDTAVYYCARQGSNYGGYFDVWGQGTLVTVSS |
| A2A-33 | 572 | EVQLVESGGGLVKPGGSLRLSCAASGFPFSSYAMTWVRQAPG |
| | | KGLEWVSGISGSGGRTYYADSVKGRFTISRDNSKNTLYLQMNS |
| | | LRAEDTAVYYCAAARGYWRWRLGRRADYWGQGTLVTVSS |
| A2A-34 | 573 | EVQLVESGGGLVKPGGSLRLSCAASGFNFNTYAMNWVRQAPG |
| | | KGLEWVGVIYPGNSDTTYNDSVKGRFTISRDNSKNTLYLQMN |
| | | SLRAEDTAVYYCTGVIYYYGSSDYTLDYWGQGTLVTVSS |
| A2A-35 | 574 | EVQLVESGGGLVKPGGSLRLSCAASGFTFSTYGMSWVRQAPG |
| | | KGLEWVSGISGSGGSTNYADSVKGRFTISRDNSKNTLYLQMNS |
| | | LRAEDTAVYYCAAARGKWRWRLGRRYDYWGQGTLVTVSS |
| A2A-36 | 575 | EVQLVESGGGLVKPGGSLRLSCAASGFNIGNTYMHWFRQAPG |
| | | KGLEWVGVINPNYGTTRYNDSVKGRFTISRDNSKNTLYLQMN |
| | | SLRAEDTAVYYCARDYGSSSFDYWGQGTLVTVSS |
| A2A-37 | 576 | EVQLVESGGGLVKPGGSLRLSCAASGYTFTSYWVHWVRQAPG |
| | | KGLEWVANINHDGSNTYYLDSVKGRFTISRDNSKNTLYLQMN |
| | | SLRAEDTAVYYCASSMITRFAYWGQGTLVTVSS |
| A2A-38 | 577 | EVQLVESGGGLVKPGGSLRLSCAASGFSLTSYNIDWVRQAPGK |
| | | GLEWVGGVDPETDDTAYNDSVKGRFTISRDNSKNTLYLQMNS |
| | | LRAEDTAVYYCANYYYGSSAFAYWGQGTLVTVSS |
| A2A-39 | 578 | EVQLVESGGGLVKPGGSLRLSCAASGFTFSNYYMSWVRQAPG |
| | | KGLEWVGDINPNNGGTTYNDSVKGRFTISRDNSKNTLYLQMN |
| | | SLRAEDTAVYYCTKAYYSNYGVMYFWGQGTLVTVSS |
| A2A-40 | 579 | EVQLVESGGGLVKPGGSLRLSCAASGFNFRSYAMSWVRQAPG |
| | | KGLEWVSVISGGGGSTYYADSVKGRFTISRDNSKNTLYLQMNS |
| | | LRAEDTAVYYCAAARGGWRWRLGRRYDYWGQGTLVTVSS |
| A2A-41 | 580 | EVQLVESGGGLVKPGGSLRLSCAASGFSLSIYGISWVRQAPGK |
| | | GLEWVGDISPGSGSTNYNDSVKGRFTISRDNSKNTLYLQMNSL |
| | | RAEDTAVYYCAGPYYYGSSRYYAMDYWGQGTLVTVSS |
| A2A-42 | 581 | EVQLVESGGGLVKPGGSLRLSCAASGYTFTSYNINWVRQAPG |
| | | KGLEWVATISSGGDSIYYADSVKGRFTISRDNSKNTLYLQMNS |
| | | LRAEDTAVYYCERDYYGSSWHFDVWGQGTLVTVSS |
| A2A-43 | 582 | EVQLVESGGGLVKPGGSLRLSCAASGFTFSSYDMYWVRQAPG |
| | | KGLEWVASISDGGSYTYYVDSVKGRFTISRDNSKNTLYLQMNS |
| | | LRAEDTAVYYCARYIWYYGSSWSWYFDAWGQGTLVTVSS |
| A2A-44 | 583 | EVQLVESGGGVVQPGRSLRLSCAASGFTFSAYDIYWVRQPTGK |
| | | GLEWVSAIGTAGDTYYPGSVKGRFIISRESAKNSVYLQMNSLR |
| | | AGDTAVYYCAVGYNWIFDLWGQGTLVTVSS |
| A2A-45 | 584 | EVQLVESGGGLVKPGGSLRLSCAASGYSFTGYDISWFRQAPGK |
| | | GLEWVGEILPGSGGTNYNDSVKGRFTISRDNSKNTLYLQMNSL |
| | | RAEDTAVYYCTSGNYDAMDYWGQGTLVTVSS |
| A2A-46 | 585 | QVQLVQSGAEVKKPGASVKVSCKASGGYISSSNWWSWVRQA |
| | | TGQGLEWMANIEQDGSEKNYVQKFQGRVTMTRDTSTSTVYM |
| | | ELSSLRSEDTAVYYCARGEYSRLWYFDLWGQGTLVTVSS |
| A2A-47 | 586 | EVQLVESGGGLVKPGGSLRLSCAASGTFTDYYMKWVRQAPG |
| | | KGLEWVGEILPGSGGTNYNDSVKGRFTISRDNSKNTLYLQMNS |
| | | LRAEDTAVYYCARPYDYDFDYWGQGTLVTVSS |
| A2A-48 | 587 | EVQLVESGGGLVKPGGSLRLSCAASGYTFTSSWMHWARQAPG |
| | | KGLEWVGWLYPRDGSTEYNDSVKGRFTISRDNSKNTLYLQMN |
| | | SLRAEDTAVYYCITTVVADWYFDVWGQGTLVTVSS |
| A2A-49 | 588 | EVQLVESGGGLVKPGGSLRLSCAASGYTFNDDYTNWVRQAPG |
| | | KGLEWVGNINPNNGAMIYNDSVKGRFTISRDNSKNTLYLQMN |
| | | SLRAEDTAVYYCARKGDGGSYAAMDYWGQGTLVTVSS |
| A2A-50 | 589 | EVQLVESGGGLVKPGGSLRLSCAASGYSFTGYDISWVRQAPGK |
| | | GLEWVGWIYPKDGSTKYNDSVKGRFTISRDNSKNTLYLQMNS |
| | | LRAEDTAVYYCITTVVADWYFDVWGQGTLVTVSS |
| A2A-51 | 590 | EVQLVESGGGLVKPGGSLRLSCAASGYTFNDYYINWVRQAPG |
| | | KGLEWVGDINPNNGANIYNDSVKGRFTISRDNSKNTLYLQMN |
| | | SLRAEDTAVYYCARNYGSSYYALDYWGQGTLVTVSS |
| A2A-52 | 591 | EVQLVESGGGLVKPGGSLRLSCAASGYTFNDYYINWVRQAPG |
| | | KGLEWVGDINPNNGGTTYNDSVKGRFTISRDNSKNTLYLQMN |
| | | SLRAEDTAVYYCARQGSNYGGYFDVWGQGTLVTVSS |
| A2A-53 | 592 | EVQLVESGGGLVKPGGSLRLSCAASGFNIIDDYMHWVRQAPG |
| | | KGLEWVGMITDTGEPTDADSVKGRFTISRDNSKNTLYLQMNS |
| | | LRAEDTAVYYCVYDYIYAMDYWGQGTLVTVSS |
| A2A-54 | 593 | EVQLVESGGGLVKPGGSLRLSCAASGYTFTDYDMYWVRQAP |
| | | GKGLEWVGRIDPANGDTKYADSVKGRFTISRDNSKNTLYLQM |
| | | NSLRAEDTAVYYCARGDYGSSYAMDYWGQGTLVTVSS |
| A2A-55 | 594 | EVQLVESGGGLVKPGGSLRLSCAASGYEFSSSWMNWVRQAPG |
| | | KGLEWVGWIYPGTGNTNYNDSVKGRFTISRDNSKNTLYLQMN |
| | | SLRAEDTAVYYCANYYYGSSAFAYWGQGTLVTVSS |
| A2A-56 | 595 | EVQLVESGGGLVKPGGSLRLSCAASGFTFSSYGLPWVRQAPGK |
| | | GLEWVGAIDPGTGGTASNDSVKGRFTISRDNSKNTLYLQMNSL |
| | | RAEDTAVYYCARIYYDYSAMDYWGQGTLVTVSS |
| A2A-57 | 596 | EVQLVESGGGLVKPGGSLRLSCAASGFIFSDFYMAWVRQAPG |
| | | KGLEWVGRIDPEDGDEHADSVKGRFTISRDNSKNTLYLQMNS |
| | | LRAEDTAVYYCARDYYGSSYLDYWGQGTLVTVSS |
| A2A-58 | 597 | EVQLVESGGGLVKPGGSLRLSCAASGFNIKDYYMHWVRQAPG |
| | | KGLEWVGDINPNNGGTTYNDSVKGRFTISRDNSKNTLYLQMN |
| | | SLRAEDTAVYYCARDYYGSFHRRWYFDVWGQGTLVTVSS |
| A2A-59 | 598 | EVQLVESGGGLVKPGGSLRLSCAASGYTFTDYNIDWVRQAPG |
| | | KGLEWVGDININNGGTTYNDSVKGRFTISRDNSKNTLYLQMNS |
| | | LRAEDTAVYYCARDYHGSSFYWYFDVWGQGTLVTVSS |
| A2A-60 | 599 | EVQLVESGGGLVKPGGSLRLSCAASGYTFTEYITHWVRQAPGK |
| | | GLEWVANINFDGSSTYYLDSVKGRFTISRDNSKNTLYLQMNSL |
| | | RAEDTAVYYCARYYDSSYYAMDYWGQGTLVTVSS |
| A2A-61 | 600 | QVQLVQSGAEVKKPGASVKVSCKASGFTFSTYIMSWVRQATG |
| | | QGLEWMGHYPGDTDTRYSQKFQGRVTMTRDTSTSTVYMELSS |
| | | LRSEDTAVYYCARGIAVAGTFDYWGQGTLVTVSS |
| A2A-62 | 601 | EVQLVESGGGLVKPGGSLRLSCAASGYTFTNYLIEWVRQAPGK |
| | | GLEWVGDINPNNGGTYYNDSVKGRFTISRDNSKNTLYLQMNS |
| | | LRAEDTAVYYCVRHALLWYYYAMDYWGQGTLVTVSS |
| A2A-63 | 602 | QVQLVQSGAEVKKPGASVKVSCKASGFTFSDHYMTWVRQAT |
| | | GQGLEWMGWMNPNSGITGYAQKFQGRVTMTRDTSTSTVYME |
| | | LSSLRSEDTAVYYCARVSYSGSLHYWGQGTLVTVSS |
| A2A-64 | 603 | QVQLVQSGAEVKKPGASVKVSCKASGFTFDDYAMHWVRQAT |
| | | GQGLEWMGVINTNTGNPTYAQKFQGRVTMTRDTSTSTVYME |
| | | LSSLRSEDTAVYYCARSNWNYFDYWGQGTLVTVSS |
| A2A-65 | 604 | EVQLVESGGGLVKPGGSLRLSCAASGSAFSASWMNLVRQAPG |
| | | KGLEWVGWVDPDNGDTEYADSVKGRFTISRDNSKNTLYLQM |
| | | NSLRAEDTAVYYCSKPRDSGPSFASWGQGTLVTVSS |
| A2A-66 | 605 | EVQLVESGGGLVKPGGSLRLSCAASGFTFSSYDMYWVRQAPG |
| | | KGLEWVGWIYPKDGSTKYNDSVKGRFTISRDNSKNTLYLQMN |
| | | SLRAEDTAVYYCARSRGYYYGSSYGYYDVWGQGTLVTVSS |
| A2A-67 | 606 | EVQLVESGGGLVKPGGSLRLSCAASGHTITSYGINWVRQAPGK |
| | | GLEWVGEILPGSGTSDYNDSVKGRFTISRDNSKNTLYLQMNSL |
| | | RAEDTAVYYCATNWGFAYWGQGTLVTVSS |
| A2A-68 | 607 | EVQLVESGGGLVKPGGSLRLSCAASGYTFSGYTMHWVRQAPG |
| | | KGLEWVGEIDPSDSFANYNDSVKGRFTISRDNSKNTLYLQMNS |
| | | LRAEDTAVYYCARDYGSSYEFTYWGQGTLVTVSS |
| A2A-69 | 608 | QVQLVQSGAEVKKPGASVKVSCKASGGYISSSNWWSWVRQA |
| | | TGQGLEWMGRIKTKTDGGTIDYAQKFQGRVTMTRDTSTSTVY |
| | | MELSSLRSEDTAVYYCAKGYSGSVDYWGQGTLVTVSS |
| A2A-70 | 609 | EVQLVESGGGLVKPGGSLRLSCAASGSNIKDYYIHWVRQAPG |
| | | KGLEWVATISDGGSYIFDDSVKGRFTISRDNSKNTLYLQMNSL |
| | | RAEDTAVYYCARDATGTFAYWGQGTLVTVSS |
| A2A-71 | 610 | QVQLQESGPGLVKPSGTLSLTCAVSGGSISSSNWWSWVRQPPG |
| | | RGLEWIGEIYHSGSTNYNPSLKSRVTISVDKPKNQFSLKLSSVT |
| | | AADTAVYYCAREVVSGMIGTVFDYWGQGTLVTVSS |
| A2A-72 | 611 | QVQLVQSGGGLVQPGGSLRLSCAVSGFTISTYDIYWVRQATGK |
| | | GLEWVSAIGTAGDTYYPDSVRGRFTISREDARNSLYLQMNSLR |
| | | TGDTAVYYCATGYNWIFDYWGQGTLVTVSS |
| A2A-73 | 612 | QVQLVQSGGGLVQPGGSLRLSCAASGFTVSTYDIYWVRQTTG |
| | | KGLELVSAIGTAGDTYYPDSVKGRFTISRENARNSLYLQMNSL |
| | | RAGDTAVYYCAVGYNWIFDFWGQGTLVTVSS |
| A2A-74 | 613 | EVQLVESGGGLVQPGGSLRLSCAASGFTFTTYDMYWVRQTTG |
| | | KGLEWVSAIGTAGDTYYPDSVKGRFTISRESAKNSLYLQMNSL |
| | | RAGDTAVYYCTVGYNWIFDFWGHGTLVTVSS |
| A2A-75 | 614 | QVQLQESGPGLVKPSGTLSLTCAVSGGSISSSNWWSWVRQPPG |
| | | KGLEWIGEIYHSGNTNYNPSLKSRVTMSVDKSKNQFSLNLHSV |
| | | TAADTAVYYCAREVVSGMIGTIFDYWGQGTLVTVSS |
| A2A-76 | 615 | EVQLVESGGGLVQPGGSLRLSCAASGFTFSSYDMYWVRQPTG |
| | | KGLEWVSAIGTAGDTYYSGSVKGRFTISRESAKNSLYLQMNSL |
| | | RAGDTAVYYCAVGYNWIFDFWGQGTLVTVSS |
| A2A-77 | 616 | QVQLQESGPGLVKPSGTLSLTCAVSGGSISSSNWWSWVRQPPG |
| | | KGLEWIGEIYHSGNTNYNPSLKSRVTMSVDKSKNQFSLNLHSV |
| | | TAADTAVYYCAREVVSGMIGTIFDYWGQGTLVTVSS |
| A2A-78 | 617 | EVQLLESGGGLVQPGGSLRLSCAASGFTFSAYDIYWVRQPTGK |
| | | GLEWVSAIGTAGDTYYPGSVKGRFIISRESAKNSVYLQMNSLR |
| | | AGDTAVYYCAVGYNWVFDLWGQGTLVTVSS |
| A2A-79 | 618 | QVQLQESGGGVVRPGGSLRLSCAASGFTFDDYGMSWVRQVPG |
| | | KGLEWVSGITWNGDRSGYADSVKGRFTISRDNAKNSLYLQMN |
| | | SLRAEDTALYYCVRDGLTGIFDYWGQGTLVTVSS |
| A2A-80 | 619 | QVQLVQSGGGLVKPGGSLRLSCAASGFTISTYDIYWVRQATGK |
| | | GLEWVSAIGTAGDTYYPGSVKGRFTISRENAKNSLYLQMNSLR |
| | | AGDTAVYYCASGYNWIFDYWGQGTLVTVSS |
| A2A-81 | 620 | QVQLQESGPGLVKPSGTLSLTCAVSGGSISSSNWWSWVRQPPG |
| | | KGLEWIGEIYHSGSTNYNPSLKSRVTISVDKSKNQFSLKLGSVT |
| | | AADTAVYYCAREVVSGLYGTIFDYWGQGTLVTVSS |
| A2A-82 | 621 | EVQLQQSGPGLVKPSQSLSLTCSVTGYSITSGYYWNWIRQFPG |
| | | NKLEWMGYISYGGSNDYNPSLKNRISITRDSSKNQFFLKLNSVT |
| | | TEDTATYYCARDYDYFDYWGQGTTLTVSS |
| A2A-83 | 622 | EVQRVQSGAELVKPGASVKISCKASGYAFSSYWMNWVKQRP |
| | | GKGLEWIGQIYPGDGDTNYNGKFEGKATLTADKSSSTAYMQL |
| | | TSLTSDDSAVYYCARGAYWGQGTTLTVSS |
| A2A-84 | 623 | EVQLVESGGGLVKPGGSLRLSCAASGYTFTEYITHWVRQAPGK |
| | | GLEWVATISGGGSYTNFPDSVKGRFTISRDNAKNTLFLQMTSL |
| | | RSEDTAMYYCARPNYSGSSPFAYWGQGTLVTVSA |
| A2A-85 | 624 | EVQLQQSGPGLVAPSQSLSITCTVSGFSLTAYAISWVRQPPGKG |
| | | LEWLGVIWTGGGTNYNSALKSRLSISKDNSKSQVFLKMNSLQT |
| | | DDTARYYCARSRGYYYGSSYGYFDVWGTGTTVTVSS |
| A2A-86 | 625 | EVQLQESGPGLAKPSQTLPLTCSVIGYSITSDYWNWIRKFPGNK |
| | | LEYMGYINYSGSTYYNPSLKSRISITRDTSKNQYYLQLNSVTTE |
| | | DTATYYCTRKLDWDGYFDVWGTGTTVTVSS |
| A2A-87 | 626 | EVQLQQSEAELVRPGAPVKLSCTASGFNIKNTYMHWVKQRPE |
| | | QGLEWIGRIDPANGNTKYAPKFQGKATITADTSSNTAYLQLSS |
| | | LASEDSAVYFCARGSPYGYDGHYVMDYWGQGTSVTVSS |
| A2A-88 | 627 | EVQLVESGGGLVKPGGSLRLSCAASGFTFRTYGMHWVRQAPG |
| | | KGLEWVAVISAEGSNKYYADSVKGRFTISRDNSKNTLYLQMN |
| | | SLRAEDTAVYYCARDGRGSLPRPKGGFIGALSFHWPFGRWLG |
| | | GSYGTYDSSEDSGGAFDIWGQGTLVTVSS |
| A2A-89 | 628 | QVQLVESGGGVVQPGRSLRLSCAASGFTFNNYGMHWVRQAP |
| | | GKGLEWVAVISYGGSDKYYADSVKGRFTISRDNSKNTLYLQM |
| | | NSLRAEDTAVYYCARDGRGSLPRPKGGFIGDLSFHWPFGRWL |
| | | GKSYGTYDSSEDSGGAFDIWGQGTLVTVSS |

TABLE 18
Variable Light Chain

| A2AR Variant | SEQ ID NO | Sequence |
|---|---|---|
| A2A-1 | 629 | ELVMTQSPLSLPVTLGQPASISCRSSQSLVYSDGNTYLNWFQ |
| | | QRPGQSPRRLIYKVSNRDSGVPDRFSGSGSGTDFTLKISRVEA |
| | | EDVGVYYCMQGTHWPRTFGQGTKVDIK |
| A2A-2 | 630 | ELTLTQSPAFMSATPGDKVNISCKASQDIDDDMNWYQQKPG |
| | | EAAIFIIQEATTLVPGIPPRFSGSGYGTDFTLTINNIESEDAAYY |
| | | FCLQHDNFPMYTFGQGTKLEIK |
| A2A-3 | 631 | ELVLTQSPDSLAVSLGERATFNCKSSQSVLYSSNNKNYLAW |
| | | YQQKPGQPPKLLIYWASTRESGVPDRFSGSGSGTDFTLTISSL |
| | | QAEDVAVYYCQQYYSTPYTFGQGTKVDIK |
| A2A-4 | 632 | ELTLTQSPATLSVSPGERATLSCRASQSVSSNLAWYQQKPGQ |
| | | APRLLIYGASTRATGIPARFSGSGSGTEFTLTISSLQAEDVAV |
| | | YYCQQYYSTPLTFGGGTKVEIK |
| A2A-5 | 633 | ELTLTQSPAFMSATPGDKVNISCKASQDIDDDMNWYQQKPG |
| | | EAAIFIIQEATTLVPGIPPRFSGSGYGTDFTLTINNIESEDAAYY |
| | | FCLQHDNFPWTFGQGTKVDTK |
| A2A-6 | 634 | ELQMTQSPSSVSASVGDKVTITCRASQGISSWLAWYQQKPG |
| | | KGPKLLIYAASSLQSGVPSRFSGSGSGTDFTPTISSLQPEDFAT |
| | | YYCQQTNSFPRTLGQGTKLEIK |
| A2A-7 | 635 | ELTLTQSPAFMSATPGDKVNISCKASQDVDDDMNWYQQKP |
| | | GEAAIFIIQEATTLVPGIPPRFSGSGYGTDFTLTINNIESEDAAY |
| | | YFCLQHDNFPWTFGQGTRLEIK |
| A2A-8 | 636 | DIVMTQAQKFMSTSVGDRVSVTCKASQNVGTNVAWYQQKP |
| | | GQSPKALIYSASYRYSGVPDRFTGSGSGTDFTLTVTNVQSED |
| | | LAEYFCQRFNNYPLTFGAGTKLEIK |
| A2A-9 | 637 | DIVMTQSQKFMSTSVGDRVSATCKASQNVGSSVAWFQQKP |
| | | GQSPKALIYSTSYRYSGVPDRFTGSGSGTDFTLTISNVQSEDL |
| | | AEYFCQQYNSYPLTFGAGTKLEIK |
| A2A-10 | 638 | DIVMTQAPATLSVTPGDRVSLSCRASQSISDYLHWYQQKSHE |
| | | SPRLLIKYASQSISGIPSRFSGSGSGSDFTLSINSVEPEDVGVYY |
| | | CQNGHSFPLTFGAGTKLEIK |
| A2A-11 | 639 | DIVMTQSQKFMSTSVGDRVSVTCKASRNVGTNVAWYQQKL |
| | | GQSPKTLIYSASYRYSGVPDRFTGSGSGTDFTLTISNVQSEDL |
| | | AEYFCQQYNSYPLTFGAGTKLEIK |
| A2A-12 | 640 | DIQMTQTPATLSVTPGDRVSLSCRASQSISDYLHWYQQKSHE |
| | | SPRLLIKYASQSISGIPSRFSGSGSGSDFTLSINSVEPEDVGVYY |
| | | CQNGHSFPHTLGSGTKLEIK |
| A2A-13 | 641 | DIQMIQSQKFMSTSVGDRVSVTCKASQNVGTNVAWYQQKP |
| | | GQSPKALIYSASYRYSGVPDRFTGSGSGTDFTLTIGNVQSEDL |
| | | VEYFCQQYNIYPLTFGAGTKLELK |
| A2A-14 | 642 | DIVMTQSPATLSVTPGDSVSLSCRASQSISNYLHWYQQKSHE |
| | | SPRLLIKYASQSISGIPSRFSGSGSGSDFTLSINSVEPEDVGVYY |
| | | CQNGHSFPLTFGGGTKLELK |
| A2A-15 | 643 | DIQMTQSPSSLSASVGDRVTITCRASQSIGRYLNWYQQKPGK |
| | | APKLLIYAASSLHSGVPSRFSGSGSGTDFTLTISSLQPEDFATY |
| | | YCQQSYVTPWTFGGGTKLEIK |
| A2A-16 | 644 | DIQMTQSPSSLSASVGDRVTITCRASQSIGTYLNWYQQKPGK |
| | | APKLLIYGASTLHSGVPSRFSGSGSGTDFTLTISSLQPEDFATY |
| | | YCQQSYSAPWTFGGGTKVEIK |
| A2A-17 | 645 | DIQMTQSPSSLSASVGDRVTITCKASQSVRNDVVWYQQKPG |
| | | KAPKLLIYRGNTLRPGVPSRFSGSGSGTDFTLTISSLQPEDFAT |
| | | YYCQQYYGIPLTFGQGTKLEIK |
| A2A-18 | 646 | DIQMTQSPSSLSASVGDRVTITCRASQSVTTYLNWYQQKPGK |
| | | APKLLIYSASSLQSGVPSRFSGSGSGTDFTLTISSLQPEDFATY |
| | | YCQQTYATPWTFGGGTKVEIK |
| A2A-19 | 647 | DIQMTQSPSSLSASVGDRVTITCRASQSISDYLNWYQQKPGK |
| | | APKLLIYTASTLQSGVPSRFSGSGSGTDFTLTISSLQPEDFATY |
| | | YCEQSYSTPWTFGGGTKLEIK |
| A2A-20 | 648 | DIQMTQSPSSLSASVGDRVTITCKASHSVDYDGDNYMNWYQ |
| | | QKPGKAPKLLIYWASTRLTGVPSRFSGSGSGTDFTLTISSLQP |
| | | EDFATYYCLQHIEYPFTFGQGTKLEIK |
| A2A-21 | 649 | DIQMTQSPSSLSASVGDRVTITCKSSQSVLYSSNNKNYFAWY |
| | | QQKPGKAPKLLIYDAPNRATGVPSRFSGSGSGTDFTLTISSLQ |
| | | PEDFATYYCQQGYTTPYTFGGGTKVEIK |
| A2A-22 | 650 | DIQMTQSPSSLSASVGDRVTITCRASQDIGRSLSWYQQKPGK |
| | | APKLLIYDASRFISGVPSRFSGSGSGTDFTLTISSLQPEDFATY |
| | | YCQWSNSWPYTFGQGTKLEIK |
| A2A-23 | 651 | DIQMTQSPSSLSASVGDRVTITCRASQSIGRYLNWYQQKPGK |
| | | APKLLIYAASSLHSGVPSRFSGSGSGTDFTLTISSLQPEDFATY |
| | | YCQQSYVTPWTFGGGTKLEIK |
| A2A-24 | 652 | DIQMTQSPSSLSASVGDRVTITCKASQSVRNDVVWYQQKPG |
| | | KAPKLLIYRGNTLRPGVPSRFSGSGSGTDFTLTISSLQPEDFAT |
| | | YYCQQYYGIPLTFGQGTKLEIK |
| A2A-25 | 653 | DIQMTQSPSSLSASVGDRVTITCKASQSVDYDGDSYMNWYQ |
| | | QKPGKAPKLLIYRANRLVDGVPSRFSGSGSGTDFTLTISSLQP |
| | | EDFATYYCQNGHSFPLTFGQGTKLEIK |
| A2A-26 | 654 | DIQMTQSPSSLSASVGDRVTITCRASQTISRYLNWYQQKPGK |
| | | APKLLIYSASTLQSGVPSRFSGSGSGTDFTLTISSLQPEDFATY |
| | | YCQQSYSTPHTFGGGTKVEIK |
| A2A-27 | 655 | DIQMTQSPSSLSASVGDRVTITCRASQSIGSYLNWYQQKPGK |
| | | APKLLIYGASNLQSGVPSRFSGSGSGTDFTLTISSLQPEDFATY |
| | | YCQQGYSAPRTFGGGTKLEIK |
| A2A-28 | 656 | DIQMTQSPSSLSASVGDRVTITCRASRSISSYLNWYQQKPGK |
| | | APKLLIYAASSLPSGVPSRFSGSGSGTDFTLTISSLQPEDFATY |
| | | YCQQSYSTPRTFGGGTKLEIK |
| A2A-29 | 657 | DIQMTQSPSSLSASVGDRVTITCKVSQDVRTAVAWYQQKPG |
| | | KAPKLLIYDTSYLASGVPSRFSGSGSGTDFTLTISSLQPEDFAT |
| | | YYCQQSYSWSLTFGQGTKLEIK |
| A2A-30 | 658 | DIQMTQSPSSLSASVGDRVTITCGGGNDIGSSMYWYQQKPG |
| | | KAPKLLIYWMSNLASGVPSRFSGSGSGTDFTLTISSLQPEDFA |
| | | TYYCQQYSTYPFALGQGTKLEIK |
| A2A-31 | 659 | DIQMTQSPSSLSASVGDRVTITCRASQSISDYLNWYQQKPGK |
| | | APKLLIYGASPRESGVPSRFSGSGSGTDFTLTISSLQPEDFATY |
| | | YCQQDNIWPYTFGQGTKLEIK |
| A2A-32 | 660 | DIQMTQSPSSLSASVGDRVTITCGGGNDIGSSMYWYQQKPG |
| | | KAPKLLIYDASRFISGVPSRFSGSGSGTDFTLTISSLQPEDFAT |
| | | YYCQQSNEDPPFTFGQGTKLEIK |
| A2A-33 | 661 | DIQMTQSPSSLSASVGDRVTITCRASESVDSFGNNFMNWYQ |
| | | QKPGKAPKLLIYHTSRLNSGVPSRFSGSGSGTDFTLTISSLQPE |
| | | DFATYYCQQNNEVPRTFGQGTKLEIK |
| A2A-34 | 662 | DIQMTQSPSSLSASVGDRVTITCRASSSVTYIHWYQQKPGKA |
| | | PKLLIYAVSRLDSGVPSRFSGSGSGTDFTLTISSLQPEDFATYY |
| | | CHQSNEDPYTFGQGTKLEIK |
| A2A-35 | 663 | DIQMTQSPSSLSASVGDRVTITCRASQSIGRYLNWYQQKPGK |
| | | APKLLIYAASSLHSGVPSRFSGSGSGTDFTLTISSLQPEDFATY |
| | | YCQQSYVTPWTFGGGTKVEIK |
| A2A-36 | 664 | DIQMTQSPSSLSASVGDRVTITCKASQSVRNDVVWYQQKPG |
| | | KAPKLLIYRGNTLRPGVPSRFSGSGSGTDFTLTISSLQPEDFAT |
| | | YYCQQYYGIPLTFGQGTKLEIK |
| A2A-37 | 665 | DIQMTQSPSSLSASVGDRVTITCKASHSVDYDGDNYMNWYQ |
| | | QKPGKAPKLLIYDASRFISGVPSRFSGSGSGTDFTLTISSLQPE |
| | | DFATYYCLRYASYRTFGQGTKLEIK |
| A2A-38 | 666 | DIQMTQSPSSLSASVGDRVTITCRASESVNSYGNSFMHWYQ |
| | | QKPGKAPKLLIYDASRFISGVPSRFSGSGSGTDFTLTISSLQPE |
| | | DFATYYCLQYGESPLTFGQGTKLEIK |
| A2A-39 | 667 | DIQMTQSPSSLSASVGDRVTITCRSSKSLLHSSGNAYVYWYQ |
| | | QKPGKAPKLLIYYTSKPNSGVPSRFSGSGSGTDFTLTISSLQPE |
| | | DFATYYCQHHYGIPLTFGQGTKLEIK |
| A2A-40 | 668 | DIQMTQSPSSLSASVGDRVTITCRASQSIGTYLNWYQQKPGK |
| | | APKLLIYAASSLESGVPSRFSGSGSGTDFTLTISSLQPEDFATY |
| | | YCQQTYNTPWTFGGGTKVEIK |
| A2A-41 | 669 | DIQMTQSPSSLSASVGDRVTITCRASSRVSSSYLYWYQQKPG |
| | | KAPKLLIYATYSLDYGVPSRFSGSGSGTDFTLTISSLQPEDFA |
| | | TYYCLQHGERPLTFGQGTKLEIK |
| A2A-42 | 670 | DIQMTQSPSSLSASVGDRVTITCGASQSIGTIIHWYQQKPGKA |
| | | PKLLIYDTSYLASGVPSRFSGSGSGTDFTLTISSLQPEDFATYY |
| | | CQQGNTRPWTFGQGTKLEIK |
| A2A-43 | 671 | DIQMTQSPSSLSASVGDRVTITCRASENIYVPLNWYQQKPGK |
| | | APKLLIYDASRFISGVPSRFSGSGSGTDFTLTISSLQPEDFATY |
| | | YCQQYNSFPLYTFGQGTKLEIK |
| A2A-44 | 672 | ELVLTQSPGTLSLSPGERATLSCRASQSVSSSYLAWYQQKPG |
| | | QAPRLLIYGASSRATGIPDRFSGSGSGTDFTLTISRLEPEDFAV |
| | | YYCQQYGSSPITFGQGTKVDIK |
| A2A-45 | 673 | DIQMTQSPSSLSASVGDRVTITCKSSQSLLYSGEKTYPYWYQ |
| | | QKPGKAPKLLIYWASTRLTGVPSRFSGSGSGTDFTLTISSLQP |
| | | EDFATYYCQQSNEDSWTFGQGTKLEIK |
| A2A-46 | 674 | DIQMTQSPSSLSASVGDRVTITCQSSQDIFNYLEWYQQKPGK |
| | | APKLLIYTASNLDTGVPSRFSGSGSGTDFTLTISSLQPEDFATY |
| | | YCQQGYSTPPEITFGGGTKVEIK |
| A2A-47 | 675 | DIQMTQSPSSLSASVGDRVTITCRSTRNILSNMPWYQQKPGK |
| | | APKLLIYNANTLAEGVPSRFSGSGSGTDFTLTISSLQPEDFAT |
| | | YYCLQHWNYPYMFGQGTKLEIK |
| A2A-48 | 676 | DIQMTQSPSSLSASVGDRVTITCRASQDISNNLHWYQQKPGK |
| | | APKLLIYEISGWLSGVPSRFSGSGSGTDFTLTISSLQPEDFATY |
| | | YCQQSNSWSLLTFGQGTKLEIK |
| A2A-49 | 677 | DIQMTQSPSSLSASVGDRVTITCSASQSMSNNLHWYQQKPG |
| | | KAPKLLIYLASNLGYGVPSRFSGSGSGTDFTLTISSLQPEDFA |
| | | TYYCRQNGHSFPLTFGQGTKLEIK |
| A2A-50 | 678 | DIQMTQSPSSLSASVGDRVTITCRASQDISNNLHWYQQKPGK |
| | | APKLLIYWASTRLTGVPSRFSGSGSGTDFTLTISSLQPEDFAT |
| | | YYCQQWSDYPFTFGQGTKLEIK |
| A2A-51 | 679 | DIQMTQSPSSLSASVGDRVTITCSASSSLSYMHWYQQKPGKA |
| | | PKLLIYGASPRESGVPSRFSGSGSGTDFTLTISSLQPEDFATYY |
| | | CRQMSSYPPTFGQGTKLEIK |
| A2A-52 | 680 | DIQMTQSPSSLSASVGDRVTITCSASSSVSYMNWYQQKPGK |
| | | APKLLIYEISGWLSGVPSRFSGSGSGTDFTLTISSLQPEDFATY |
| | | YCLRYASYRTFGQGTKLEIK |
| A2A-53 | 681 | DIQMTQSPSSLSASVGDRVTITCKASQNMGSNVAWYQQKPG |
| | | KAPKLLIYSASHRSSGVPSRFSGSGSGTDFTLTISSLQPEDFAT |
| | | YYCQQWNYPRITFGQGTKLEIK |
| A2A-54 | 682 | DIQMTQSPSSLSASVGDRVTITCKASQNGGTNVDWYQQKPG |
| | | KAPKLLIYEISGWLSGVPSRFSGSGSGTDFTLTISSLQPEDFAT |
| | | YYCQHYYSWPPTFGQGTKLEIK |
| A2A-55 | 683 | DIQMTQSPSSLSASVGDRVTITCRASENIYVPLNWYQQKPGK |
| | | APKLLIYLASYRFTGVPSRFSGSGSGTDFTLTISSLQPEDFATY |
| | | YCQQINGWPYTFGQGTKLEIK |
| A2A-56 | 684 | DIQMTQSPSSLSASVGDRVTITCKASQNMGSNVAWYQQKPG |
| | | KAPKLLIYAATRLADGVPSRFSGSGSGTDFTLTISSLQPEDFA |
| | | TYYCRQHYSSPPTFGQGTKLEIK |
| A2A-57 | 685 | DIQMTQSPSSLSASVGDRVTITCKASQNGGTNVDWYQQKPG |
| | | KAPKLLIYVASNQGTGVPSRFSGSGSGTDFTLTISSLQPEDFA |
| | | TYYCQQYYTYPLTFGQGTKLEIK |
| A2A-58 | 686 | DIQMTQSPSSLSASVGDRVTITCKASQGVDTNVAWYQQKPG |
| | | KAPKLLIYSSSISGVPSRFSGSGSGTDFTLTISSLQPEDFATYY |
| | | CAQNRELPFTFGQGTKLEIK |
| A2A-59 | 687 | DIQMTQSPSSLSASVGDRVTITCKASQDVGTAITWYQQKPGK |
| | | APKLLIYSASKRNTGVPSRFSGSGSGTDFTLTISSLQPEDFATY |
| | | YCLHHYGTPYTFGQGTKLEIK |
| A2A-60 | 688 | DIQMTQSPSSLSTSVGDRVTITCKASQDVGTSVAWYQQKPG |
| | | KAPKLLIYPASYRSSGVPSRFSGSGSGTDFTLTISSLQPEDFAT |
| | | YYCQQGSSNPLTFGQGTKLEIK |
| A2A-61 | 689 | DIQMTQSPSSLSASVGDRVTITCRASQVIDDDINWYQQKPGK |
| | | APKLLIYLGSNRAPGVPSRFSGSGSGTDFTLTISSLQPEDFATY |
| | | YCHQSYTTPHTFGGGTKVEIK |
| A2A-62 | 690 | DIQMTQSPSSLSASVGDRVTITCRASQEISGYLTWYQQKPGK |
| | | APKLLIYSASHRSSGVPSRFSGSGSGTDFTLTISSLQPEDFATY |
| | | YCQQWDNNPYTFGQGTKLEIK |
| A2A-63 | 691 | DIQMTQSPSSLSASVGDRVTITCRASQSISRYLNWYQQKPGK |
| | | APKLLIYKASSLERGVPSRFSGSGSGTDFTLTISSLQPEDFATY |
| | | YCLQPNSYPWTFGGGTKVEIK |
| A2A-64 | 692 | DIQMTQSPSSLSASVGDRVTITCRASQGISSWLDWYQQKPGK |
| | | APKLLIYTPFSLQSGVPSRFSGSGSGTDFTLTISSLQPEDFATY |
| | | YCQHYDDLPLTFGGGTKVEIK |
| A2A-65 | 693 | DIQMTQSPSSLSASVGDRVTITCKASQNMGSNVAWYQQKPG |
| | | KAPKLLIYEASTRFSGVPSRFSGSGSGTDFTLTISSLQPEDFAT |
| | | YYCQQYSSYPLRFGQGTKLEIK |
| A2A-66 | 694 | DIQMTQSPSSLSASVGDRVTITCRASQGILGYLNWYQQKPGK |
| | | APKLLIYSTSNLLLGVPSRFSGSGSGTDFTLTISSLQPEDFATY |
| | | YCRQLSSNPLTFGQGTKLEIK |
| A2A-67 | 695 | DIQMTQSPSSLSASVGDRVTITCRASESVDNYGISFMSWYQQ |
| | | KPGKAPKLLIYDASRFISGVPSRFSGSGSGTDFTLTISSLQPED |
| | | FATYYCQQINSWPLTFGQGTKLEIK |
| A2A-68 | 696 | DIQMTQSPSSLSASVGDRVTITCKSSQSLLYSGEKTYPYWYQ |
| | | QKPGKAPKLLIYEASNRYTGVPSRFSGSGSGTDFTLTISSLQP |
| | | EDFATYYCQQWSSYPPIAFGQGTKLEIK |
| A2A-69 | 697 | DIQMTQSPSSLSASVGDRVTITCRASQGLRHDLGWYQQKPG |
| | | KAPKLLIYWASNRESGVPSRFSGSGSGTDFTLTISSLQPEDFA |
| | | TYYCQKYSSTPYTFGGGTKVEIK |
| A2A-70 | 698 | DIQMTQSPSSLSASVGDRVTITCHASESVSVAGTSLLHWYQQ |
| | | KPGKAPKLLIYAASNRESGVPSRFSGSGSGTDFTLTISSLQPE |
| | | DFATYYCQHWSSFPLTFGQGTKLEIK |
| A2A-71 | 699 | ELQMTQSPSSLSASVGDRVTITCRVSQGISNYLNWYQQKPGK |
| | | APKLLIYAASSLQSGVPSRFSGSGSGTDFTLTISSLQPEDFATY |
| | | YCQQSYSTPLTFGGGTKVEIK |
| A2A-72 | 700 | ELTLTQSPATLSVSPGERATLSCRASQSVSSNLAWYQQKPGQ |
| | | APRLLIYGASSRATGIPDRFSGSGSGTDFTLTISRLEPEDFAVY |
| | | YCQQYGSSPPTFGQGTRLEIK |
| A2A-73 | 701 | ELTLTQSPATLSVSPGERATLSCRASQSVSSNLAWYQQKPGQ |
| | | APRLLIYGASSRATGIPDRFSGSGSGTDFTLTISRLEPEDFAVY |
| | | YCQQYGSSPLTFGPGTKVDIK |
| A2A-74 | 702 | ELVMTQSPGTLSLSPGERATLSCRASQSVSSSYLAWYQQKPG |
| | | QAPRLLIYGASSRATGIPDRFSGSGSGTDFTLTISSLQAEDVA |
| | | VYYCQQYYSTPLTFGGGTKVEIK |
| A2A-75 | 703 | ELVMTQFPSSLSASVGDRVTITCRASQSISSYLNWYQQKPGK |
| | | APKLLIYAASSLQSGVPSRFSGSGSGTDFTLTISSLQPEDFATY |
| | | YCQQANSFPITFGQGTRLEIK |
| A2A-76 | 704 | ELVMTQSPATLSVSLGERATLSCRASQSVSSNLAWYQQKPG |
| | | QAPRLLIYDASNRATGIPARFSGSGSGTDFTLTISRLGPEDFA |
| | | VYYCQQYGSSPLTFGGGTKVEIK |
| A2A-77 | 705 | ELVMTQSPSSLSASVGDRVTITCRASQRISSYLNWYQQKPGK |
| | | APKLLIYAASSLQSRVPSRFSGSGSGTDFTLTISSLQPEDFATY |
| | | YCQQSYSTPLTFGGGTKLETK |
| A2A-78 | 706 | ELTLTQSPATLSLSPGERATLSCRAIQSVSSSYLAWYQQKPG |
| | | QAPRLLIYGASSRATGIPDRFSGSGSGTDFTLTISRLEPEDFAV |
| | | YYCQQYGSSPLTFGGGTRLEIK |
| A2A-79 | 707 | ELTLTQSPATLSVSPGERATLSCRASQSVSSYLAWYQQKPGQ |
| | | APRLLIYGASSRATGIPDRFSGSGSGTDFTLTISRLEPEDFAVY |
| | | YCQQYGNSYTFGQGTKVDIK |
| A2A-80 | 708 | ELTLTQSPGTLSLSPGERATLSCRASQSVSSNLAWYQQKPGQ |
| | | APRLLIYGASTRATGTPARFSGSGSGTEFTLTISSLQSEDFAVY |
| | | YCQQYGSSPPTFGQGTRLEIK |
| A2A-81 | 709 | ELVMTQSPSSLSASVGDRVTITCRASQSISSYLNWYQQKPGK |
| | | APKLLIYAASSLQSGVPSRFSGSGSGTDFTLTISSLQPEDFATY |
| | | YCQQSYSTPITFGGGTKLEIK |
| A2A-82 | 710 | DIVITQAPKFLLVSAGDRVTITCKASQSVSNDVAWYQQKPG |
| | | QSPKLLIYYASNRYTGVPDRFSGSGYGTDFTFTISTVQAEDLA |
| | | VYFCQQDYRSPLTFGAGTKLELK |
| A2A-83 | 711 | DIQMKQSQKFMSTSVGDRVSVTCKASQNVGTNVAWYQQKP |
| | | GQSPKALIYSASYRYSGVPDRFTGSGSGTDFTLTISNVQSGDL |
| | | AEYFCQQYNSYPLTFGAGTKLEIK |
| A2A-84 | 712 | DIVMTQAPAIMSASPGEKVTMTCSASSSVSYMYWYQQKPGS |
| | | SPRFLIYDTSNLASGVPVRFSGSGSGTSYSLTISSMEAEDAAT |
| | | YYCQQWNSNPLTFSAGTKLEIK |
| A2A-85 | 713 | DIVMTQSPATLSVTPGDRVSLSCRASQSISDYLHWYQQKSHE |
| | | SPRLLIKYASQSISGIPSRFSGSGSGTDFTLSINSVEPEDVGVY |
| | | YCQNGHSFPLTFGAGTKLELK |
| A2A-86 | 714 | DIKITQSPSSLSASLGDTITITCHASQNINVWLNRYQQKPGNIP |
| | | KLLIYKASNLHTGVPSRFSGSGSGTGFTLTISSLQPEDIATYYC |
| | | QQGQSYPLTFGAGTKLEIK |
| A2A-87 | 715 | DIQMNQSQKFMSTSVGDRVSVTCKASQNVGSNVAWYQQKP |
| | | GQSPKALIYSASYRYSGVPDRFTGSGSGTDFTPTISNVQSEDL |
| | | AEYFCQQYNSYPLTFGAGTKLELK |
| A2A-88 | 716 | QSVLTQPPSVSAAPGQKVTISCSGISSNIGNNYVSWYQQLPGT |
| | | APKLLIYDNNKRASGIPDRFSGSKSGTSATLGITGLQTGDEAD |
| | | YYCGTWDTSLSAGVFGGGTKLTVL |
| A2A-89 | 717 | QSVLTQPPSVSAAPGQKVTISCSGSSSNIGNHYVSWYQQLPG |
| | | TAPKLLIYDNTKRPSGIPDRFSGSKSGTSATLGITGLQTGDEA |
| | | DYYCGTWDASLSTWVFGGGTKLTVL |

### Example 23: In Vivo Cell Analysis of A2A-77 and A2A-81

### Cell Binding Assay

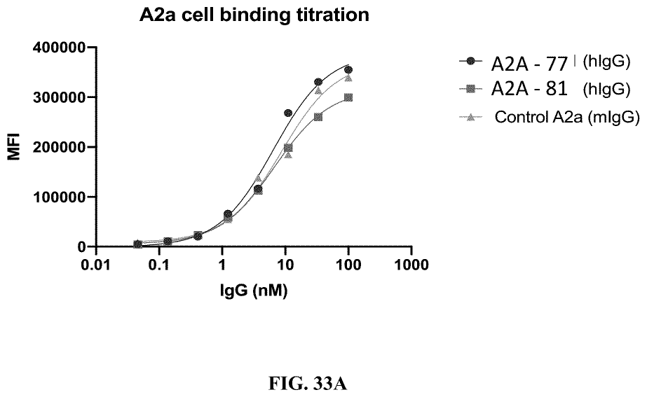

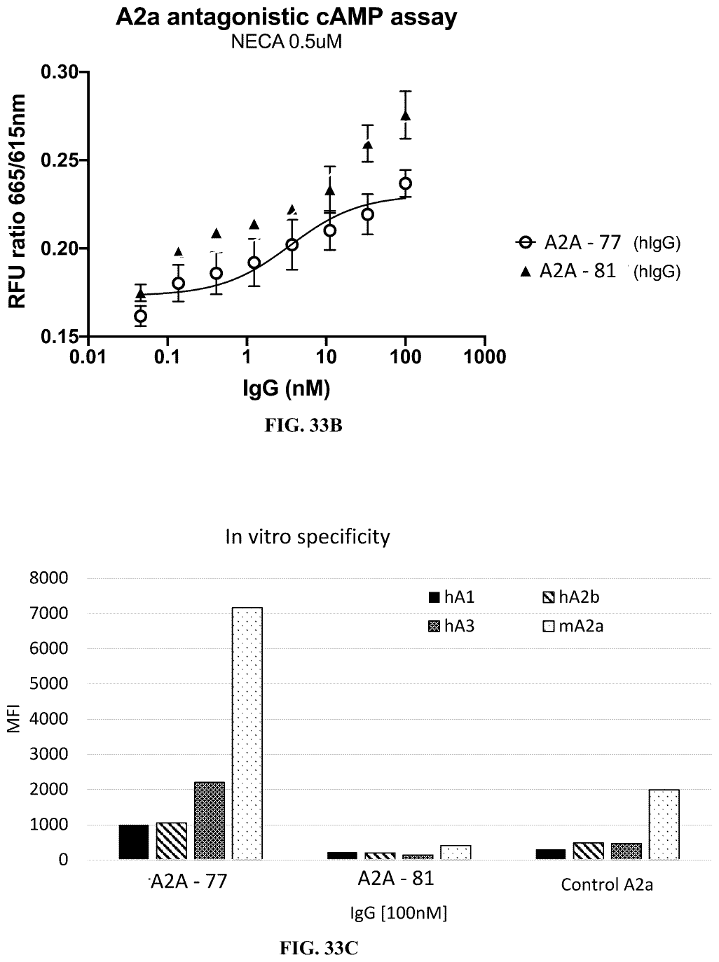

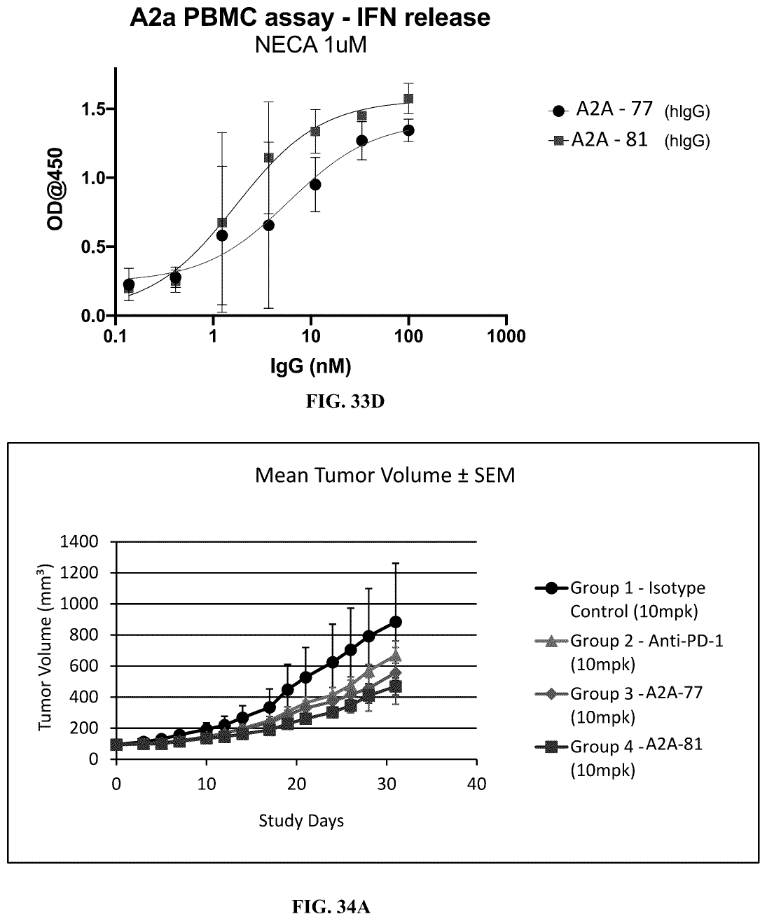

A2A-77 and A2A-81 were assessed for binding at concentrations titrated from 100 nM. Resulting curves are shown in FIG. 33A and results are shown in Table 19. Binding curves are plotted with IgG concentration vs. MFI (mean fluorescence intensity). Both A2A-77 and A2A-81 were high affinity binders to hA2a receptor.

TABLE 19

| | A2A-77 | A2A-81 | Control A2A |
|---|---|---|---|
| IC50 | 6.436 | 6.813 | 8.723 |

A2A Antagonistic cAMP Assay

Immunoglobulins were titrated in triplicate and incubated on cells for 1 hour, followed by incubation with 0.5 μM NECA for 30 minutes. Binding curves showing relative fluorescent units (RFU) ratio at 665 nm/615 nm versus nM IgG on a log scale are shown in FIG. 33B. Absolute IC50 is shown in Table 20, indicating that A2A-77 and A2A-81 were functional agonists in vitro.

TABLE 20

| | A2A-77 | A2A-81 |
|---|---|---|
| IC50 | 3.53 | 8.67 |

### Cross Reactivity

A2A-77 and A2A-81 were assayed for cross reactivity with HA1, hA2b, hA3 and mA2 receptor. Results are depicted in FIG. 33C. Both A2A-77 and A2A-81 showed in vitro specificity.

### Primary T-Cell Activation Assay

A primary T-cell activation assay was performed as described above. Data is seen in FIG. 33D and Table 21. T-cell activation was observed with variants A2A-77 and A2A-81. A2A-81 showed improved activity over A2A-77.

TABLE 21

| | A2A-77 | A2A-81 |
|---|---|---|
| EC50 | 5.92 | 1.71 |

### Example 24: In Vivo Study in Colon Carcinoma Model

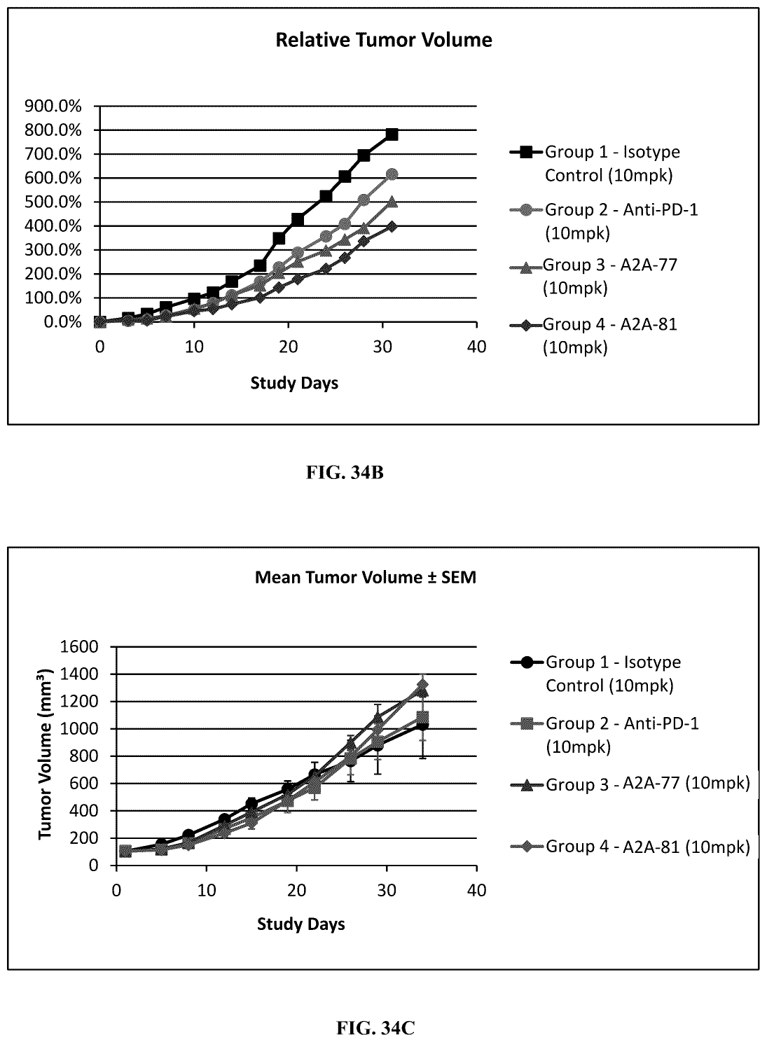

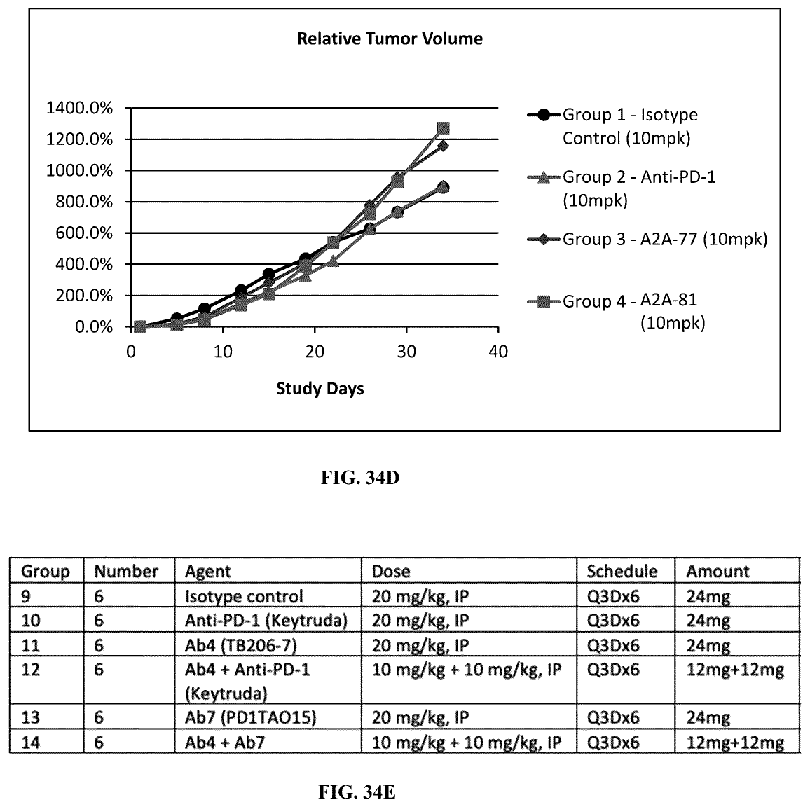

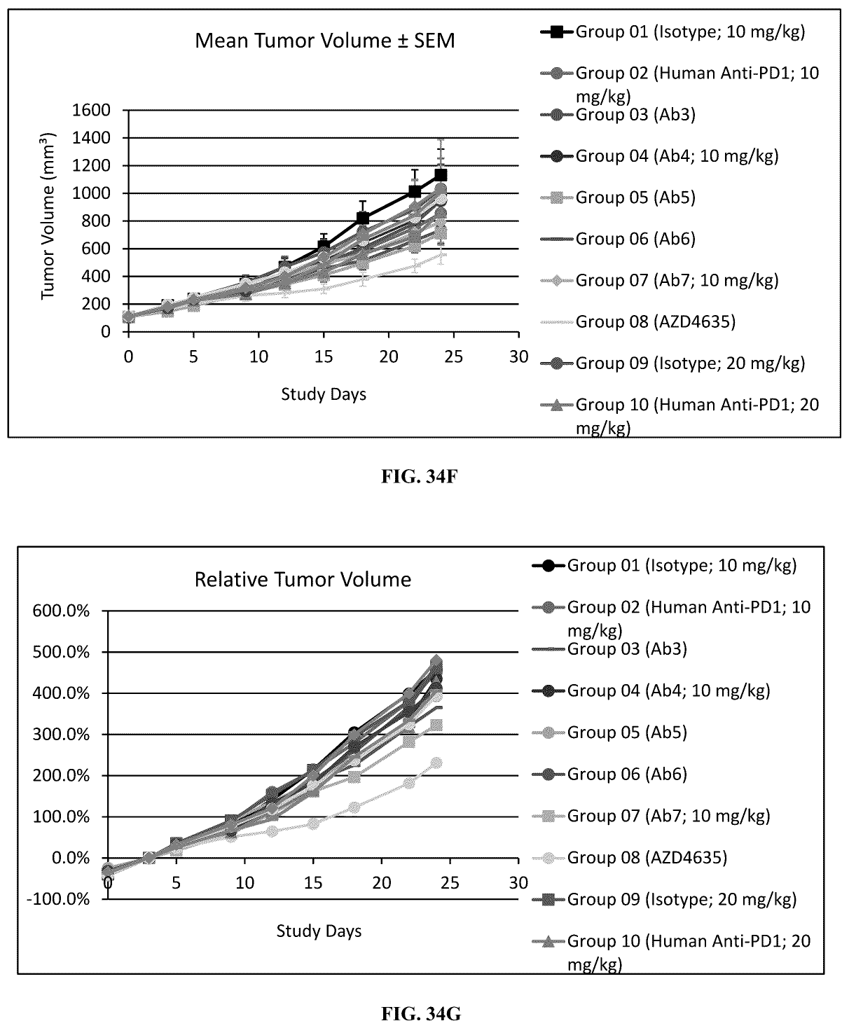

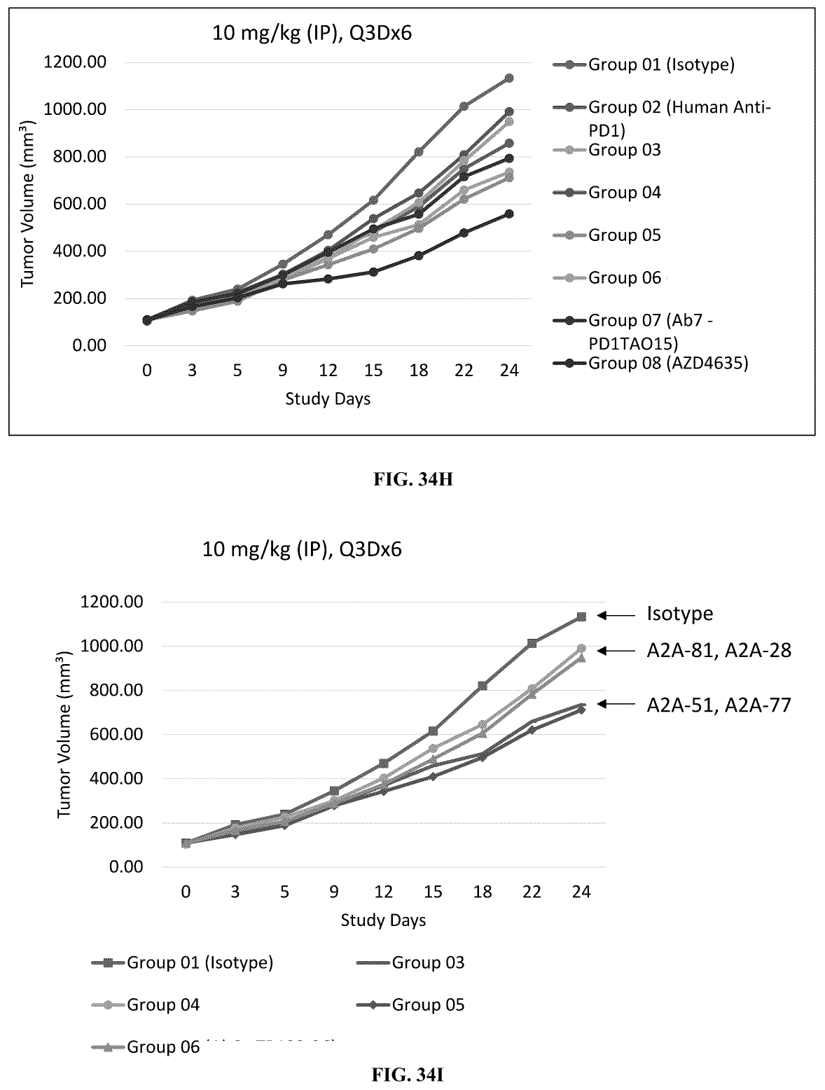

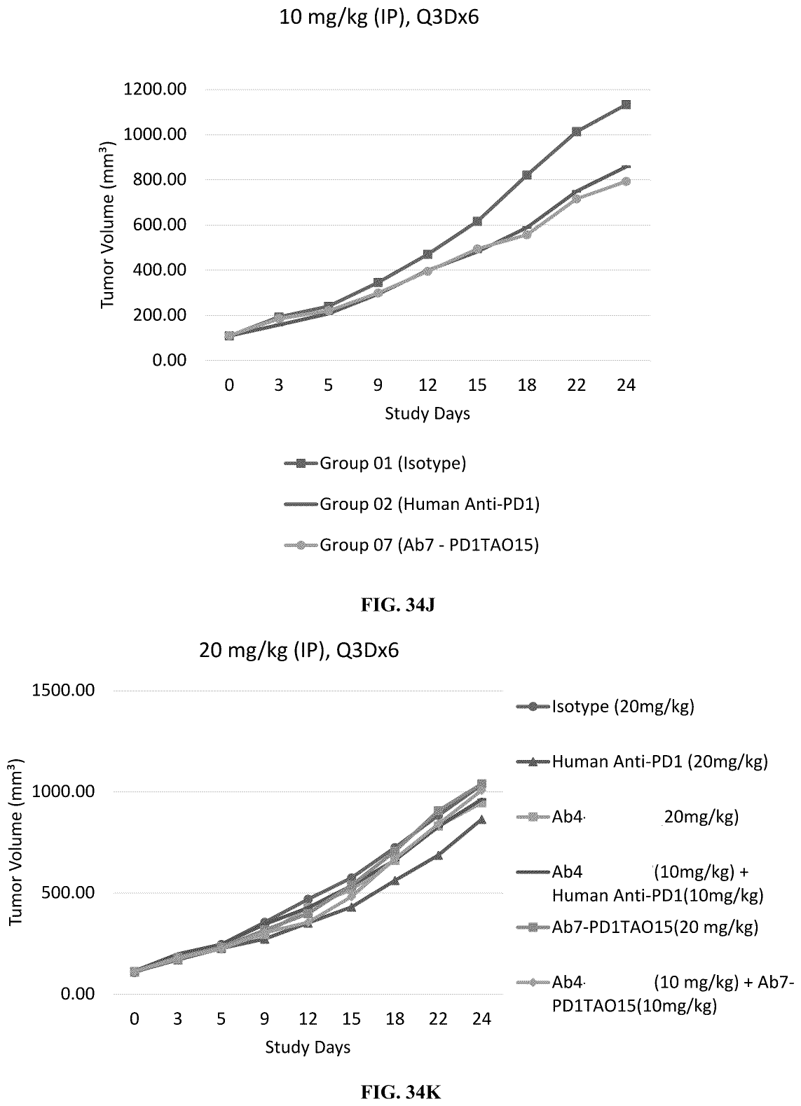

Mice with human colon carcinoma (Colo205) were divided into 4 groups. Group 1 was the isotype control, Group 2 mice were treated with Anti-PD1, Group 3 mice were treated with variant A2A-77 and Group 4 mice were treated with variant A2A-81. Tumor volume is measured over 30 days. Results are depicted in FIGS. 34A-34D. Variant A2A-81 regressed tumor size better than variant A2A-77 or the anti-PD-1 antibody.

Additional studies were performed with mice treated with 10 mg/kg variant A2A-51 (Group 5), A2A-28 (Group 6), Ab7/PDITAO15 (Group 7), and AZD4635 (Group 8). Mice were also treated with 20 mg/kg according to the schedule in FIG. 34E. Data is seen in FIGS. 34F-34K.

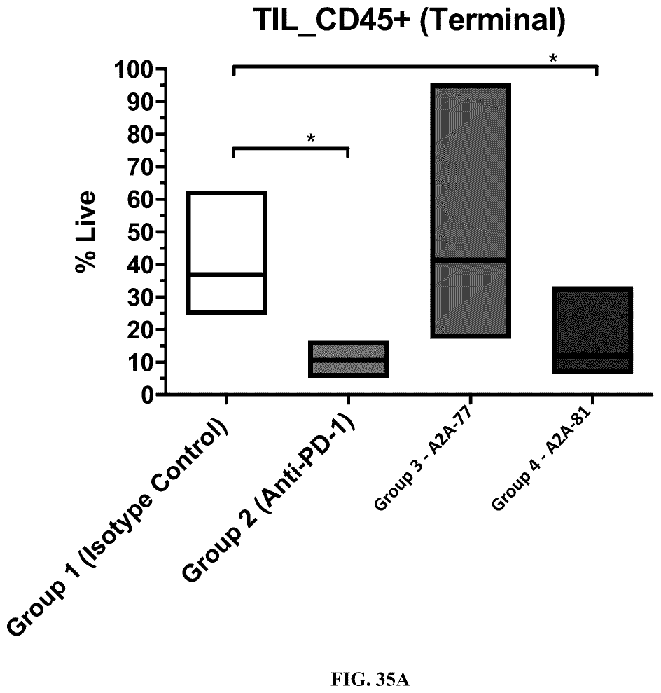

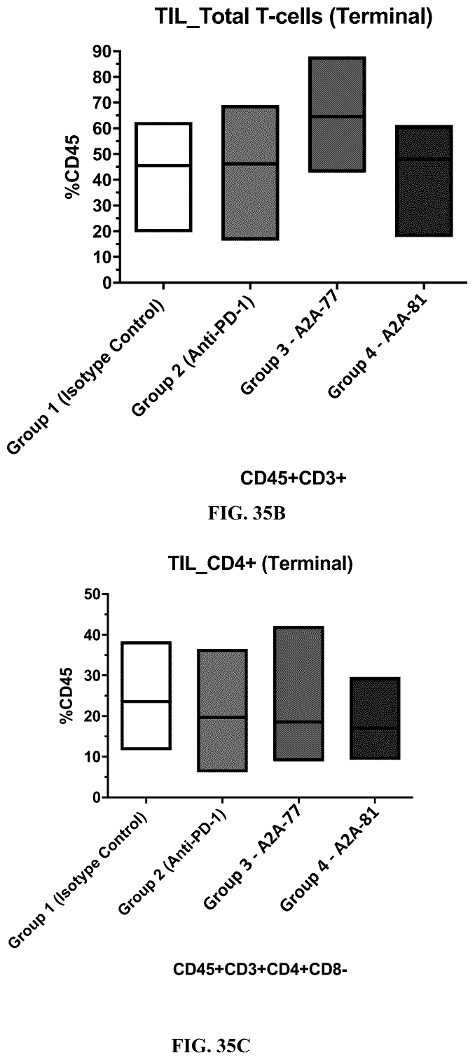

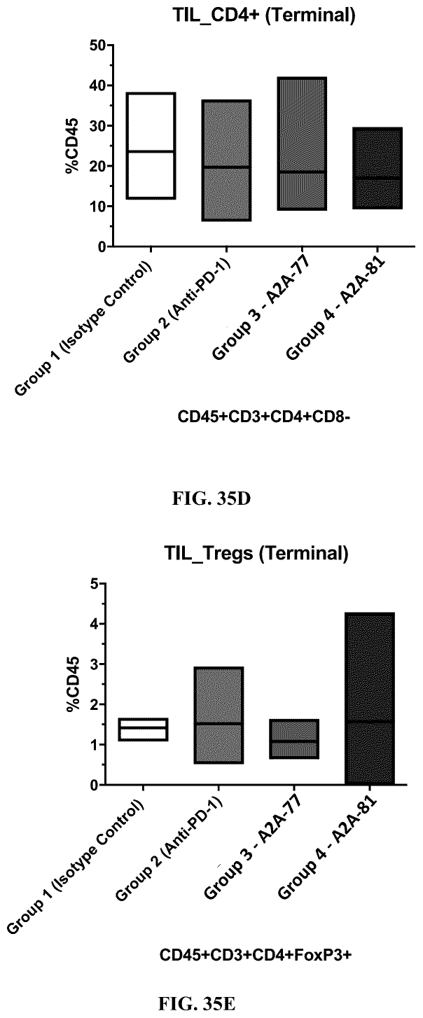

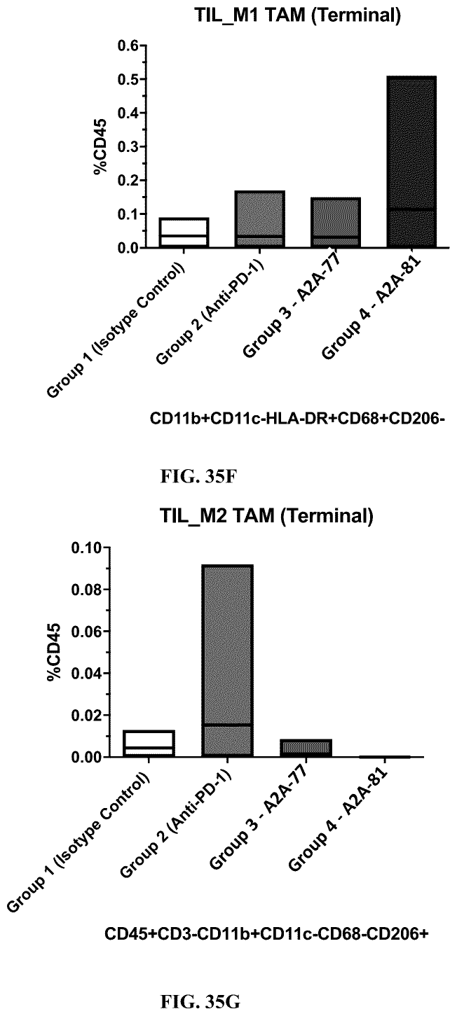

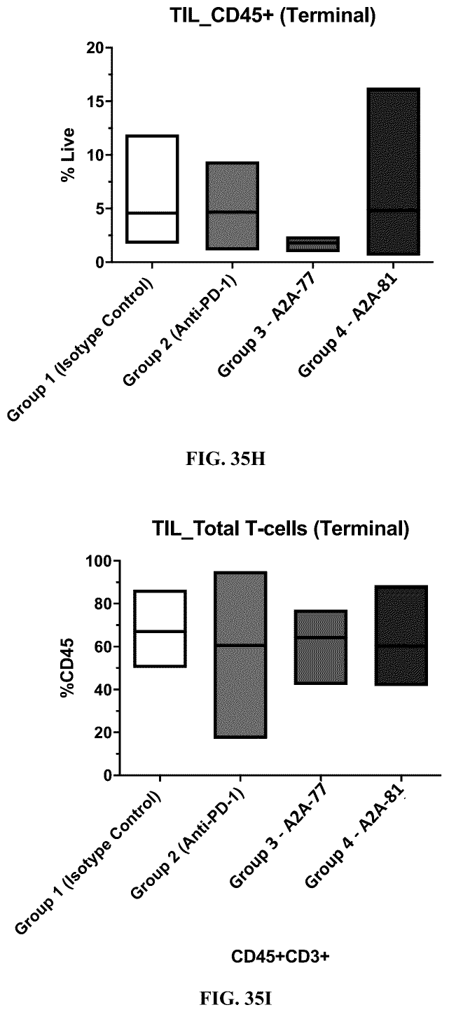

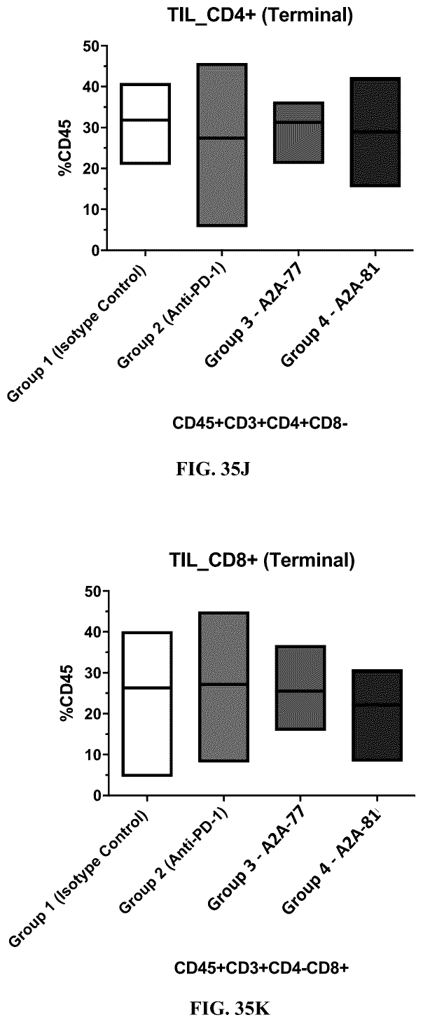

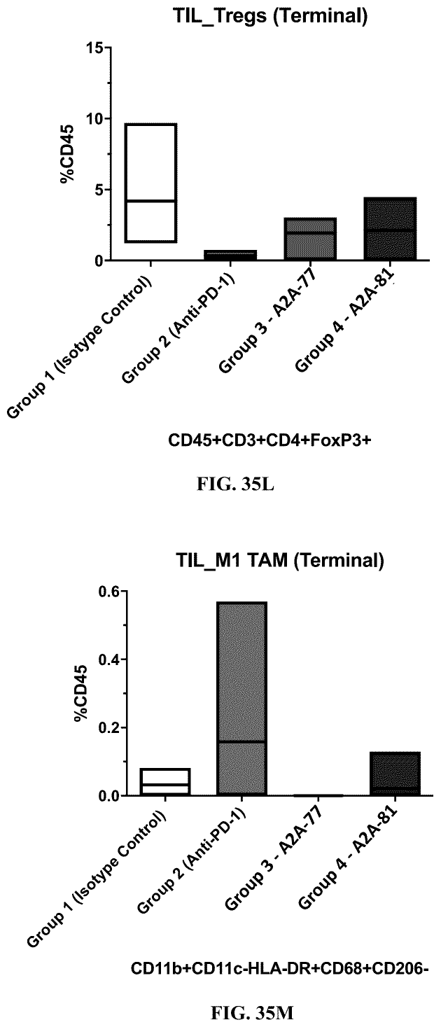

The data shows that A2A-77 and A2A-51 exhibited ability to reduce tumor volume and PDITAO15 exhibited similar results to comparator Anti-PD1 antibody. No difference was observed in combination treatments as compared to single treatment or Anti-PD1 antibody treatment. See FIG. 35K.

Tumor infiltrating lymphocytes (TIL) in both the lymphoid and myeloid compartments were measured in each of the treatment groups. Results are depicted in FIGS. 35A-35M. TIL CD8+ cells increased more in the group treated with the A2A-77 variant than the A2A-81 variant. TIL-M1 tumor associated macrophages increased more in the A2A-81 variant than the A2A-77 variant.

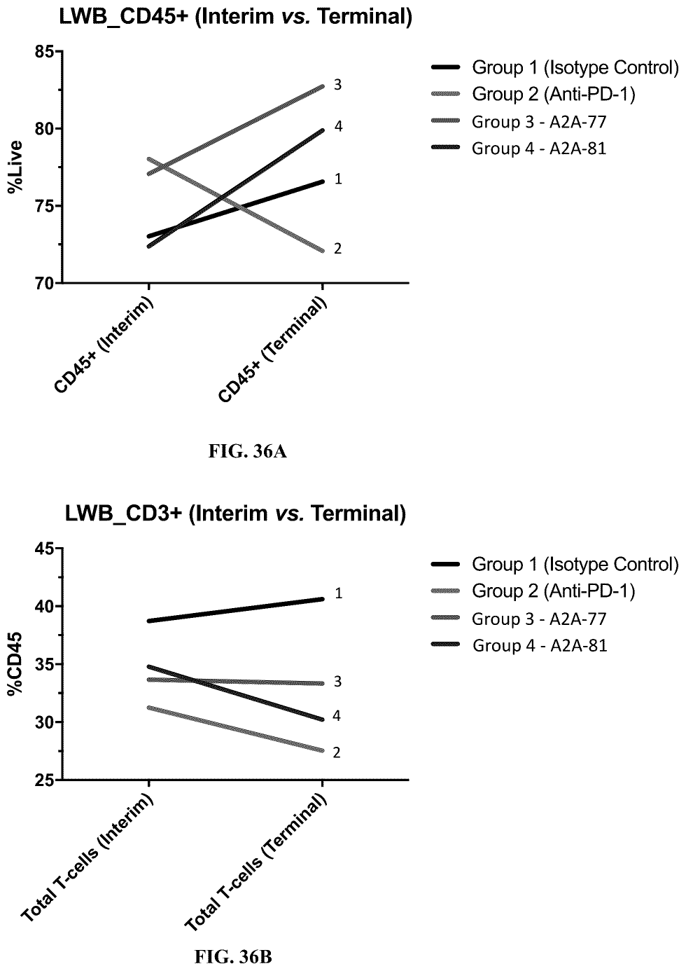

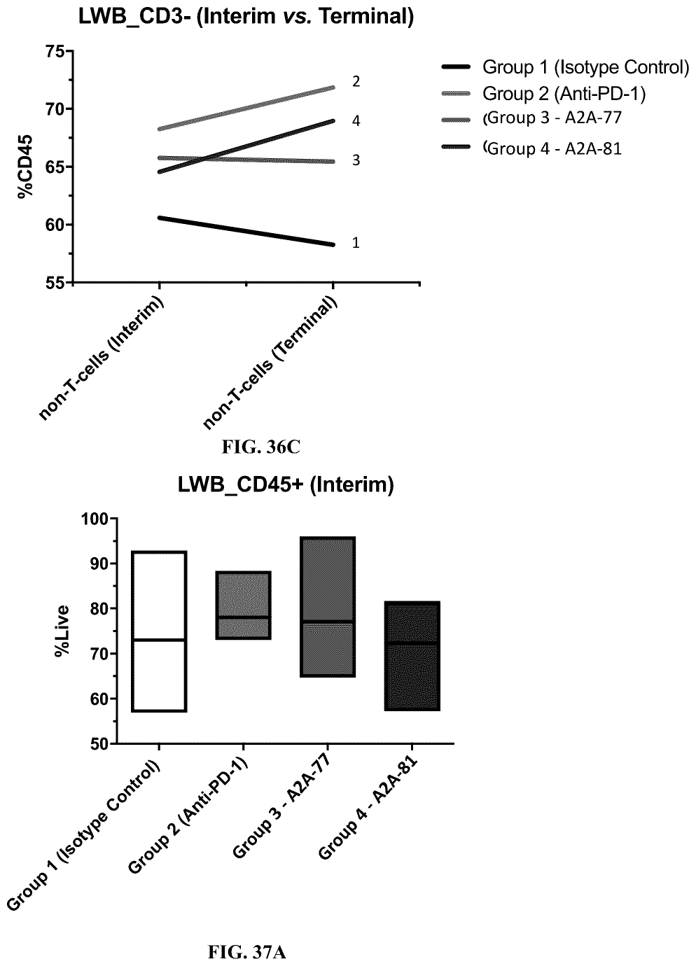

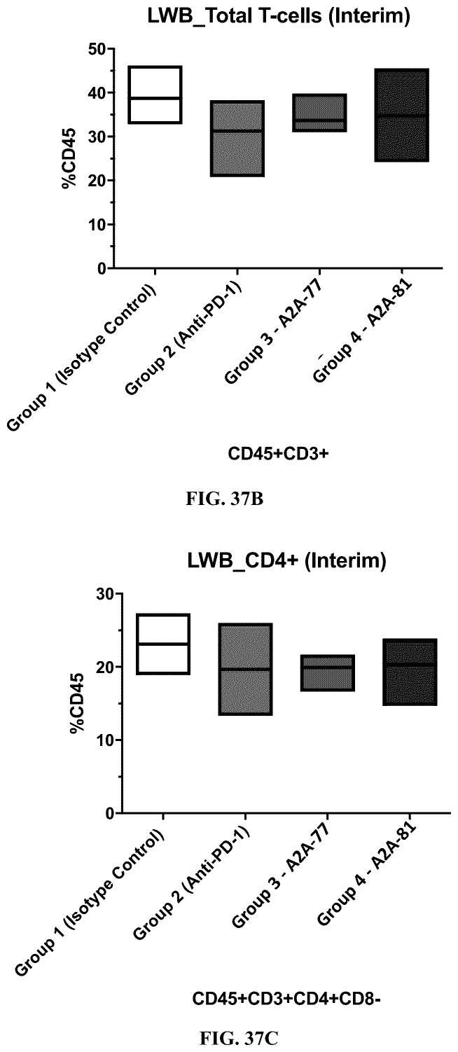

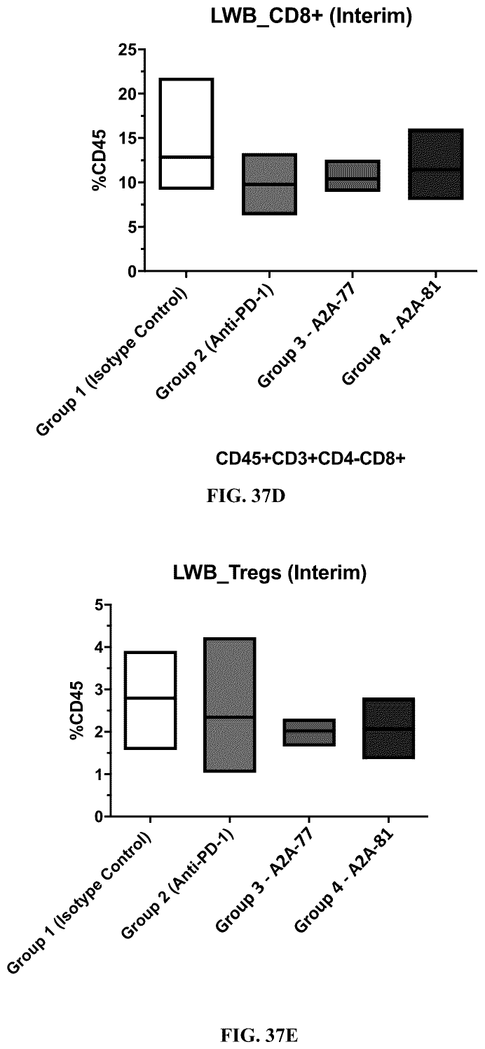

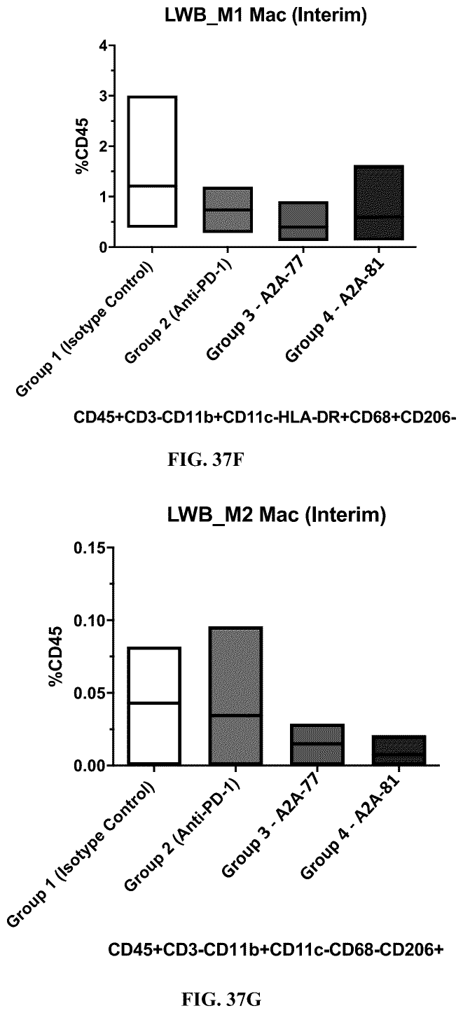

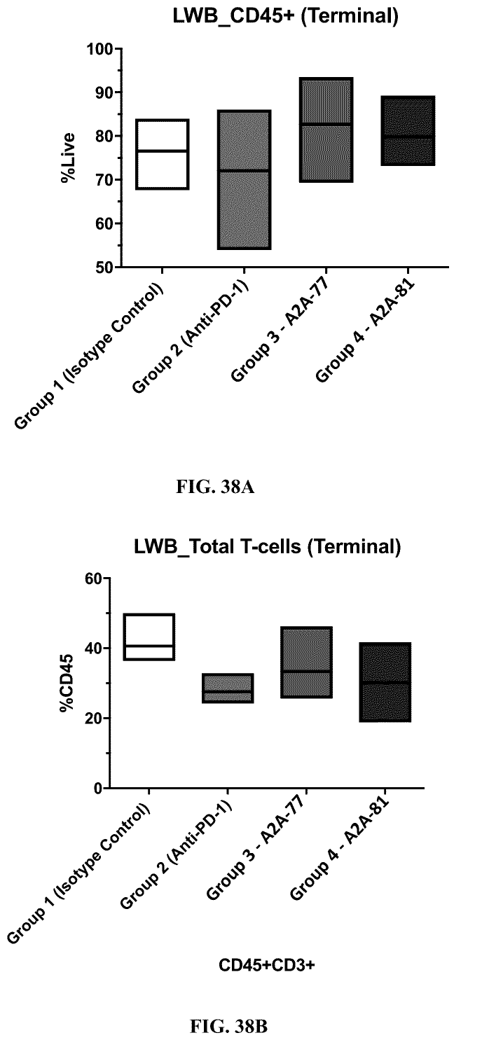

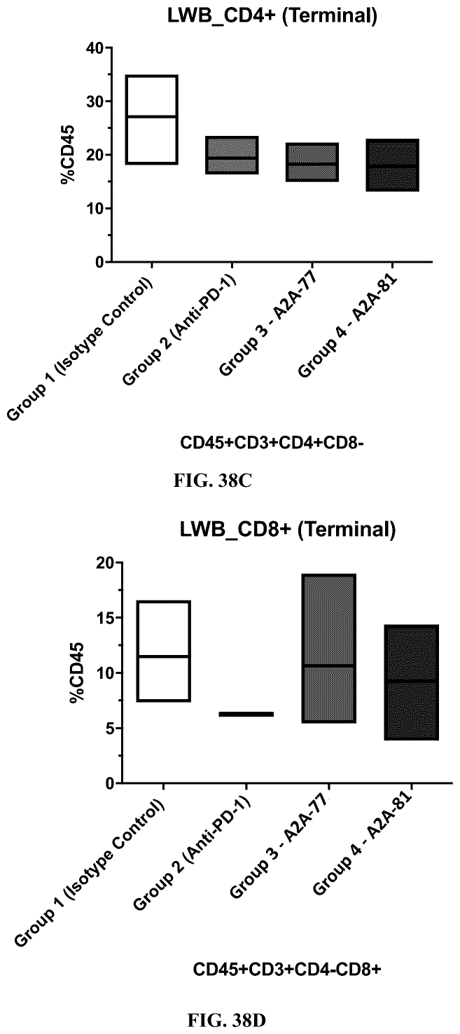

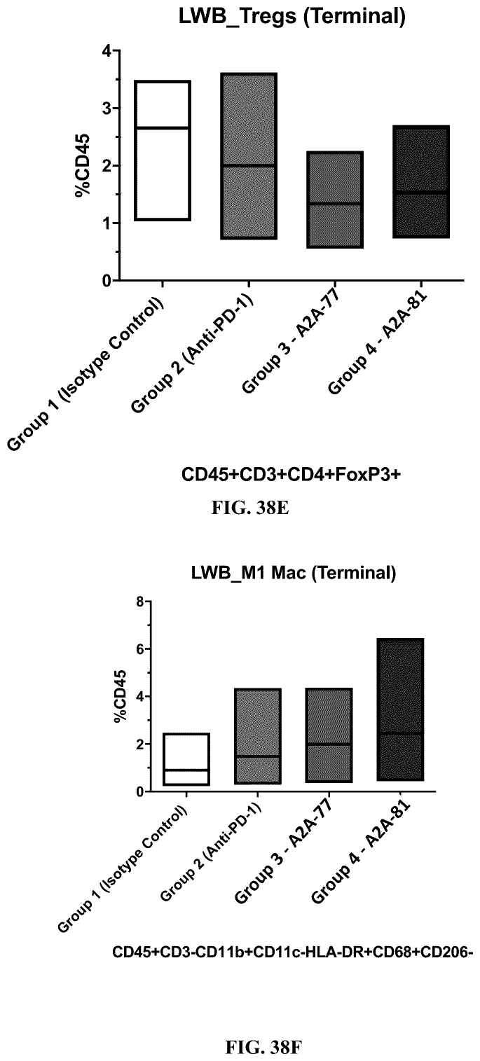

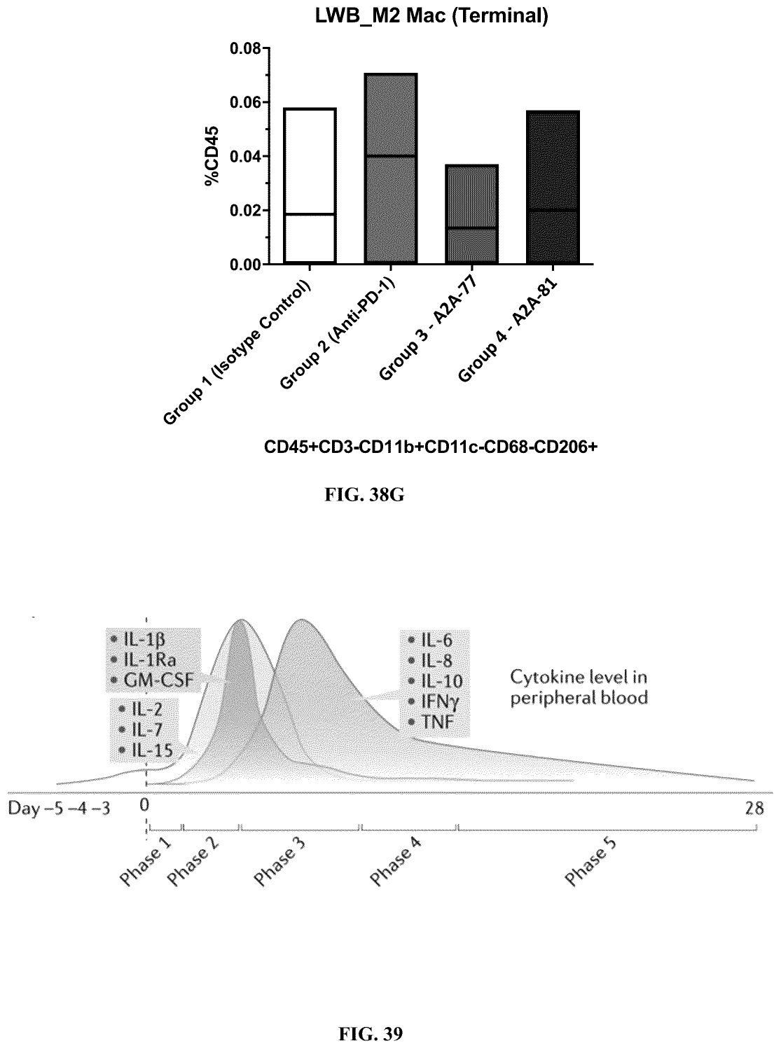

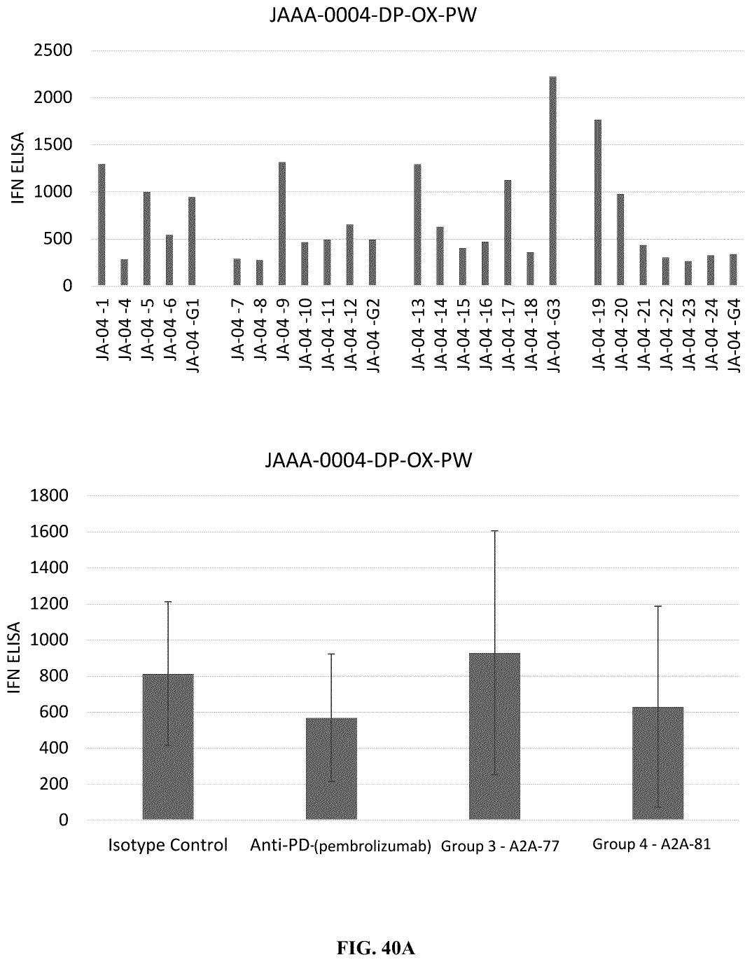

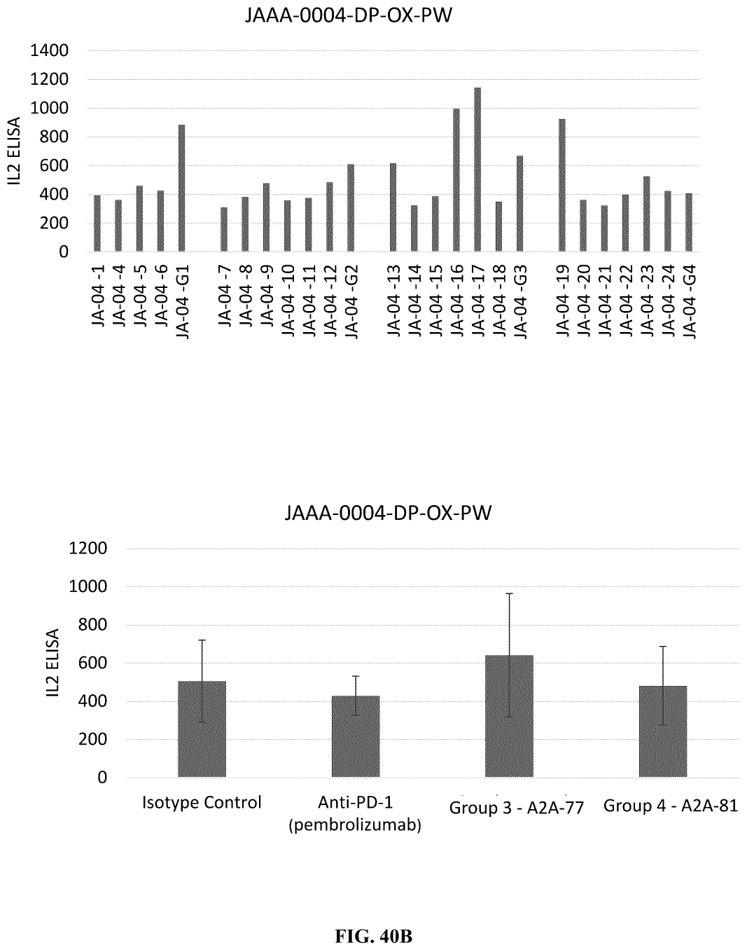

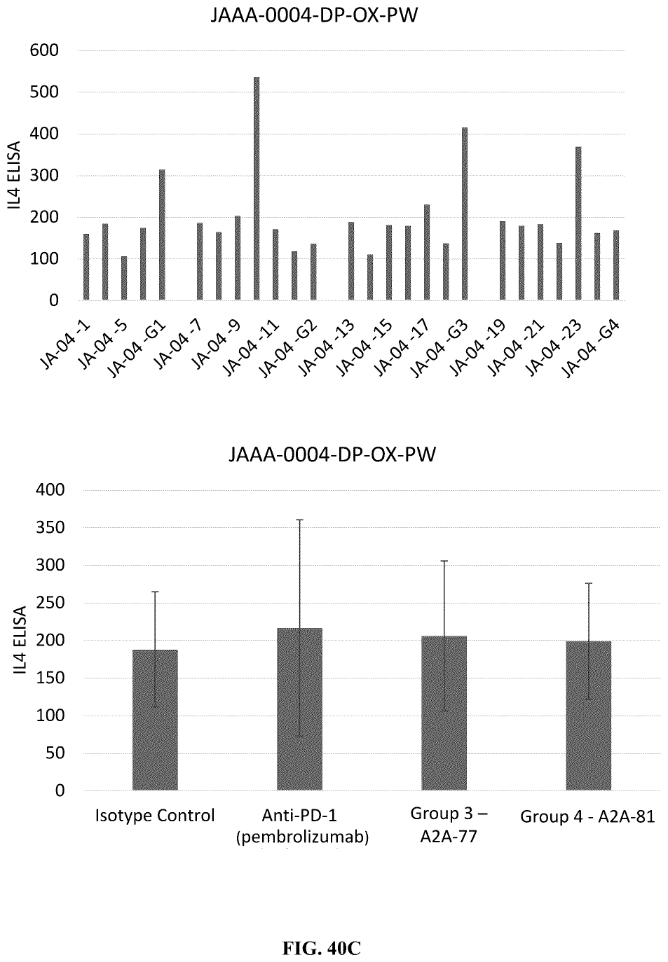

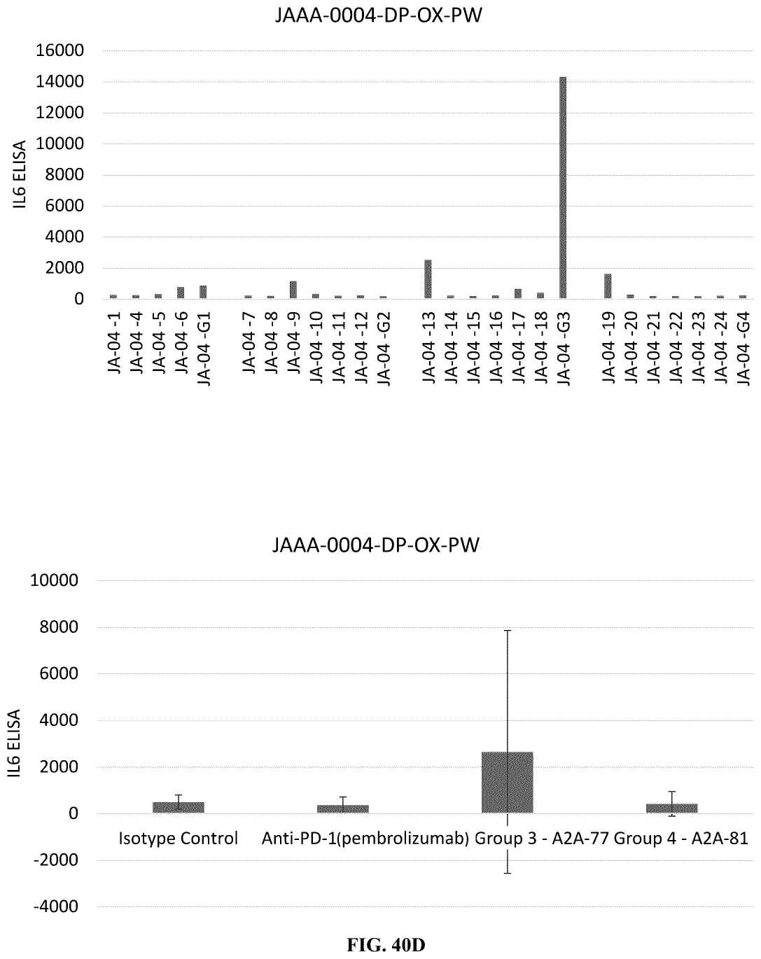

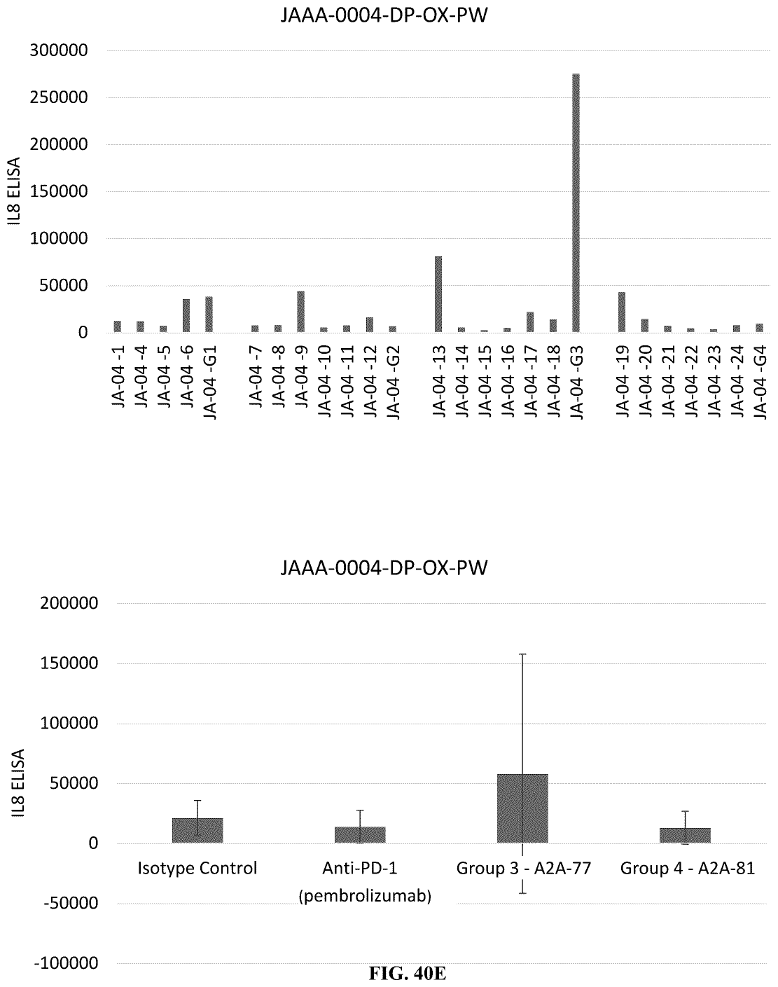

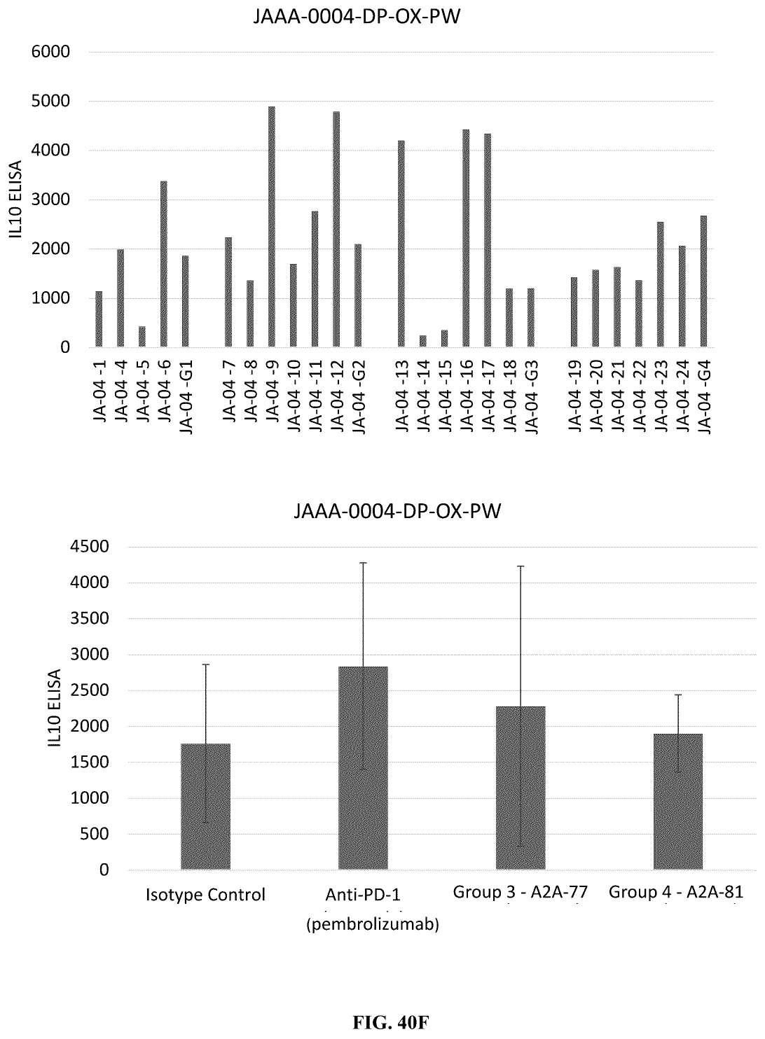

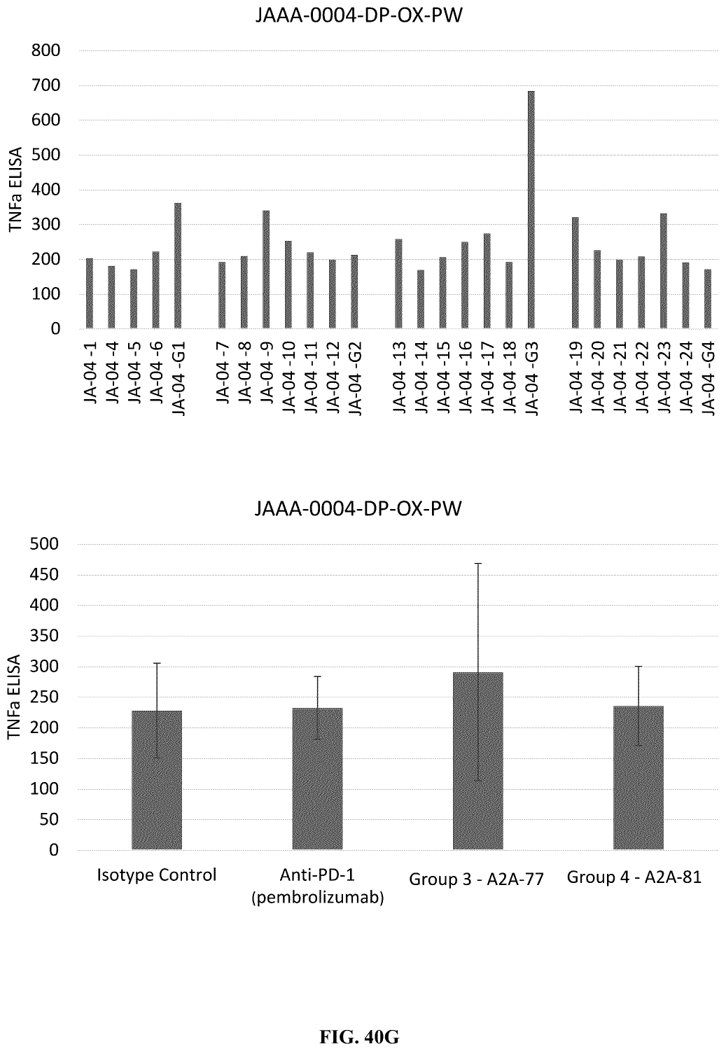

The cell profile of lysed whole blood (LWB) of peripheral blood was measured in interim and terminal samples. The results are depicted in FIGS. 36A-36C, FIGS. 37A-37G, and FIGS. 38A-38G. Cytokine levels in peripheral blood after T cell activation is depicted in FIG. 39. Results of the cytokine levels in terminal serum is depicted in FIGS. 40A-40G.

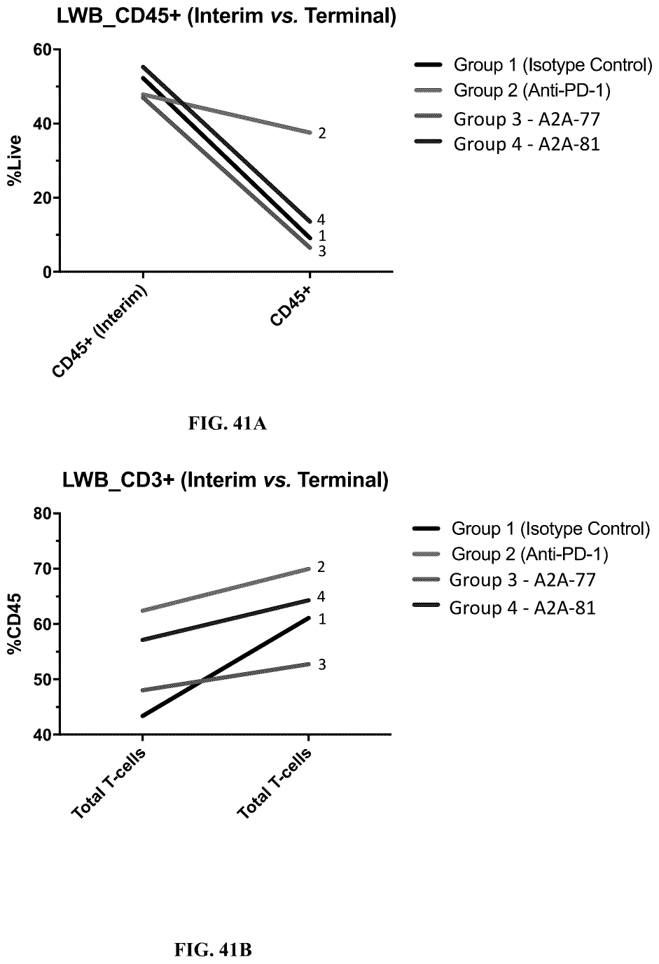

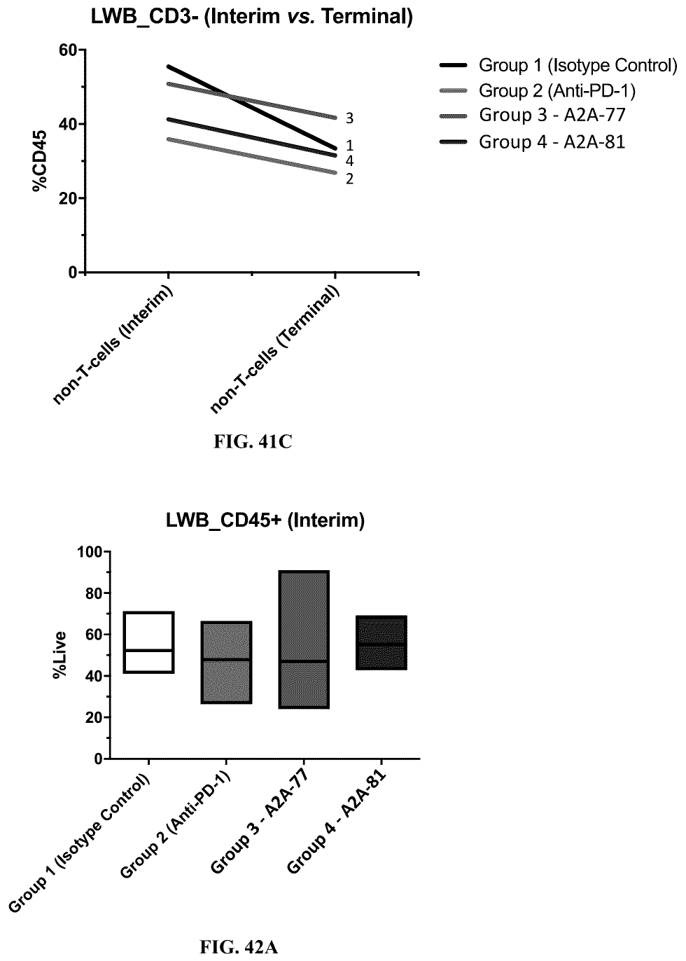

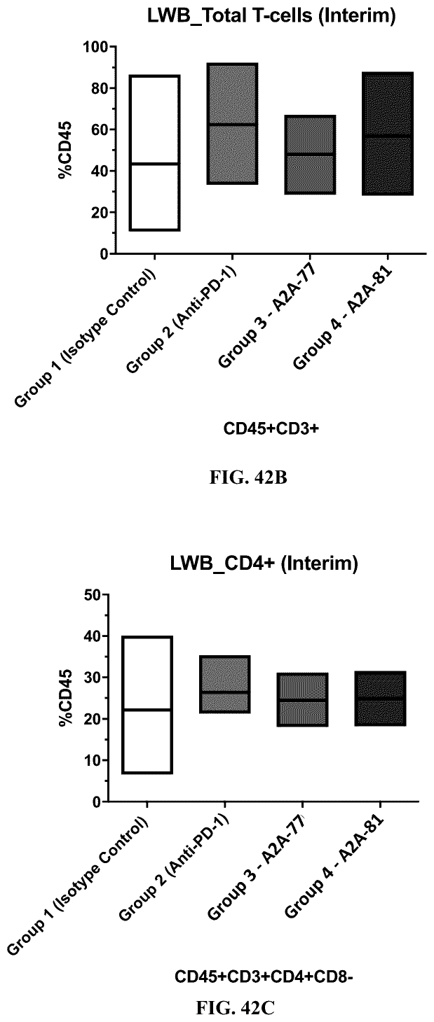

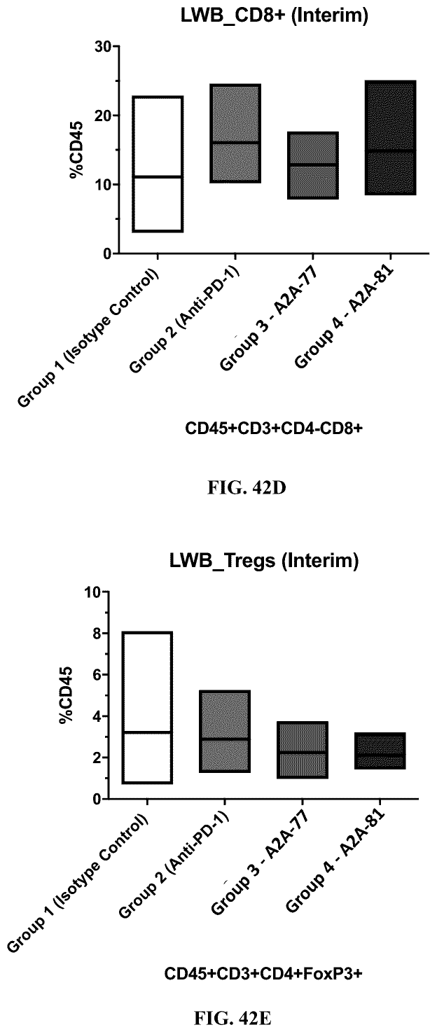

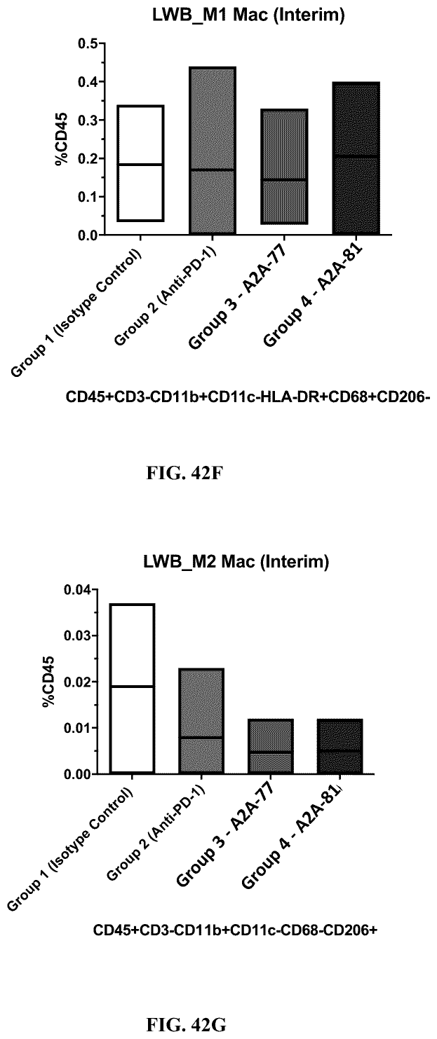

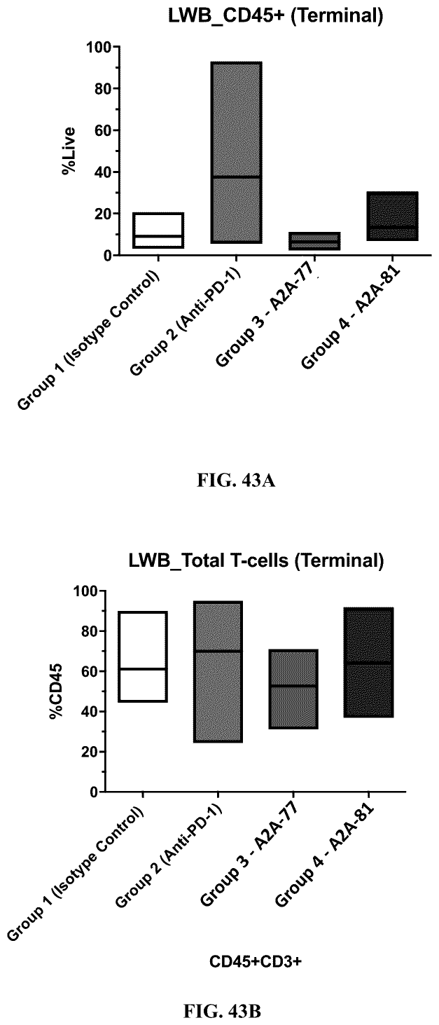

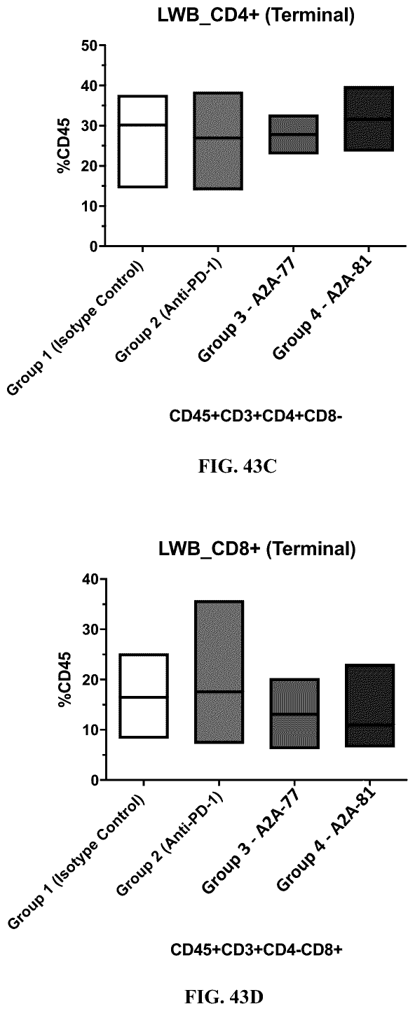

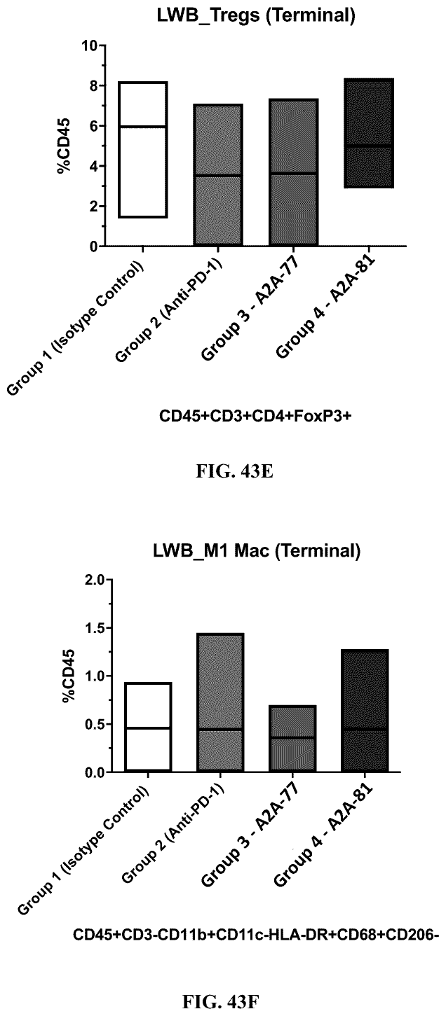

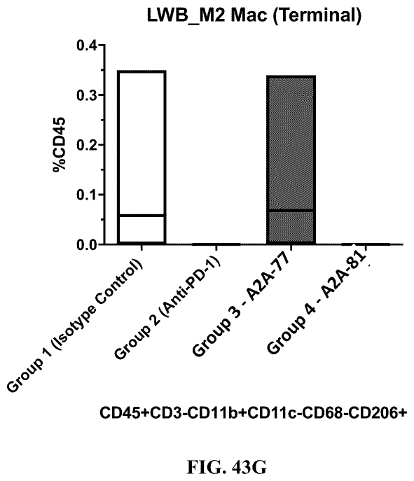

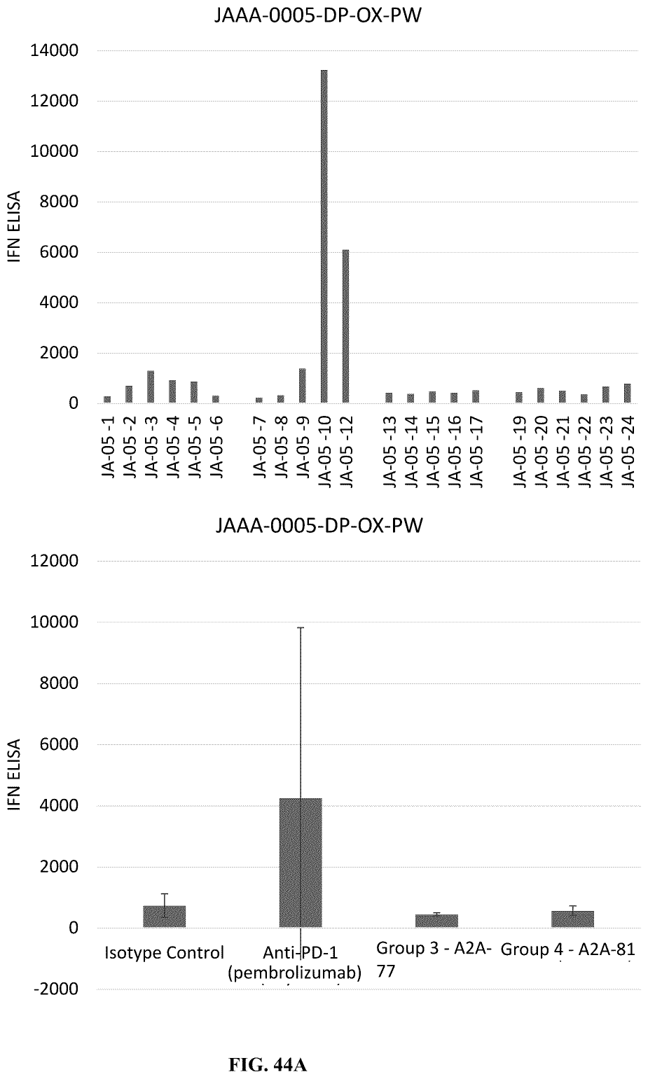

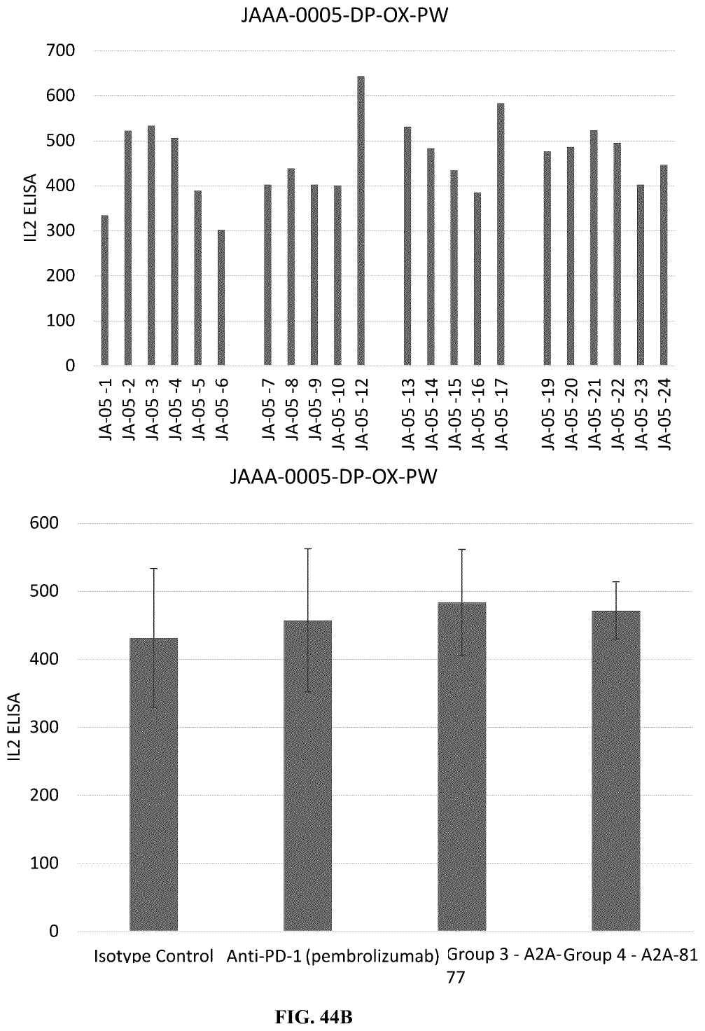

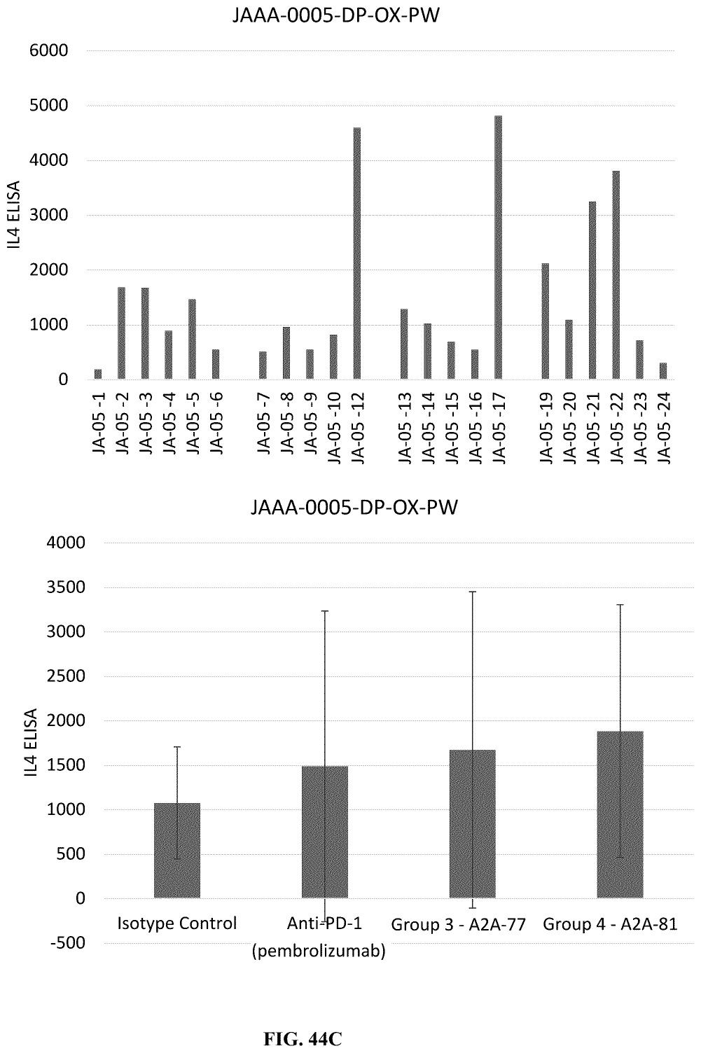

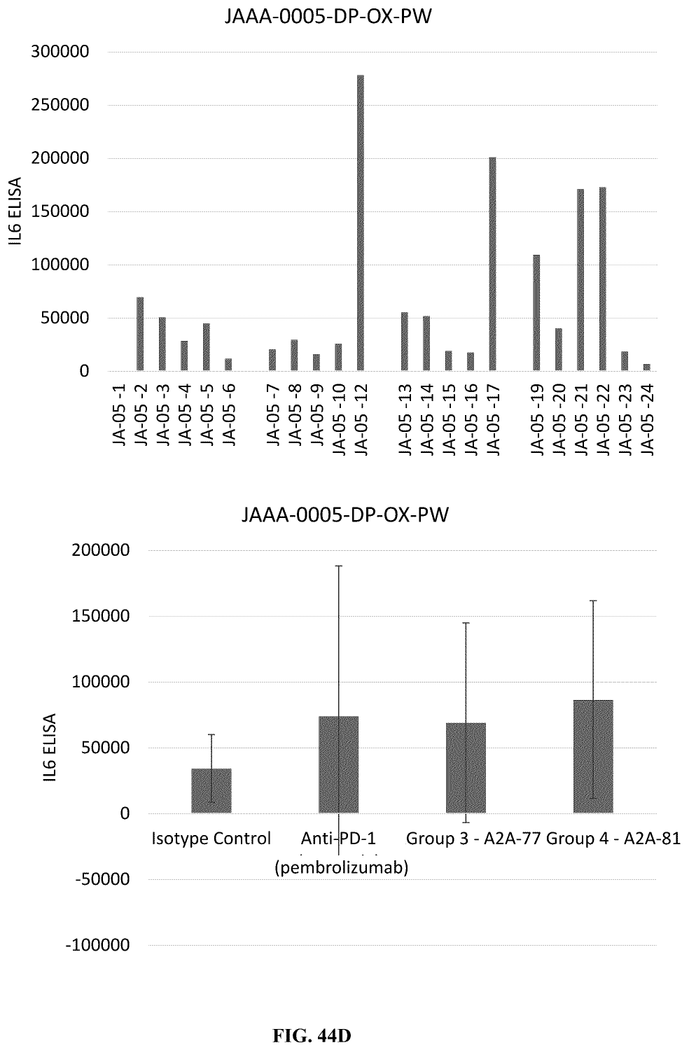

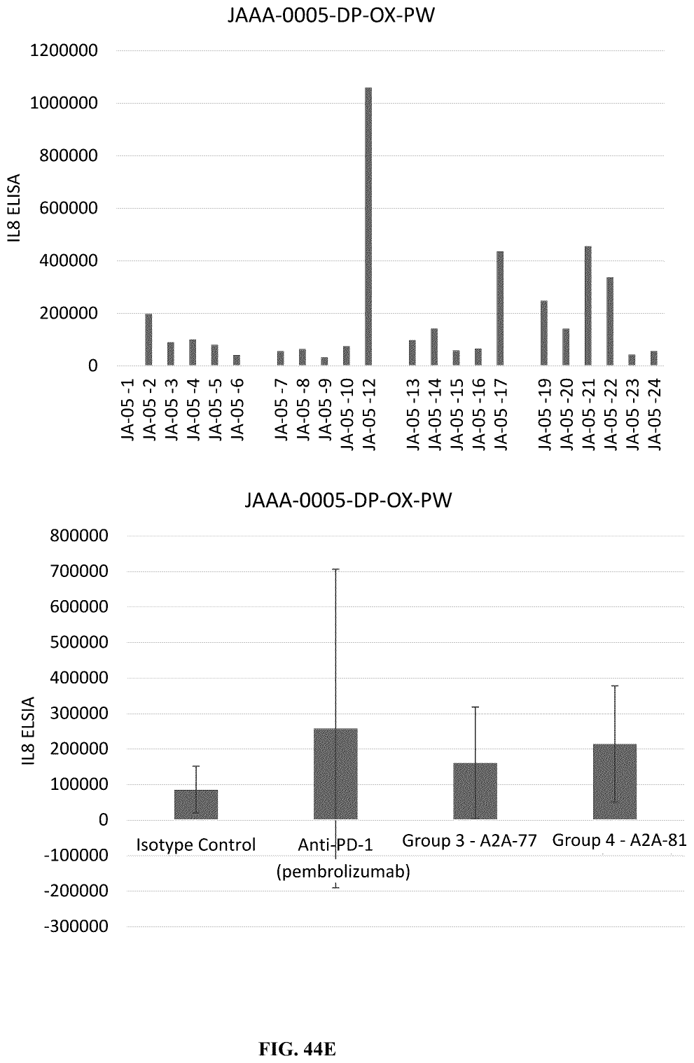

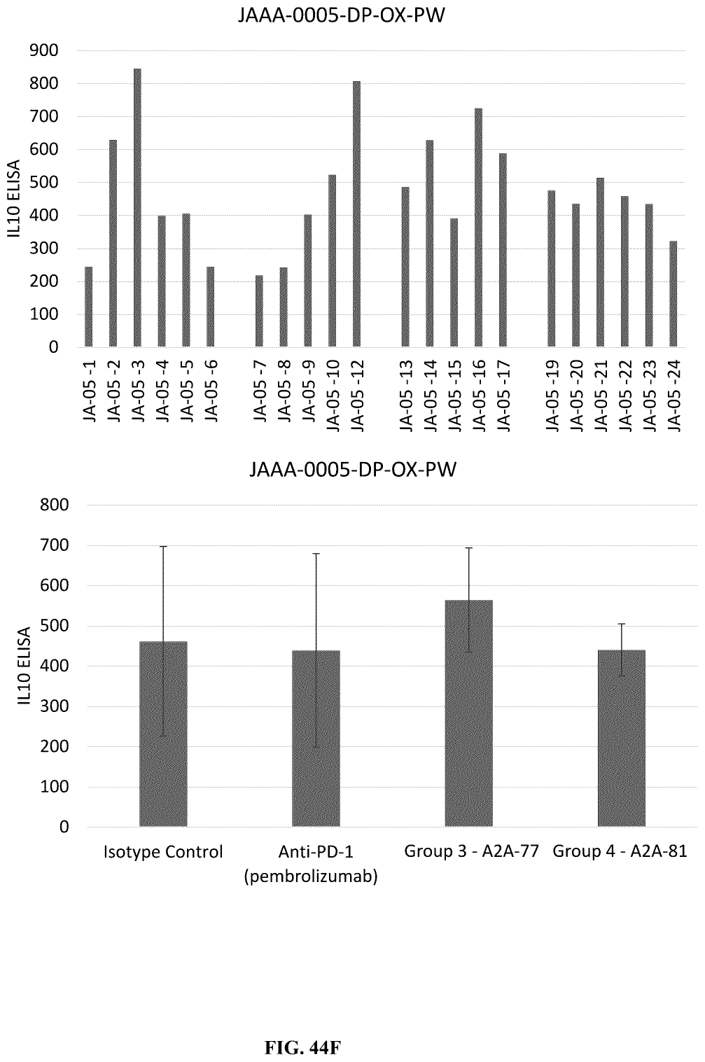

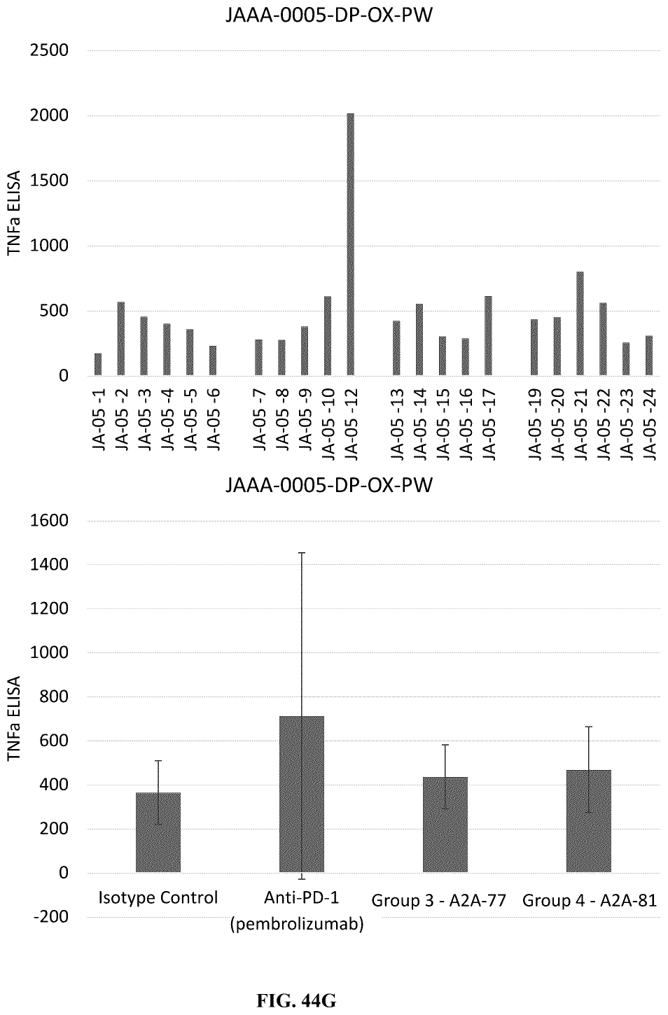

The cell profile of lysed whole blood (LWB) of peripheral blood was measured in interim and terminal samples. The results are depicted in FIGS. 41A-41C, 42A-42G and 43A-43G. Results of the cytokine levels in terminal serum samples is depicted in FIGS. 44A-44G.

### Example 25: A2bR Cell Functional cAMP Assay

### Cross Reactivity

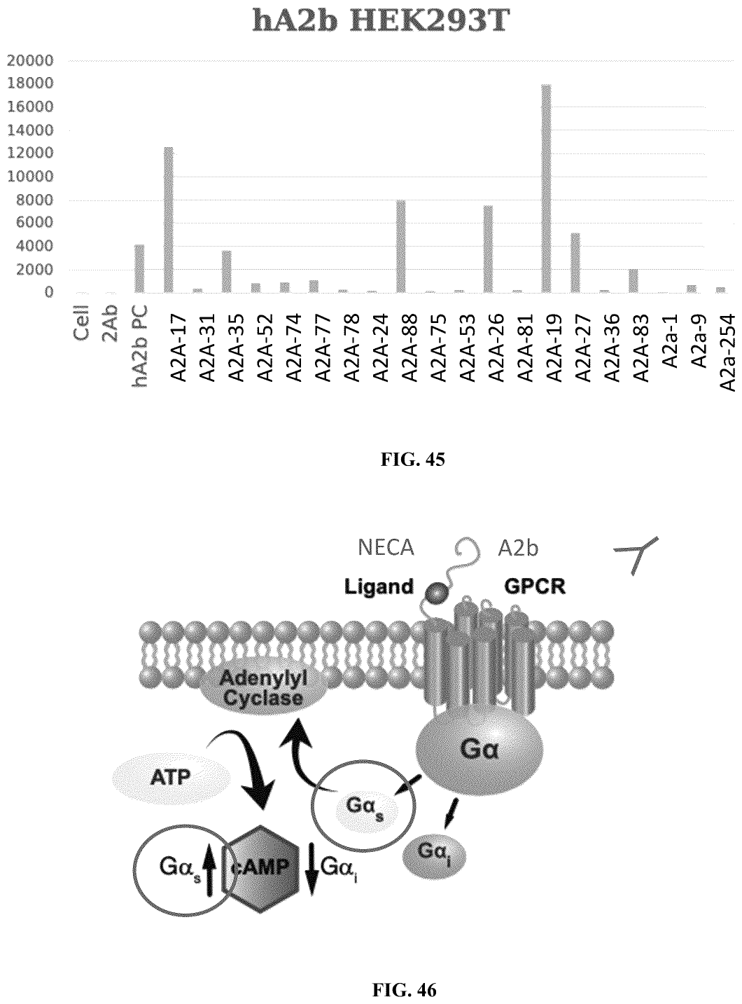

A2b cross binders were assessed for specificity in HEK293T cells were assayed for cross reactivity. Results are depicted in FIG. 45 and Table 22.

TABLE 22

| | A2a antagonists | |
|---|---|---|
| wA2a antagonists (1^{st} | (narrow | |
| screen) | down leads) | |
| Functional | Functional | Cross react hA1, |
| PE antagonistic cAMP | DiscoverX cAMP | A2b, A3 |
| A2A-17, A2A-19 | A2A-19 | A2A-17, A2A-19 |
| A2A-24, A2A-26, A2A-27, | A2A-24, A2A-26, | A2A-26, A2A-27 |
| | A2A-27 | |
| A2A-35, A2A-36, A2A-37 | A2A-37 | A2A-35, A2A-37 |
| A2A-51, A2A-52, A2A-53 | A2A-51, A2A-53 | |
| A2A-74, A2A-75 | A2A-75 | |
| A2A-77, A2A-78, A2A-81 | A2A-78, A2A-81 | |
| A2A-83, A2A-84 | A2A-83, A2A-84 | A2A-83 |

FIG. 46 depicts the functional cAMP assay performed on selected A2b antibodies. CHO-K1 cells were incubated with an A2b antibody. Next, the cells were stimulated using NECA. Activation of A2b was monitored based on 3′-5′-cyclic adenosine monophosphate (cAMP) production in the cell lines.

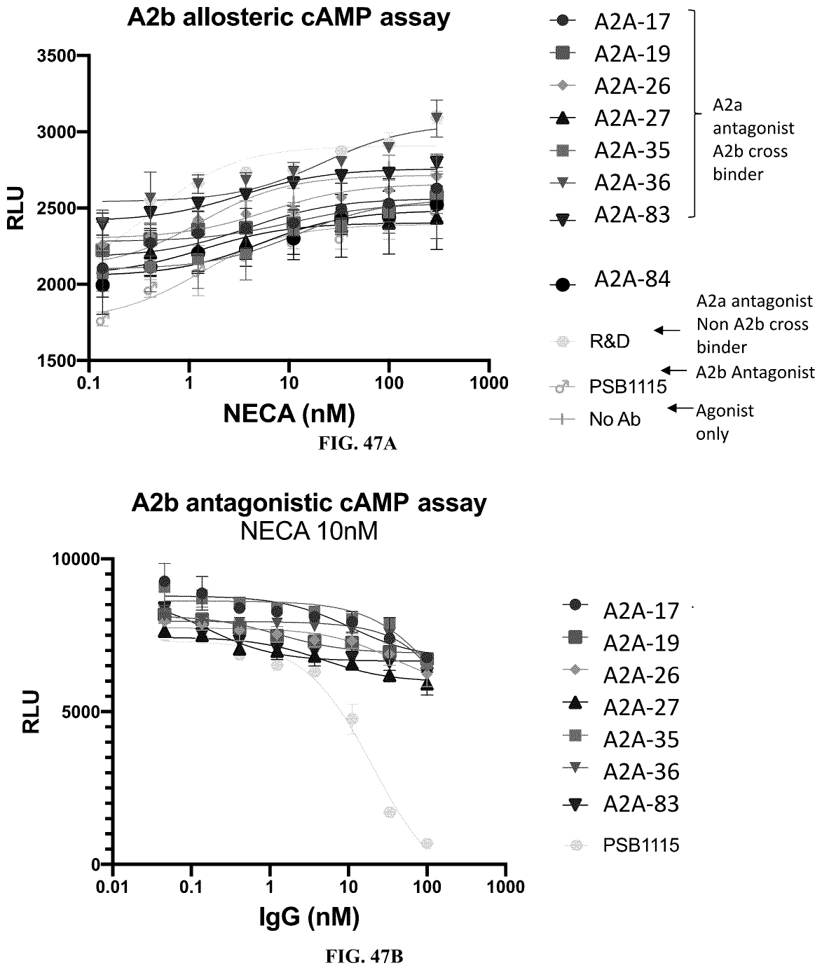

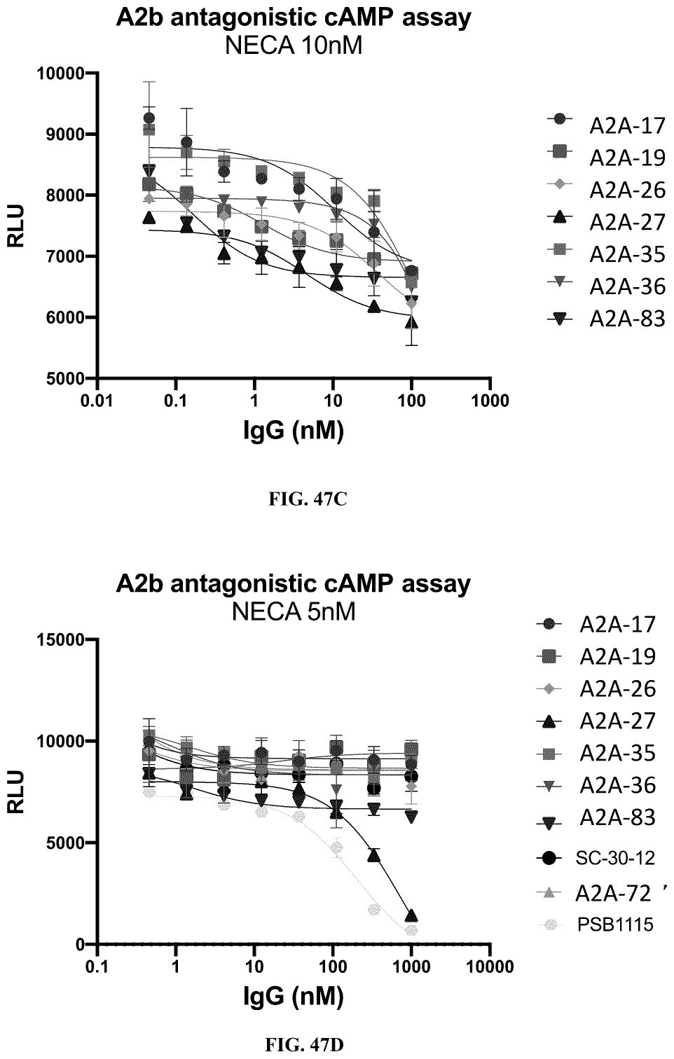

A functional allosteric cAMP assay was performed. Cells were pre-incubated with anti-A2A-17, A2A-19, A2A-26, A2A-27, A2A-35, A2A-36, A2A-83, and A2A-84 at 100 nM, followed by NECA stimulation at a 3× titration from 300 nM. The results are depicted in FIG. 47A.

A functional antagonistic cAMP assay was performed. First, cells were pre-incubated with A2A-17, A2A-19, A2A-26, A2A-27, A2A-35, A2A-36, and A2A-83 at a 3× titration starting at 100 nM. Next NECA stimulation at a concentration of 10 nM was performed. The results are depicted in FIGS. 47B-47C.

An antagonistic CAMP assay at high levels of IgC and low ligand levels was performed. Cells were pre-incubated with A2A-17, A2A-19, A2A-26, A2A-27, A2A-35, A2A-36, A2A-83, and A2A-72 at a 3× titration starting at 1000 nM. Next, NECA stimulation was performed at a 5 nM concentration. The results are depicted in FIG. 47D. A2A-27 showed properties of being an A2a antagonist that cross links with A2b, while also being an A2b antagonist. Properties of A2b are depicted in Table 23.

TABLE 23

| Cross | | | | | | |
|---|---|---|---|---|---|---|
| reactive | | | | | | |
| hA1, | | | | | | |
| A2b, A3 | HCDR1 | HCDR2 | HCDR3 | LCDR1 | LCDR2 | LCDR3 |
| A2A-27 | GFTFSSY | SGSGGS | ARGYVVRWR | RASQSIG | GASNLQS | QQGYSAPRT |
| (Alloy) | (SEQ ID | (SEQ ID | LWRRYDY | SYLN | (SEQ ID | (SEQ ID NO: |
| | NO: 10) | NO: 101) | (SEQ ID NO: | (SEQ ID | NO: 388) | 477) |
| | | | 210) | NO: 299) | | |

### Example 26. IgG1 and IgG4 Reformatted A2AR Antibodies

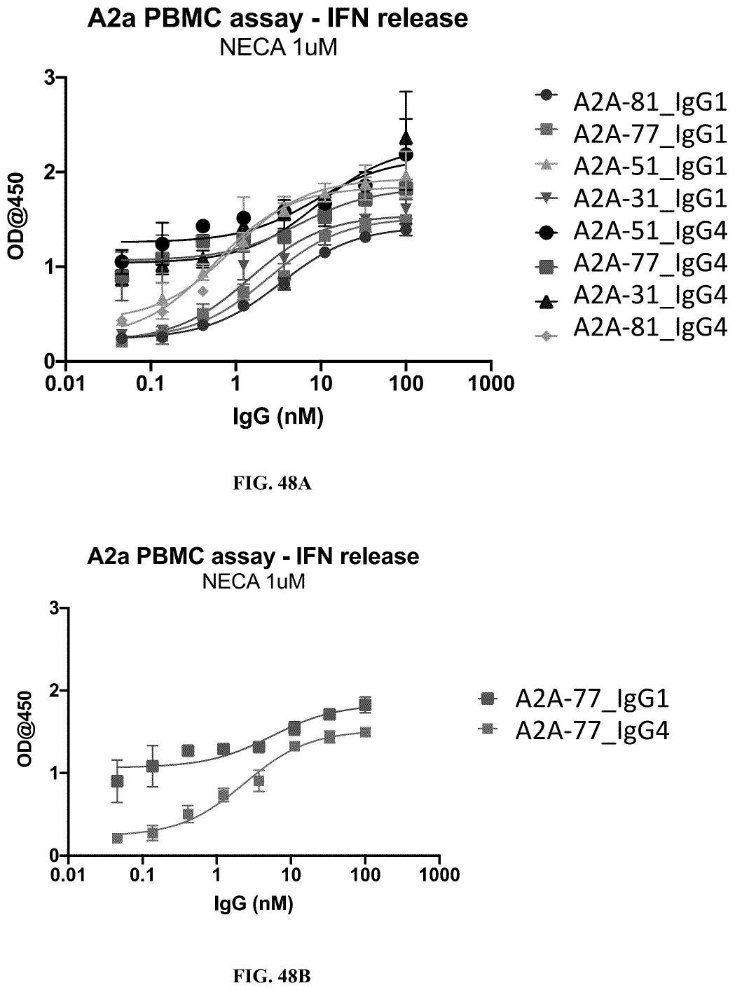

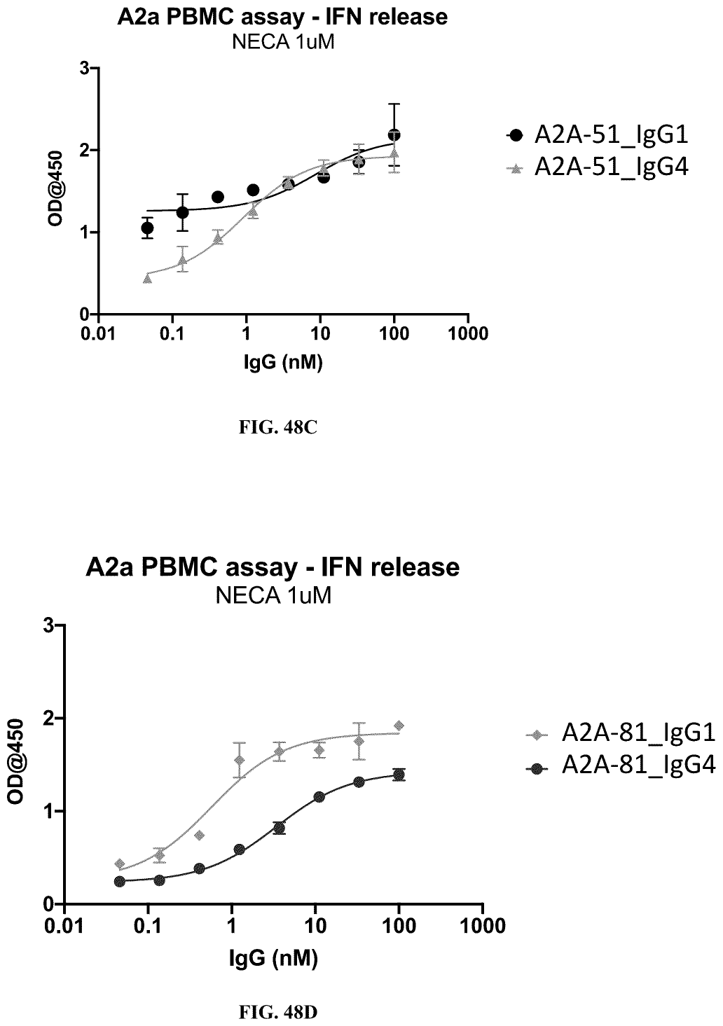

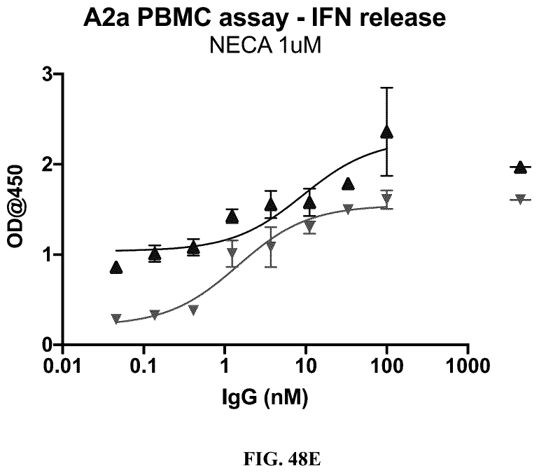

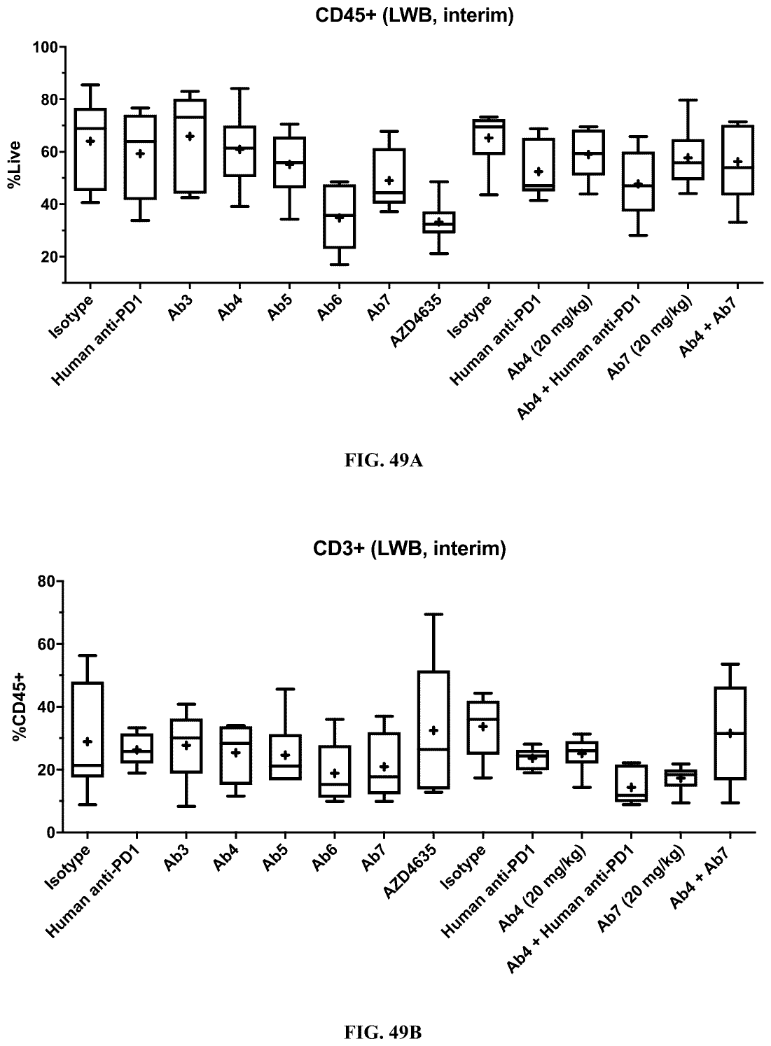

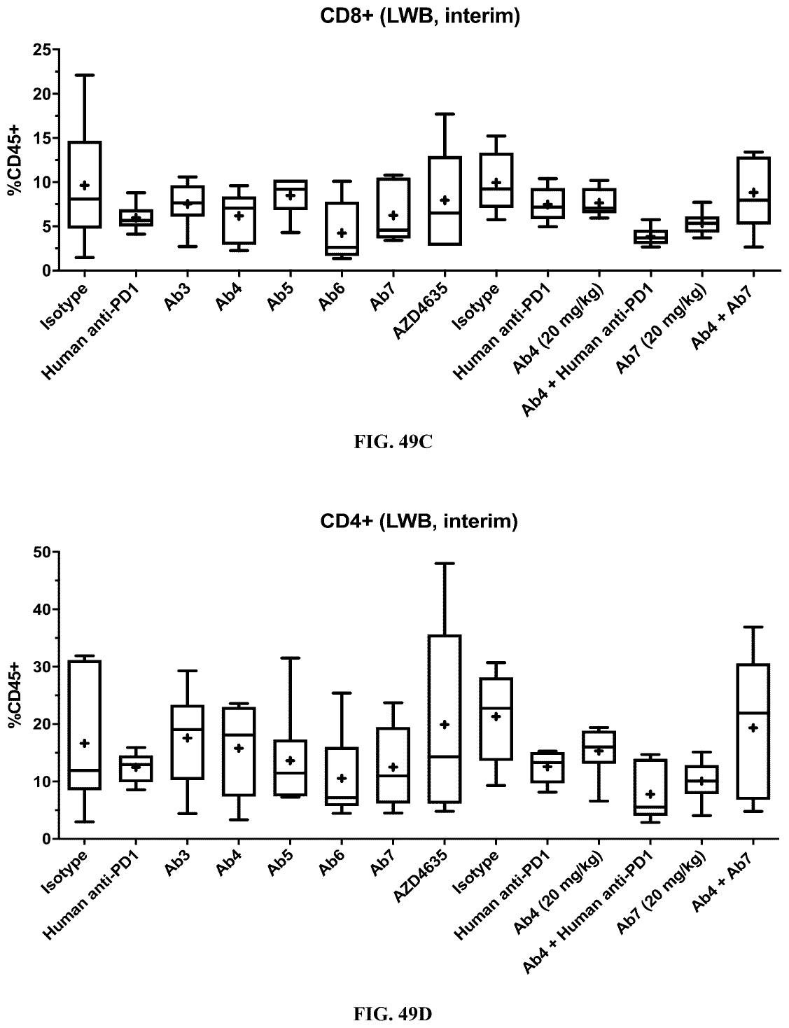

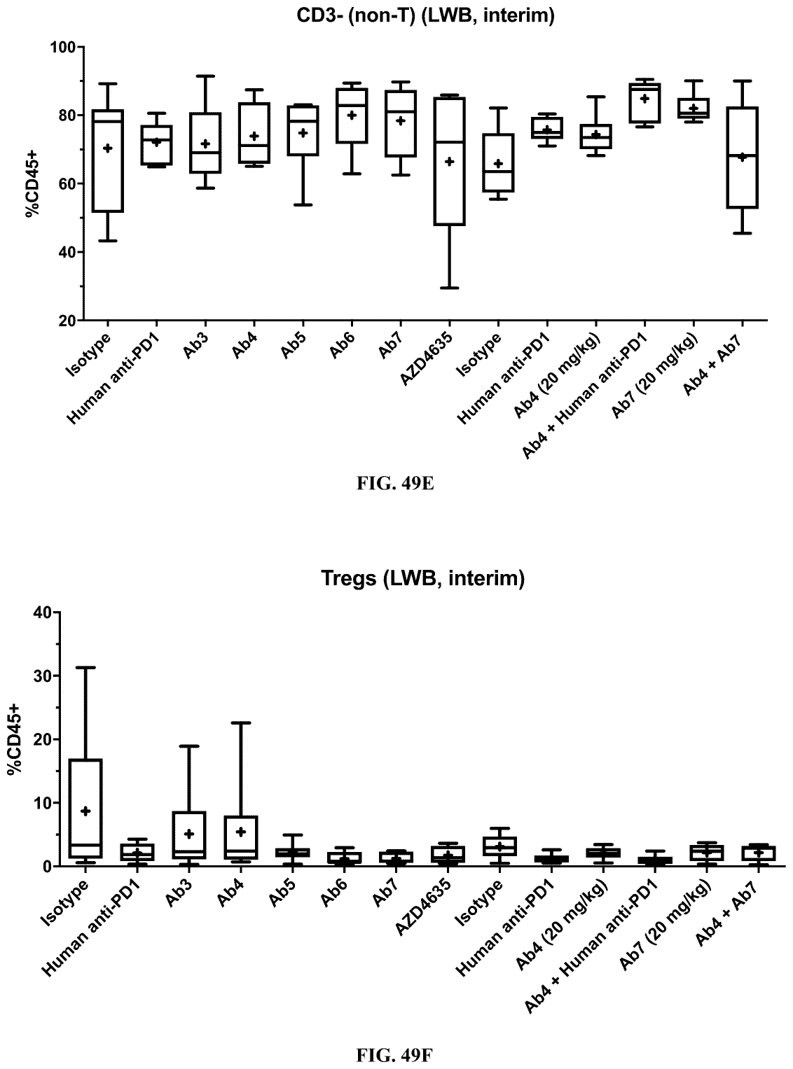

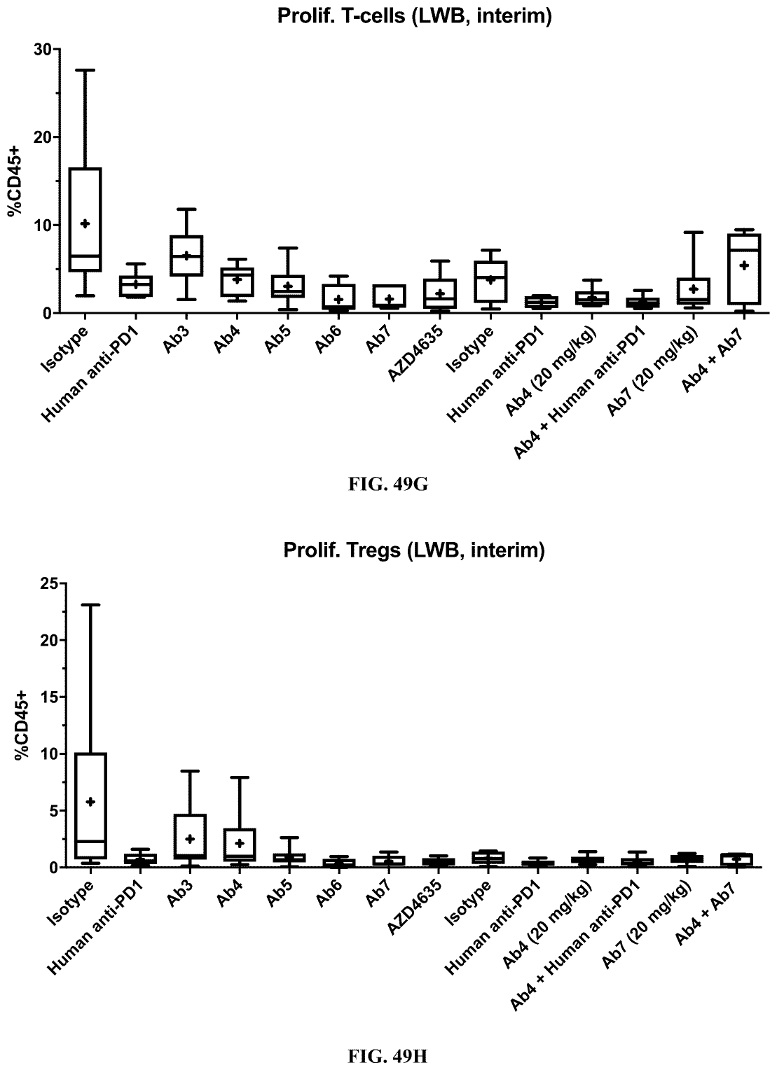

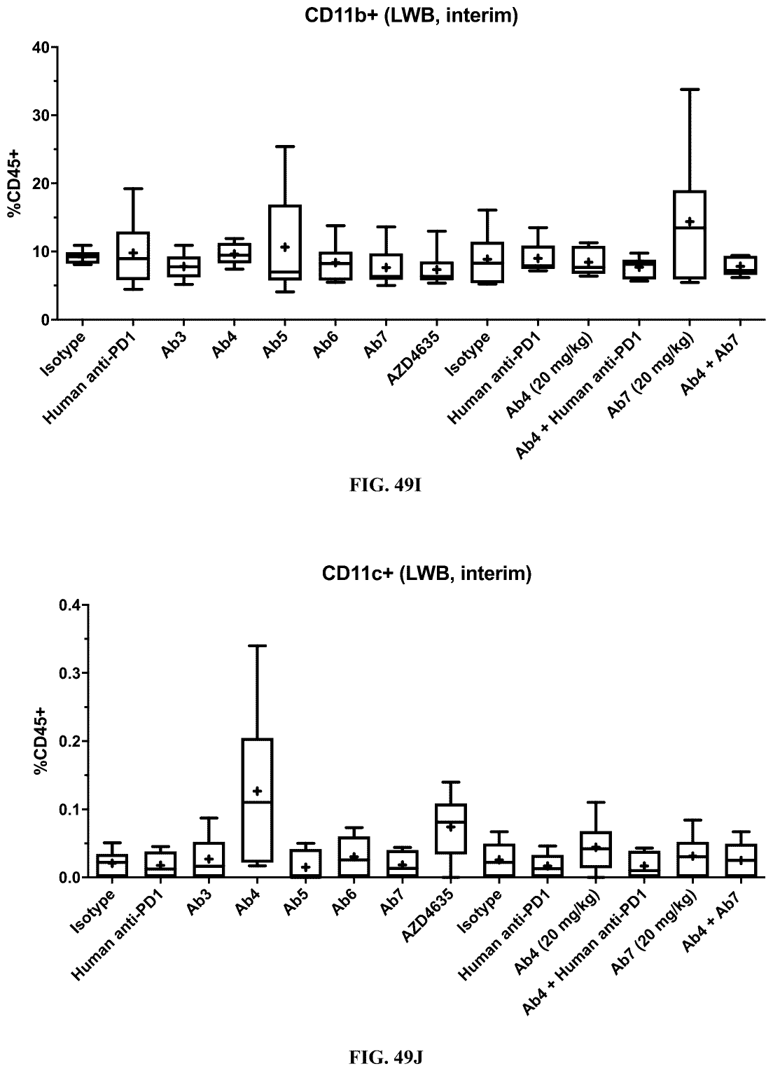

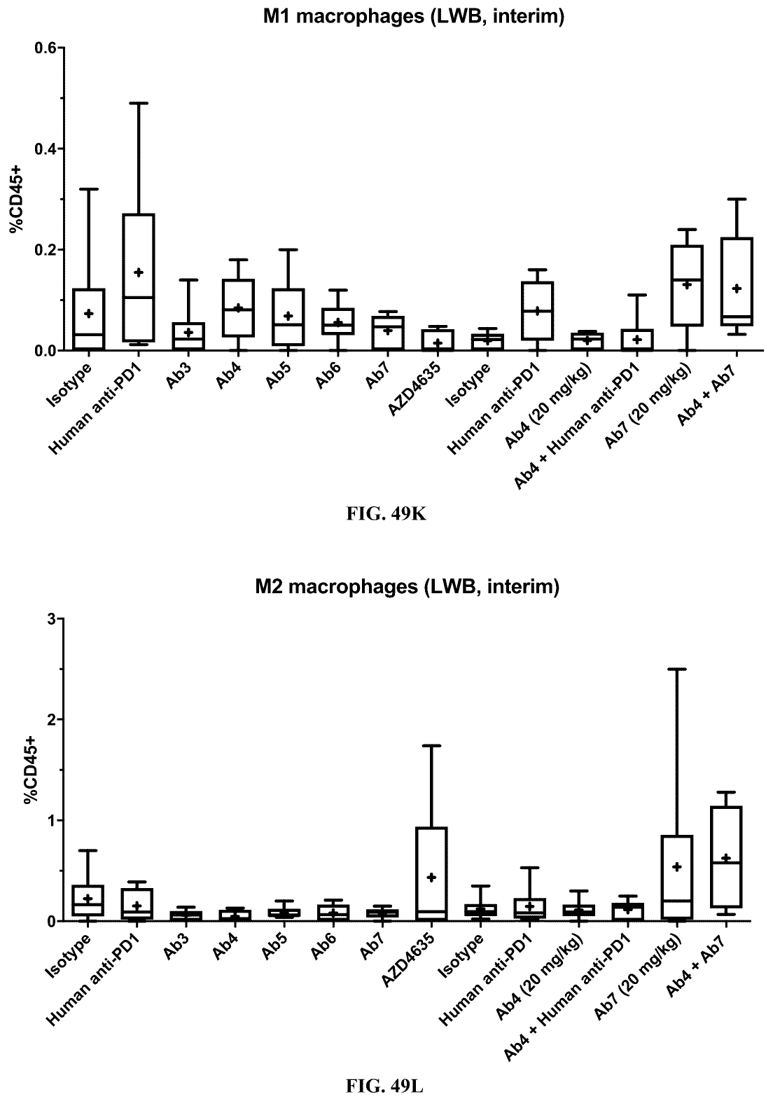

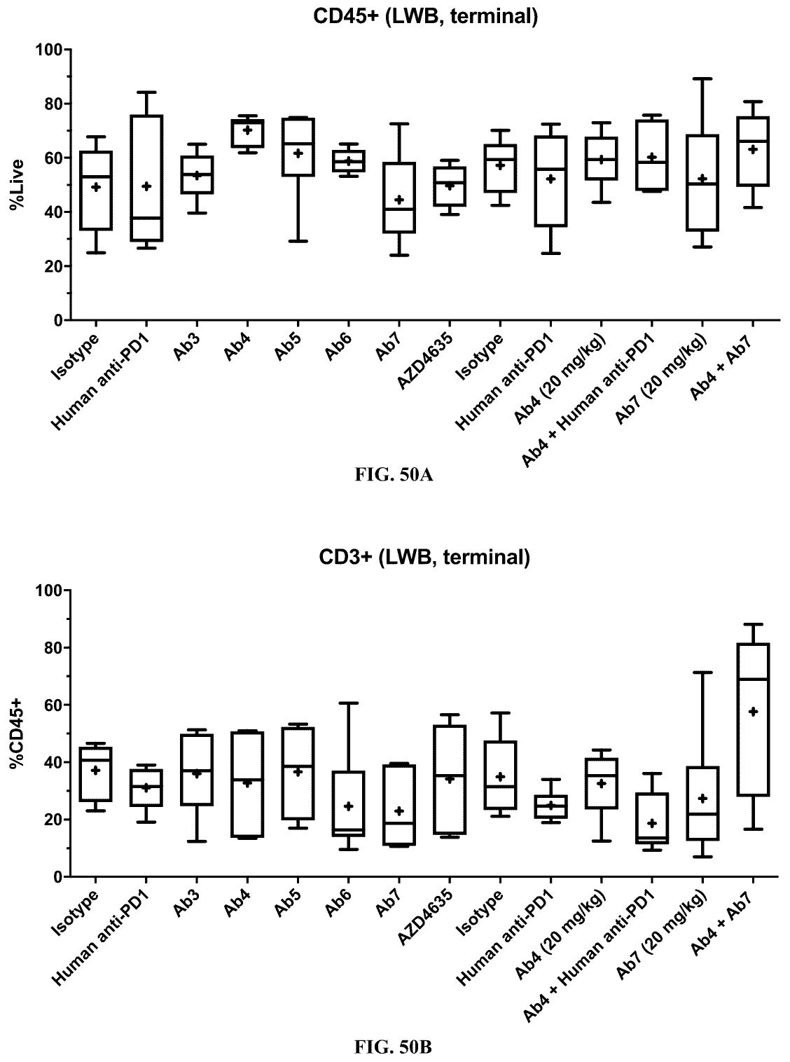

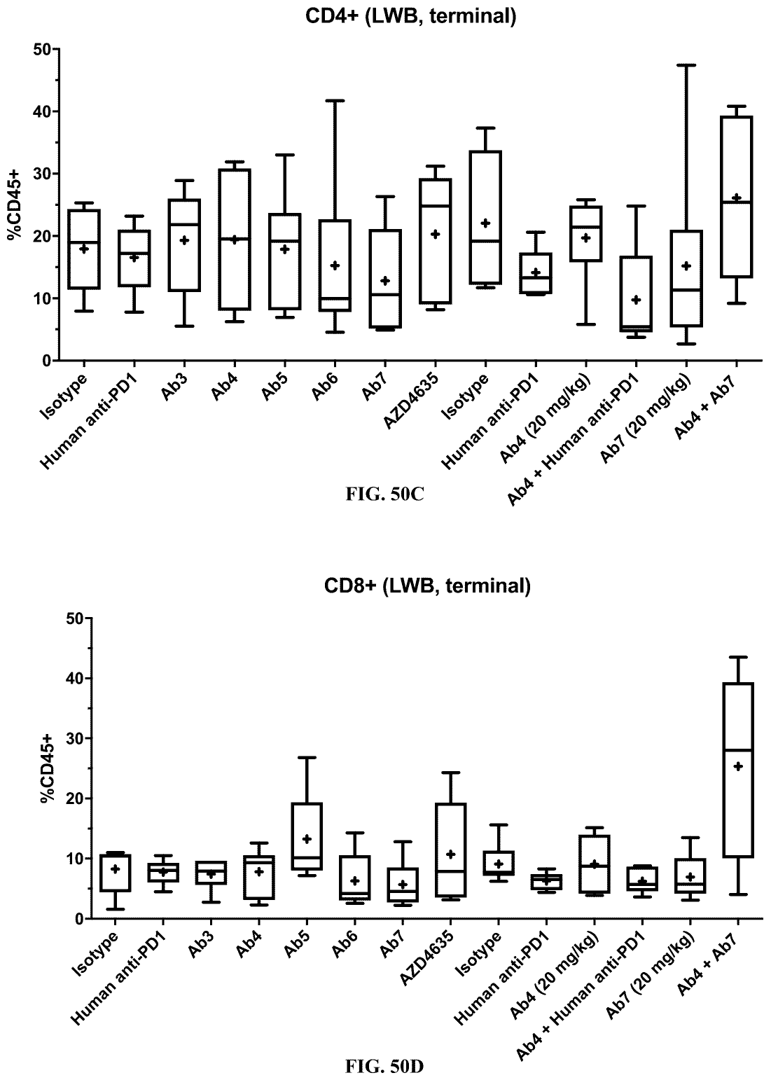

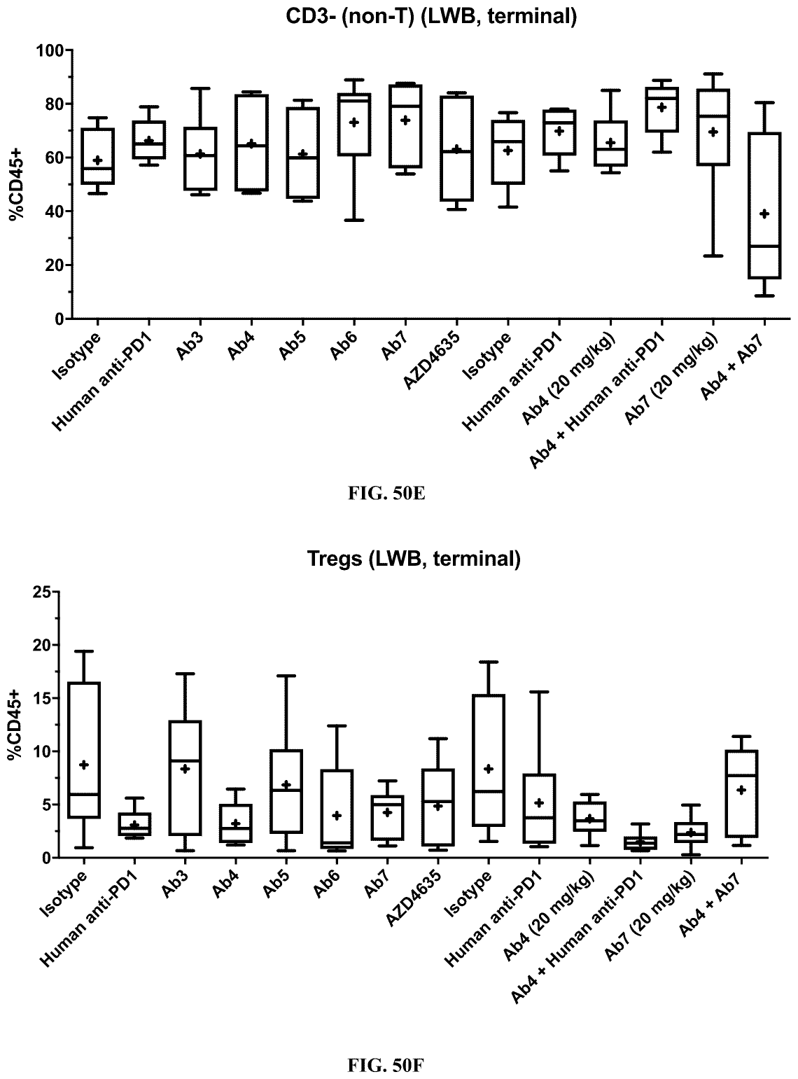

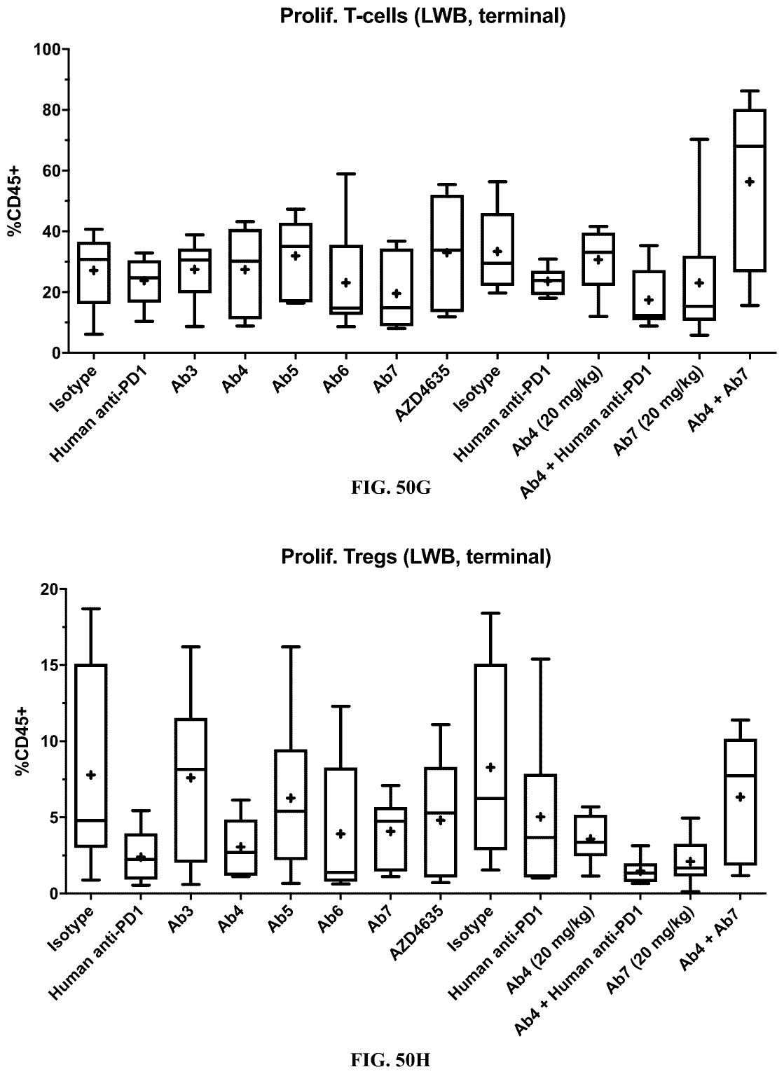

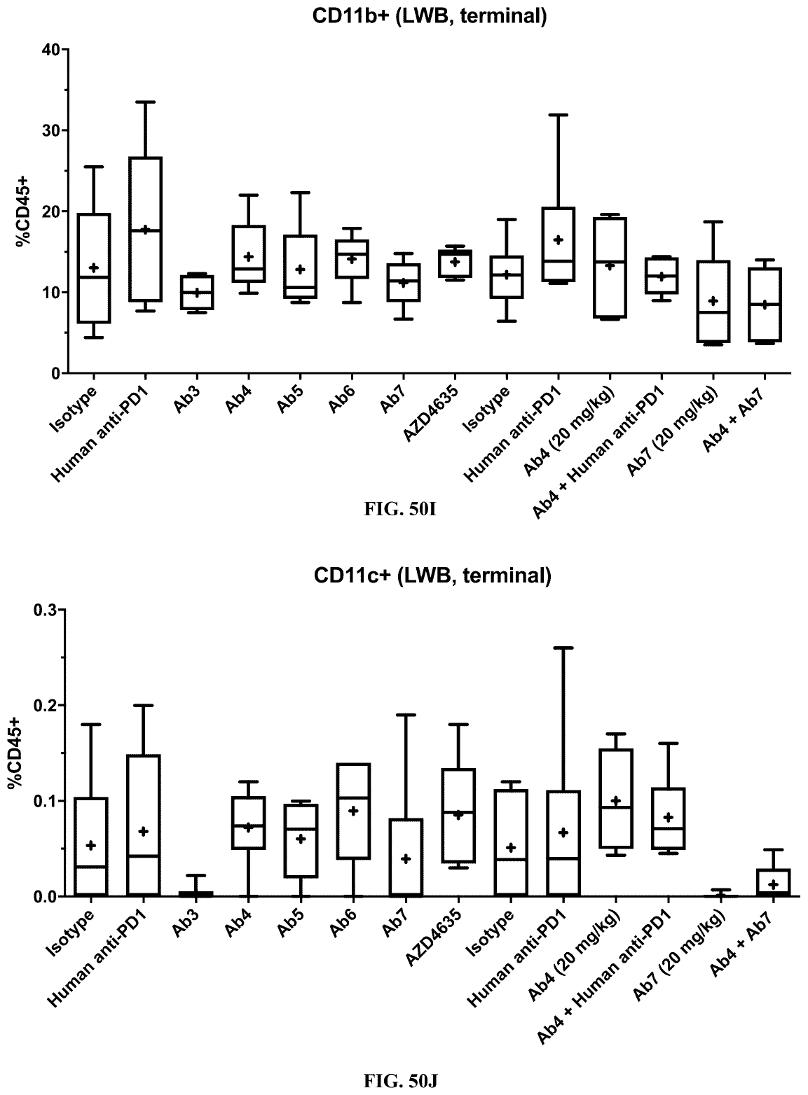

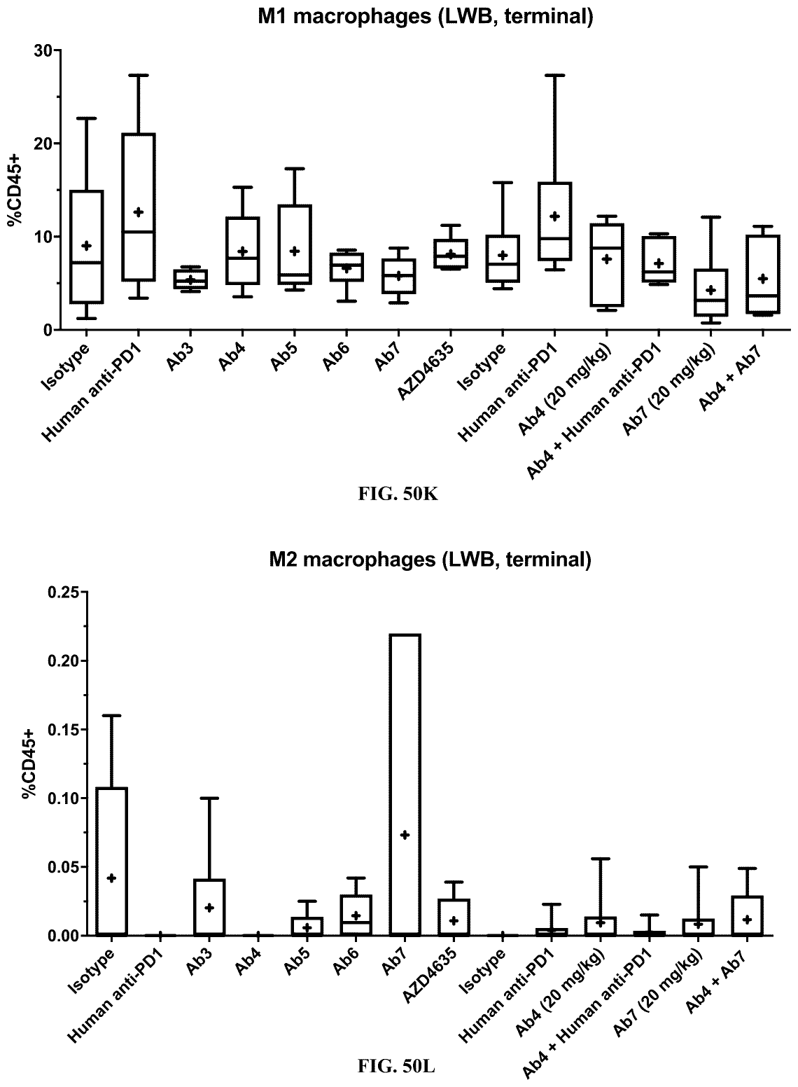

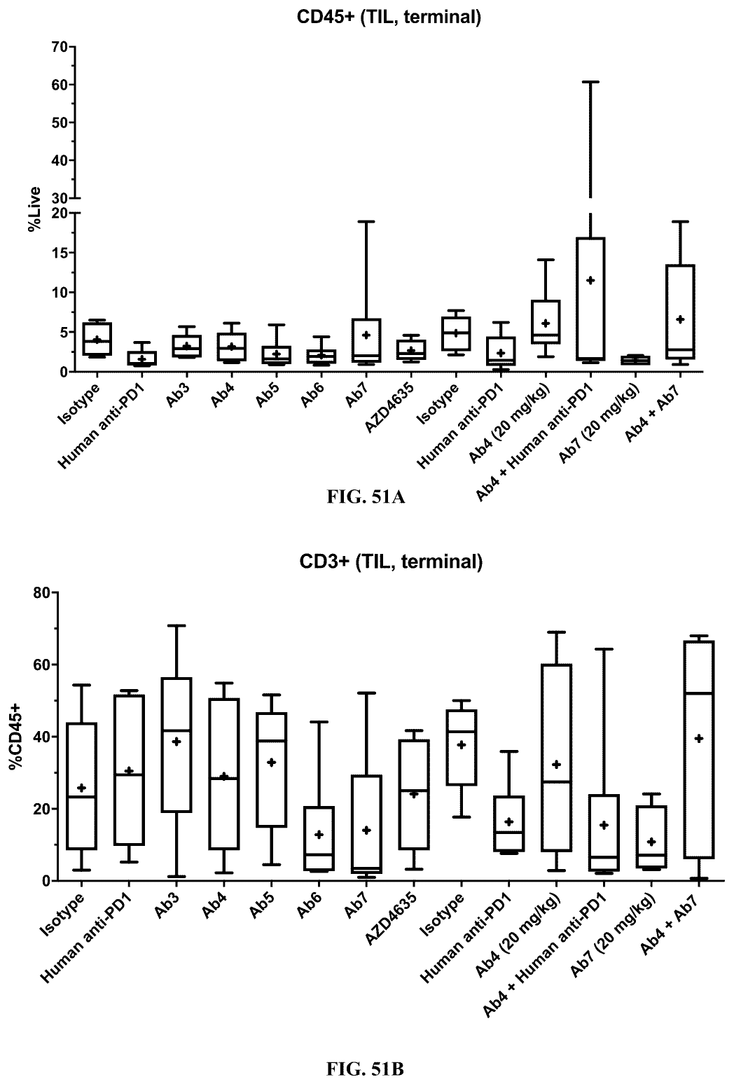

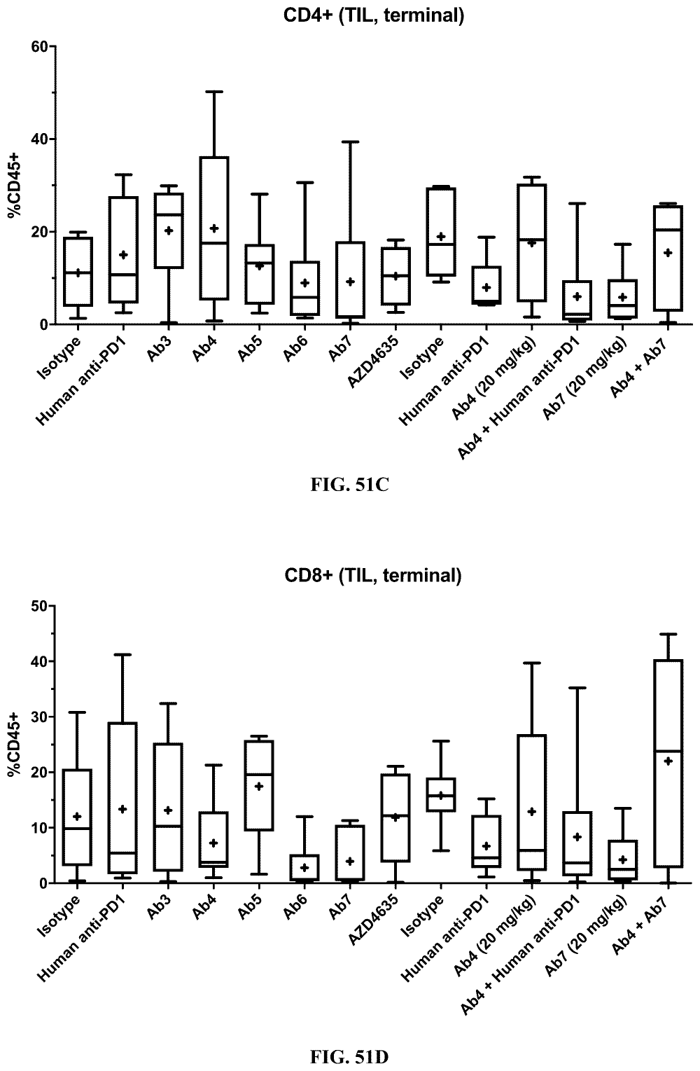

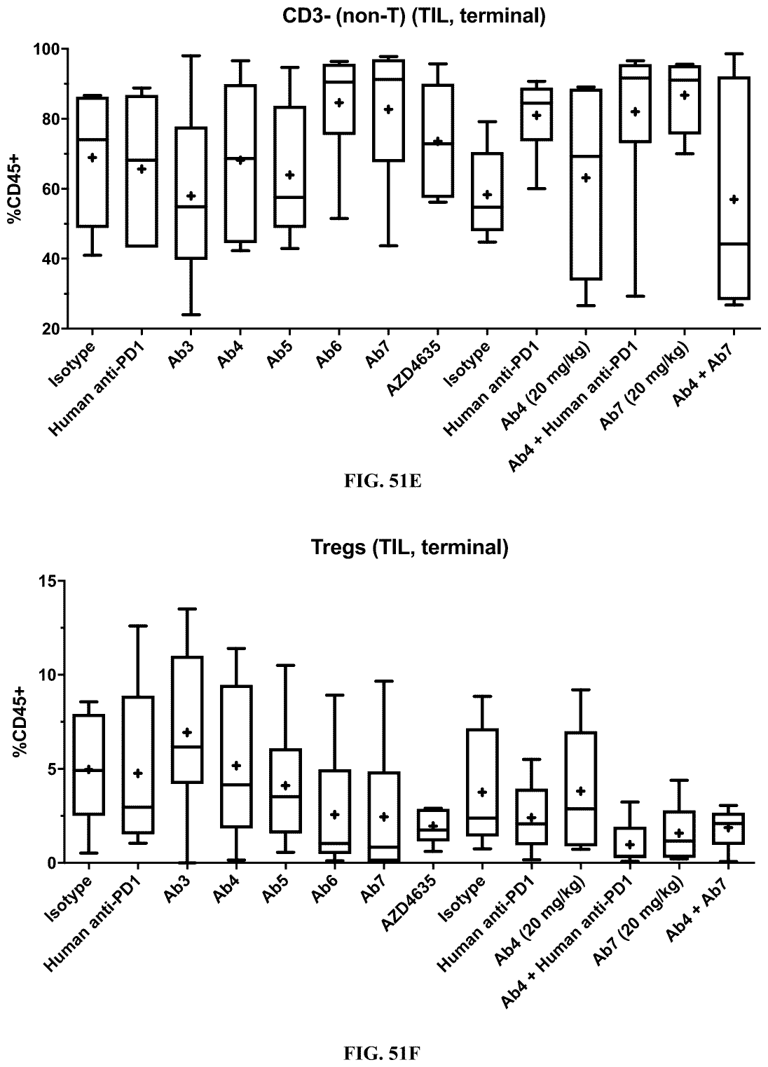

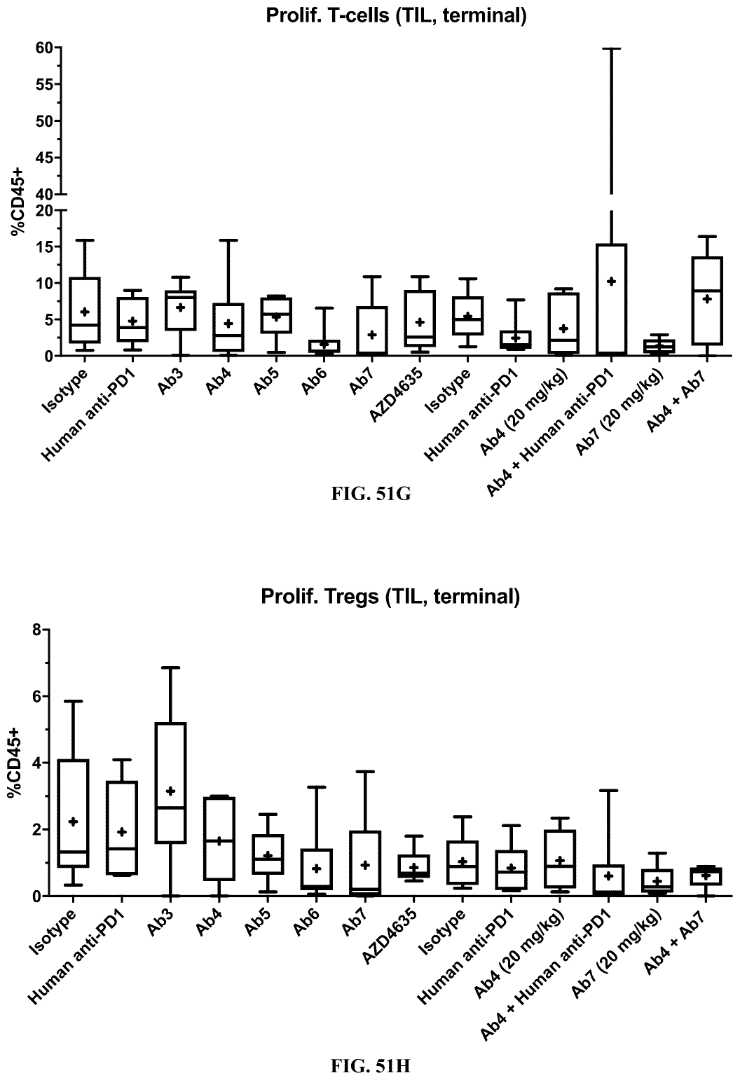

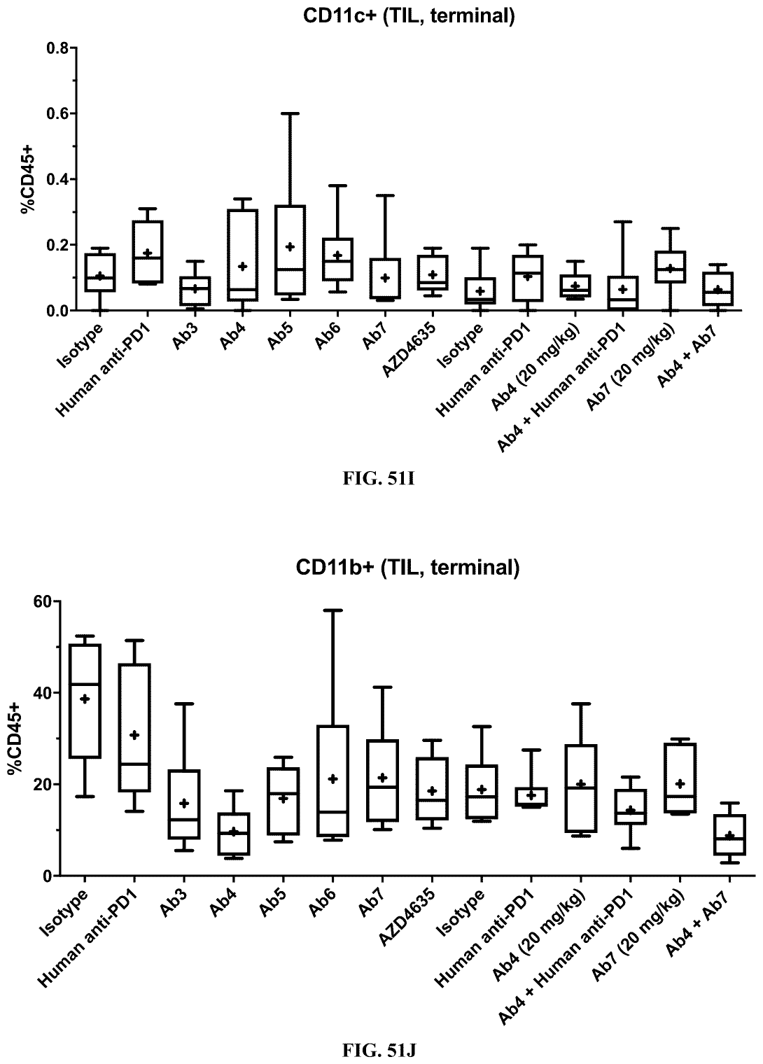

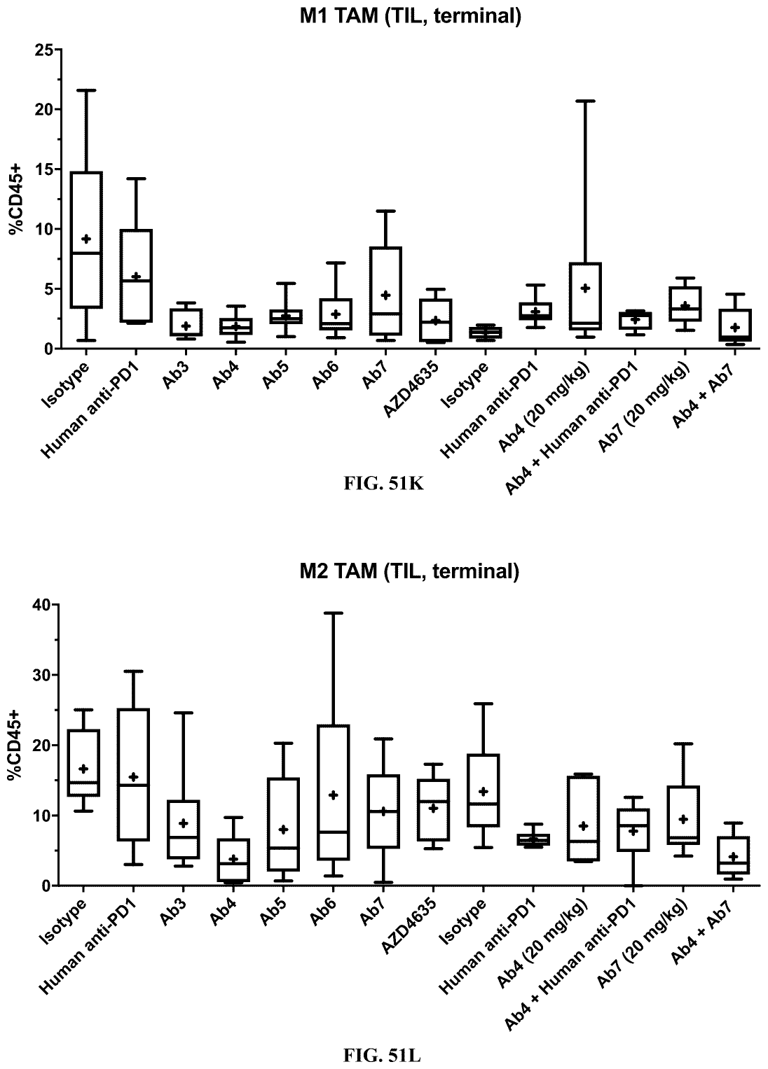

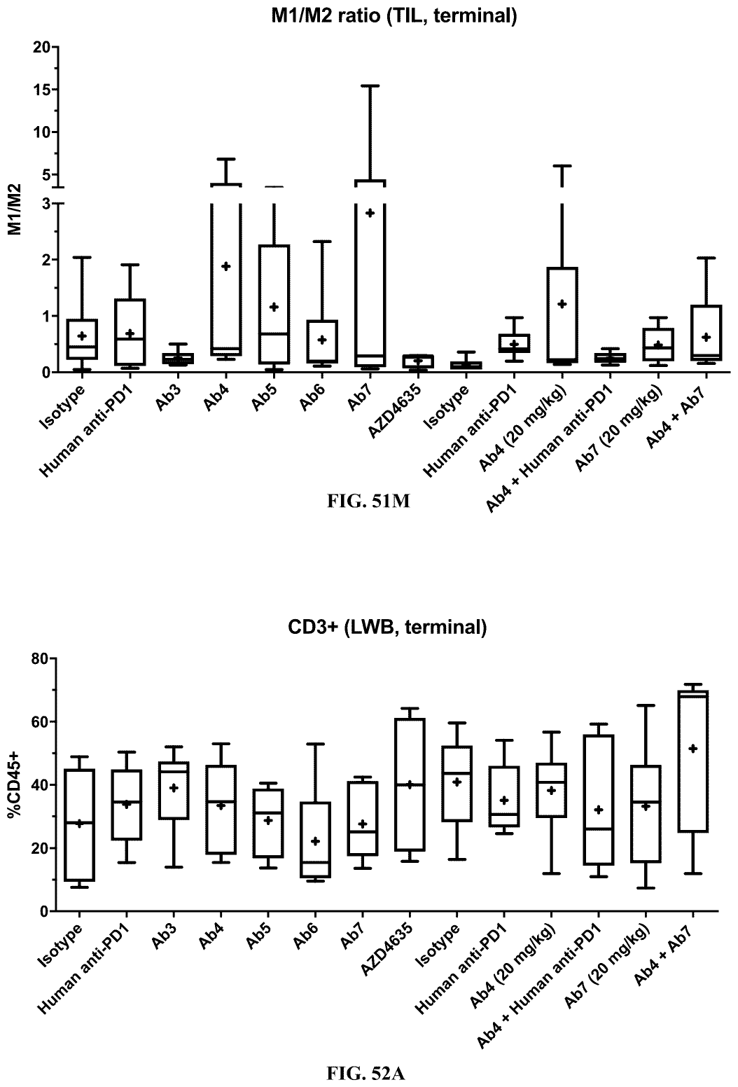

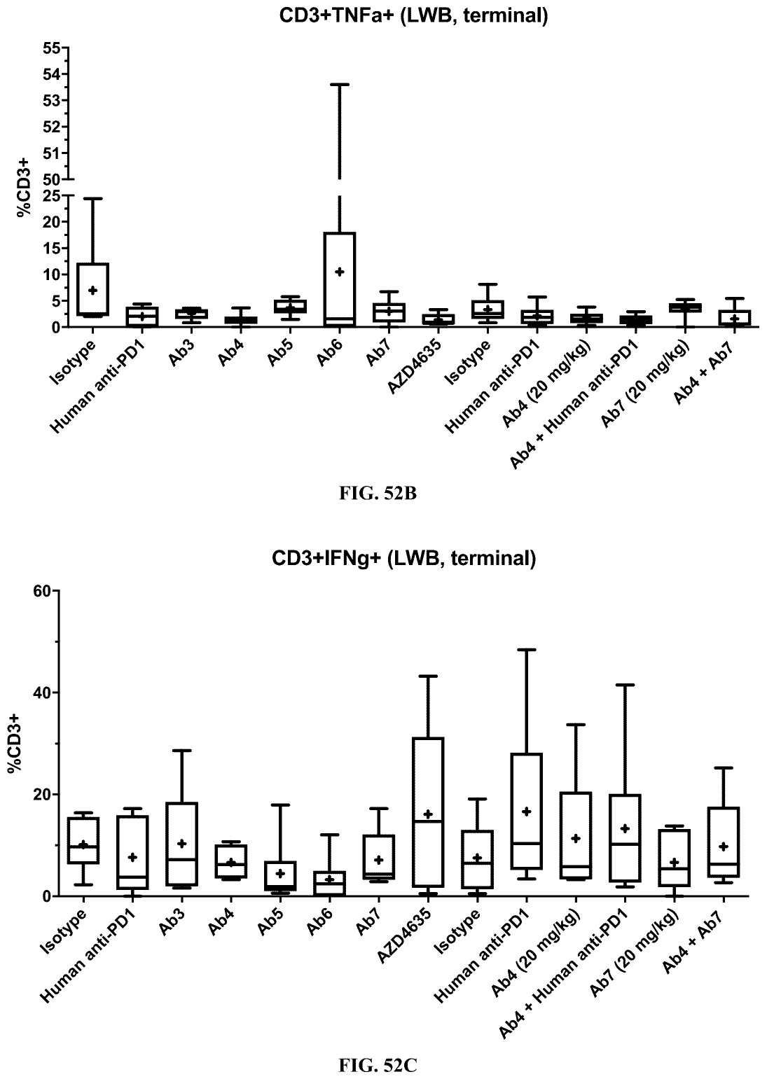

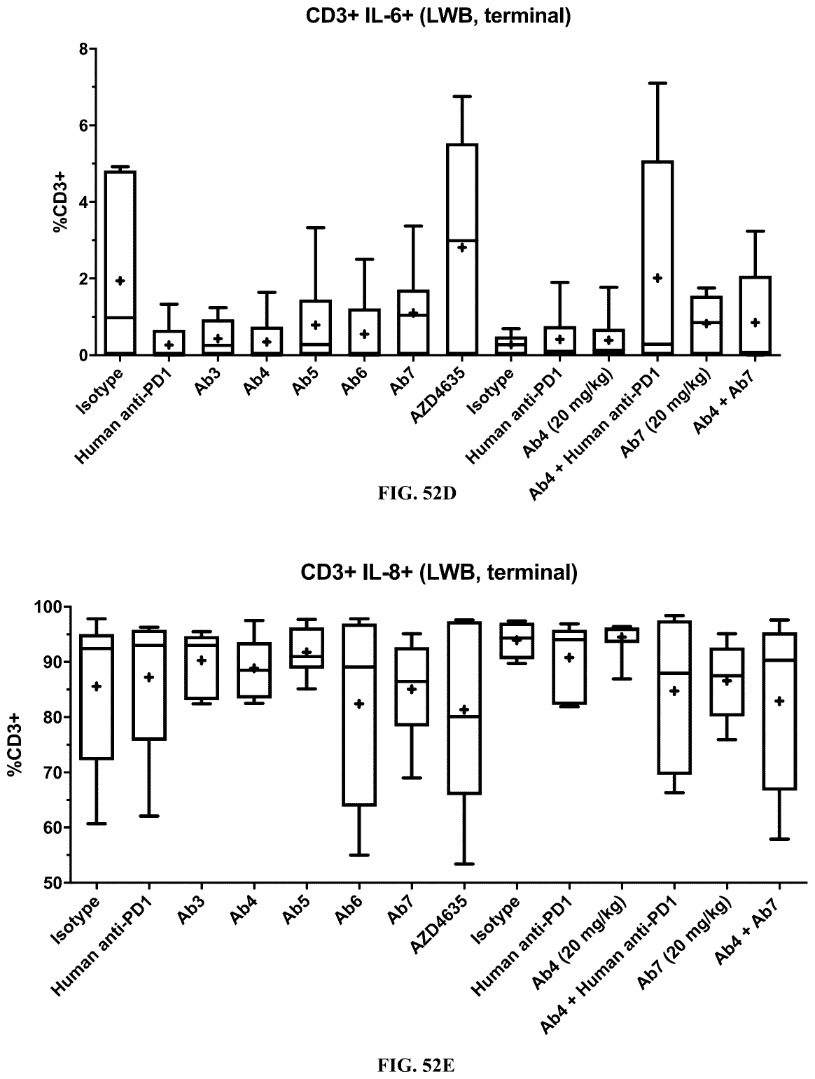

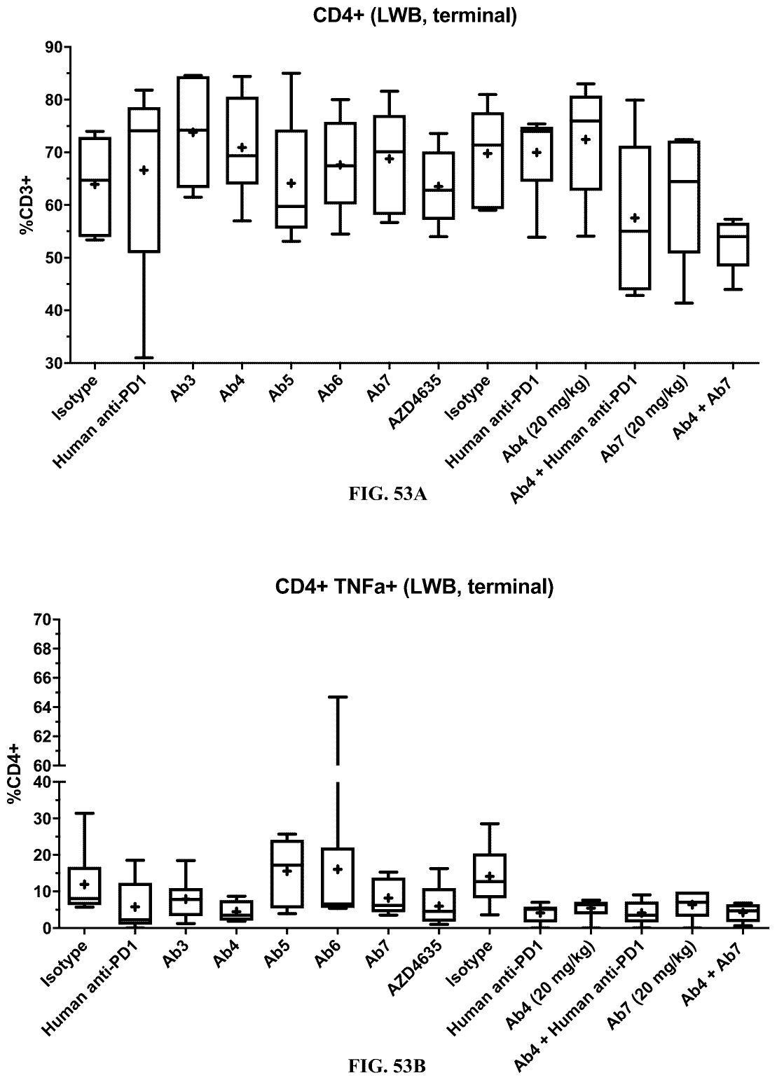

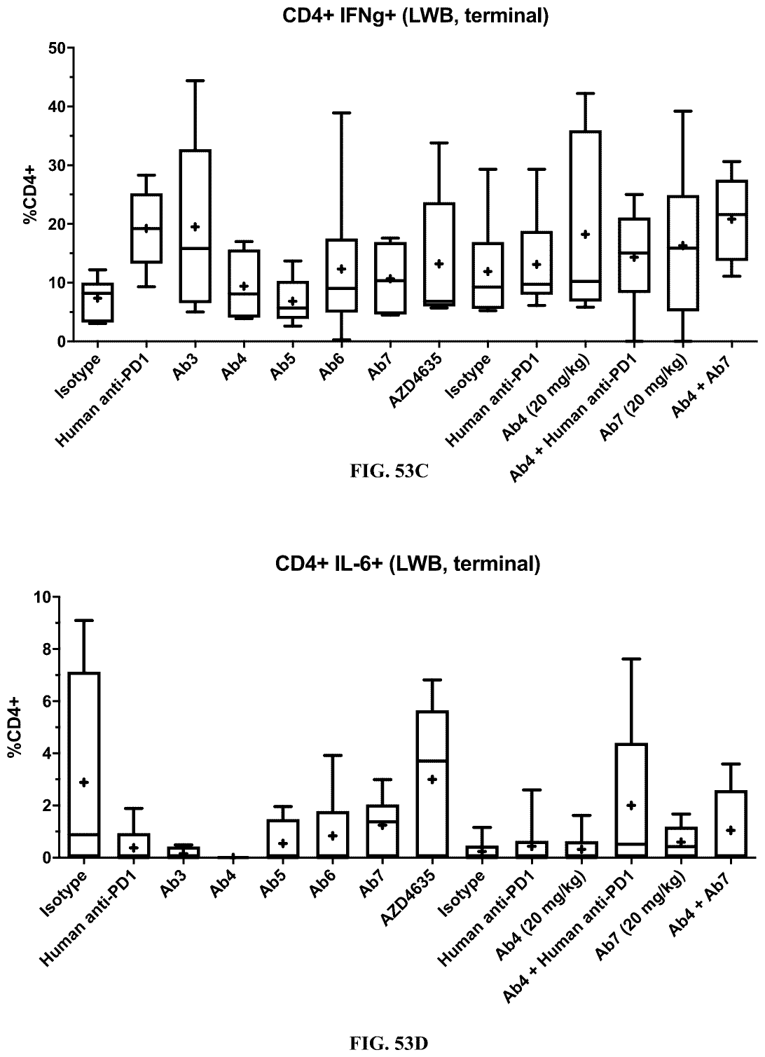

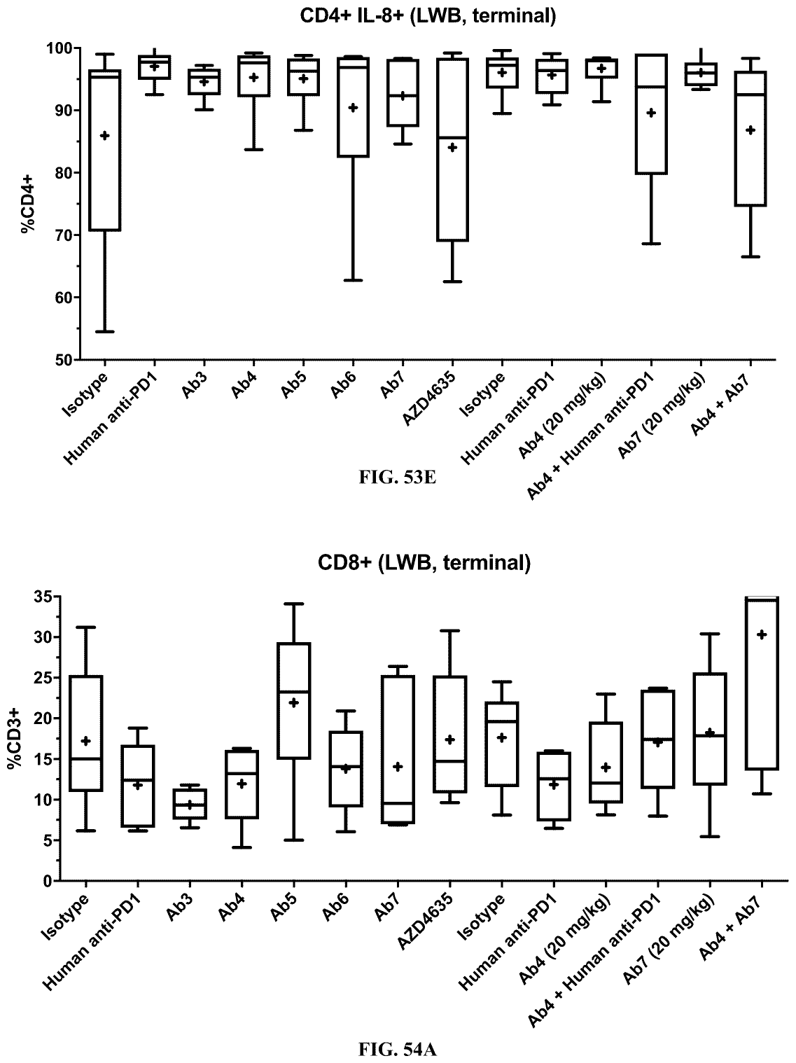

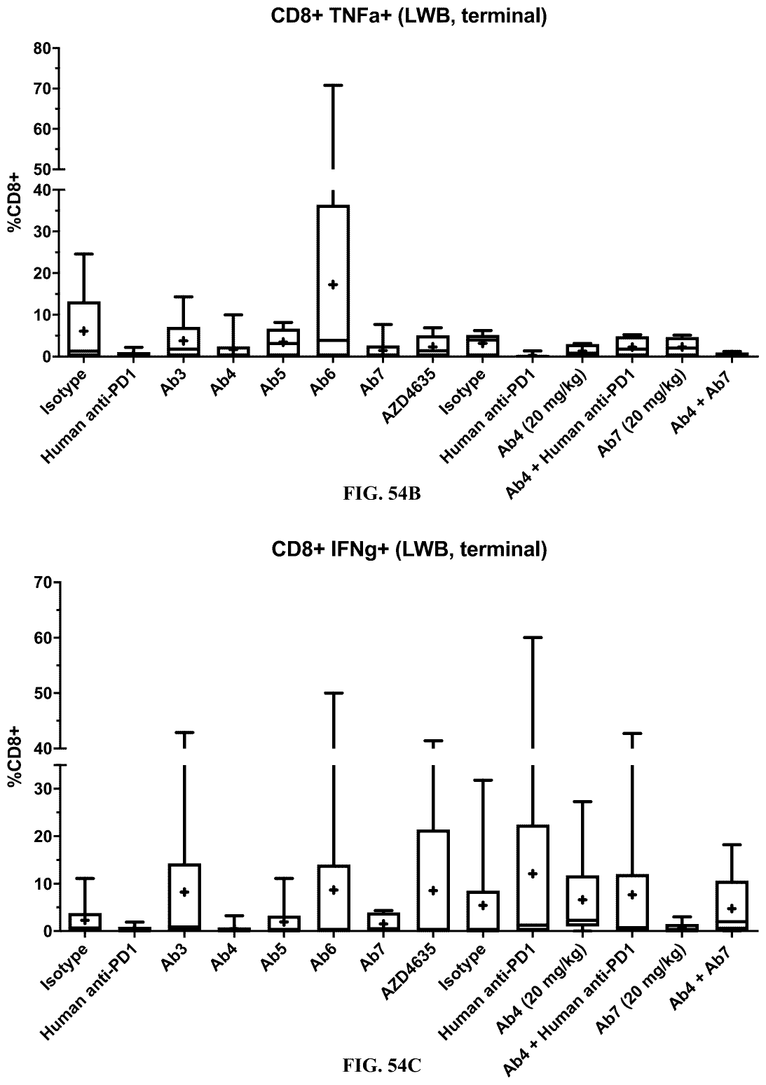

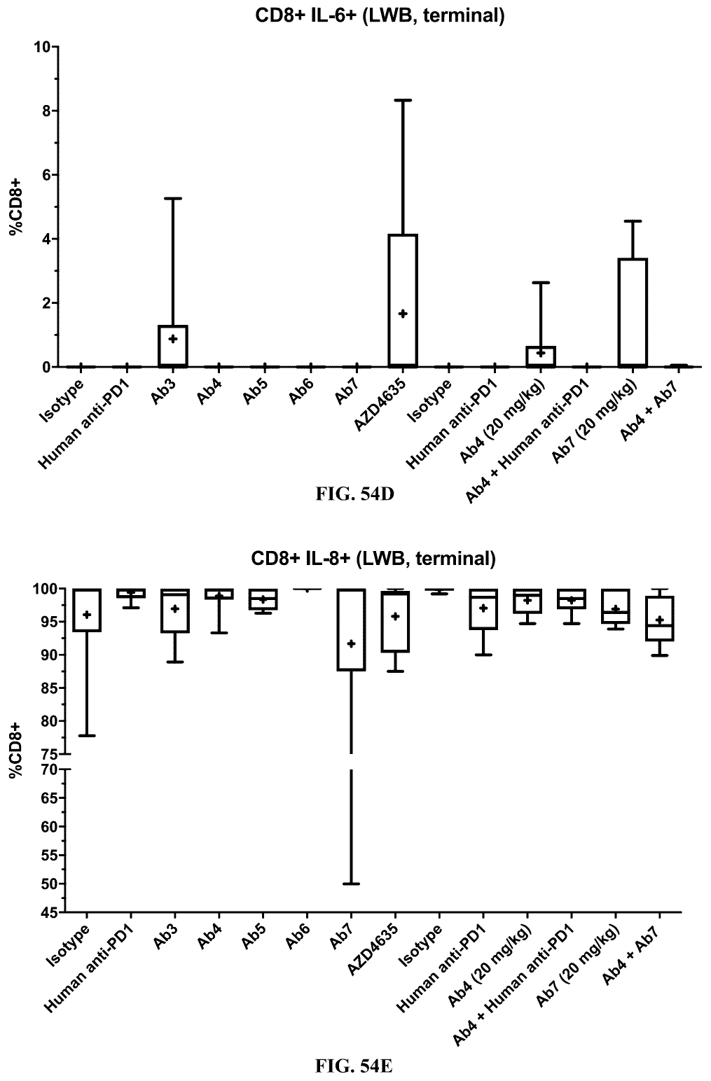

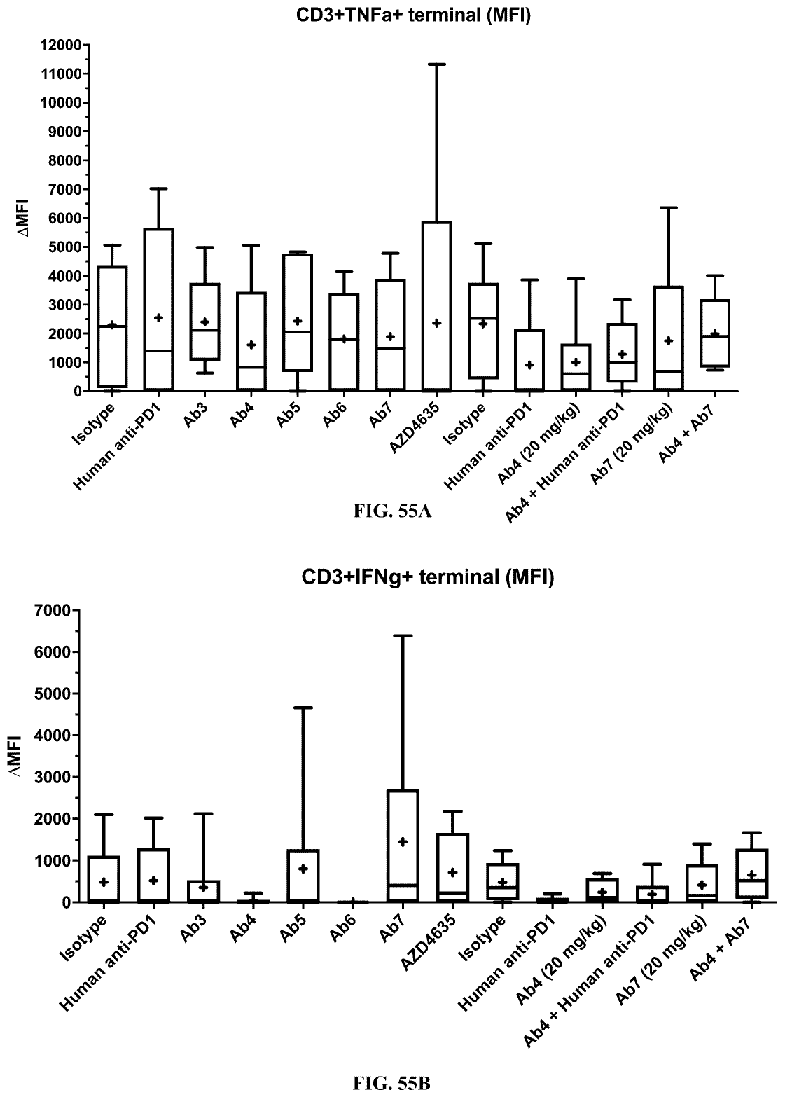

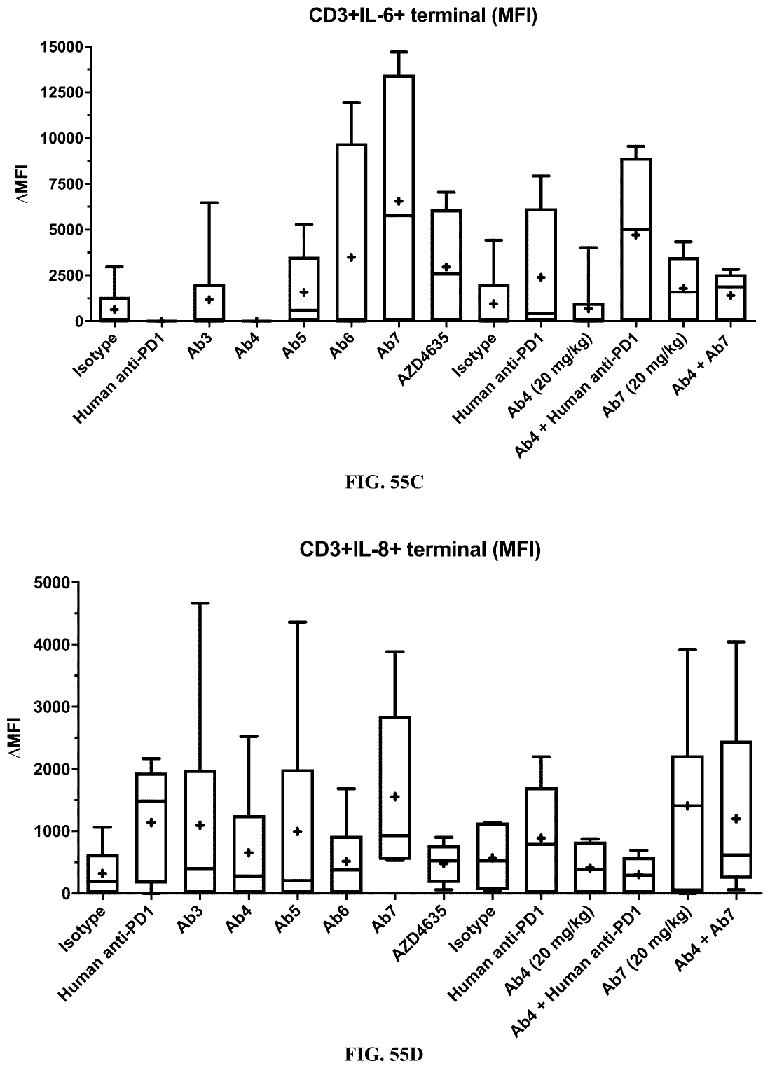

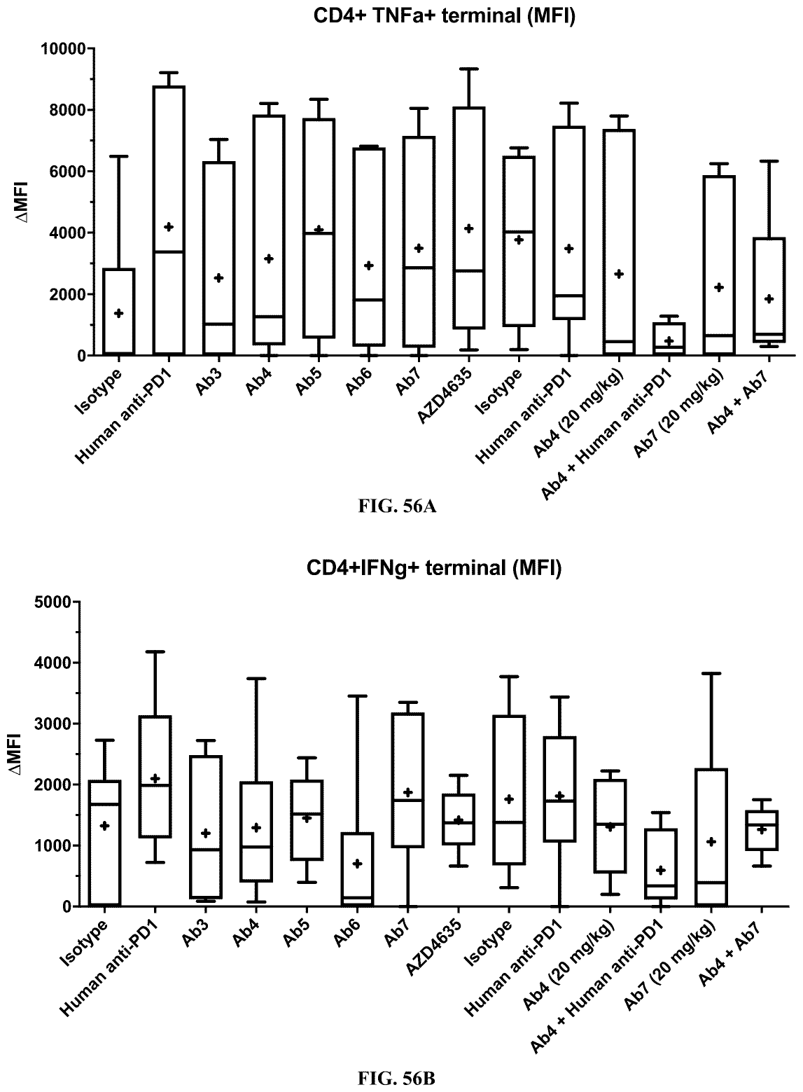

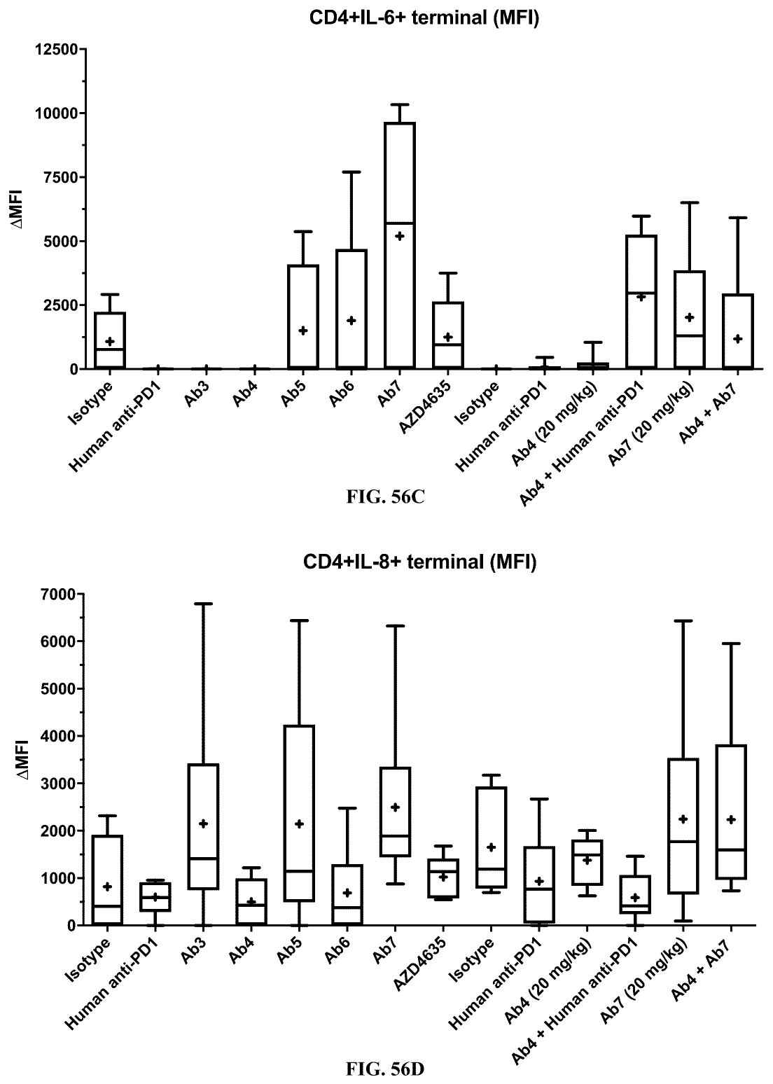

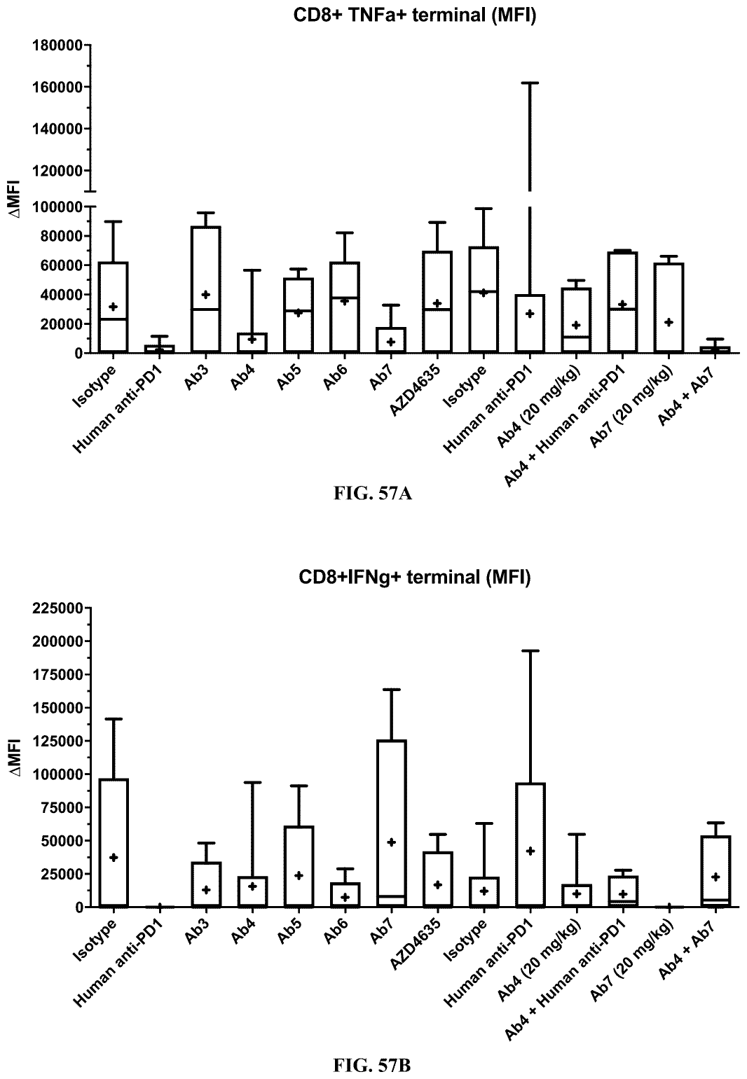

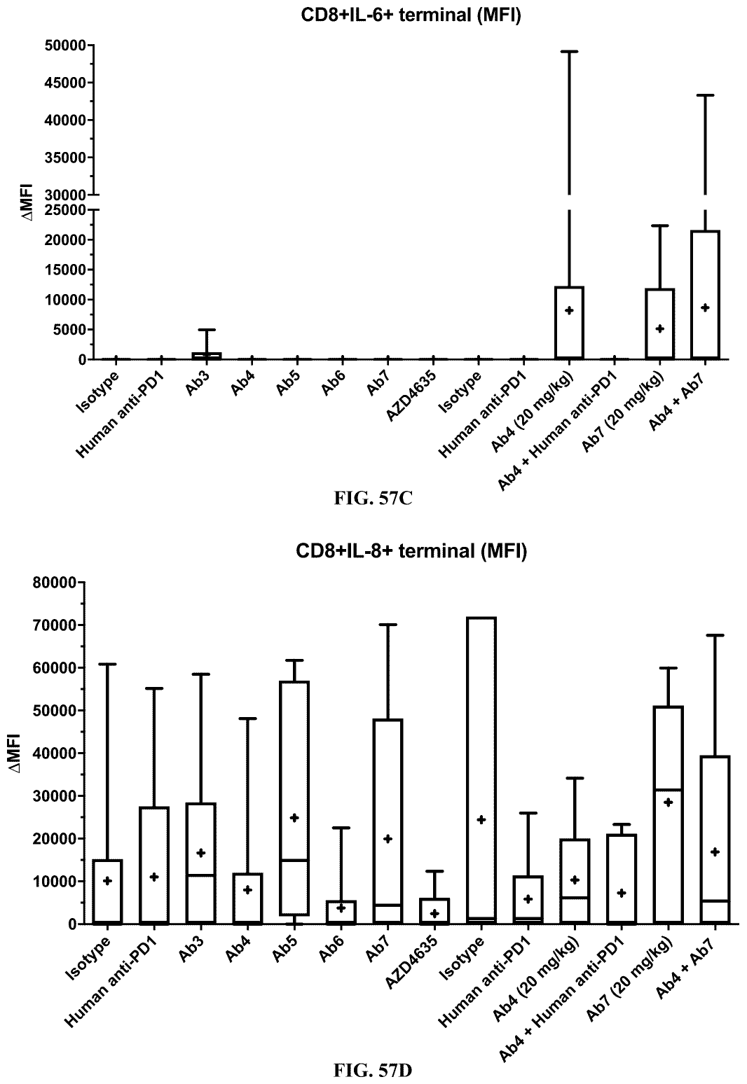

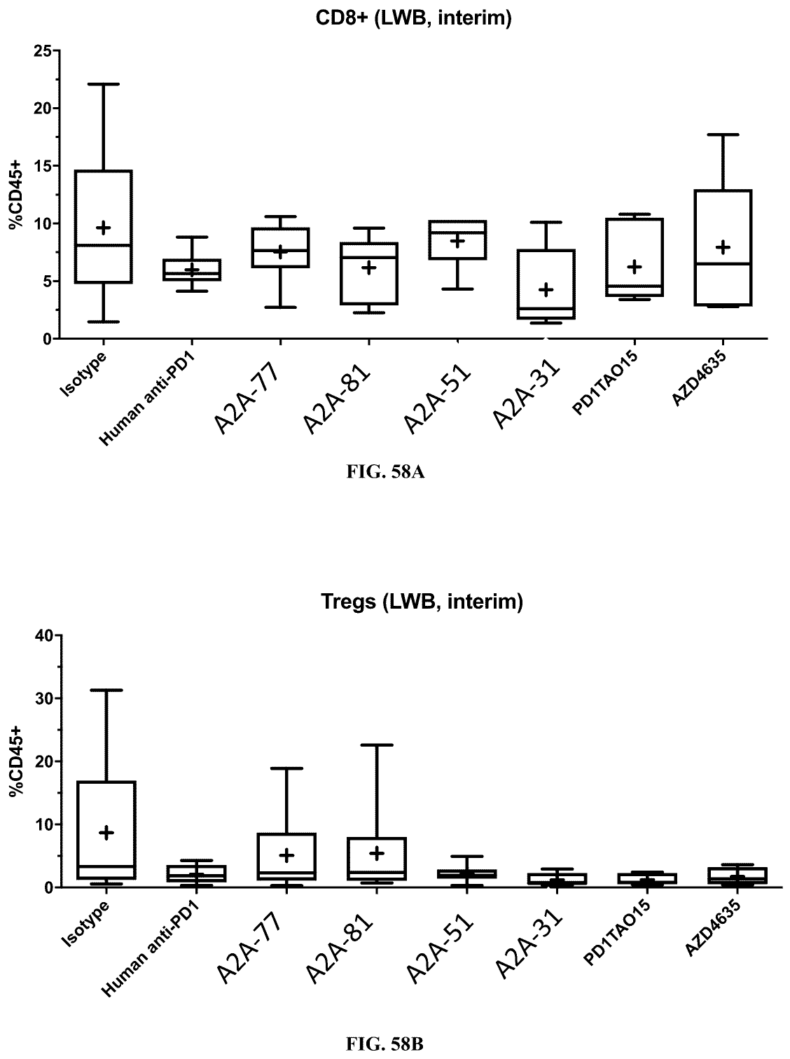

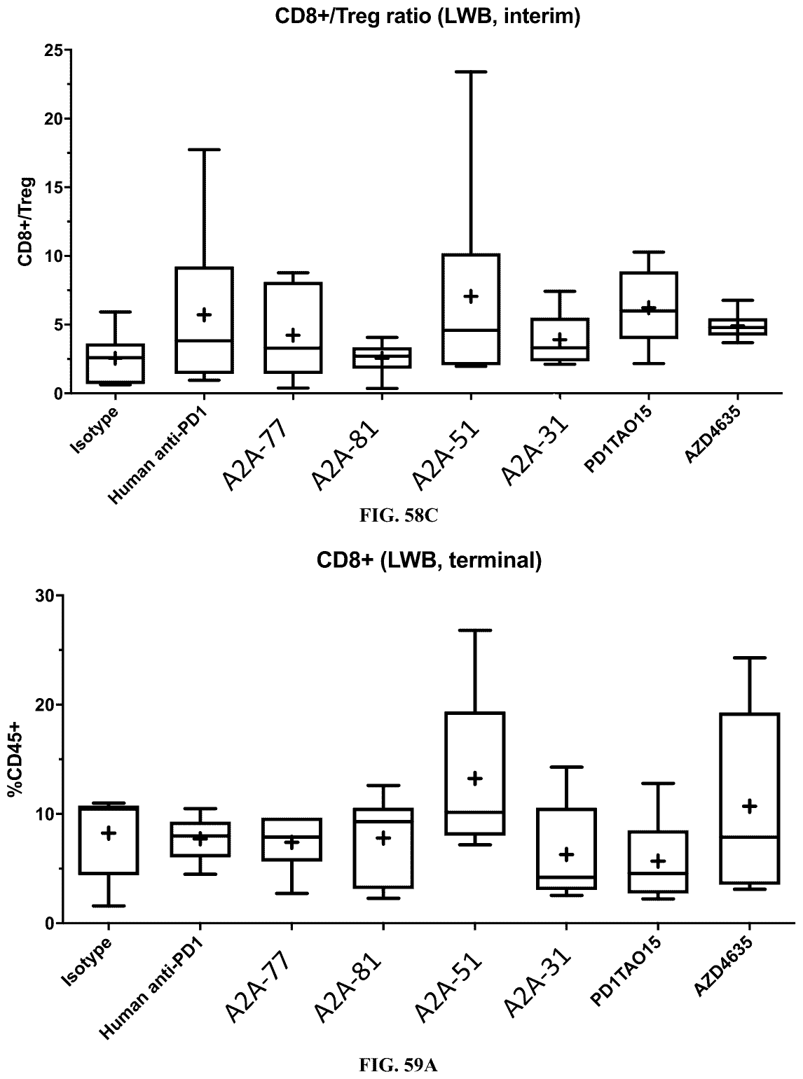

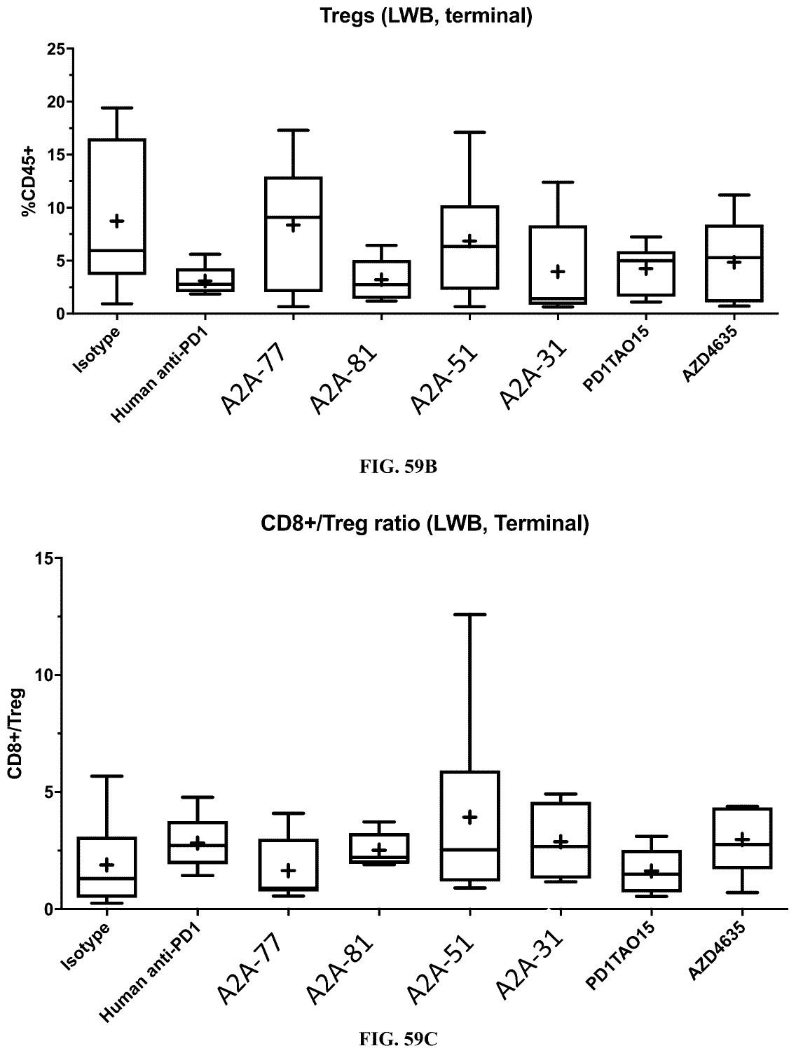

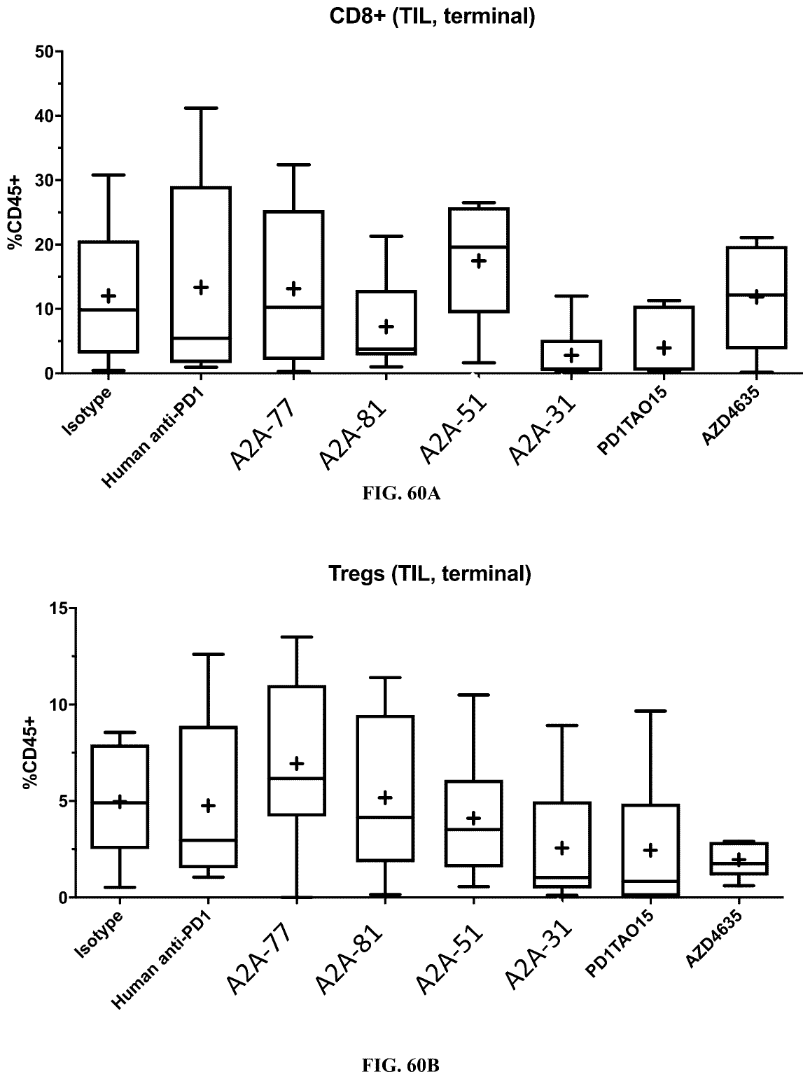

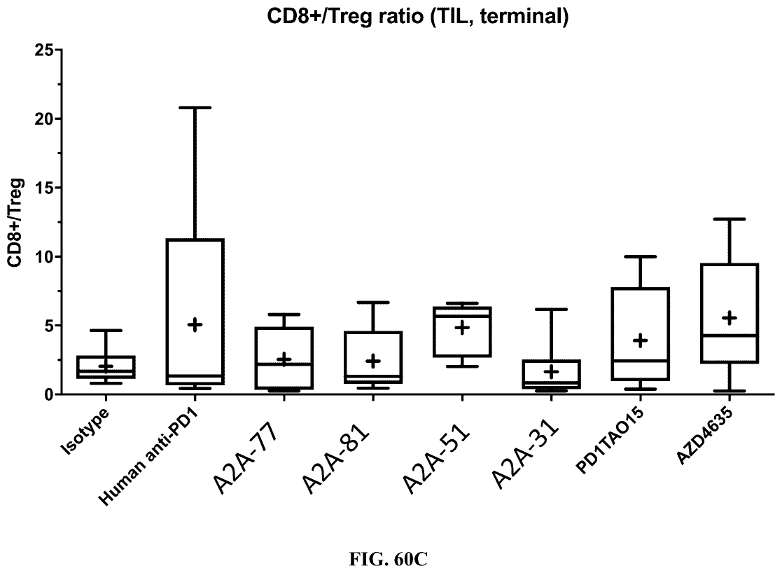

Antibodies were reformed into either IgG1 or IgG4. The reformatted antibodies were then assayed in primary T cell activation assays, which measures cytokine release. Data is seen in FIGS. 48A-48E. As seen in the data, after reformatting the leads into IgG4, IgG4s have better T cell activation activities than IgG1s.

### Example 27: In Vivo Study in Colon Carcinoma Model

Mice with human colon carcinoma (HuCD34NCG-Colo205) were divided into two sets: Set 1 was divided into eight groups and Set 2 was divided into six groups. In Set 1, Group 1 was the isotype control, Group 2 mice were treated with Anti-PD1, Group 3 mice were treated with variant Ab3, Group 4 mice were treated with variant Ab4, Group 5 mice were treated with variant Ab5, Group 6 mice were treated with variant Ab6, Group 7 mice were treated with variant Ab7, Group 8 mice were treated with AZD4635. Each group in Set 1 was given a 10 mg/kg dose on a Q3D×6 schedule except for group 8, which was given 50 mg/kg on a twice daily schedule. In Set 2, Group 1 was the isotype control, Group 2 mice were treated with Anti-PD1, Group 3 mice were treated with Ab4, Group 4 mice were treated with Ab4+Anti-PD1, Group 5 mice were treated with AB7, and Group 6 mice were treated with AB4+Ab7. Each group in Set 2 was given a 20 mg/kg total dose on a Q3D×6 schedule. Tumor volume was measured over 30 days.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1-24. (canceled)

25. An antibody or antibody fragment that binds to adenosine 2A receptor, wherein the antibody or antigen fragment comprises a heavy chain comprising a heavy chain variable region (VH) and a light chain comprising a light chain variable region (VL), wherein:
a. the VH comprises a CDRH1 comprising the amino acid sequence of SEQ ID NO: 82, CDRH2 comprising the amino acid sequence of SEQ ID NO: 171, and CDRH3 comprising the amino acid sequence of SEQ ID NO: 260, and wherein the VL comprises CDRL1 comprising the amino acid sequence of SEQ ID NO: 349, CDRL2 comprising the amino acid sequence of SEQ ID NO: 438, and CDRL3 comprising the amino acid sequence of SEQ ID NO: 527; or
b. the VH comprises a CDRH1 comprising the amino acid sequence of SEQ ID NO: 86, CDRH2 comprising the amino acid sequence of SEQ ID NO: 175, and CDRH3 comprising the amino acid sequence of SEQ ID NO: 264, and wherein the VL comprises CDRL1 comprising the amino acid sequence of SEQ ID NO: 353, CDRL2 comprising the amino acid sequence of SEQ ID NO: 442, and CDRL3 comprising the amino acid sequence of SEQ ID NO: 531.

26. The antibody or antibody fragment of claim 25, wherein:
a. the VH comprises a CDRH1 comprising the amino acid sequence of SEQ ID NO: 82, a CDRH2 comprising the amino acid sequence of SEQ ID NO: 171, a CDRH3 comprising the amino acid sequence of SEQ ID NO: 260, and the VH comprises an amino acid sequence at least 90% identical to the amino acid sequence of SEQ ID NO: 616; and wherein the VL comprises a CDRL1 comprising the amino acid sequence of SEQ ID NO: 349, CDRL2 comprising the amino acid sequence of SEQ ID NO: 438, and a CDRL3 comprising the amino acid sequence of SEQ ID NO: 527, and the VL comprises an amino acid sequence at least 90% identical to the amino acid sequence of SEQ ID NO: 705; or
b. the VH comprises a CDRH1 comprising the amino acid sequence of SEQ ID NO: 86, a CDRH2 comprising the amino acid sequence of SEQ ID NO: 175, a CDRH3 comprising the amino acid sequence of SEQ ID NO: 264, and the VH comprises an amino acid sequence at least 90% identical to the amino acid sequence of SEQ ID NO: 620; and wherein the VL comprises a CDRL1 comprising the amino acid sequence of SEQ ID NO: 353, CDRL2 comprising the amino acid sequence of SEQ ID NO: 442, and a CDRL3 comprising the amino acid sequence of SEQ ID NO: 531, and the VL comprises an amino acid sequence at least 90% identical to the amino acid sequence of SEQ ID NO: 709.

27. The antibody or antibody fragment of claim 25, wherein:
a. the VH comprises an amino acid sequence at least 90% identical to the amino acid sequence of SEQ ID NO: 616, and the VL comprises an amino acid sequence at least 90% identical to the amino acid sequence of SEQ ID NO: 705; or
b. the VH comprises an amino acid sequence at least 90% identical to the amino acid sequence of SEQ ID NO: 620, and the VL comprises an amino acid sequence at least 90% identical to the amino acid sequence of SEQ ID NO: 709.

28. The antibody or antibody fragment of claim 25, wherein:
a. the VH comprises the amino acid sequence of SEQ ID NO: 616, and the VL comprises the amino acid sequence of SEQ ID NO: 705; or
b. the VH comprises the amino acid sequence of SEQ ID NO: 620, and the VL comprises the amino acid sequence of SEQ ID NO: 709.

29. The antibody or antibody fragment of claim 25, wherein the antibody is a monoclonal antibody, a bi-specific antibody, a multispecific antibody, a grafted antibody, a humanized antibody, a synthetic antibody, a chimeric antibody, a single-chain Fvs (scFv), a Fab fragment, a F(ab′)2 fragment, a Fv fragment, a diabody, a disulfide-linked Fvs (sdFv), an intrabody, or an antigen-binding fragment thereof.

30. The antibody or antibody fragment of claim 25, wherein the antibody or antibody fragment binds to adenosine 2A receptor with a K_{D} of less than about 75 nM, 50 nM, 25 nM, or 10 nM.

31. The antibody or antibody fragment of claim 25, wherein the antibody or antibody fragment comprises an IC50 of less than about 20 nM, 10 nM, 7.5 nM, or 5 nM in a T cell activation assay.

32. A nucleic acid that encodes the antibody or antibody fragment of claim 15.

33. An expression vector comprising the nucleic acid of claim 32.

34. An isolated cell comprising the nucleic acid of claim 32 or the expression vector of claim 33.

35. A method for activating T cells, comprising administering the antibody or antibody fragment of claim 25 to a subject.

36. A method of treating cancer, comprising administering the antibody or antibody fragment of claim 25 to a subject.

37. The method of claim 36, wherein the cancer is colon cancer.

38. A method of treating a neurological disease or disorder, comprising administering the antibody or antibody fragment of claim 25 to a subject.

39. The method of claim 35, wherein:
a. the VH comprises an amino acid sequence at least 90% identical to the amino acid sequence of SEQ ID NO: 616 and a VL comprising an amino acid sequence at least 90% identical to the amino acid sequence of SEQ ID NO: 705; or
b. the VH comprises an amino acid sequence at least 90% identical to the amino acid sequence of SEQ ID NO: 620, and the VL comprises an amino acid sequence at least 90% identical to the amino acid sequence of SEQ ID NO: 709.

40. The method of claim 35, wherein:
a. the VH comprising the amino acid sequence of SEQ ID NO: 616 and a VL comprising the amino acid sequence of SEQ ID NO: 705; or
b. the VH comprises the amino acid sequence of SEQ ID NO: 620, and the VL comprises the amino acid sequence of SEQ ID NO: 709.

41. The method of claim 36, wherein:
a. the VH comprises an amino acid sequence at least 90% identical to the amino acid sequence of SEQ ID NO: 616 and a VL comprising an amino acid sequence at least 90% identical to the amino acid sequence of SEQ ID NO: 705; or
b. the VH comprises an amino acid sequence at least 90% identical to the amino acid sequence of SEQ ID NO: 620, and the VL comprises an amino acid sequence at least 90% identical to the amino acid sequence of SEQ ID NO: 709.

42. The method of claim 36, wherein:
a. the VH comprising the amino acid sequence of SEQ ID NO: 616 and a VL comprising the amino acid sequence of SEQ ID NO: 705; or
b. the VH comprises the amino acid sequence of SEQ ID NO: 620, and the VL comprises the amino acid sequence of SEQ ID NO: 709.

43. The method of claim 37 wherein:
a. the VH comprises an amino acid sequence at least 90% identical to the amino acid sequence of SEQ ID NO: 616 and a VL comprising an amino acid sequence at least 90% identical to the amino acid sequence of SEQ ID NO: 705; or
b. the VH comprises an amino acid sequence at least 90% identical to the amino acid sequence of SEQ ID NO: 620, and the VL comprises an amino acid sequence at least 90% identical to the amino acid sequence of SEQ ID NO: 709.

44. The method of claim 37, wherein:
a. the VH comprising the amino acid sequence of SEQ ID NO: 616 and a VL comprising the amino acid sequence of SEQ ID NO: 705; or
b. the VH comprises the amino acid sequence of SEQ ID NO: 620, and the VL comprises the amino acid sequence of SEQ ID NO: 709.
